(19) United States
(12) Patent Application Publication
(10) Pub. No.: US 2026/0137783 A1
(43) Pub. Date: May 21, 2026

(54) CHIMERIC ANTIGEN RECEPTOR SPECIFIC TO CD117

(71) Applicant: CRISPR THERAPEUTICS AG, Zug (CH)

(72) Inventors: Jason SAGERT, Cambridge, MA (US); Peter HALLOCK, Cambridge, MA (US)

(21) Appl. No.: 18/877,006

(22) PCT Filed: Jun. 20, 2023
(86) PCT No.: PCT/IB2023/056372

§ 371 Date: Dec. 19, 2024

Related U.S. Application Data
(60) Provisional application No. 63/353,732, filed on Jun. 20, 2022.
(60) Provisional application No. 63/377,302, filed on Sep. 27, 2022.

(51) Int. Cl. A61K 40/31; A61K 35/14; A61K 40/42; C07K 14/705; C07K 14/725; C07K 16/28; C12N 5/078; C12N 5/10; C12N 15/62

(52) U.S. Cl. CPC A61K 40/31; A61K 35/14; A61K 40/4215; C07K 14/7051; C07K 14/70517; C07K 14/70521; C07K 14/70575; C07K 16/2803; C12N 5/0634; C12N 5/10; C12N 15/625; C07K 2317/565; C07K 2317/569; C07K 2317/622; C07K 2319/02; C07K 2319/03; C12N 2510/00

(57) ABSTRACT

A chimeric antigen receptor (CAR) that binds CD117 (anti-CD117 CAR), wherein the anti-CD117 CAR comprises: (a) an ectodomain that binds CD 117; (b) a transmembrane domain; and (c) an endodomain comprising a costimulatory domain and a CD3ζ signaling domain. The ectodomain comprises a single chain variable fragment (scFv) that binds CD 117 (anti-CD117 scFv), or comprises a single domain antibody fragment such as a VHH fragment. Also provided herein are genetically engineered T cells expressing such anti-CD117 CAR, which can be used for inhibiting CD117+ cells such as cancer cells.

### BACKGROUND OF THE INVENTION

Cluster of differentiation 117 (CD117), also known as tyrosine-protein kinase (cKIT) or mast/stem cell growth factor receptor (SCFR), is a cytokine receptor expressed on hematopoietic stem cells and on other types of cells. Upon binding to stem cell factor (SCF), the tyrosine kinase activity of CD117 is activated, leading to phosphorylating and activating downstream molecules and propagating the signaling pathway mediated by CD117 in cells. The CD117-mediated cell signaling plays roles in various biological processes, including cell survival, proliferation, and differentiation.

CD117 was reported to be associated with various types of cancer, for example, gastrointestinal stromal tumors, testicular seminoma, mast cell disease, and hematopoietic malignancies, such as melanoma and acute myeloid leukemia. Accordingly, CD117 can be used as a treatment target for such cancers.

### SUMMARY OF THE INVENTION

The present disclosure is based, at least in part, on the development of anti-CD117 CAR-T cells (with either anti-CD117 scFv or anti-CD117 VHH), which showed cytotoxicity against CD117+ cells (e.g., HSCs) both in vitro and in vivo. Exemplary anti-CD117 CAR-T cells, which may express the anti-CD117 CAR in a transient manner, showed successful inhibition of CD34/CD117+ cells in vitro and successful ablation of bone marrow cells and enhanced engraftment of new CD34+ donor cells as observed in a mouse model. Accordingly, disclosed herein are anti-CD117 CAR polypeptides and genetically engineered T cells expressing such, which optionally can have disrupted TRAC and β2M genes, methods of using such genetically engineered T cells for inhibiting CD117+ cells and thus for treating diseases associated with CD117+ cells, as well as methods for producing any of the genetically engineered T cells disclosed herein.

Accordingly, in some aspects, the present disclosure features a chimeric antigen receptor (CAR) that binds CD117 (anti-CD117 CAR), wherein the anti-CD117 CAR comprises: (a) an ectodomain that binds CD117; (b) a transmembrane domain; and (c) an endodomain comprising a costimulatory domain and a CD3ζ signaling domain.

In some embodiments, the ectodomain comprises comprising a single chain variable fragment (scFv) that binds CD117 (anti-CD117 scFv), which comprises a heavy chain variable region (V_{H}) and a light chain variable region (V_{L}), the V_{H} comprising the same heavy chain complementarity determining regions (CDRs) and the same light chain CDRs as those in Reference anti-CD117 antibody 1 (anti-CD117 Ab-1; see Table 2) or Reference anti-CD117 antibody 2 (anti-CD117 Ab-2; see also Table 2).

In some examples, the anti-CD117 scFv comprises a V_{H} comprising SEQ ID NO: 108, and a V_{L} comprising SEQ ID NO: 107. In other examples, the anti-CD117 scFv comprises a V_{H} comprising SEQ ID NO: 130, and a V_{L} comprising SEQ ID NO: 129. In specific examples, the scFv comprises the amino acid sequence of any one of SEQ ID NOs: 109, 110, 131, and 132. In one example, the scFv comprises SEQ ID NO: 109.

Any of the anti-CD117 CARs disclosed herein, comprising an anti-CD117 scFv as disclosed herein, may comprise a transmembrane domain, which can be a CD8 transmembrane domain. Alternatively or in addition, the anti-CD117 CAR may comprise a CD28 co-stimulatory domain or a 4-1BB co-stimulatory domain. Exemplary anti-CD117 CARs comprising an anti-CD117 scFv fragment are provided in Table 2 herein, all of which are within the scope of the present disclosure. In one example, the anti-CD117 CAR is CTX2840. In another example, the anti-CD117 CAR is CTX2841.

In other embodiments, the ectodomain comprises a single domain antibody fragment comprising:
(i) a CDR1 set forth as GX₁X₂TX₃X₄X₅X₆X₇, in which X₁ is T, R, G, H, or D; X₂ is T or absent; X₃ is F, L, S, or V; X₄ is G, S, or T; X₅ is I, N, S, T or Y; X₆ is D, V, or Y; and X₇ is A, F, P, S, V or W;
(ii) CDR2 set forth as X₈X₉X₁₀X₁₁X₁₂X₁₃X₁₄X₁₅, in which X₈ is I or V; X₉ is A, G, H, L, R, S, T, or V, X₁₀ is R, S, or W; X₁₁ is G, N, S, or Y; X₁₂ is A, G, L, or absent; X₁₃ is A, D, G, L, or S; X₁₄ is G, M, S, T, or V; and X₁₅ is A, L, or T; and
(iii) CDR3 set forth as: (a) GRFHPIRVDTA (SEQ ID NO: 70); (b) ASGSNWRLGAIDEY (SEQ ID NO: 71); (c) GQHLSGLGGSAWSIEG (SEQ ID NO: 72); (d) RQYVGSGSYYLKKEGGY (SEQ ID NO: 73); (e) DSTGVYGTGYVSSRKGRY (SEQ ID NO: 74); (f) AFTPEFRDGGIWDDASV (SEQ ID NO: 75); (g) VRRRWLIWQEEEY (SEQ ID NO: 78); (h) DQRGVPAYYSDYALY (SEQ ID NO: 80); (i) DESFPAYYSDYALY (SEQ ID NO: 81); (j) VLRTGM (SEQ ID NO: 69); (k) SDSYFYASPHLY (SEQ ID NO: 76); (1) SDTYFYASPHLY (SEQ ID NO: 77); or (m) RRGTILVVQEYEY (SEQ ID NO: 79).

In some embodiments, the CDR3 in the anti-CD117 antibody is set forth as any one of (a)-(i) listed above. In some examples, the CDR3 is AFTPEFRDGGIWDDASV (SEQ ID NO: 75). In some examples, the CDR3 is DQRGVPAYYSDYALY (SEQ ID NO: 80). In some examples, the CDR3 is DESFPAYYSDYALY (SEQ ID NO: 81).

In some embodiments, X₁ in CDR1 can be D, G, H, or R (e.g., R); X₂ in CDR1 can be absent; X₃ in CDR1 can be F, L, or S (e.g., F or S); X₄ in CDR1 can be G, S, or T; X₅ in CDR1 can be S or Y; X₆ in CDR1 can be D or Y; and X₇ in CDR1 can be A or V. In some examples, the CDR1 of the anti-CD117 antibody can be one of the following: (a) GRTTFSTYW (SEQ ID NO: 16); (b) GGTFSIYP (SEQ ID NO: 17); (c) GRTLSNYF (SEQ ID NO: 18); (d) GRTFSSYA (SEQ ID NO: 19); (e) GHTFSNYA (SEQ ID NO: 20); (f) GDTFSSYS (SEQ ID NO: 24); (g) GRTSGSYV (SEQ ID NO: 26); and (h) GRTFTYDA (SEQ ID NO: 27). In one example, the CDR1 is GRTFSSYA (SEQ ID NO: 19). In another example, the CDR1 is GRTSGSYV (SEQ ID NO: 26). In yet another example, the CDR1 is GRTFTYDA (SEQ ID NO: 27).

In some embodiments, X₈ in the CDR2 of the anti-CD117 antibody can be I; X₉ in the CDR2 can be G, H, L, R, S, or T (e.g., L, or S); X₁₀ in the CDR2 can be S or W; X₁₁ in the CDR2 can be N, S, or Y (e.g., N or S); X₁₂ in the CDR2 can be A, G, or L (e.g., A or G); X₁₃ in the CDR2 can be G, L, or S; X₁₄ in the CDR2 can be G, M, T, or V (e.g., M, S, or T); and X₁₅ in the CDR2 can be is A or T (e.g., T). In some examples, the CDR2 can be one of the following: (a) ISWSAGMA (SEQ ID NO: 43); (b) IGWSASGT (SEQ ID NO: 44); (c) IHWSLGST (SEQ ID NO: 45); (d) ITSSGLVA (SEQ ID NO: 46); (e) ISWSGGST (SEQ ID NO: 47); (f) ILSNGLTT (SEQ ID NO: 48); (g) IRWSGGTT (SEQ ID NO: 51); and (h) ISWSAGMT (SEQ ID NO: 53). In one example, the CDR2 can be ILSNGLTT (SEQ ID NO: 48). In another example, the CDR2 can be ISWSAGMT (SEQ ID NO: 53). In yet another example, the CDR2 can be ISWSGGST (SEQ ID NO: 47).

In specific examples, the single domain antibody fragment comprises the same CDR1, same CDR2, and same CDR3 as a reference antibody of P10, P12, P27, P29, P31, P32, P35, P37, or P38. In one example, the single domain antibody fragment comprises the same CDR1, same CDR2, and same CDR3 as those of P10. In another example, the single domain antibody fragment comprises the same CDR1, same CDR2, and same CDR3 as those of P31. In yet another example, the single domain antibody fragment comprises the same CDR1, same CDR2, and same CDR3 as those of P38.

Any of the single domain antibody fragments disclosed herein can be a heavy chain variable domain antibody (VHH). In some embodiments, the single domain antibody fragment has the structure of, from N-terminus to C-terminus, framework (FR) 1 (FR1)-CDR1-FR2-CDR2-FR3-CDR3-FR4, and wherein:
(a) the FR1 is set forth as Z₁VQLVESGGGLVZ₂AGZ₃SLRLSCZ₄Z5S (SEQ ID NO: 1), in which Z₁ is E or Q; Z₂ is Q or R; Z₃ is G or D; Z₄ is A, T, or V; and Z₅ is A, G, or V; (b) the FR2 is set forth as Z₆Z₇WZ₈RQZ₉PGKZ₁₀REZ₁₁VZ₁₂Z₁₃ (SEQ ID NO: 28), in which Z₆ is L, M, R, or V; Z₇ is A, G, or H; Z₈ is F, L, or Y; Z₉ is A or G; Z₁₀ is E, N, Q, or R; Z₁₁ is F or L; Z₁₂ is A, G, or S; and Z₁₃ is A, G or S;
(c) the FR3 is set forth as Z₁₄YZ₁₅DSZ₁₆Z₁₇GRFTISRDZ₁₈Z₁₉Z₂₀Z₂₁TVYLZ₂₂MZ₂₃ SLKPEDTAZ₂₄YYCAA (SEQ ID NO: 55), in which Z₁₄ is L, N, or Y; Z₁₅ is A, G, L, P, or Q; Z₁₆ is M, V, or absent; Z₁₇ is E or K; Z₁₈ is G, K, or N; Z₁₉ is A, G, T, or V; Z₂₀ is E, K, or R; Z₂₁ is D, N, or S; Z₂₂ is H, Q or R; Z₂₃ is D, N, or S; and Z₂₄ is N, T, or V; and
(d) the FR4 is set forth as Z₂₅Z₂₆WZ₂₇QGTZ₂₈VTVSS (SEQ ID NO: 82), in which Z₂₅ is D, E, L, R, or T; Z₂₆ is D, S, or Y; Z₂₇ is G or A; and Z₂₈ is L or Q.

In some examples, the FR1 may comprise one of the following:

(a)
(SEQ ID NO: 3)
EVQLVESGGGLVRAGGSLRLSCAAS;
(b)
(SEQ ID NO: 4)
QVQLVESGGGLVQAGGSLRLSCAAS;
(c)
(SEQ ID NO: 5)
QVQLVESGGGLVQAGDSLRLSCAVS;
(d)
(SEQ ID NO: 6)
QVQLVESGGGLVQAGDSLRLSCAAS;
(e)
(SEQ ID NO: 7)
QVHLVESGGGLVQAGGSLGLSCAAS;
(f)
(SEQ ID NO: 8)
QVQLVESGGGLVQAGGSLRLSCVAS;
(g)
(SEQ ID NO: 11)
QVQLVESGGGLVQAGGSLRLSCTAS;
and
(h)
(SEQ ID NO: 13)
EVQLVESGGGLVQAGGSLRLSCAAS.

Alternatively or in addition, the FR2 may comprise one or the following:

(a)
(SEQ ID NO: 30)
LGWFRQAPGKNREFVAA;
(b)
(SEQ ID NO: 31)
VGWFRQAPGKQREFVAA;
(c)
(SEQ ID NO: 32)
MAWLRQAPGKEREFVAA;
(d)
(SEQ ID NO: 33)
MGWFRQAPGKEREFVAS;
(e)
(SEQ ID NO: 34)
MGWFRQAPGKEREFVAA;
(f)
(SEQ ID NO: 35)
MGWFRQGPGKEREFAAA;
(g)
(SEQ ID NO: 38)
MGWFRQGPGKEREFVGG;
or
(h)
(SEQ ID NO: 41)
RAWFRQAPGKEREFVAA.

Alternatively or in addition, the FR3 may comprise one of the following:

(a)
(SEQ ID NO: 57)
YYQDSKGRFTISRDNTKNTAYLQMNSLOPEDTAVYYCAA;
(b)
(SEQ ID NO: 58)
YYGDSVEGRFTVSRDNARSTVYLRMSSLKPDDTAVYYCAA;
(c)
(SEQ ID NO: 59)
YYQDPVKGRFTISRDKAKNTVYLQMNTLKPEDTATYICAA;
(d)
(SEQ ID NO: 60)
YYGDSVEARFTISRDNAKNTVYLQMDSLKPEDTAVYYCAA;
(e)
(SEQ ID NO: 61)
LYADSVKGRFTISRDNGENTVYLQMNSLKPEDTAVYYCAL;
(f)
(SEQ ID NO: 62)
YYADSVKGRFTISRDNAKDTVYLQMNSLKPEDTANYYCAA;
(g)
(SEQ ID NO: 65)
YYADSVKGRFTISRDNAKNTVYLQMNSLKREDTAVYYCAA;
(h)
(SEQ ID NO: 63)
YYADSVKGRFTISRDNAKNTVYLQMNSLKPEDTAVYYCAA;
or
(i)
(SEQ ID NO: 68)
YYPNSMKGRFTISRDNAKNTVYLQMNSLKPEDTAVYYCAA.

Alternatively or in addition, the FR4 may comprise one of the following:

(a)
(SEQ ID NO: 84)
LYWAQGTQVTVSS;
(b)
(SEQ ID NO: 85)
DSWGQGTQVTVSS;
(c)
(SEQ ID NO: 86)
DYWGQGTQVTVSS;
(d)
(SEQ ID NO: 87)
EYWGPGTQVTVSS;
(e)
(SEQ ID NO: 88)
EYWGQGTLVTVSS;
(f)
(SEQ ID NO: 83)
DYWGQGTLVTVSS;
or
(g)
(SEQ ID NO: 95)
RYWGQGTLVTVSS.

In some examples, the single domain antibody fragment has the same FR1, same FR2, same FR3, and same FR4 as a reference antibody of P10, P12, P27, P29, P31, P32, P35, P37, or P38 (e.g., P10, P31, or P38). In specific examples, the single domain antibody fragment comprises P10, P12, P27, P29, P31, P32, P35, P37, or P38. In one example, the single domain antibody fragment is P10. In another example, the single domain antibody fragment is P31. In yet another example, the single domain antibody fragment is P38.

Any of the anti-CD117 CARs comprising a VHH fragment may further comprise a transmembrane domain, which can be a CD8 transmembrane domain. Alternatively or in addition, the anti-CD117 CAR may comprise a co-stimulatory domain, which can be a CD28 co-stimulatory domain or a 4-1BB co-stimulatory domain. Examples of VHH-containing anti-CD117 CAR polypeptides can be found in Table 2 below, all of which are within the scope of the present disclosure.

Further, the present disclosure provides a nucleic acid comprising a nucleotide sequence encoding any of the anti-CD117 CARs disclosed herein. In some instances, the nucleic acid is a vector, for example, a lentiviral vector or an adeno-associated viral (AAV) vector. Alternatively, the nucleic acid is a messenger RNA (mRNA).

In other aspects, the present disclosure provides a population of genetically engineered immune cells, wherein the genetically engineered immune cells comprise a nucleic acid encoding a chimeric antigen receptor (CAR) that binds CD117 (anti-CD117 CAR) as disclosed herein and express the anti-CD117 CAR. The genetically engineered immune cells comprise genetically engineered T cells. In some examples, the nucleic acid comprises the coding sequences provided in Table 2 below for each of the corresponding anti-CD117 CAR.

In some embodiments, the population of genetically engineered immune cells disclosed herein comprises genetically engineered immune cells further comprise a disrupted T cell receptor alpha chain constant region (TRAC) gene, a disrupted beta-2-microglobulin (β2M) gene, or a combination thereof. In some examples, the nucleic acid encoding the anti-CD117 CAR is inserted at a genomic locus of interest. In one example, the nucleic acid encoding the anti-CD117 CAR is inserted into the disrupted TRAC gene.

In other embodiments, the population of genetically engineered immune cells disclosed herein comprises genetically engineered immune cells comprise a wild-type TRAC gene. Alternatively or in addition, the population of genetically engineered immune cells disclosed herein comprises genetically engineered immune cells comprise a wild-type β2M gene.

Any of the anti-CD117 CAR-T cells disclosed herein may express the anti-CD117 CAR in a permanent manner (e.g., having the CAR-coding nucleic acid incorporated into a genomic site). Alternatively, the anti-CD117 CAR-T cells may express the anti-CD117 CAR in a transient manner (e.g., having the CAR-coding nucleic acid, e.g., mRNA molecules, eliminated from the T cells overtime).

In yet other aspects, the present disclosure provides a method for inhibiting CD117+ cells in a subject, the method comprising administering to a subject in need thereof an effective amount of a population of the genetically engineered immune cells disclosed herein. In some embodiments, the subject is a human patient having a disease associated with CD117+ cells. For example, the human patient has a hematopoietic disease, which may be a hematopoietic malignancy. In specific examples, the human patient has leukemia, e.g., acute myeloid leukemia.

In other examples, the human patient may have melanoma.

In some embodiments, the subject can be a human patient in need of hematopoietic stem cell transplantation. Such a method may further comprise administering to the human patient a population of hematopoietic stem cells.

In some embodiments, the population of genetically engineered immune cells is autologous to the subject. In other embodiments, the population of genetically engineered immune cells is allogeneic to the subject.

In addition, the present disclosure provides a method for preparing a population of genetically engineered immune cells, the method comprising: (a) delivering to a plurality of immune cells a nucleic acid (e.g., a vector or a mRNA molecule) encoding any of the anti-CD117 CARs disclosed herein; and (b) producing a population of genetically engineered immune cells expressing the anti-CD117 CAR.

In some embodiments, the method may further comprise delivering to the immune cells (i) an RNA-guided nuclease or a nucleic acid encoding the nuclease and (ii) a gRNA targeting a TRAC gene, a gRNA targeting a β2M gene, or a combination thereof. the genetically engineered immune cells thus produced may express the anti-CD117 CAR and have a disrupted TRAC gene, a disrupted 82M gene, or a combination thereof.

In some examples, the gRNA targeting the TRAC gene is specific to a target sequence in the TRAC gene, which comprises the nucleotide sequence of SEQ ID NO: 12. The gRNA targeting the TRAC gene may comprise a spacer of SEQ ID NO: 23. Alternatively or in addition, the gRNA targeting the β2M gene is specific to a target sequence in the β2M gene, which comprises the nucleotide sequence of SEQ ID NO: 200. The gRNA targeting the β2M gene may comprise a spacer of SEQ ID NO: 202. Any of the gRNAs targeting the TRAC gene and/or the gRNAs targeting the β2M gene may comprise a scaffold sequence. Alternatively or in addition, the gRNA targeting the TRAC gene and/or the gRNA targeting the β2M gene comprise one or more modifications. In specific examples, the gRNA targeting the TRAC gene comprises the nucleotide sequence of SEQ ID NO: 21 or SEQ ID NO: 40. The gRNA targeting the β2M gene comprise the nucleotide sequence of SEQ ID NO: 204 or SEQ ID NO: 205. See Table 3.

In any of the methods disclosed herein, steps (i) and (ii) can be delivered to the immune cells concurrently with the vector. In some embodiments, the vector comprises the nucleic acid encoding the anti-CD117 CAR, the nucleic acid encoding the RNA-guided nuclease, and optionally a nucleic acid encoding the gRNA(s). In some embodiments, the method comprises delivering to the immune cells a ribonucleoprotein (RNP) complex comprising the RNA-guided nuclease and the gRNA(s). In some examples, the vector comprises an upstream fragment and a downstream fragment flanking the nucleic acid encoding the anti-CD117 CAR. The upstream fragment and the downstream fragment are homologous to a genomic locus of interest, for example, the TRAC gene.

Also within the scope of the present disclosure is any of the populations of genetically engineered immune cells (e.g., T cells) that express an anti-CD117 CAR as disclosed herein for use in inhibiting CD117+ cells and treating diseases associated with the CD117+ cells (e.g., the various types of cancer disclosed herein); as well as uses of such genetically engineered immune cells for manufacturing a medicament for use to achieve the intended therapeutic purposes.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIGS. 1A-1E include diagrams showing characterization of genetically engineered T cells expressing an anti-CD117 CAR (scFv) as indicated and having disrupted TRAC and β2M genes.

FIG. 1A: percentage of cells expressing TRAC and β2M. FIG. 1B: percentage of TRAC and β2M negative cells. FIG. 1C: percentage of CAR-expressing T cells. FIG. 1D: percentage of CD4+ cells. FIG. 1E: percentage of CD8+ cells. For each panel from left to right: T-B-cKIT CTX-2840 AAV+, T-B-cKIT CTX-2841 AAV+, T-B-cKIT CTX-2842 AAV+, T-B-cKIT CTX-2843 AAV+, T-B-cKIT CTX-2844 AAV+, T-B-cKIT CTX-2845 AAV+, T-B-cKIT CTX-2846 AAV+, T-B-cKIT CTX-2847 AAV+, T-B-AAV−, RNP−, and EP−.

FIGS. 2A-2B include diagrams showing in vitro cytotoxicity of TRAC−/β2M− anti-CD117 CAR-T cells as indicated against CD117+ and CD117− target cells. FIG. 2A: SKMEL-3 target cells (CD117+) in a 20-hr co-culture incubation. FIG. 2B: CAL-27 target cells (CD117−) in a 20-hr co-culture incubation.

FIGS. 3A-3B include diagrams showing in vitro cytotoxicity of TRAC−/β2M− anti-CD117 CAR-T cells (CX-2840 and CTX-2841) against CD117+ and CD117− target cells. FIG. 3A: Kasumi-1 target cells (CD117+) in a 20-hr co-culture incubation. FIG. 3B: Cal-27 target cells (CD117−) in a 20-hr co-culture incubation.

FIGS. 4A-4F include diagrams showing in vitro cytotoxicity of TRAC−/β2M− anti-CD117 CAR-T cells (CX-2840 and CTX-2841) against CD117+ and CD117− target cells. FIG. 4A: CD34+ target cells (CD117+) in a 20-hr co-culture incubation. FIG. 4B: CTX-2841 against CD34+ target cells in a 20-hr co-culture incubation. FIG. 4C: Kasumi-1 target cells (CD117+) in a 20-hr co-culture incubation. FIG. 4D: CTX-2841 against Kasumi-1 target cells in a 20-hr co-culture incubation. FIG. 4E: CAL-27 target cells (CD117−) in a 20-hr co-culture incubation. FIG. 4F: CTX-2841 against CAL-27 (CD117−) target cells in a 20-hr co-culture incubation.

FIGS. 5A-5E include diagrams showing characterization of genetically engineered T cells expressing an anti-CD117 CAR (VHH) as indicated and having disrupted TRAC and β2M genes. FIG. 5A: percentage of cells expressing TRAC and β2M. FIG. 5B: percentage of TRAC and β2M negative cells. FIG. 5C: percentage of CAR-expressing T cells. FIG. 5D: percentage of CD4+ cells. FIG. 5E: percentage of CD8+ cells. For each panel from left to right: T-B-CTX-2867, T-B-CTX-2868, T-B-CTX-2869, T-B-CTX-2870, T-B-CTX-2871, T-B-CTX-2872, T-B-CTX-2873, T-B-CTX-2874, T-B-CTX-2875, T-B-CTX-2876, T-B-CTX-2877, T-B-CTX-2878, T-B-CTX-2879, T-B-CTX-2880, T-B-CTX-2881, T-B-CTX-2882, AAV−, RNP−, and EP−.

FIGS. 6A-6F include diagrams showing in vitro cytotoxicity of TRAC−/β2M− anti-CD117 CAR(VHH)-T cells as indicated against CD117+ and CD117− target cells. FIGS. 6A-6B: comparison of cytotoxicity against CD34+ HSPC target cells between anti-CD117 CAR containing the CD28 co-stimulatory and counterpart CARs containing the 4-1BB costimulatory domain in an 18-hr co-culture incubation. FIGS. 6C-6D: Kasumi-1 target cells (CD117+) in an 18-hr co-culture incubation. FIGS. 6E-6F: CAL-27 target cells (CD117−) in an 18-hr co-culture incubation.

FIGS. 7A-7D include diagrams showing in vivo cytotoxicity of TRAC−/β2M− anti-CD117 CAR-T cells against CD117+ HSCs in an animal model. FIGS. 7A-7B: % huCD117+ HSCs (FIG. 7A) and total huCD117+ HSCs (FIG. 7B) on Day 7. FIGS. 7C-7D: % huCD117+ HSCs (FIG. 7C) and total huCD117+ HSCs (FIG. 7D) on Day 14.

FIG. 8 is a diagram showing viability of CAR T cells transfected with mRNA molecules encoding an anti-CD117 CAR (CTX-2840).

FIG. 9 is a diagram showing viability of TRAC⁻/β2M⁻ CAR T cells post electroporation of mRNAs encoding anti-CD117 CARs as indicated.

FIGS. 10A-10D include diagrams showing impact T cell subtypes and expression of T cell activation and differentiation markers in TRAC⁻/β2M⁻ anti-CD117 CAR T cells transiently expressing anti-CD117 CAR. FIG. 10A: percentage of CD4⁺ cells and CD4⁺ cells. FIG. 10B: percentage of differentiated CD27+ cells vs CD45RO⁺ cells. FIG. 10C: percentages of T cell subpopulations in CD4+ cells transfected with mRNAs encoding an anti-CD117 CAR at various concentrations as indicated. FIG. 10D: percentages of T cell subpopulations of CD8+ cells transfected with mRNAs encoding an anti-CD117 CAR at various concentrations as indicated.

FIGS. 11A-11B include diagrams showing expression of TCR and T cell exhaustion markers in TRAC⁻/β2M⁻ T cells transfected with mRNAs encoding an anti-CD117 CAR at various concentrations as indicated. FIG. 11A: percentage of TCR⁻ cells. FIG. 11B: percentages of Lag3⁺, PD-1⁺, and TIM3⁺ cells.

FIGS. 12A-12B include diagrams showing assessment of myeloablation efficacy in humanized NSG mice injected with TRAC⁻/β2M⁻ anti-CD117 CAR T cells transiently expressing an anti-CD117 CAR 72 hours. FIG. 12A: chimerism analysis in blood cells from bone marrow. FIG. 12B: percentages of CD34⁺/CD117⁺ cells in blood cells from bone marrow.

FIGS. 13A-13C include diagrams showing results of dose optimization studies for TRAC⁻/β2M⁻ anti-CD117 CAR T cells transiently expressing anti-CD117 CAR. FIG. 13A: chimerism analysis in bone marrow blood cells at the indicated CAR T cell doses. FIG. 13B: percentages of hCD45⁺ cells in bone marrow blood cells at the indicated CAR T cell doses. FIG. 13C: percentages of CD34⁺/CD117⁺ cells in bone marrow blood cells at the indicated CAR T cell doses.

FIGS. 14A-14F include diagrams showing effects on myeloablation and engraftment of anti-CD117 CAR-T cells in humanized NSG mice. FIG. 14A: chimerism analysis in blood cells from bone marrow blood cells. FIG. 14B: viabilities of cells in bone marrow blood cells. FIG. 14C: total cell count in bone marrow blood cells. FIG. 14D: percentage of hCD45⁺ cells in bone marrow blood cells. FIG. 14E: percentage of CD34⁺/cKit⁺ in the hCD45⁺ cell subpopulation. FIG. 14F: percentages CD34⁺/CD117⁺ live cells.

FIGS. 15A-15B include diagrams showing engraftment of CCR5 edited human CD34+ cells in humanized NSG mice treated with anti-CD117 CAR-T cells. FIG. 15A: chimerism analysis in bone marrow blood cells. FIG. 15B: CCR5 editing efficiency in the indicated groups.

### DETAILED DESCRIPTION OF THE INVENTION

CD117 (a.k.a., cKIT or SCFR) is known to be an important cell surface marker for various types of disease cells, including cancer cells such as hematopoietic cancer cells. Accordingly, Cell therapy targeting CD117 would be promising in eliminating CD117+ disease cells, thereby benefiting treatment of diseases associated with the CD117+ cells (e.g., various types of cancers disclosed herein, including hematopoietic malignancies).

The present disclosure is based, at least in part, on the development of anti-CD117 CAR-T cells (with either anti-CD117 scFv or anti-CD117 VHH), which showed cytotoxicity against CD117+ cells (e.g., HSCs) both in vitro and in vivo. Further, CAR-T cells transiently expressing an anti-CD117 CAR, including both TRAC/B2M wild-type and TRAC/B2M disrupted T cells, showed myeloablation effects both in vitro and in vivo, and treatment with the anti-CD117 CAR-T cells enhanced engraftment of new donor CD34+ cells in an animal model. Accordingly, disclosed herein are anti-CD117 CAR polypeptides and genetically engineered T cells expressing such, which optionally can have disrupted TRAC and β2M genes, methods of using such genetically engineered T cells for inhibiting CD117+ cells and thus for treating diseases associated with CD117+ cells or enhancing engraftment of transplanted hematopoietic stem cells, as well as methods for producing any of the genetically engineered T cells disclosed herein.

### I. Chimeric Antigen Receptor Specific to CD117

A chimeric antigen receptor (CAR), as used herein, refers to an artificial immune cell receptor that is engineered to recognize and bind to an antigen expressed by undesired cells, for example, disease cells such as cancer cells. A CAR polypeptide can be introduced into immune cells such as T cells for surface expression to produce CAR T cell. CARs have the ability to redirect T-cell specificity and reactivity toward a selected target in a non-MHC-restricted manner. The non-MHC-restricted antigen recognition gives CAR-T cells the ability to recognize an antigen independent of antigen processing, thus bypassing a major mechanism of tumor escape. Moreover, when expressed on T-cells, CARs advantageously do not dimerize with endogenous T-cell receptor (TCR) alpha and beta chains.

There are various designs of CARs, each of which contains different components. In some embodiments, CARs may join an antibody-derived scFv to the CD3zeta (CD3ζ) intracellular signaling domain of the T-cell receptor through hinge and transmembrane domains. In some embodiments, CARs incorporate an additional co-stimulatory domain, e.g., CD28, 4-1BB (41BB), or ICOS, to supply a costimulatory signal. In other embodiments, CARs contain two costimulatory domains (e.g., a combination of CD27, CD28, 4-1BB, ICOS, or OX40) fused with the TCR CD3ζ chain. Maude et al., Blood. 2015; 125(26):4017-4023; Kakarla and Gottschalk, Cancer J. 2014; 20(2):151-155). Any of the various generations of CAR constructs is within the scope of the present disclosure.

In some instances, a CAR can be a fusion polypeptide comprising an extracellular antigen binding domain that recognizes a target antigen (e.g., a single chain variable fragment (scFv) of an antibody or other antibody fragment) and an intracellular domain comprising a signaling domain of the T-cell receptor (TCR) complex (e.g., CD3ζ) and, in most cases, a co-stimulatory domain. (Enblad et al., Human Gene Therapy. 2015; 26(8):498-505). A CAR construct may further comprise a hinge and transmembrane domain between the extracellular domain and the intracellular domain. In some instances, a signal peptide may be located at the N-terminus of the CAR to facilitate cell surface expression. Examples of signal peptides include MLLLVTSLLLCELPHPAFLLIP (SEQ ID NO: 229) and MALPVTALLLPLALLLHAARP (SEQ ID NO: 96). Other signal peptides may be used.

The anti-CD117 chimeric antigen receptor (CAR) disclosed herein comprises an extracellular antigen binding domain (e.g., a single chain variable fragment (scFv) or a single domain antibody fragment (e.g., VHH))) specific to a CD117 antigen (e.g., the human CD117 antigen).

### (a) Antigen Extracellular Binding Domain

The extracellular antigen binding domain is the region of any anti-CD117 CARs disclosed herein that is exposed to the extracellular fluid when the CAR is expressed on cell surface. In some instances, the extracellular antigen binding domain may be an antibody fragment that binds CD117, for example, a single chain variable fragment (scFv) or a single domain antibody fragment such as a heavy chain-only antibody fragment (VHH).

Anti-CD17 scFv Fragments

In some embodiments, the antigen binding domain can be a single-chain variable fragment (scFv, which may include an antibody heavy chain variable region (V_{H}) and an antibody light chain variable region (V_{L}) (in either orientation). In some instances, the V_{H} and V_{L} fragment may be linked via a peptide linker. The linker, in some embodiments, includes hydrophilic residues with stretches of glycine and serine for flexibility as well as stretches of glutamate and lysine for added solubility. The scFv fragment retains the antigen-binding specificity of the parent antibody, from which the scFv fragment is derived. In some embodiments, the scFv may comprise humanized V_{H} and/or V_{L} domains. In other embodiments, the V_{H} and/or V_{L} domains of the scFv are fully human.

In some examples, the extracellular antigen-binding domain in the CAR polypeptide disclosed herein is specific to CD117 (e.g., human CD117). In some examples, the extracellular antigen binding domain may comprise a scFv extracellular domain capable of binding to the CD117 antigen. The anti-CD117 scFv may be derived from reference antibody anti-CD117 Ab-1. Alternatively, the anti-CD117 scFv may be derived from reference antibody anti-CD117 Ab-2. Structures of both reference antibodies are provided in sequence Table 1 below.

In some embodiments, an anti-CD117 scFv derived from Ab-1 may comprise a heavy chain variable domain (V_{H}) having the same heavy chain complementary determining regions (CDRs) as those in Antibody Ab-1 and/or a light chain variable domain (V_{L}) having the same light chain CDRs as those in Ab-1. In other embodiments, an anti-CD117 scFv derived from Ab-2 may comprise a heavy chain variable domain (V_{H}) having the same heavy chain complementary determining regions (CDRs) as those in Antibody Ab-2 and/or a light chain variable domain (V_{L}) having the same light chain CDRs as those in Ab-2.

Two antibodies having the same V_{H} and/or V_{L} CDRs means that their CDRs are identical when determined by the same approach (e.g., the Kabat approach, the Chothia approach, the AbM approach, the Contact approach, or the IMGT approach as known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinf.org.uk/abs. bioinf.org.uk/abs/.

In other embodiments, an anti-CD117 scFv derived from Ab-1 or Ab-2 may be a functional variant of Ab-1 or Ab-2. Such a functional variant is substantially similar to Ab-1 or Ab-2, respectively, both structurally and functionally. A functional variant comprises substantially the same V_{H} and V_{L} CDRs as Ab-1 or Ab-2. For example, it may comprise only up to 8 (e.g., 8, 7, 6, 5, 4, 3, 2, or 1) amino acid residue variations in the total CDR regions relative to those in Ab-1 or Ab-2 and binds the same epitope of CD117 with substantially similar affinity (e.g., having a K_{D} value in the same order). In some instances, the functional variants may have the same heavy chain CDR3 as Ab-1 or Ab-2, and optionally the same light chain CDR3 as Ab-1 or Ab-2. Such an anti-CD117 scFv may comprise a V_{H} fragment having CDR amino acid residue variations (e.g., up to 5, for example, 5, 4, 3, 2, and 1) in only the heavy chain CDR1 and/or CDR2 as compared with the V_{H} of Ab-1 or Ab-2. Alternatively or in addition, the anti-scFv antibody may further comprise a V_{L} fragment having CDR amino acid residue variations (e.g., up to 5, for example, 5, 4, 3, 2, and 1) in only the light chain CDR1 and/or CDR2 as compared with the V_{L} of Ab-1 or Ab-2. In some examples, the amino acid residue variations can be conservative amino acid residue substitutions.

In some examples, any of the variations in one or more of the CDR regions can be conservative substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

In some embodiments, the anti-CD117 scFv derived from Ab-1 may be in the format of, from N-terminus to C-terminus, V_{H}-linker-V_{L}. In some examples, the anti-CD117 scFv comprises a V_{H} fragment of SEQ ID NO: 108 and a V_{L} fragment of SEQ ID NO: 107. In specific examples, the anti-CD117 scFv in any of the anti-CD117 CAR may comprise the amino acid sequence of SEQ ID NO: 110. Alternatively, the anti-CD117 scFv derived from Ab-1 may be in the format of, from N-terminus to C-terminus, V_{L}-linker-V_{H}. In some examples, the anti-CD117 scFv in any of the anti-CD117 CAR may comprise the amino acid sequence of SEQ ID NO: 109. In some instances, the anti-CD117 scFv may share at least 85% sequence identity (e.g., at least 90%, at least 95% or above) to SEQ ID NO: 109 or SEQ ID NO: 110.

In some embodiments, the anti-CD117 scFv derived from Ab-2 may be in the format of, from N-terminus to C-terminus, V_{H}-linker-V_{L}. In some examples, the anti-CD117 scFv comprises a V_{H} fragment of SEQ ID NO: 130 and a V_{L} fragment of SEQ ID NO: 129. In specific examples, the anti-CD117 scFv in any of the anti-CD117 CAR may comprise the amino acid sequence of SEQ ID NO: 132. Alternatively, the anti-CD117 scFv derived from Ab-2 may be in the format of, from N-terminus to C-terminus, V_{L}-linker-V_{H}. In some examples, the anti-CD117 scFv in any of the anti-CD117 CAR may comprise the amino acid sequence of SEQ ID NO: 131. In some instances, the anti-CD117 scFv may share at least 85% sequence identity (e.g., at least 90%, at least 95% or above) to SEQ ID NO: 131 or SEQ ID NO: 132.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

### Anti-CD117 Single Domain Antibody Fragments

In some embodiments, the antigen binding domain can be a single-domain antibody fragment. Single-domain antibodies, also known as nanobodies, are small antigen-binding fragments containing only one heavy or light chain variable region (as opposed to conventional antibodies having both heavy and light chain variable regions). In some instances, the single domain antibodies provided herein are heavy chain only antibodies (VHH antibodies) containing a single heavy chain variable region.

Like conventional antibodies, a single domain antibody such as a VHH antibody contain regions of hypervariability, also known as “complementarity determining regions” (“CDR”), interspersed with regions that are more conserved, which are known as “framework regions” (“FR”). A VHH antibody is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinf.org.uk/abs).

In some embodiments, an antibody moiety disclosed herein may share the same complementary determining regions (CDRs) as a reference antibody. In some embodiments, an antibody moiety disclosed herein may share a certain level of sequence identity (e.g., at least 80% such as at least 85%, at least 90%, at least 95% or above) as compared with a reference sequence.

In some embodiments, an antibody moiety disclosed herein may have one or more amino acid variations relative to a reference antibody. The amino acid residue variations as disclosed in the present disclosure (e.g., in framework regions and/or in CDRs) can be conservative amino acid residue substitutions.

In some aspects, the present disclosure features an antibody that binds CD117, comprising a single domain antibody fragment, which comprises:
(i) a complementarity determining region 1 (CDR1) set forth as GX₁X₂TX₃X₄X₅X₆X₇, in which X₁ is D, G, H, R, or T; X₂ is T or absent; X₃ is F, L, S, or V; X₄ is G, S, or T; X₅ is I, N, S, T or Y; X₆ is D, V, or Y; and X₇ is A, F, P, S, V or W;
(ii) a complementarity determining region 2 (CDR2) set forth as X₈X₉X₁₀X₁₁X₁₂X₁₃X₁₄X₁₅, in which X₈ is I or V; X₉ is A, G, H, L, R, S, T, or V, X₁₀ is R, S, or W; X₁₁ is G, N, S, or Y; X₁₂ is A, G, L, or absent; X₁₃ is A, D, G, L, or S; X₁₄ is G, M, S, T, or V; and X₁₅ is A, L, or T; and
(iii) a complementarity determining region 3 (CDR3) set forth as: (a) GRFHPIRVDTA (SEQ ID NO: 70); (b) ASGSNWRLGAIDEY (SEQ ID NO: 71); (c) GQHLSGLGGSAWSIEG (SEQ ID NO: 72); (d) RQYVGSGSYYLKKEGGY (SEQ ID NO: 73);
(e) DSTGVYGTGYVSSRKGRY (SEQ ID NO: 74); (f) AFTPEFRDGGIWDDASV (SEQ ID NO: 75); (g) VRRRWLIWQEEEY (SEQ ID NO: 78);
(h) DQRGVPAYYSDYALY (SEQ ID NO: 80); (i) DESFPAYYSDYALY (SEQ ID NO: 81); (j) VLRTGM (SEQ ID NO: 69); (k) SDSYFYASPHLY (SEQ ID NO: 76);
(1) SDTYFYASPHLY (SEQ ID NO: 77); or (m) RRGTILVVQEYEY (SEQ ID NO: 79).

Any of the single domain antibody fragments disclosed herein can be a heavy chain variable domain antibody (VHH). In some embodiments, the single domain antibody fragment has the structure of, from N-terminus to C-terminus, framework (FR) 1 (FR1)-CDR1-FR2-CDR2-FR3-CDR3-FR4, and wherein:
(a) the FR1 is set forth as ZIVQLVESGGGLVZ₂AGZ₃SLRLSCZ₄Z₅S (SEQ ID NO: 1), in which Z₁ is E or Q; Z₂ is Q or R; Z₃ is G or D; Z₄ is A, T, or V; and Z₅ is A, G, or V;
(b) the FR2 is set forth as Z₆Z₇WZ₈RQZ₉PGKZ₁₀REZ₁₁VZ₁₂Z₁₃ (SEQ ID NO: 28), in which Z₆ is L, M, R, or V; Z₇ is A, G, or H; Z₈ is F, L, or Y; Z₉ is A or G; Z₁₀ is E, N, Q, or R; Z₁₁ is F or L; Z₁₂ is A, G, or S; and Z₁₃ is A, G or S;
(c) the FR3 is set forth as Z₁₄YZ₁₅DSZ₁₆Z₁₇GRFTISRDZ₁₈Z₁₉Z₂₀Z₂₁TVYLZ₂₂MZ₂₃ SLKPEDTAZ₂₄YYCAA (SEQ ID NO: 55), in which Z₁₄ is L, N, or Y; Z₁₅ is A, G, L, P, or Q;
Z₁₆ is M, V, or absent; Z₁₇ is E or K; Z₁₈ is G, K, or N; Z₁₉ is A, G, T, or V; Z₂₀ is E, K, or R; Z₂₁ is D, N, or S; Z₂₂ is H, Q or R; Z₂₃ is D, N, or S; and Z₂₄ is N, T, or V; and
(d) the FR4 is set forth as Z₂₅Z₂₆WZ₂₇QGTZ₂₈VTVSS (SEQ ID NO: 82), in which Z₂₅ is D, E, L, R, or T; Z₂₆ is D, S, or Y; Z₂₇ is G or A; and Z₂₈ is L or Q.

In some embodiments, the anti-CD117 single domain antibody disclosed herein comprises the consensus sequence of each of FR1-CDR1-FR2-CDR2-FR3-CDR3-FR4 listed in Table 1 below and disclosed herein as well. Exemplary sequences of each of these domains in an anti-CD117 antibody as disclosed herein are also provided in Table 1. The anti-CD117 antibody provided herein may contain one or more such sequences.

TABLE 1
Consensus Sequences and Exemplary Sequences of Anti-CD₁₁7 VHH

| Antibodies | FR1 | CDR1 | FR2 |
|---|---|---|---|
| Consensus | Z₁VQLVESGGGLVZ₂AGZ₃S | GX₁X₂TX₃X₄X₅X₆X₇ | Z₆Z₇WZ₈RQZ₉PG |
| Sequence | LRLSCZ₄Z₅S | | KZ₁₀REZ₁₁VZ₁₂Z₁₃ |
| | (SEQ ID NO: 1) | | (SEQ ID NO: 28) |
| MP16 | EVQLVESGGGLVQAGGSL | GTTSGIVA | MHWYRQAPGK |
| | RLSCVAS | (SEQ ID NO: 15) | RRELVAS |
| | (SEQ ID NO: 2) | | (SEQ ID NO: 29) |
| P27 | EVQLVESGGGLVRAGGSL | GRTTFSTYW | LGWFRQAPGKN |
| | RLSCAAS¹ | (SEQ ID NO: 16) | REFVAA |
| | (SEQ ID NO: 3) | | (SEQ ID NO: 30) |
| P35 | QVQLVESGGGLVQAGGSL | GGTFSIYP | VGWFRQAPGKQ |
| | RLSCAAS² | (SEQ ID NO: 17) | REFVAA |
| | (SEQ ID NO: 4) | | (SEQ ID NO: 31) |
| P37 | QVQLVESGGGLVQAGDSL | GRTLSNYF | MAWLRQAPGKE |
| | RLSCAVS | (SEQ ID NO: 18) | REFVAA |
| | (SEQ ID NO: 5) | | (SEQ ID NO: 32) |
| P32 | QVQLVESGGGLVQAGDSL | GRTFSSYA1 | MGWFRQAPGKE |
| | RLSCAAS | (SEQ ID NO: 19) | REFVAS |
| | (SEQ ID NO: 6) | | (SEQ ID NO: 33) |
| P29 | QVHLVESGGGLVQAGGSL | GHTFSNYA | MGWFRQAPGKE |
| | GLSCAAS | (SEQ ID NO: 20) | REFVAA1 |
| | (SEQ ID NO: 7) | | (SEQ ID NO: 34) |
| P10 | QVQLVESGGGLVQAGGSL | GRTFSSYA1 | MGWFRQGPGKE |
| | RLSCVAS | (SEQ ID NO: 19) | REFAAA |
| | (SEQ ID NO: 8) | | (SEQ ID NO: 35) |
| MP14 | QVQLVESGGGLVQAGGSL | GRTVSTYA2 | MGWFRQAPGKE |
| | RLSCAGS | (SEQ ID NO: 22) | REFVSA |
| | (SEQ ID NO: 9) | | (SEQ ID NO: 36) |
| MP6 | QVQLVESGGGLVQAGGSL | GRTVSTYA2 | MGWFRQAPGKE |
| | RLSCVGS | (SEQ ID NO: 22) | RELVAA |
| | (SEQ ID NO: 10) | | (SEQ ID NO: 37) |
| P12 | QVQLVESGGGLVQAGGSL | GDTFSSYS | MGWFRQGPGKE |
| | RLSCTAS | (SEQ ID NO: 24) | REFVGG |
| | (SEQ ID NO: 11) | | (SEQ ID NO: 38) |
| MP7 | QVQLVESGGGLVQAGGSL | GRTFSSYV | MGWFRQAPGKE |
| | RLSCAAS² | (SEQ ID NO: 25) | REFVAA1 |
| | (SEQ ID NO: 4) | | (SEQ ID NO: 34) |
| P38 | EVQLVESGGGLVQAGGSL | GRTSGSYV | MGWFRQAPGKE |
| | RLSCAAS | (SEQ ID NO: 26) | REFVAA1 |
| | (SEQ ID NO: 13) | | (SEQ ID NO: 34) |
| P31 | EVQLVESGGGLVRAGGSL | GRTFTYDA | RAWFRQAPGKE |
| | RLSCAAS¹ | (SEQ ID NO: 27) | REFVAA |
| | (SEQ ID NO: 3) | | (SEQ ID NO: 41) |

| | CDR2 | FR3 |
|---|---|---|
| Consensus | X₈X₉X₁₀X₁₁X₁₂X₁₃X₁₄X₁₅ | Z₁₄YZ₁₅DSZ₁₆Z₁₇GRFTISRDZ₁₈Z₁₉Z₂₀Z₂₁TV |
| Sequence | | YLZ₂₂MZ₂₃SLKPEDTAZ₂₄YYCAA |
| | | (SEQ ID NO: 55) |
| MP16 | VVRGASL | NYLDSVKGRFIISEDGARNTVYLQMNSL |
| | (SEQ ID NO: 42) | KPEDTAVYYCNA |
| | | (SEQ ID NO: 56) |
| P27 | ISWSAGMA | YYQDSKGRFTISRDNTKNTAYLQMNSLQ |
| | (SEQ ID NO: 43) | PEDTAVYYCAA |
| | | (SEQ ID NO: 57) |
| P35 | IGWSASGT | YYGDSVEGRFTVSRDNARSTVYLRMSSL |
| | (SEQ ID NO: 44) | KPDDTAVYYCAA |
| | | (SEQ ID NO: 58) |
| P37 | IHWSLGST | YYQDPVKGRFTISRDKAKNTVYLQMNT |
| | (SEQ ID NO: 45) | LKPEDTATYICAA |
| | | (SEQ ID NO: 59) |
| P32 | ITSSGLVA | YYGDSVEARFTISRDNAKNTVYLQMDSL |
| | (SEQ ID NO: 46) | KPEDTAVYYCAA |
| | | (SEQ ID NO: 60) |
| P29 | ISWSGGST1 | LYADSVKGRFTISRDNGENTVYLQMNSL |
| | (SEQ ID NO: 47) | KPEDTAVYYCAL |
| | | (SEQ ID NO: 61) |
| P10 | ILSNGLTT | YYADSVKGRFTISRDNAKDTVYLQMNS |
| | (SEQ ID NO: 48) | LKPEDTANYYCAA |
| | | (SEQ ID NO: 62) |
| MP14 | IARSGGGT | YYADSVKGRFTISRDNAKNTVYLQMNS |
| | (SEQ ID NO: 49) | LKPEDTAVYYCAA1 |
| | | (SEQ ID NO: 63) |
| MP6 | IGRSGGST | YYADSVKGRFTISRDNVRNTVYLQMNS |
| | (SEQ ID NO: 50) | LKPEDAAVYYCAA |
| | | (SEQ ID NO: 64) |
| P12 | IRWSGGTT | YYADSVKGRFTISRDNAKNTVYLQMNS |
| | (SEQ ID NO: 51) | LKREDTAVYYCAA |
| | | (SEQ ID NO: 65) |
| MP7 | ISWYGDST | YYADSVKGRFTISRDNAKNTVYLHMNS |
| | (SEQ ID NO: 52) | LKPEDTAVYYCAA |
| | | (SEQ ID NO: 66) |
| P38 | ISWSAGMT | YYADSVKGRFTISRDNAKNTVYLQMNS |
| | (SEQ ID NO: 53) | LKPEDTAVYYCAA1 |
| | | (SEQ ID NO: 63) |
| P31 | ISWSGGST1 | YYPNSMKGRFTISRDNAKNTVYLQMNS |
| | (SEQ ID NO: 47) | LKPEDTAVYYCAA |
| | | (SEQ ID NO: 68) |
| | CDR3 | FR4 |
| Consensus | | Z₂₅Z₂₆WZ₂₇QGTZ₂₈VT |
| Sequence | | VSS |
| | | (SEQ ID NO: 82) |
| MP16 | VLRTGM | DYWGQGTLVTVSS¹ |
| | (SEQ ID NO: 69) | (SEQ ID NO: 83) |
| P27 | GRFHPIRVDTA | LYWAQGTQVTVSS |
| | (SEQ ID NO: 70) | (SEQ ID NO: 84) |
| P35 | ASGSNWRLGAIDEY | DSWGQGTQVTVSS |
| | (SEQ ID NO: 71) | (SEQ ID NO: 85) |
| P37 | GQHLSGLGGSAWSIEG | DYWGQGTQVTVSS² |
| | (SEQ ID NO: 72) | (SEQ ID NO: 86) |
| P32 | RQYVGSGSYYLKKEGGY | EYWGPGTQVTVSS |
| | (SEQ ID NO: 73) | (SEQ ID NO: 87) |
| P29 | DSTGVYGTGYVSSRKGRY | EYWGQGTLVTVSS |
| | (SEQ ID NO: 74) | (SEQ ID NO: 88) |
| P10 | AFTPEFRDGGIWDDASV | DYWGQGTQVTVSS² |
| | (SEQ ID NO: 75) | (SEQ ID NO: 86) |
| MP14 | SDSYFYASPHLY | DYWGQGTLVTVSS¹ |
| | (SEQ ID NO: 76) | (SEQ ID NO: 83) |
| MP6 | SDTYFYASPHLY | TDWGQGTQVTVSS |
| | (SEQ ID NO: 77) | (SEQ ID NO: 91) |
| P12 | VRRRWLIWQEEEY | DYWGQGTLVTVSS¹ |
| | (SEQ ID NO: 78) | (SEQ ID NO: 83) |
| MP7 | RRGTILVVQEYEY | DYWGQGTLVTVSS¹ |
| | (SEQ ID NO: 79) | (SEQ ID NO: 83) |
| P38 | DQRGVPAYYSDYALY | DYWGQGTQVTVSS² |
| | (SEQ ID NO: 80) | (SEQ ID NO: 86) |
| P31 | DESFPAYYSDYALY | RYWGQGTLVTVSS |
| | (SEQ ID NO: 81) | (SEQ ID NO: 95) |

* Sequences labeled “1” or “2” are identical sequences
* In consensus sequence of CDR1: X₁ is D, G, H, R, or T; X₂ is T or absent; X₃ is F, L, S, or V; X₄ is G, S, or T; X₅ is I, N, S, T or Y; X₆ is D, V, or Y; and X₇ is A, F, P, S, V or W.
* In consensus sequence of CDR2: X₈ is I or V; X₉ is A, G, H, L, R, S, T, or V, X₁₀ is R, S, or W; X₁₁ is G, N, S, or Y; X₁₂ is A, G, L, or absent; X₁₃ is A, D, G, L, or S; X₁₄ is G, M, S, T, or V; and X₁₅ is A, L, or T.
* In consensus sequence of FR1: Z1 is E or Q; Z2 is Q or R; Z3 is G or D; Z4 is A, T, or V; and Z5 is A, G, or V.
* In consensus sequence of FR2: Z₆ is L, M, R, or V; Z₇ is A, G, or H; Z₈ is F, L, or Y; Z₉ is A or G; Z₁₀ is E, N, Q, or R; Z₁₁ is For L; Z₁₂ is A, G, or S; and Z₁₃ is A, G or S.
* In consensus sequence of FR3: Z₁₄ is L, N, or Y; Z₁₅ is A, G, L, P, or Q; Z₁₆ is M, V, or absent; Z₁₇ is E or K; Z₁₈ is G, K, or N; Z₁₉ is A, G, T, or V; Z₂₀ is E, K, or R; Z₂₁ is D, N, or S; Z₂₂ is H, Q or R; Z₂₃ is D, N, or S; and Z₂₄ is N, Tor V
* In consensus sequence of FR4: Z25 is D, E, L, R, or T; Z26 is D, S, or Y; Z27 is G or A; and Z28 is L or Q.

### (b) Transmembrane Domain

The anti-CD117 CAR polypeptide disclosed herein may contain a transmembrane domain, which can be a hydrophobic alpha helix that spans the membrane. As used herein, a “transmembrane domain” refers to any protein structure that is thermodynamically stable in a cell membrane, preferably a eukaryotic cell membrane. The transmembrane domain can provide stability of the CAR containing such.

In some embodiments, the transmembrane domain of a CAR as provided herein can be a CD8 transmembrane domain. In other embodiments, the transmembrane domain can be a CD28 transmembrane domain. In yet other embodiments, the transmembrane domain is a chimera of a CD8 and CD28 transmembrane domain. Other transmembrane domains may be used as provided herein. In one specific example, the transmembrane domain in the anti-CD117 CAR is a CD8a transmembrane domain having the amino acid sequence of SEQ ID NO: 97.

### (c) Hinge Domain

In some embodiments, a hinge domain may be located between an extracellular domain (comprising the antigen binding domain) and a transmembrane domain of a CAR, or between a cytoplasmic domain and a transmembrane domain of the CAR. A hinge domain can be any oligopeptide or polypeptide that functions to link the transmembrane domain to the extracellular domain and/or the cytoplasmic domain in the polypeptide chain. A hinge domain may function to provide flexibility to the CAR, or domains thereof, or to prevent steric hindrance of the CAR, or domains thereof.

In some embodiments, a hinge domain may comprise up to 300 amino acids (e.g., 10 to 100 amino acids, or 5 to 20 amino acids). In some embodiments, one or more hinge domain(s) may be included in other regions of a CAR. In some embodiments, the hinge domain may be a CD8 hinge domain. Other hinge domains may be used.

### (d) Intracellular Signaling Domains

Any of the anti-CD117 CAR constructs disclosed herein contain one or more intracellular signaling domains (e.g., CD3ζ, and optionally one or more co-stimulatory domains), which are the functional end of the receptor. Following antigen recognition, receptors cluster and a signal is transmitted to the cell.

CD3ζ is the cytoplasmic signaling domain of the T cell receptor complex. CD3ζ contains three (3) immunoreceptor tyrosine-based activation motif (ITAM)s, which transmit an activation signal to the T cell after the T cell is engaged with a cognate antigen. In many cases, CD3ζ provides a primary T cell activation signal but not a fully competent activation signal, which requires a co-stimulatory signaling. In some examples, the anti-CD117 CAR construct disclosed herein comprise a CD3ζ cytoplasmic signaling domain, which may have the amino acid sequence of SEQ ID NO: 100.

In some embodiments, the anti-CD117 CAR polypeptides disclosed herein may further comprise one or more co-stimulatory signaling domains. For example, the co-stimulatory domains of CD28 and/or 4-1BB may be used to transmit a full proliferative/survival signal, together with the primary signaling mediated by CD3ζ. In some examples, the CAR disclosed herein comprises a CD28 co-stimulatory molecule, for example, a CD28 co-stimulatory signaling domain having the amino acid sequence of SEQ ID NO: 98. In other examples, the CAR disclosed herein comprises a 4-1BB co-stimulatory molecule, for example, a 4-1BB co-stimulatory signaling domain having the amino acid sequence of SEQ ID NO: 99.

In specific examples, an anti-CD117 CAR disclosed herein may include a CD3ζ signaling domain (e.g., SEQ ID NO: 100) and a CD28 co-stimulatory domain (e.g., SEQ ID NO: 98).

It should be understood that methods described herein encompasses more than one suitable CAR that can be used to produce genetically engineered T cells expressing the CAR, for example, those known in the art or disclosed herein. Examples can be found in, e.g., International Patent Application No. PCT/IB2019/059585, filed Nov. 7, 2019 and U.S. patent application Ser. No. 16/677,207, filed Nov. 7, 2020, the relevant disclosures of each of the prior applications are incorporated by reference herein for the purpose and subject matter referenced herein.

In specific examples, the anti-CD117 CAR disclosed herein may be any one of those provided in Table 2 below. Amino acid sequences of the components of exemplary anti-CD117 CARs are provided in Table 2 below as well. In one example, the anti-CD117 CAR is CTX2840. In another example, the anti-CD117 CAR is CTX2841.

TABLE 2
Anti-CD117 CAR Components and Sequences Thereof

| | | | SEQ |
|---|---|---|---|
| Description | | Sequence | ID |
| Signal peptide 1 | | MALPVTALLLPLALLLHAARP | 96 |
| Signal peptide 2 | | MLLLVTSLLLCELPHPAFLLIP | 229 |
| CD8a (extracellular | | FVPVFLPAKPTTTPAPRPPTPAPTIASQPLSLRPEACRPAAG | 97 |
| + transmembrane | | GAVHTRGLDFACDIYIWAPLAGTCGVLLLSLVITLYCNHRNR | |
| domain) | | | |
| CD28 Costimulatory | | SKRSRLLHSDYMNMTPRRPGPTRKHYQPYAPPRDFAAYRS | 98 |
| Domain | | | |
| 4-1BB | | KRGRKKLLYIFKQPFMRPVQTTQEEDGCSCRFPEEEEGGCEL | 99 |
| Costimulatory | | | |
| Domain | | | |
| CD3-zeta Signaling | | RVKFSRSADAPAYQQGQNQLYNELNLGRREEYDVLDKRRGRD | 100 |
| Domain | | PEMGGKPRRKNPQEGLYNELQKDKMAEAYSEIGMKGERRRGK | |
| | | GHDGLYQGLSTATKDTYDALHMQALPPR | |
| Anti- | LCDR1 | RASESVDIYGNSFMH | 101 |
| CD117 | LCDR2 | LASNLES | 102 |
| Ab-1 | LCDR3 | QQNNEDPYT | 103 |
| | HCDR1 | SYNMH | 104 |
| | HCDR2 | VIYSGNGDTSYNQKFKG | 105 |
| | HCDR3 | ERDTRFGN | 106 |
| | VL | DIVMTQSPDSLAVSLGERATINCRASESVDIYGNSFMHWYQQK | 107 |
| | | PGQPPKLLIYLASNLESGVPDRESGSGSGTDFTLTISSLQAED | |
| | | VAVYYCQQNNEDPYTFGGGTKVEIK | |
| | VH | QVQLVQSGAEVKKPGASVKVSCKASGYTFTSYNMHWVRQAP | 108 |
| | | GQGLEWMGVIYSGNGDTSYNQKFKGRVTITADKSTSTAYMEL | |
| | | SSLRSEDTAVYYCARERDTRFGNWGQGTLVTVSS | |
| | scFv | DIVMTQSPDSLAVSLGERATINCRASESVDIYGNSFMHWYQQKP | 109 |
| | (VL/VH) | GQPPKLLIYLASNLESGVPDRFSGSGSGTDFTLTISSLQAEDVA | |
| | Linker | VYYCQQNNEDPYTFGGGTKVEIKGGGGSGGGGSGGGGSQVQL | |
| | underlined | VQSGAEVKKPGASVKVSCKASGYTFTSYNMHWVRQAPGQGLE | |
| | | WMGVIYSGNGDTSYNQKFKGRVTITADKSTSTAYMELSSLRSE | |
| | | DTAVYYCARERDTRFGNWGQGTLVTVSS | |
| | scFv | QVQLVQSGAEVKKPGASVKVSCKASGYTFTSYNMHWVRQAPG | 110 |
| | (VH/VL) | QGLEWMGVIYSGNGDTSYNQKFKGRVTITADKSTSTAYMELS | |
| | Linker | SLRSEDTAVYYCARERDTRFGNWGQGTLVTVSSGGGGSGGGGS | |
| | underlined | GGGGSDIVMTQSPDSLAVSLGERATINCRASESVDIYGNSFMH | |
| | | WYQQKPGQPPKLLIYLASNLESGVPDRFSGSGSGTDFTLTISSL | |
| | | QAEDVAVYYCQQNNEDPYTFGGGTKVEIK | |
| CTX-2840 | | MALPVTALLLPLALLLHAARPDIVMTQSPDSLAVSLGERATINC | 111 |
| (Ab-1 VL/VH; | | RASESVDIYGNSFMHWYQQKPGQPPKLLIYLASNLESGVPDRES | |
| CD28 CoS with | | GSGSGTDFTLTISSLQAEDVAVYYCQQNNEDPYTFGGGTKVEIK | |
| signal peptide) | | GGGGSGGGGSGGGGSQVQLVQSGAEVKKPGASVKVSCKASGY | |
| | | TFTSYNMHWVRQAPGQGLEWMGVIYSGNGDTSYNQKFKGRVTI | |
| | | TADKSTSTAYMELSSLRSEDTAVYYCARERDTREGNWGQGTLV | |
| | | TVSSSAAAFVPVFLPAKPTTTPAPRPPTPAPTIASQPLSLRPE | |
| | | ACRPAAGGAVHTRGLDFACDIYIWAPLAGTCGVLLLSLVITLY | |
| | | CNHRNRSKRSRLLHSDYMNMTPRRPGPTRKHYQPYAPPRDFAA | |
| | | YRSRVKFSRSADAPAYQQGQNQLYNELNLGRREEYDVLDKRR | |
| | | GRDPEMGGKPRRKNPQEGLYNELQKDKMAEAYSEIGMKGERR | |
| | | RGKGHDGLYQGLSTATKDTYDALHMQALPPR | |
| CTX-2840 | | DIVMTQSPDSLAVSLGERATINCRASESVDIYGNSFMHWYQQK | 112 |
| (no signal peptide) | | PGQPPKLLIYLASNLESGVPDRFSGSGSGTDFTLTISSLQAED | |
| | | VAVYYCQQNNEDPYTFGGGTKVEIKGGGGSGGGGSGGGGSQVQ | |
| | | LVQSGAEVKKPGASVKVSCKASGYTFTSYNMHWVRQAPGQGLE | |
| | | WMGVIYSGNGDTSYNQKFKGRVTITADKSTSTAYMELSSLRSE | |
| | | DTAVYYCARERDTREGNWGQGTLVTVSSSAAAFVPVFLPAKPT | |
| | | TTPAPRPPTPAPTIASQPLSLRPEACRPAAGGAVHTRGLDFAC | |
| | | DIYIWAPLAGTCGVLLLSLVITLYCNHRNRSKRSRLLHSDYMN | |
| | | MTPRRPGPTRKHYQPYAPPRDFAAYRSRVKFSRSADAPAYQQG | |
| | | QNQLYNELNLGRREEYDVLDKRRGRDPEMGGKPRRKNPQEGL | |
| | | YNELQKDKMAEAYSEIGMKGERRRGKGHDGLYQGLSTATKDT | |
| | | YDALHMQALPPR | |
| CTX-2840 coding | | ATGGCGCTTCCGGTGACAGCACTGCTCCTCCCCTTGGCGCTG | 113 |
| | | TTGCTCCACGCAGCAAGGCCGGACATAGTTATGACCCAATCA | |
| | | CCAGACTCACTCGCGGTCAGCTTGGGGGAGCGCGCCACAATT | |
| | | AACTGTCGCGCGTCTGAGTCTGTCGACATCTACGGTAATTCT | |
| | | TTTATGCATTGGTATCAACAAAAGCCAGGACAGCCGCCGAA | |
| | | ATTGTTGATATATCTCGCTAGCAACCTTGAGTCAGGTGTGCC | |
| | | TGATCGGTTCAGTGGTTCTGGGTCAGGGACCGACTTTACGCT | |
| | | GACAATTAGTTCTCTGCAAGCAGAAGATGTGGCAGTGTATTA | |
| | | TTGCCAGCAAAATAATGAGGACCCCTACACCTTCGGGGGTG | |
| | | GCACGAAAGTCGAAATTAAAGGGGGAGGCGGCAGTGGCGGT | |
| | | GGCGGTAGCGGAGGTGGTGGGTCTCAGGTCCAACTGGTTCA | |
| | | ATCCGGAGCGGAAGTTAAGAAGCCAGGTGCAAGTGTCAAAG | |
| | | TCTCATGCAAAGCGTCCGGGTACACCTTCACGTCCTATAACA | |
| | | TGCATTGGGTTCGGCAGGCGCCTGGCCAGGGGCTCGAGTGG | |
| | | ATGGGCGTCATCTACTCCGGAAATGGCGATACCAGTTACAAT | |
| | | CAGAAGTTTAAGGGCCGAGTAACCATTACCGCTGACAAAAG | |
| | | TACTTCAACCGCTTACATGGAACTGTCATCACTTAGGTCAGA | |
| | | AGACACGGCAGTTTATTACTGCGCGCGCGAGAGGGACACAA | |
| | | GGTTTGGCAATTGGGGGCAAGGCACACTCGTTACTGTAAGTA | |
| | | GTAGTGCTGCTGCCTTTGTCCCGGTATTTCTCCCAGCCAAACC | |
| | | GACCACGACTCCCGCCCCGCGCCCTCCGACACCCGCTCCCAC | |
| | | CATCGCCTCTCAACCTCTTAGTCTTCGCCCCGAGGCATGCCG | |
| | | ACCCGCCGCCGGGGGTGCTGTTCATACGAGGGGCTTGGACTT | |
| | | CGCTTGTGATATTTACATTTGGGCTCCGTTGGCGGGTACGTG | |
| | | CGGCGTCCTTTTGTTGTCACTCGTTATTACTTTGTATTGTAAT | |
| | | CACAGGAATCGCTCAAAGCGGAGTAGGTTGTTGCATTCCGAT | |
| | | TACATGAATATGACTCCTCGCCGGCCTGGGCCGACAAGAAA | |
| | | ACATTACCAACCCTATGCCCCCCCACGAGACTTCGCTGCGTA | |
| | | CAGGTCCCGAGTGAAGTTTTCCCGAAGCGCAGACGCTCCGGC | |
| | | ATATCAGCAAGGACAGAATCAGCTGTATAACGAACTGAATT | |
| | | TGGGACGCCGCGAGGAGTATGACGTGCTTGATAAACGCCGG | |
| | | GGGAGAGACCCGGAAATGGGGGGTAAACCCCGAAGAAAGA | |
| | | ATCCCCAAGAAGGACTCTACAATGAACTCCAGAAGGATAAG | |
| | | ATGGCGGAGGCCTACTCAGAAATAGGTATGAAGGGCGAACG | |
| | | ACGACGGGGAAAAGGTCACGATGGCCTCTACCAAGGGTTGA | |
| | | GTACGGCAACCAAAGATACGTACGATGCACTGCATATGCAG | |
| | | GCCCTGCCTCCCAGA | |
| CTX-2841 | | MALPVTALLLPLALLLHAARPDIVMTQSPDSLAVSLGERATINCR | 114 |
| (Ab-1, VL/VH; and | | ASESVDIYGNSFMHWYQQKPGQPPKLLIYLASNLESGVPDRFSG | |
| 4-1B CoS with | | SGSGTDFTLTISSLQAEDVAVYYCQQNNEDPYTFGGGTKVEIK | |
| signal peptide) | | GGGGSGGGGSGGGGSQVQLVQSGAEVKKPGASVKVSCKASGY | |
| | | TFTSYNMHWVRQAPGQGLEWMGVIYSGNGDTSYNQKFKGR | |
| | | VTITADKSTSTAYMELSSLRSEDTAVYYCARERDTRFGNWGQ | |
| | | GTLVTVSSSAAAFVPVFLPAKPTTTPAPRPPTPAPTIASQPLSLRP | |
| | | EACRPAAGGAVHTRGLDFACDIYIWAPLAGTCGVLLLSLVITLY | |
| | | CNHRNRKRGRKKLLYIFKQPFMRPVQTTQEEDGCSCRFPEEEEG | |
| | | GCELRVKFSRSADAPAYQQGQNQLYNELNLGRREEYDVLDKR | |
| | | RGRDPEMGGKPRRKNPQEGLYNELQKDKMAEAYSEIGMKGER | |
| | | RRGKGHDGLYQGLSTATKDTYDALHMQALPPR | |
| CTX-2841 | | DIVMTQSPDSLAVSLGERATINCRASESVDIYGNSFMHWYQQK | 115 |
| (no signal peptide) | | PGQPPKLLIYLASNLESGVPDRFSGSGSGTDFTLTISSLQAEDVA | |
| | | VYYCQQNNEDPYTFGGGTKVEIKGGGGSGGGGSGGGGSQVQL | |
| | | VQSGAEVKKPGASVKVSCKASGYTFTSYNMHWVRQAPGQGL | |
| | | EWMGVIYSGNGDTSYNQKFKGRVTITADKSTSTAYMELSSLR | |
| | | SEDTAVYYCARERDTRFGNWGQGTLVTVSSSAAAFVPVFLPA | |
| | | KPTTTPAPRPPTPAPTIASQPLSLRPEACRPAAGGAVHTRGLDFA | |
| | | CDIYIWAPLAGTCGVLLLSLVITLYCNHRNRKRGRKKLLYIFKQ | |
| | | PFMRPVQTTQEEDGCSCRFPEEEEGGCELRVKFSRSADAPAYQQ | |
| | | GQNQLYNELNLGRREEYDVLDKRRGRDPEMGGKPRRKNPQEG | |
| | | LYNELQKDKMAEAYSEIGMKGERRRGKGHDGLYQGLSTATKD | |
| | | TYDALHMQALPPR | |
| CTX-2841 coding | | ATGGCGCTTCCGGTGACAGCACTGCTCCTCCCCTTGGCGCTG | 116 |
| | | TTGCTCCACGCAGCAAGGCCGGACATAGTTATGACCCAATCA | |
| | | CCAGACTCACTCGCGGTCAGCTTGGGGGAGCGCGCCACAATT | |
| | | AACTGTCGCGCGTCTGAGTCTGTCGACATCTACGGTAATTCT | |
| | | TTTATGCATTGGTATCAACAAAAGCCAGGACAGCCGCCGAA | |
| | | ATTGTTGATATATCTCGCTAGCAACCTTGAGTCAGGTGTGCC | |
| | | TGATCGGTTCAGTGGTTCTGGGTCAGGGACCGACTTTACGCT | |
| | | GACAATTAGTTCTCTGCAAGCAGAAGATGTGGCAGTGTATTA | |
| | | TTGCCAGCAAAATAATGAGGACCCCTACACCTTCGGGGGTG | |
| | | GCACGAAAGTCGAAATTAAAGGGGGAGGCGGCAGTGGCGGT | |
| | | GGCGGTAGCGGAGGTGGTGGGTCTCAGGTCCAACTGGTTCA | |
| | | ATCCGGAGCGGAAGTTAAGAAGCCAGGTGCAAGTGTCAAAG | |
| | | TCTCATGCAAAGCGTCCGGGTACACCTTCACGTCCTATAACA | |
| | | TGCATTGGGTTCGGCAGGCGCCTGGCCAGGGGCTCGAGTGG | |
| | | ATGGGCGTCATCTACTCCGGAAATGGCGATACCAGTTACAAT | |
| | | CAGAAGTTTAAGGGCCGAGTAACCATTACCGCTGACAAAAG | |
| | | TACTTCAACCGCTTACATGGAACTGTCATCACTTAGGTCAGA | |
| | | AGACACGGCAGTTTATTACTGCGCGCGCGAGAGGGACACAA | |
| | | GGTTTGGCAATTGGGGGCAAGGCACACTCGTTACTGTAAGTA | |
| | | GTAGTGCTGCTGCCTTTGTCCCGGTATTTCTCCCAGCCAAACC | |
| | | GACCACGACTCCCGCCCCGCGCCCTCCGACACCCGCTCCCAC | |
| | | CATCGCCTCTCAACCTCTTAGTCTTCGCCCCGAGGCATGCCG | |
| | | ACCCGCCGCCGGGGGTGCTGTTCATACGAGGGGCTTGGACTT | |
| | | CGCTTGTGATATTTACATTTGGGCTCCGTTGGCGGGTACGTG | |
| | | CGGCGTCCTTTTGTTGTCACTCGTTATTACTTTGTATTGTAAT | |
| | | CACAGGAATCGCAAACGGGGCAGAAAGAAACTCCTGTATAT | |
| | | ATTCAAACAACCATTTATGAGACCAGTACAAACTACTCAAGA | |
| | | GGAAGATGGCTGTAGCTGCCGATTTCCAGAAGAAGAAGAAG | |
| | | GAGGATGTGAACTGCGAGTGAAGTTTTCCCGAAGCGCAGAC | |
| | | GCTCCGGCATATCAGCAAGGACAGAATCAGCTGTATAACGA | |
| | | ACTGAATTTGGGACGCCGCGAGGAGTATGACGTGCTTGATA | |
| | | AACGCCGGGGGAGAGACCCGGAAATGGGGGGTAAACCCCG | |
| | | AAGAAAGAATCCCCAAGAAGGACTCTACAATGAACTCCAGA | |
| | | AGGATAAGATGGCGGAGGCCTACTCAGAAATAGGTATGAAG | |
| | | GGCGAACGACGACGGGGAAAAGGTCACGATGGCCTCTACCA | |
| | | AGGGTTGAGTACGGCAACCAAAGATACGTACGATGCACTGC | |
| | | ATATGCAGGCCCTGCCTCCCAGA | |
| CTX-2842 | | MALPVTALLLPLALLLHAARPQVQLVQSGAEVKKPGASVKVSCK | 117 |
| (Ab-1 VH/VL; | | ASGYTFTSYNMHWVRQAPGQGLEWMGVIYSGNGDTSYNQKF | |
| CD28 CoS with | | KGRVTITADKSTSTAYMELSSLRSEDTAVYYCARERDTREGN | |
| signal peptide) | | WGQGTLVTVSSGGGGSGGGGSGGGGSDIVMTQSPDSLAVSLGE | |
| | | RATINCRASESVDIYGNSFMHWYQQKPGQPPKLLIYLASNLES | |
| | | GVPDRFSGSGSGTDFTLTISSLQAEDVAVYYCQQNNEDPYTFG | |
| | | GGTKVEIKSAAAFVPVFLPAKPTTTPAPRPPTPAPTIASQPLSLRP | |
| | | EACRPAAGGAVHTRGLDFACDIYIWAPLAGTCGVLLLSLVITLY | |
| | | CNHRNRSKRSRLLHSDYMNMTPRRPGPTRKHYQPYAPPRDFAA | |
| | | YRSRVKFSRSADAPAYQQGQNQLYNELNLGRREEYDVLDKRR | |
| | | GRDPEMGGKPRRKNPQEGLYNELQKDKMAEAYSEIGMKGERR | |
| | | RGKGHDGLYQGLSTATKDTYDALHMQALPPR | |
| CTX-2842 | | QVQLVQSGAEVKKPGASVKVSCKASGYTFTSYNMHWVRQAP | 118 |
| (no signal peptide) | | GQGLEWMGVIYSGNGDTSYNQKFKGRVTITADKSTSTAYMEL | |
| | | SSLRSEDTAVYYCARERDTRFGNWGQGTLVTVSSGGGGSGGG | |
| | | GSGGGGSDIVMTQSPDSLAVSLGERATINCRASESVDIYGNSFM | |
| | | HWYQQKPGQPPKLLIYLASNLESGVPDRFSGSGSGTDFTLTISS | |
| | | LQAEDVAVYYCQQNNEDPYTFGGGTKVEIKSAAAFVPVFLPA | |
| | | KPTTTPAPRPPTPAPTIASQPLSLRPEACRPAAGGAVHTRGLDFA | |
| | | CDIYIWAPLAGTCGVLLLSLVITLYCNHRNRSKRSRLLHSDYMN | |
| | | MTPRRPGPTRKHYQPYAPPRDFAAYRSRVKFSRSADAPAYQQG | |
| | | QNQLYNELNLGRREEYDVLDKRRGRDPEMGGKPRRKNPQEGL | |
| | | YNELQKDKMAEAYSEIGMKGERRRGKGHDGLYQGLSTATKDT | |
| | | YDALHMQALPPR | |
| CTX-2842 coding | | ATGGCGCTTCCGGTGACAGCACTGCTCCTCCCCTTGGCGCTG | 119 |
| | | TTGCTCCACGCAGCAAGGCCGCAGGTTCAGCTTGTCCAATCA | |
| | | GGAGCAGAGGTGAAAAAGCCAGGAGCGAGCGTCAAAGTAA | |
| | | GTTGCAAAGCCAGCGGGTACACATTTACCAGCTACAACATGC | |
| | | ACTGGGTAAGACAAGCGCCAGGACAAGGCCTCGAGTGGATG | |
| | | GGCGTGATATATAGCGGTAACGGTGACACCAGTTATAACCA | |
| | | GAAATTCAAGGGTCGGGTCACAATAACCGCCGATAAAAGCA | |
| | | CTTCTACAGCATATATGGAGCTCTCATCCCTGAGATCAGAAG | |
| | | ACACTGCGGTGTATTACTGCGCGCGCGAGAGGGATACAAGG | |
| | | TTCGGCAATTGGGGCCAAGGTACTTTGGTCACCGTGAGTAGC | |
| | | GGTGGTGGTGGTAGTGGTGGCGGTGGCAGTGGTGGGGGTGG | |
| | | TTCAGACATCGTAATGACCCAATCACCTGATAGCCTGGCAGT | |
| | | AAGCCTCGGCGAACGGGCCACGATAAACTGTAGAGCATCCG | |
| | | AGTCAGTGGACATTTATGGCAATTCTTTCATGCATTGGTACC | |
| | | AGCAAAAGCCAGGTCAGCCCCCGAAATTGCTTATCTATCTTG | |
| | | CGAGTAATCTTGAATCAGGGGTACCCGACCGCTTCTCTGGCT | |
| | | CTGGCAGTGGCACTGATTTTACCCTGACGATCTCCAGCCTCC | |
| | | AAGCTGAAGACGTGGCCGTATATTACTGCCAGCAGAATAAC | |
| | | GAGGATCCCTATACGTTCGGAGGTGGTACGAAGGTAGAGAT | |
| | | AAAAAGTGCTGCTGCCTTTGTCCCGGTATTTCTCCCAGCCAA | |
| | | ACCGACCACGACTCCCGCCCCGCGCCCTCCGACACCCGCTCC | |
| | | CACCATCGCCTCTCAACCTCTTAGTCTTCGCCCCGAGGCATG | |
| | | CCGACCCGCCGCCGGGGGTGCTGTTCATACGAGGGGCTTGG | |
| | | ACTTCGCTTGTGATATTTACATTTGGGCTCCGTTGGCGGGTAC | |
| | | GTGCGGCGTCCTTTTGTTGTCACTCGTTATTACTTTGTATTGT | |
| | | AATCACAGGAATCGCTCAAAGCGGAGTAGGTTGTTGCATTCC | |
| | | GATTACATGAATATGACTCCTCGCCGGCCTGGGCCGACAAGA | |
| | | AAACATTACCAACCCTATGCCCCCCCACGAGACTTCGCTGCG | |
| | | TACAGGTCCCGAGTGAAGTTTTCCCGAAGCGCAGACGCTCCG | |
| | | GCATATCAGCAAGGACAGAATCAGCTGTATAACGAACTGAA | |
| | | TTTGGGACGCCGCGAGGAGTATGACGTGCTTGATAAACGCC | |
| | | GGGGGAGAGACCCGGAAATGGGGGGTAAACCCCGAAGAAA | |
| | | GAATCCCCAAGAAGGACTCTACAATGAACTCCAGAAGGATA | |
| | | AGATGGCGGAGGCCTACTCAGAAATAGGTATGAAGGGCGAA | |
| | | CGACGACGGGGAAAAGGTCACGATGGCCTCTACCAAGGGTT | |
| | | GAGTACGGCAACCAAAGATACGTACGATGCACTGCATATGC | |
| | | AGGCCCTGCCTCCCAGA | |
| CTX-2843 | | MALPVTALLLPLALLLHAARPQVQLVQSGAEVKKPGASVKVSCK | 120 |
| (Ab-1 VH/VL; and | | ASGYTFTSYNMHWVRQAPGQGLEWMGVIYSGNGDTSYNQKF | |
| 4-1B CoS with | | KGRVTITADKSTSTAYMELSSLRSEDTAVYYCARERDTREGN | |
| signal peptide) | | WGQGTLVTVSSGGGGSGGGGSGGGGSDIVMTQSPDSLAVSLGE | |
| | | RATINCRASESVDIYGNSFMHWYQQKPGQPPKLLIYLASNLES | |
| | | GVPDRFSGSGSGTDFTLTISSLQAEDVAVYYCQQNNEDPYTFG | |
| | | GGTKVEIKSAAAFVPVFLPAKPTTTPAPRPPTPAPTIASQPLSLRP | |
| | | EACRPAAGGAVHTRGLDFACDIYIWAPLAGTCGVLLLSLVITLY | |
| | | CNHRNRKRGRKKLLYIFKQPFMRPVQTTQEEDGCSCRFPEEEEG | |
| | | GCELRVKFSRSADAPAYQQGQNQLYNELNLGRREEYDVLDKR | |
| | | RGRDPEMGGKPRRKNPQEGLYNELQKDKMAEAYSEIGMKGER | |
| | | RRGKGHDGLYQGLSTATKDTYDALHMQALPPR | |
| CTX-2843 | | QVQLVQSGAEVKKPGASVKVSCKASGYTFTSYNMHWVRQAP | 121 |
| (no signal peptide) | | GQGLEWMGVIYSGNGDTSYNQKFKGRVTITADKSTSTAYMEL | |
| | | SSLRSEDTAVYYCARERDTREGNWGQGTLVTVSSGGGGSGGG | |
| | | GSGGGGSDIVMTQSPDSLAVSLGERATINCRASESVDIYGNSFM | |
| | | HWYQQKPGQPPKLLIYLASNLESGVPDRESGSGSGTDFTLTISS | |
| | | LQAEDVAVYYCQQNNEDPYTFGGGTKVEIKSAAAFVPVFLPA | |
| | | KPTTTPAPRPPTPAPTIASQPLSLRPEACRPAAGGAVHTRGLDFA | |
| | | CDIYIWAPLAGTCGVLLLSLVITLYCNHRNRKRGRKKLLYIFKQ | |
| | | PFMRPVQTTQEEDGCSCRFPEEEEGGCELRVKFSRSADAPAYQQ | |
| | | GQNQLYNELNLGRREEYDVLDKRRGRDPEMGGKPRRKNPQEG | |
| | | LYNELQKDKMAEAYSEIGMKGERRRGKGHDGLYQGLSTATKD | |
| | | TYDALHMQALPPR | |
| CTX-2843 coding | | ATGGCGCTTCCGGTGACAGCACTGCTCCTCCCCTTGGCGCTG | 122 |
| | | TTGCTCCACGCAGCAAGGCCGCAGGTTCAGCTTGTCCAATCA | |
| | | GGAGCAGAGGTGAAAAAGCCAGGAGCGAGCGTCAAAGTAA | |
| | | GTTGCAAAGCCAGCGGGTACACATTTACCAGCTACAACATGC | |
| | | ACTGGGTAAGACAAGCGCCAGGACAAGGCCTCGAGTGGATG | |
| | | GGCGTGATATATAGCGGTAACGGTGACACCAGTTATAACCA | |
| | | GAAATTCAAGGGTCGGGTCACAATAACCGCCGATAAAAGCA | |
| | | CTTCTACAGCATATATGGAGCTCTCATCCCTGAGATCAGAAG | |
| | | ACACTGCGGTGTATTACTGCGCGCGCGAGAGGGATACAAGG | |
| | | TTCGGCAATTGGGGCCAAGGTACTTTGGTCACCGTGAGTAGC | |
| | | GGTGGTGGTGGTAGTGGTGGCGGTGGCAGTGGTGGGGGTGG | |
| | | TTCAGACATCGTAATGACCCAATCACCTGATAGCCTGGCAGT | |
| | | AAGCCTCGGCGAACGGGCCACGATAAACTGTAGAGCATCCG | |
| | | AGTCAGTGGACATTTATGGCAATTCTTTCATGCATTGGTACC | |
| | | AGCAAAAGCCAGGTCAGCCCCCGAAATTGCTTATCTATCTTG | |
| | | CGAGTAATCTTGAATCAGGGGTACCCGACCGCTTCTCTGGCT | |
| | | CTGGCAGTGGCACTGATTTTACCCTGACGATCTCCAGCCTCC | |
| | | AAGCTGAAGACGTGGCCGTATATTACTGCCAGCAGAATAAC | |
| | | GAGGATCCCTATACGTTCGGAGGTGGTACGAAGGTAGAGAT | |
| | | AAAAAGTGCTGCTGCCTTTGTCCCGGTATTTCTCCCAGCCAA | |
| | | ACCGACCACGACTCCCGCCCCGCGCCCTCCGACACCCGCTCC | |
| | | CACCATCGCCTCTCAACCTCTTAGTCTTCGCCCCGAGGCATG | |
| | | CCGACCCGCCGCCGGGGGTGCTGTTCATACGAGGGGCTTGG | |
| | | ACTTCGCTTGTGATATTTACATTTGGGCTCCGTTGGCGGGTAC | |
| | | GTGCGGCGTCCTTTTGTTGTCACTCGTTATTACTTTGTATTGT | |
| | | AATCACAGGAATCGCAAACGGGGCAGAAAGAAACTCCTGTA | |
| | | TATATTCAAACAACCATTTATGAGACCAGTACAAACTACTCA | |
| | | AGAGGAAGATGGCTGTAGCTGCCGATTTCCAGAAGAAGAAG | |
| | | AAGGAGGATGTGAACTGCGAGTGAAGTTTTCCCGAAGCGCA | |
| | | GACGCTCCGGCATATCAGCAAGGACAGAATCAGCTGTATAA | |
| | | CGAACTGAATTTGGGACGCCGCGAGGAGTATGACGTGCTTG | |
| | | ATAAACGCCGGGGGAGAGACCCGGAAATGGGGGGTAAACCC | |
| | | CGAAGAAAGAATCCCCAAGAAGGACTCTACAATGAACTCCA | |
| | | GAAGGATAAGATGGCGGAGGCCTACTCAGAAATAGGTATGA | |
| | | AGGGCGAACGACGACGGGGAAAAGGTCACGATGGCCTCTAC | |
| | | CAAGGGTTGAGTACGGCAACCAAAGATACGTACGATGCACT | |
| | | GCATATGCAGGCCCTGCCTCCCAGA | |
| Anti- | LCDR1 | RASQGISSALA | 123 |
| CD117 | LCDR2 | DASSLES | 124 |
| Ab-2 | LCDR3 | QQFNSYPLT | 125 |
| | HCDR1 | SYWIG | 126 |
| | HCDR2 | IIYPGDSDTRYSPSFQG | 127 |
| | HCDR3 | HGRGYNGYEGAFDI | 128 |
| | VL | AIQLTQSPSSLSASVGDRVTITCRASQGISSALAWYQQKPGKAP | 129 |
| | | KLLIYDASSLESGVPSRFSGSGSGTDFTLTISSLQPEDFATYYC | |
| | | QQFNSYPLTFGGGTKVEIK | |
| | VH | QVQLVQSGAAVKKPGESLKISCKGSGYRFTSYWIGWVRQMPGK | 130 |
| | | GLEWMGIIYPGDSDTRYSPSFQGQVTISAGKSISTAYLQWSSL | |
| | | KASDTAMYYCARHGRGYNGYEGAFDIWGQGTMVTVSS | |
| | scFv | AIQLTQSPSSLSASVGDRVTITCRASQGISSALAWYQQKPGKAP | 131 |
| | (VL/VH) | KLLIYDASSLESGVPSRFSGSGSGTDFTLTISSLQPEDFATYYC | |
| | Linker | QQFNSYPLTFGGGTKVEIKGGGGSGGGGSGGGGSQVQLVQSGAAV | |
| | underlined | KKPGESLKISCKGSGYRFTSYWIGWVRQMPGKGLEWMGIIYPG | |
| | | DSDTRYSPSFQGQVTISAGKSISTAYLQWSSLKASDTAMYYCAR | |
| | | HGRGYNGYEGAFDIWGQGTMVTVSS | |
| | scFv | QVQLVQSGAAVKKPGESLKISCKGSGYRFTSYWIGWVRQMPG | 132 |
| | (VH/VL) | KGLEWMGIIYPGDSDTRYSPSFQGQVTISAGKSISTAYLQWSSL | |
| | Linker | KASDTAMYYCARHGRGYNGYEGAFDIWGQGTMVTVSSGGGG | |
| | underlined | SGGGGSGGGGSAIQLTQSPSSLSASVGDRVTITCRASQGISSALA | |
| | | WYQQKPGKAPKLLIYDASSLESGVPSRFSGSGSGTDFTLTISSLQ | |
| | | PEDFATYYCQQFNSYPLTFGGGTKVEIK | |
| CTX-2844 | | MALPVTALLLPLALLLHAARPAIQLTQSPSSLSASVGDRVTITCR | 133 |
| (Ab-2 VL/VH; and | | ASQGISSALAWYQQKPGKAPKLLIYDASSLESGVPSRFSGSGSGT | |
| CD28 CoS with | | DFTLTISSLQPEDFATYYCQQFNSYPLTFGGGTKVEIKGGGGSG | |
| signal peptide) | | GGGSGGGGSQVQLVQSGAAVKKPGESLKISCKGSGYRFTSYWI | |
| | | GWVRQMPGKGLEWMGIIYPGDSDTRYSPSFQGQVTISAGKSIS | |
| | | TAYLQWSSLKASDTAMYYCARHGRGYNGYEGAFDIWGQGTMVT | |
| | | VSSSAAAFVPVFLPAKPTTTPAPRPPTPAPTIASQPLSLRPEA | |
| | | CRPAAGGAVHTRGLDFACDIYIWAPLAGTCGVLLLSLVITLYCN | |
| | | HRNRSKRSRLLHSDYMNMTPRRPGPTRKHYQPYAPPRDFAAYR | |
| | | SRVKFSRSADAPAYQQGQNQLYNELNLGRREEYDVLDKRRGR | |
| | | DPEMGGKPRRKNPQEGLYNELQKDKMAEAYSEIGMKGERRRG | |
| | | KGHDGLYQGLSTATKDTYDALHMQALPPR | |
| CTX-2844 | | AIQLTQSPSSLSASVGDRVTITCRASQGISSALAWYQQKPGKAP | 134 |
| (no signal peptide) | | KLLIYDASSLESGVPSRFSGSGSGTDFTLTISSLQPEDFATYYC | |
| | | QQFNSYPLTFGGGTKVEIKGGGGSGGGGSGGGGSQVQLVQSGA | |
| | | AVKKPGESLKISCKGSGYRFTSYWIGWVRQMPGKGLEWMGII | |
| | | YPGDSDTRYSPSFQGQVTISAGKSISTAYLQWSSLKASDTAMY | |
| | | YCARHGRGYNGYEGAFDIWGQGTMVTVSSSAAAFVPVFLPAKP | |
| | | TTTPAPRPPTPAPTIASQPLSLRPEACRPAAGGAVHTRGLDFA | |
| | | CDIYIWAPLAGTCGVLLLSLVITLYCNHRNRSKRSRLLHSDYMN | |
| | | MTPRRPGPTRKHYQPYAPPRDFAAYRSRVKFSRSADAPAYQQG | |
| | | QNQLYNELNLGRREEYDVLDKRRGRDPEMGGKPRRKNPQEGL | |
| | | YNELQKDKMAEAYSEIGMKGERRRGKGHDGLYQGLSTATKDT | |
| | | YDALHMQALPPR | |
| CTX-2844 coding | | ATGGCGCTTCCGGTGACAGCACTGCTCCTCCCCTTGGCGCTG | 135 |
| | | TTGCTCCACGCAGCAAGGCCGGCCATACAACTGACTCAAAG | |
| | | CCCCTCATCTCTGTCAGCATCAGTGGGCGACCGAGTTACTAT | |
| | | TACGTGCCGCGCCAGTCAAGGTATTTCCAGCGCTCTTGCTTG | |
| | | GTATCAGCAAAAGCCCGGTAAGGCCCCCAAACTCCTGATAT | |
| | | ACGACGCCAGTTCATTGGAGTCCGGAGTACCGTCACGGTTCT | |
| | | CTGGATCTGGGTCAGGCACCGACTTTACGCTTACAATTAGCT | |
| | | CACTCCAACCGGAAGATTTTGCTACCTACTATTGCCAGCAGT | |
| | | TCAATTCATACCCACTTACGTTTGGGGGAGGAACGAAAGTAG | |
| | | AAATTAAAGGAGGTGGCGGGAGTGGTGGCGGTGGTTCTGGC | |
| | | GGCGGCGGTTCTCAGGTTCAGCTTGTGCAATCAGGGGCTGCG | |
| | | GTAAAAAAACCCGGGGAATCACTTAAGATTTCCTGTAAAGG | |
| | | CTCAGGGTATCGATTCACTTCATATTGGATAGGGTGGGTTCG | |
| | | CCAGATGCCAGGAAAAGGGCTGGAGTGGATGGGCATAATTT | |
| | | ACCCAGGAGACAGCGATACCAGATATAGTCCGTCCTTTCAGG | |
| | | GCCAGGTTACAATTAGCGCCGGGAAGAGTATCAGCACAGCG | |
| | | TACCTGCAATGGTCATCCCTCAAAGCCTCAGATACAGCAATG | |
| | | TATTACTGTGCGCGACACGGCAGGGGTTACAATGGGTACGA | |
| | | AGGGGCTTTCGATATTTGGGGGCAGGGCACCATGGTTACGGT | |
| | | CAGCTCAAGTGCTGCTGCCTTTGTCCCGGTATTTCTCCCAGCC | |
| | | AAACCGACCACGACTCCCGCCCCGCGCCCTCCGACACCCGCT | |
| | | CCCACCATCGCCTCTCAACCTCTTAGTCTTCGCCCCGAGGCA | |
| | | TGCCGACCCGCCGCCGGGGGTGCTGTTCATACGAGGGGCTTG | |
| | | GACTTCGCTTGTGATATTTACATTTGGGCTCCGTTGGCGGGT | |
| | | ACGTGCGGCGTCCTTTTGTTGTCACTCGTTATTACTTTGTATT | |
| | | GTAATCACAGGAATCGCTCAAAGCGGAGTAGGTTGTTGCATT | |
| | | CCGATTACATGAATATGACTCCTCGCCGGCCTGGGCCGACAA | |
| | | GAAAACATTACCAACCCTATGCCCCCCCACGAGACTTCGCTG | |
| | | CGTACAGGTCCCGAGTGAAGTTTTCCCGAAGCGCAGACGCTC | |
| | | CGGCATATCAGCAAGGACAGAATCAGCTGTATAACGAACTG | |
| | | AATTTGGGACGCCGCGAGGAGTATGACGTGCTTGATAAACG | |
| | | CCGGGGGAGAGACCCGGAAATGGGGGGTAAACCCCGAAGA | |
| | | AAGAATCCCCAAGAAGGACTCTACAATGAACTCCAGAAGGA | |
| | | TAAGATGGCGGAGGCCTACTCAGAAATAGGTATGAAGGGCG | |
| | | AACGACGACGGGGAAAAGGTCACGATGGCCTCTACCAAGGG | |
| | | TTGAGTACGGCAACCAAAGATACGTACGATGCACTGCATAT | |
| | | GCAGGCCCTGCCTCCCAGA | |
| CTX-2845 | | MALPVTALLLPLALLLHAARPAIQLTQSPSSLSASVGDRVTITCR | 136 |
| (Ab-2 VL/VH; and | | ASQGISSALAWYQQKPGKAPKLLIYDASSLESGVPSRFSGSGSGT | |
| 4-1BB CoS with | | DFTLTISSLQPEDFATYYCQQFNSYPLTFGGGTKVEIKGGGGSG | |
| signal peptide) | | GGGSGGGGSQVQLVQSGAAVKKPGESLKISCKGSGYRFTSYWI | |
| | | GWVRQMPGKGLEWMGIIYPGDSDTRYSPSFQGQVTISAGKSIS | |
| | | TAYLQWSSLKASDTAMYYCARHGRGYNGYEGAFDIWGQGTMVT | |
| | | VSSSAAAFVPVFLPAKPTTTPAPRPPTPAPTIASQPLSLRPEA | |
| | | CRPAAGGAVHTRGLDFACDIYIWAPLAGTCGVLLLSLVITLYCN | |
| | | HRNRKRGRKKLLYIFKQPFMRPVQTTQEEDGCSCRFPEEEEGGC | |
| | | ELRVKFSRSADAPAYQQGQNQLYNELNLGRREEYDVLDKRRG | |
| | | RDPEMGGKPRRKNPQEGLYNELQKDKMAEAYSEIGMKGERRR | |
| | | GKGHDGLYQGLSTATKDTYDALHMQALPPR | |
| CTX-2845 | | AIQLTQSPSSLSASVGDRVTITCRASQGISSALAWYQQKPGKAP | 137 |
| (no signal peptide) | | KLLIYDASSLESGVPSRFSGSGSGTDFTLTISSLQPEDFATYYC | |
| | | QQFNSYPLTFGGGTKVEIKGGGGSGGGGSGGGGSQVQLVQSGA | |
| | | AVKKPGESLKISCKGSGYRFTSYWIGWVRQMPGKGLEWMGII | |
| | | YPGDSDTRYSPSFQGQVTISAGKSISTAYLQWSSLKASDTAMY | |
| | | YCARHGRGYNGYEGAFDIWGQGTMVTVSSSAAAFVPVFLPAK | |
| | | PTTTPAPRPPTPAPTIASQPLSLRPEACRPAAGGAVHTRGLDFA | |
| | | CDIYIWAPLAGTCGVLLLSLVITLYCNHRNRKRGRKKLLYIFKQ | |
| | | PFMRPVQTTQEEDGCSCRFPEEEEGGCELRVKFSRSADAPAYQQ | |
| | | GQNQLYNELNLGRREEYDVLDKRRGRDPEMGGKPRRKNPQEG | |
| | | LYNELQKDKMAEAYSEIGMKGERRRGKGHDGLYQGLSTATKD | |
| | | TYDALHMQALPPR | |
| CTX-2845 coding | | ATGGCGCTTCCGGTGACAGCACTGCTCCTCCCCTTGGCGCTG | 138 |
| | | TTGCTCCACGCAGCAAGGCCGGCCATACAACTGACTCAAAG | |
| | | CCCCTCATCTCTGTCAGCATCAGTGGGCGACCGAGTTACTAT | |
| | | TACGTGCCGCGCCAGTCAAGGTATTTCCAGCGCTCTTGCTTG | |
| | | GTATCAGCAAAAGCCCGGTAAGGCCCCCAAACTCCTGATAT | |
| | | ACGACGCCAGTTCATTGGAGTCCGGAGTACCGTCACGGTTCT | |
| | | CTGGATCTGGGTCAGGCACCGACTTTACGCTTACAATTAGCT | |
| | | CACTCCAACCGGAAGATTTTGCTACCTACTATTGCCAGCAGT | |
| | | TCAATTCATACCCACTTACGTTTGGGGGAGGAACGAAAGTAG | |
| | | AAATTAAAGGAGGTGGCGGGAGTGGTGGCGGTGGTTCTGGC | |
| | | GGCGGCGGTTCTCAGGTTCAGCTTGTGCAATCAGGGGCTGCG | |
| | | GTAAAAAAACCCGGGGAATCACTTAAGATTTCCTGTAAAGG | |
| | | CTCAGGGTATCGATTCACTTCATATTGGATAGGGTGGGTTCG | |
| | | CCAGATGCCAGGAAAAGGGCTGGAGTGGATGGGCATAATTT | |
| | | ACCCAGGAGACAGCGATACCAGATATAGTCCGTCCTTTCAGG | |
| | | GCCAGGTTACAATTAGCGCCGGGAAGAGTATCAGCACAGCG | |
| | | TACCTGCAATGGTCATCCCTCAAAGCCTCAGATACAGCAATG | |
| | | TATTACTGTGCGCGACACGGCAGGGGTTACAATGGGTACGA | |
| | | AGGGGCTTTCGATATTTGGGGGCAGGGCACCATGGTTACGGT | |
| | | CAGCTCAAGTGCTGCTGCCTTTGTCCCGGTATTTCTCCCAGCC | |
| | | AAACCGACCACGACTCCCGCCCCGCGCCCTCCGACACCCGCT | |
| | | CCCACCATCGCCTCTCAACCTCTTAGTCTTCGCCCCGAGGCA | |
| | | TGCCGACCCGCCGCCGGGGGTGCTGTTCATACGAGGGGCTTG | |
| | | GACTTCGCTTGTGATATTTACATTTGGGCTCCGTTGGCGGGT | |
| | | ACGTGCGGCGTCCTTTTGTTGTCACTCGTTATTACTTTGTATT | |
| | | GTAATCACAGGAATCGCAAACGGGGCAGAAAGAAACTCCTG | |
| | | TATATATTCAAACAACCATTTATGAGACCAGTACAAACTACT | |
| | | CAAGAGGAAGATGGCTGTAGCTGCCGATTTCCAGAAGAAGA | |
| | | AGAAGGAGGATGTGAACTGCGAGTGAAGTTTTCCCGAAGCG | |
| | | CAGACGCTCCGGCATATCAGCAAGGACAGAATCAGCTGTAT | |
| | | AACGAACTGAATTTGGGACGCCGCGAGGAGTATGACGTGCT | |
| | | TGATAAACGCCGGGGGAGAGACCCGGAAATGGGGGGTAAAC | |
| | | CCCGAAGAAAGAATCCCCAAGAAGGACTCTACAATGAACTC | |
| | | CAGAAGGATAAGATGGCGGAGGCCTACTCAGAAATAGGTAT | |
| | | GAAGGGCGAACGACGACGGGGAAAAGGTCACGATGGCCTCT | |
| | | ACCAAGGGTTGAGTACGGCAACCAAAGATACGTACGATGCA | |
| | | CTGCATATGCAGGCCCTGCCTCCCAGA | |
| CTX-2846 | | MALPVTALLLPLALLLHAARPQVQLVQSGAAVKKPGESLKISCKG | 139 |
| (Ab-2 VH/VL; and | | SGYRFTSYWIGWVRQMPGKGLEWMGIIYPGDSDTRYSPSFQG | |
| CD28 CoS with | | QVTISAGKSISTAYLQWSSLKASDTAMYYCARHGRGYNGYEG | |
| signal peptide) | | AFDIWGQGTMVTVSSGGGGSGGGGSGGGGSAIQLTQSPSSLSA | |
| | | SVGDRVTITCRASQGISSALAWYQQKPGKAPKLLIYDASSLESG | |
| | | VPSRFSGSGSGTDFTLTISSLQPEDFATYYCQQFNSYPLTFGGGT | |
| | | KVEIKSAAAFVPVFLPAKPTTTPAPRPPTPAPTIASQPLSLRPEAC | |
| | | RPAAGGAVHTRGLDFACDIYIWAPLAGTCGVLLLSLVITLYCNH | |
| | | RNRSKRSRLLHSDYMNMTPRRPGPTRKHYQPYAPPRDFAAYRS | |
| | | RVKFSRSADAPAYQQGQNQLYNELNLGRREEYDVLDKRRGRD | |
| | | PEMGGKPRRKNPQEGLYNELQKDKMAEAYSEIGMKGERRRGK | |
| | | GHDGLYQGLSTATKDTYDALHMQALPPR | |
| CTX-2846 | | QVQLVQSGAAVKKPGESLKISCKGSGYRFTSYWIGWVRQMPG | 140 |
| (no signal peptide) | | KGLEWMGIIYPGDSDTRYSPSFQGQVTISAGKSISTAYLQWSSL | |
| | | KASDTAMYYCARHGRGYNGYEGAFDIWGQGTMVTVSSGGGGS | |
| | | GGGGSGGGGSAIQLTQSPSSLSASVGDRVTITCRASQGISSAL | |
| | | AWYQQKPGKAPKLLIYDASSLESGVPSRFSGSGSGTDFTLTISSL | |
| | | QPEDFATYYCQQFNSYPLTFGGGTKVEIKSAAAFVPVFLPAKPT | |
| | | TTPAPRPPTPAPTIASQPLSLRPEACRPAAGGAVHTRGLDFACDI | |
| | | YIWAPLAGTCGVLLLSLVITLYCNHRNRSKRSRLLHSDYMNMT | |
| | | PRRPGPTRKHYQPYAPPRDFAAYRSRVKFSRSADAPAYQQGQN | |
| | | QLYNELNLGRREEYDVLDKRRGRDPEMGGKPRRKNPQEGLYN | |
| | | ELQKDKMAEAYSEIGMKGERRRGKGHDGLYQGLSTATKDTYD | |
| | | ALHMQALPPR | |
| CTX-2846 coding | | ATGGCGCTTCCGGTGACAGCACTGCTCCTCCCCTTGGCGCTG | 141 |
| | | TTGCTCCACGCAGCAAGGCCGCAAGTTCAACTCGTACAATCT | |
| | | GGTGCAGCAGTGAAAAAACCCGGTGAATCTTTGAAAATCAG | |
| | | CTGTAAAGGCTCTGGCTACAGGTTTACAAGCTATTGGATCGG | |
| | | CTGGGTCCGACAAATGCCCGGCAAGGGTCTGGAGTGGATGG | |
| | | GCATCATATATCCGGGAGACTCCGATACGCGATATAGTCCCA | |
| | | GCTTCCAAGGACAGGTTACGATCTCCGCGGGTAAAAGTATAT | |
| | | CTACGGCGTACCTTCAGTGGTCTTCTCTCAAAGCCTCAGATA | |
| | | CGGCTATGTATTACTGTGCCCGCCACGGTCGAGGATACAATG | |
| | | GTTACGAAGGCGCTTTCGATATATGGGGACAGGGTACCATG | |
| | | GTAACCGTTAGCAGTGGTGGAGGTGGGAGTGGTGGTGGTGG | |
| | | GTCAGGCGGGGGAGGTAGCGCAATCCAGTTGACTCAGAGCC | |
| | | CCTCATCTCTTTCCGCGTCTGTAGGCGACCGCGTTACCATAA | |
| | | CCTGCAGAGCCTCTCAGGGCATCTCAAGTGCACTTGCCTGGT | |
| | | ATCAACAGAAGCCAGGTAAGGCCCCCAAGCTCCTGATTTATG | |
| | | ATGCCAGTAGTCTTGAGTCTGGTGTCCCTAGCCGCTTTAGCG | |
| | | GTTCTGGCTCTGGTACTGACTTCACGCTGACGATCTCAAGCC | |
| | | TCCAGCCGGAGGACTTCGCAACATACTATTGTCAACAGTTTA | |
| | | ACTCATACCCGCTGACGTTCGGGGGTGGTACAAAAGTTGAG | |
| | | ATCAAAAGTGCTGCTGCCTTTGTCCCGGTATTTCTCCCAGCC | |
| | | AAACCGACCACGACTCCCGCCCCGCGCCCTCCGACACCCGCT | |
| | | CCCACCATCGCCTCTCAACCTCTTAGTCTTCGCCCCGAGGCA | |
| | | TGCCGACCCGCCGCCGGGGGTGCTGTTCATACGAGGGGCTTG | |
| | | GACTTCGCTTGTGATATTTACATTTGGGCTCCGTTGGCGGGT | |
| | | ACGTGCGGCGTCCTTTTGTTGTCACTCGTTATTACTTTGTATT | |
| | | GTAATCACAGGAATCGCTCAAAGCGGAGTAGGTTGTTGCATT | |
| | | CCGATTACATGAATATGACTCCTCGCCGGCCTGGGCCGACAA | |
| | | GAAAACATTACCAACCCTATGCCCCCCCACGAGACTTCGCTG | |
| | | CGTACAGGTCCCGAGTGAAGTTTTCCCGAAGCGCAGACGCTC | |
| | | CGGCATATCAGCAAGGACAGAATCAGCTGTATAACGAACTG | |
| | | AATTTGGGACGCCGCGAGGAGTATGACGTGCTTGATAAACG | |
| | | CCGGGGGAGAGACCCGGAAATGGGGGGTAAACCCCGAAGA | |
| | | AAGAATCCCCAAGAAGGACTCTACAATGAACTCCAGAAGGA | |
| | | TAAGATGGCGGAGGCCTACTCAGAAATAGGTATGAAGGGCG | |
| | | AACGACGACGGGGAAAAGGTCACGATGGCCTCTACCAAGGG | |
| | | TTGAGTACGGCAACCAAAGATACGTACGATGCACTGCATAT | |
| | | GCAGGCCCTGCCTCCCAGA | |
| CTX-2847 | | MALPVTALLLPLALLLHAARPQVQLVQSGAAVKKPGESLKISCKG | 142 |
| (Ab-2 VH/VL; and | | SGYRFTSYWIGWVRQMPGKGLEWMGIIYPGDSDTRYSPSFQG | |
| 4-1BB CoS with | | QVTISAGKSISTAYLQWSSLKASDTAMYYCARHGRGYNGYEG | |
| signal peptide) | | AFDIWGQGTMVTVSSGGGGSGGGGSGGGGSAIQLTQSPSSLSA | |
| | | SVGDRVTITCRASQGISSALAWYQQKPGKAPKLLIYDASSLESG | |
| | | VPSRFSGSGSGTDFTLTISSLQPEDFATYYCQQFNSYPLTFGGGT | |
| | | KVEIKSAAAFVPVFLPAKPTTTPAPRPPTPAPTIASQPLSLRPEAC | |
| | | RPAAGGAVHTRGLDFACDIYIWAPLAGTCGVLLLSLVITLYCNH | |
| | | RNRKRGRKKLLYIFKQPFMRPVQTTQEEDGCSCRFPEEEEGGCE | |
| | | LRVKFSRSADAPAYQQGQNQLYNELNLGRREEYDVLDKRRGR | |
| | | DPEMGGKPRRKNPQEGLYNELQKDKMAEAYSEIGMKGERRRG | |
| | | KGHDGLYQGLSTATKDTYDALHMQALPPR | |
| CTX-2847 | | QVQLVQSGAAVKKPGESLKISCKGSGYRFTSYWIGWVRQMPG | 143 |
| (no signal peptide) | | KGLEWMGIIYPGDSDTRYSPSFQGQVTISAGKSISTAYLQWSSL | |
| | | KASDTAMYYCARHGRGYNGYEGAFDIWGQGTMVTVSSGGG | |
| | | GSGGGGSGGGGSAIQLTQSPSSLSASVGDRVTITCRASQGISSAL | |
| | | AWYQQKPGKAPKLLIYDASSLESGVPSRFSGSGSGTDFTLTISSL | |
| | | QPEDFATYYCQQFNSYPLTFGGGTKVEIKSAAAFVPVFLPAKPT | |
| | | TTPAPRPPTPAPTIASQPLSLRPEACRPAAGGAVHTRGLDFACDI | |
| | | YIWAPLAGTCGVLLLSLVITLYCNHRNRKRGRKKLLYIFKQPFM | |
| | | RPVQTTQEEDGCSCRFPEEEEGGCELRVKFSRSADAPAYQQGQ | |
| | | NQLYNELNLGRREEYDVLDKRRGRDPEMGGKPRRKNPQEGLY | |
| | | NELQKDKMAEAYSEIGMKGERRRGKGHDGLYQGLSTATKDTY | |
| | | DALHMQALPPR | |
| | | ATGGCGCTTCCGGTGACAGCACTGCTCCTCCCCTTGGCGCTG | 144 |
| CTX-2847 coding | | TTGCTCCACGCAGCAAGGCCGCAAGTTCAACTCGTACAATCT | |
| | | GGTGCAGCAGTGAAAAAACCCGGTGAATCTTTGAAAATCAG | |
| | | CTGTAAAGGCTCTGGCTACAGGTTTACAAGCTATTGGATCGG | |
| | | CTGGGTCCGACAAATGCCCGGCAAGGGTCTGGAGTGGATGG | |
| | | GCATCATATATCCGGGAGACTCCGATACGCGATATAGTCCCA | |
| | | GCTTCCAAGGACAGGTTACGATCTCCGCGGGTAAAAGTATAT | |
| | | CTACGGCGTACCTTCAGTGGTCTTCTCTCAAAGCCTCAGATA | |
| | | CGGCTATGTATTACTGTGCCCGCCACGGTCGAGGATACAATG | |
| | | GTTACGAAGGCGCTTTCGATATATGGGGACAGGGTACCATG | |
| | | GTAACCGTTAGCAGTGGTGGAGGTGGGAGTGGTGGTGGTGG | |
| | | GTCAGGCGGGGGAGGTAGCGCAATCCAGTTGACTCAGAGCC | |
| | | CCTCATCTCTTTCCGCGTCTGTAGGCGACCGCGTTACCATAA | |
| | | CCTGCAGAGCCTCTCAGGGCATCTCAAGTGCACTTGCCTGGT | |
| | | ATCAACAGAAGCCAGGTAAGGCCCCCAAGCTCCTGATTTATG | |
| | | ATGCCAGTAGTCTTGAGTCTGGTGTCCCTAGCCGCTTTAGCG | |
| | | GTTCTGGCTCTGGTACTGACTTCACGCTGACGATCTCAAGCC | |
| | | TCCAGCCGGAGGACTTCGCAACATACTATTGTCAACAGTTTA | |
| | | ACTCATACCCGCTGACGTTCGGGGGTGGTACAAAAGTTGAG | |
| | | ATCAAAAGTGCTGCTGCCTTTGTCCCGGTATTTCTCCCAGCC | |
| | | AAACCGACCACGACTCCCGCCCCGCGCCCTCCGACACCCGCT | |
| | | CCCACCATCGCCTCTCAACCTCTTAGTCTTCGCCCCGAGGCA | |
| | | TGCCGACCCGCCGCCGGGGGTGCTGTTCATACGAGGGGCTTG | |
| | | GACTTCGCTTGTGATATTTACATTTGGGCTCCGTTGGCGGGT | |
| | | ACGTGCGGCGTCCTTTTGTTGTCACTCGTTATTACTTTGTATT | |
| | | GTAATCACAGGAATCGCAAACGGGGCAGAAAGAAACTCCTG | |
| | | TATATATTCAAACAACCATTTATGAGACCAGTACAAACTACT | |
| | | CAAGAGGAAGATGGCTGTAGCTGCCGATTTCCAGAAGAAGA | |
| | | AGAAGGAGGATGTGAACTGCGAGTGAAGTTTTCCCGAAGCG | |
| | | CAGACGCTCCGGCATATCAGCAAGGACAGAATCAGCTGTAT | |
| | | AACGAACTGAATTTGGGACGCCGCGAGGAGTATGACGTGCT | |
| | | TGATAAACGCCGGGGGAGAGACCCGGAAATGGGGGGTAAAC | |
| | | CCCGAAGAAAGAATCCCCAAGAAGGACTCTACAATGAACTC | |
| | | CAGAAGGATAAGATGGCGGAGGCCTACTCAGAAATAGGTAT | |
| | | GAAGGGCGAACGACGACGGGGAAAAGGTCACGATGGCCTCT | |
| | | ACCAAGGGTTGAGTACGGCAACCAAAGATACGTACGATGCA | |
| | | CTGCATATGCAGGCCCTGCCTCCCAGA | |
| Anti- | HCDR1 | GRTTFSTYW | 16 |
| CD117 | HCDR2 | ISWSAGMA | 43 |
| VHH | HCDR3 | GRFHPIRVDTA | 70 |
| P27 | VHH | EVQLVESGGGLVRAGGSLRLSCAASGRTTFSTYWLGWFRQAP | 145 |
| | | GKNREFVAAISWSAGMAYYQDSKGRFTISRDNTKNTAYLQMN | |
| | | SLQPEDTAVYYCAAGRFHPIRVDTALYWAQGTQVTVSS | |
| CTX-2867 | | MALPVTALLLPLALLLHAARPEVQLVESGGGLVRAGGSLRLSCA | 146 |
| (P27 VHH; CD28 | | ASGRTTFSTYWLGWFRQAPGKNREFVAAISWSAGMAYYQDSKG | |
| CoS with signal | | RFTISRDNTKNTAYLQMNSLQPEDTAVYYCAAGRFHPIRVDTA | |
| peptide) | | LYWAQGTQVTVSSSAAAFVPVFLPAKPTTTPAPRPPTPAPTIASQ | |
| | | PLSLRPEACRPAAGGAVHTRGLDFACDIYIWAPLAGTCGVLLLS | |
| | | LVITLYCNHRNRSKRSRLLHSDYMNMTPRRPGPTRKHYQPYAP | |
| | | PRDFAAYRSRVKFSRSADAPAYQQGQNQLYNELNLGRREEYD | |
| | | VLDKRRGRDPEMGGKPRRKNPQEGLYNELQKDKMAEAYSEIG | |
| | | MKGERRRGKGHDGLYQGLSTATKDTYDALHMQALPPR | |
| CTX-2867 | | EVQLVESGGGLVRAGGSLRLSCAASGRTTFSTYWLGWFRQAPG | 147 |
| (no signal peptide) | | KNREFVAAISWSAGMAYYQDSKGRFTISRDNTKNTAYLQMNS | |
| | | LQPEDTAVYYCAAGRFHPIRVDTALYWAQGTQVTVSSSAAAFV | |
| | | PVFLPAKPTTTPAPRPPTPAPTIASQPLSLRPEACRPAAGGAVHT | |
| | | RGLDFACDIYIWAPLAGTCGVLLLSLVITLYCNHRNRSKRSRLL | |
| | | HSDYMNMTPRRPGPTRKHYQPYAPPRDFAAYRSRVKFSRSADA | |
| | | PAYQQGQNQLYNELNLGRREEYDVLDKRRGRDPEMGGKPRRK | |
| | | NPQEGLYNELQKDKMAEAYSEIGMKGERRRGKGHDGLYQGLS | |
| | | TATKDTYDALHMQALPPR | |
| CTX-2867 coding | | ATGGCGCTTCCGGTGACAGCACTGCTCCTCCCCTTGGCGCTG | 148 |
| | | TTGCTCCACGCAGCAAGGCCGGAGGTTCAGCTTGTGGAATCA | |
| | | GGCGGGGGCCTCGTGCGGGCCGGTGGTAGTTTGCGACTTAGC | |
| | | TGTGCGGCGTCAGGGAGGACAACATTCAGCACATATTGGCTT | |
| | | GGGTGGTTCCGGCAGGCTCCGGGAAAGAATAGAGAGTTCGT | |
| | | CGCCGCAATATCATGGTCAGCCGGGATGGCTTATTATCAGGA | |
| | | TAGCAAGGGTCGGTTCACCATCTCCCGCGACAATACAAAGA | |
| | | ATACGGCTTATCTCCAGATGAACAGTCTCCAACCGGAAGATA | |
| | | CCGCTGTATATTACTGCGCTGCCGGTCGCTTTCACCCCATCCG | |
| | | CGTAGATACGGCATTGTACTGGGCGCAAGGCACTCAAGTGA | |
| | | CAGTAAGCAGTAGTGCTGCTGCCTTTGTCCCGGTATTTCTCCC | |
| | | AGCCAAACCGACCACGACTCCCGCCCCGCGCCCTCCGACACC | |
| | | CGCTCCCACCATCGCCTCTCAACCTCTTAGTCTTCGCCCCGAG | |
| | | GCATGCCGACCCGCCGCCGGGGGTGCTGTTCATACGAGGGG | |
| | | CTTGGACTTCGCTTGTGATATTTACATTTGGGCTCCGTTGGCG | |
| | | GGTACGTGCGGCGTCCTTTTGTTGTCACTCGTTATTACTTTGT | |
| | | ATTGTAATCACAGGAATCGCTCAAAGCGGAGTAGGTTGTTGC | |
| | | ATTCCGATTACATGAATATGACTCCTCGCCGGCCTGGGCCGA | |
| | | CAAGAAAACATTACCAACCCTATGCCCCCCCACGAGACTTCG | |
| | | CTGCGTACAGGTCCCGAGTGAAGTTTTCCCGAAGCGCAGACG | |
| | | CTCCGGCATATCAGCAAGGACAGAATCAGCTGTATAACGAA | |
| | | CTGAATTTGGGACGCCGCGAGGAGTATGACGTGCTTGATAA | |
| | | ACGCCGGGGGAGAGACCCGGAAATGGGGGGTAAACCCCGA | |
| | | AGAAAGAATCCCCAAGAAGGACTCTACAATGAACTCCAGAA | |
| | | GGATAAGATGGCGGAGGCCTACTCAGAAATAGGTATGAAGG | |
| | | GCGAACGACGACGGGGAAAAGGTCACGATGGCCTCTACCAA | |
| | | GGGTTGAGTACGGCAACCAAAGATACGTACGATGCACTGCA | |
| | | TATGCAGGCCCTGCCTCCCAGA | |
| CTX-2868 | | MALPVTALLLPLALLLHAARPEVQLVESGGGLVRAGGSLRLSCAA | 149 |
| (P27 VHH; 4-1BB | | SGRTTFSTYWLGWFRQAPGKNREFVAAISWSAGMAYYQDSKG | |
| CoS with signal | | RFTISRDNTKNTAYLQMNSLQPEDTAVYYCAAGRFHPIRVDTA | |
| peptide) | | LYWAQGTQVTVSSSAAAFVPVFLPAKPTTTPAPRPPTPAPTIASQ | |
| | | PLSLRPEACRPAAGGAVHTRGLDFACDIYIWAPLAGTCGVLLLS | |
| | | LVITLYCNHRNRKRGRKKLLYIFKQPFMRPVQTTQEEDGCSCRF | |
| | | PEEEEGGCELRVKFSRSADAPAYQQGQNQLYNELNLGRREEYD | |
| | | VLDKRRGRDPEMGGKPRRKNPQEGLYNELQKDKMAEAYSEIG | |
| | | MKGERRRGKGHDGLYQGLSTATKDTYDALHMQALPPR | |
| CTX-2868 | | EVQLVESGGGLVRAGGSLRLSCAASGRTTFSTYWLGWFRQAPG | 227 |
| (no signal peptide) | | KNREFVAAISWSAGMAYYQDSKGRFTISRDNTKNTAYLQMNS | |
| | | LQPEDTAVYYCAAGRFHPIRVDTALYWAQGTQVTVSSSAAAFV | |
| | | PVFLPAKPTTTPAPRPPTPAPTIASQPLSLRPEACRPAAGGAVHT | |
| | | RGLDFACDIYIWAPLAGTCGVLLLSLVITLYCNHRNRKRGRKKL | |
| | | LYIFKQPFMRPVQTTQEEDGCSCRFPEEEEGGCELRVKFSRSAD | |
| | | APAYQQGQNQLYNELNLGRREEYDVLDKRRGRDPEMGGKPRR | |
| | | KNPQEGLYNELQKDKMAEAYSEIGMKGERRRGKGHDGLYQGL | |
| | | STATKDTYDALHMQALPPR | |
| CTX-2868 coding | | ATGGCGCTTCCGGTGACAGCACTGCTCCTCCCCTTGGCGCTG | 150 |
| | | TTGCTCCACGCAGCAAGGCCGGAGGTTCAGCTTGTGGAATCA | |
| | | GGCGGGGGCCTCGTGCGGGCCGGTGGTAGTTTGCGACTTAGC | |
| | | TGTGCGGCGTCAGGGAGGACAACATTCAGCACATATTGGCTT | |
| | | GGGTGGTTCCGGCAGGCTCCGGGAAAGAATAGAGAGTTCGT | |
| | | CGCCGCAATATCATGGTCAGCCGGGATGGCTTATTATCAGGA | |
| | | TAGCAAGGGTCGGTTCACCATCTCCCGCGACAATACAAAGA | |
| | | ATACGGCTTATCTCCAGATGAACAGTCTCCAACCGGAAGATA | |
| | | CCGCTGTATATTACTGCGCTGCCGGTCGCTTTCACCCCATCCG | |
| | | CGTAGATACGGCATTGTACTGGGCGCAAGGCACTCAAGTGA | |
| | | CAGTAAGCAGTAGTGCTGCTGCCTTTGTCCCGGTATTTCTCCC | |
| | | AGCCAAACCGACCACGACTCCCGCCCCGCGCCCTCCGACACC | |
| | | CGCTCCCACCATCGCCTCTCAACCTCTTAGTCTTCGCCCCGAG | |
| | | GCATGCCGACCCGCCGCCGGGGGTGCTGTTCATACGAGGGG | |
| | | CTTGGACTTCGCTTGTGATATTTACATTTGGGCTCCGTTGGCG | |
| | | GGTACGTGCGGCGTCCTTTTGTTGTCACTCGTTATTACTTTGT | |
| | | ATTGTAATCACAGGAATCGCAAACGGGGCAGAAAGAAACTC | |
| | | CTGTATATATTCAAACAACCATTTATGAGACCAGTACAAACT | |
| | | ACTCAAGAGGAAGATGGCTGTAGCTGCCGATTTCCAGAAGA | |
| | | AGAAGAAGGAGGATGTGAACTGCGAGTGAAGTTTTCCCGAA | |
| | | GCGCAGACGCTCCGGCATATCAGCAAGGACAGAATCAGCTG | |
| | | TATAACGAACTGAATTTGGGACGCCGCGAGGAGTATGACGT | |
| | | GCTTGATAAACGCCGGGGGAGAGACCCGGAAATGGGGGGTA | |
| | | AACCCCGAAGAAAGAATCCCCAAGAAGGACTCTACAATGAA | |
| | | CTCCAGAAGGATAAGATGGCGGAGGCCTACTCAGAAATAGG | |
| | | TATGAAGGGCGAACGACGACGGGGAAAAGGTCACGATGGCC | |
| | | TCTACCAAGGGTTGAGTACGGCAACCAAAGATACGTACGAT | |
| | | GCACTGCATATGCAGGCCCTGCCTCCCAGA | |
| Anti- | HCDR1 | GHTFSNYA | 20 |
| CD117 | HCDR2 | ISWSGGST | 47 |
| VHH | HCDR3 | DSTGVYGTGYVSSRKGRY | 74 |
| P29 | VHH | QVHLVESGGGLVQAGGSLGLSCAASGHTFSNYAMGWFRQAP | 151 |
| | | GKEREFVAAISWSGGSTLYADSVKGRFTISRDNGENTVYLQ | |
| | | MNSLKPEDTAVYYCALDSTGVYGTGYVSSRKGRYEYWGQGT | |
| | | LVTVSS | |
| CTX-2869 | | MALPVTALLLPLALLLHAARPQVHLVESGGGLVQAGGSLGLSC | 152 |
| (P29 VHH; CD28 | | AASGHTFSNYAMGWFRQAPGKEREFVAAISWSGGSTLYADSVK | |
| CoS with signal | | GRFTISRDNGENTVYLQMNSLKPEDTAVYYCALDSTGVYGTGY | |
| peptide) | | VSSRKGRYEYWGQGTLVTVSSSAAAFVPVFLPAKPTTTPAPRPP | |
| | | TPAPTIASQPLSLRPEACRPAAGGAVHTRGLDFACDIYIWAPLA | |
| | | GTCGVLLLSLVITLYCNHRNRSKRSRLLHSDYMNMTPRRPGPT | |
| | | RKHYQPYAPPRDFAAYRSRVKFSRSADAPAYQQGQNQLYNEL | |
| | | NLGRREEYDVLDKRRGRDPEMGGKPRRKNPQEGLYNELQKDK | |
| | | MAEAYSEIGMKGERRRGKGHDGLYQGLSTATKDTYDALHMQ | |
| | | ALPPR | |
| CTX-2869 | | QVHLVESGGGLVQAGGSLGLSCAASGHTFSNYAMGWFRQAPG | 153 |
| (no signal peptide) | | KEREFVAAISWSGGSTLYADSVKGRFTISRDNGENTVYLQMNS | |
| | | LKPEDTAVYYCALDSTGVYGTGYVSSRKGRYEYWGQGTLVTV | |
| | | SSSAAAFVPVFLPAKPTTTPAPRPPTPAPTIASQPLSLRPEACRPA | |
| | | AGGAVHTRGLDFACDIYIWAPLAGTCGVLLLSLVITLYCNHRN | |
| | | RSKRSRLLHSDYMNMTPRRPGPTRKHYQPYAPPRDFAAYRSRV | |
| | | KFSRSADAPAYQQGQNQLYNELNLGRREEYDVLDKRRGRDPE | |
| | | MGGKPRRKNPQEGLYNELQKDKMAEAYSEIGMKGERRRGKG | |
| | | HDGLYQGLSTATKDTYDALHMQALPPR | |
| CTX-2869 coding | | ATGGCGCTTCCGGTGACAGCACTGCTCCTCCCCTTGGCGCTG | 154 |
| | | TTGCTCCACGCAGCAAGGCCGCAGGTTCATTTGGTTGAGTCA | |
| | | GGCGGAGGGCTCGTGCAAGCTGGCGGGTCCCTGGGGCTGTC | |
| | | TTGTGCTGCGAGCGGTCACACCTTCTCCAACTATGCGATGGG | |
| | | ATGGTTTCGCCAAGCACCGGGTAAGGAGCGCGAGTTCGTGG | |
| | | CTGCCATTAGTTGGTCCGGTGGCTCCACGCTCTATGCTGATTC | |
| | | AGTGAAGGGTAGATTCACGATATCACGGGATAATGGCGAAA | |
| | | ATACGGTCTATTTGCAAATGAACAGTCTGAAACCAGAGGAC | |
| | | ACGGCGGTGTATTACTGCGCCCTTGATAGTACCGGTGTCTAT | |
| | | GGAACGGGATACGTGAGCTCTAGGAAAGGGAGATACGAATA | |
| | | CTGGGGACAGGGAACATTGGTAACTGTCTCATCTAGTGCTGC | |
| | | TGCCTTTGTCCCGGTATTTCTCCCAGCCAAACCGACCACGAC | |
| | | TCCCGCCCCGCGCCCTCCGACACCCGCTCCCACCATCGCCTC | |
| | | TCAACCTCTTAGTCTTCGCCCCGAGGCATGCCGACCCGCCGC | |
| | | CGGGGGTGCTGTTCATACGAGGGGCTTGGACTTCGCTTGTGA | |
| | | TATTTACATTTGGGCTCCGTTGGCGGGTACGTGCGGCGTCCT | |
| | | TTTGTTGTCACTCGTTATTACTTTGTATTGTAATCACAGGAAT | |
| | | CGCTCAAAGCGGAGTAGGTTGTTGCATTCCGATTACATGAAT | |
| | | ATGACTCCTCGCCGGCCTGGGCCGACAAGAAAACATTACCA | |
| | | ACCCTATGCCCCCCCACGAGACTTCGCTGCGTACAGGTCCCG | |
| | | AGTGAAGTTTTCCCGAAGCGCAGACGCTCCGGCATATCAGCA | |
| | | AGGACAGAATCAGCTGTATAACGAACTGAATTTGGGACGCC | |
| | | GCGAGGAGTATGACGTGCTTGATAAACGCCGGGGGAGAGAC | |
| | | CCGGAAATGGGGGGTAAACCCCGAAGAAAGAATCCCCAAGA | |
| | | AGGACTCTACAATGAACTCCAGAAGGATAAGATGGCGGAGG | |
| | | CCTACTCAGAAATAGGTATGAAGGGCGAACGACGACGGGGA | |
| | | AAAGGTCACGATGGCCTCTACCAAGGGTTGAGTACGGCAAC | |
| | | CAAAGATACGTACGATGCACTGCATATGCAGGCCCTGCCTCC | |
| | | CAGA | |
| CTX-2870 | | MALPVTALLLPLALLLHAARPQVHLVESGGGLVQAGGSLGLSC | 155 |
| (P29 VHH; 4-1BB | | AASGHTFSNYAMGWFRQAPGKEREFVAAISWSGGSTLYADSVK | |
| CoS with signal | | GRFTISRDNGENTVYLQMNSLKPEDTAVYYCALDSTGVYGTGY | |
| peptide) | | VSSRKGRYEYWGQGTLVTVSSSAAAFVPVFLPAKPTTTPAPRPP | |
| | | TPAPTIASQPLSLRPEACRPAAGGAVHTRGLDFACDIYIWAPLA | |
| | | GTCGVLLLSLVITLYCNHRNRKRGRKKLLYIFKQPFMRPVQTTQ | |
| | | EEDGCSCRFPEEEEGGCELRVKFSRSADAPAYQQGQNQLYNEL | |
| | | NLGRREEYDVLDKRRGRDPEMGGKPRRKNPQEGLYNELQKDK | |
| | | MAEAYSEIGMKGERRRGKGHDGLYQGLSTATKDTYDALHMQ | |
| | | ALPPR | |
| CTX-2870 | | QVHLVESGGGLVQAGGSLGLSCAASGHTFSNYAMGWFRQAPG | 156 |
| (no signal peptide) | | KEREFVAAISWSGGSTLYADSVKGRFTISRDNGENTVYLQMNS | |
| | | LKPEDTAVYYCALDSTGVYGTGYVSSRKGRYEYWGQGTLVTVS | |
| | | SSAAAFVPVFLPAKPTTTPAPRPPTPAPTIASQPLSLRPEACR | |
| | | PAAGGAVHTRGLDFACDIYIWAPLAGTCGVLLLSLVITLYCNH | |
| | | RNRKRGRKKLLYIFKQPFMRPVQTTQEEDGCSCRFPEEEEGGC | |
| | | ELRVKFSRSADAPAYQQGQNQLYNELNLGRREEYDVLDKRRGR | |
| | | DPEMGGKPRRKNPQEGLYNELQKDKMAEAYSEIGMKGERRRGK | |
| | | GHDGLYQGLSTATKDTYDALHMQALPPR | |
| CTX-2870 coding | | ATGGCGCTTCCGGTGACAGCACTGCTCCTCCCCTTGGCGCTG | 157 |
| | | TTGCTCCACGCAGCAAGGCCGCAGGTTCATTTGGTTGAGTCA | |
| | | GGCGGAGGGCTCGTGCAAGCTGGCGGGTCCCTGGGGCTGTC | |
| | | TTGTGCTGCGAGCGGTCACACCTTCTCCAACTATGCGATGGG | |
| | | ATGGTTTCGCCAAGCACCGGGTAAGGAGCGCGAGTTCGTGG | |
| | | CTGCCATTAGTTGGTCCGGTGGCTCCACGCTCTATGCTGATTC | |
| | | AGTGAAGGGTAGATTCACGATATCACGGGATAATGGCGAAA | |
| | | ATACGGTCTATTTGCAAATGAACAGTCTGAAACCAGAGGAC | |
| | | ACGGCGGTGTATTACTGCGCCCTTGATAGTACCGGTGTCTAT | |
| | | GGAACGGGATACGTGAGCTCTAGGAAAGGGAGATACGAATA | |
| | | CTGGGGACAGGGAACATTGGTAACTGTCTCATCTAGTGCTGC | |
| | | TGCCTTTGTCCCGGTATTTCTCCCAGCCAAACCGACCACGAC | |
| | | TCCCGCCCCGCGCCCTCCGACACCCGCTCCCACCATCGCCTC | |
| | | TCAACCTCTTAGTCTTCGCCCCGAGGCATGCCGACCCGCCGC | |
| | | CGGGGGTGCTGTTCATACGAGGGGCTTGGACTTCGCTTGTGA | |
| | | TATTTACATTTGGGCTCCGTTGGCGGGTACGTGCGGCGTCCT | |
| | | TTTGTTGTCACTCGTTATTACTTTGTATTGTAATCACAGGAAT | |
| | | CGCAAACGGGGCAGAAAGAAACTCCTGTATATATTCAAACA | |
| | | ACCATTTATGAGACCAGTACAAACTACTCAAGAGGAAGATG | |
| | | GCTGTAGCTGCCGATTTCCAGAAGAAGAAGAAGGAGGATGT | |
| | | GAACTGCGAGTGAAGTTTTCCCGAAGCGCAGACGCTCCGGC | |
| | | ATATCAGCAAGGACAGAATCAGCTGTATAACGAACTGAATT | |
| | | TGGGACGCCGCGAGGAGTATGACGTGCTTGATAAACGCCGG | |
| | | GGGAGAGACCCGGAAATGGGGGGTAAACCCCGAAGAAAGA | |
| | | ATCCCCAAGAAGGACTCTACAATGAACTCCAGAAGGATAAG | |
| | | ATGGCGGAGGCCTACTCAGAAATAGGTATGAAGGGCGAACG | |
| | | ACGACGGGGAAAAGGTCACGATGGCCTCTACCAAGGGTTGA | |
| | | GTACGGCAACCAAAGATACGTACGATGCACTGCATATGCAG | |
| | | GCCCTGCCTCCCAGA | |
| Anti- | HCDR1 | GRTFTYDA | 27 |
| CD117 | HCDR2 | ISWSGGST | 47 |
| VHH | HCDR3 | DESFPAYYSDYALY | 81 |
| P31 | VHH | EVQLVESGGGLVRAGGSLRLSCAASGRTFTYDARAWFRQAPG | 158 |
| | | KEREFVAAISWSGGSTYYPNSMKGRFTISRDNAKNTVYLQMNS | |
| | | LKPEDTAVYYCAADESFPAYYSDYALYRYWGQGTLVTVSS | |
| CTX-2871 | | MALPVTALLLPLALLLHAARPEVQLVESGGGLVRAGGSLRLSCAA | 159 |
| (P31 VHH; CD28 | | SGRTFTYDARAWFRQAPGKEREFVAAISWSGGSTYYPNSMKGR | |
| CoS with signal | | FTISRDNAKNTVYLQMNSLKPEDTAVYYCAADESFPAYYSDYA | |
| peptide) | | LYRYWGQGTLVTVSSSAAAFVPVFLPAKPTTTPAPRPPTPAPTIA | |
| | | SQPLSLRPEACRPAAGGAVHTRGLDFACDIYIWAPLAGTCGVLL | |
| | | LSLVITLYCNHRNRSKRSRLLHSDYMNMTPRRPGPTRKHYQPY | |
| | | APPRDFAAYRSRVKFSRSADAPAYQQGQNQLYNELNLGRREEY | |
| | | DVLDKRRGRDPEMGGKPRRKNPQEGLYNELQKDKMAEAYSEI | |
| | | GMKGERRRGKGHDGLYQGLSTATKDTYDALHMQALPPR | |
| CTX-2871 | | EVQLVESGGGLVRAGGSLRLSCAASGRTFTYDARAWFRQAPG | 160 |
| (no signal peptide) | | KEREFVAAISWSGGSTYYPNSMKGRFTISRDNAKNTVYLQMNS | |
| | | LKPEDTAVYYCAADESFPAYYSDYALYRYWGQGTLVTVSSSA | |
| | | AAFVPVFLPAKPTTTPAPRPPTPAPTIASQPLSLRPEACRPAAGG | |
| | | AVHTRGLDFACDIYIWAPLAGTCGVLLLSLVITLYCNHRNRSKR | |
| | | SRLLHSDYMNMTPRRPGPTRKHYQPYAPPRDFAAYRSRVKFSR | |
| | | SADAPAYQQGQNQLYNELNLGRREEYDVLDKRRGRDPEMGG | |
| | | KPRRKNPQEGLYNELQKDKMAEAYSEIGMKGERRRGKGHDGL | |
| | | YQGLSTATKDTYDALHMQALPPR | |
| CTX-2871 coding | | ATGGCGCTTCCGGTGACAGCACTGCTCCTCCCCTTGGCGCTG | 161 |
| | | TTGCTCCACGCAGCAAGGCCGGAGGTCCAGTTGGTCGAATCA | |
| | | GGTGGGGGACTTGTTCGAGCTGGCGGTTCTCTGCGGCTTTCC | |
| | | TGCGCGGCCAGTGGACGGACCTTTACCTACGATGCTAGGGCA | |
| | | TGGTTTAGACAAGCGCCCGGCAAAGAGCGCGAGTTCGTAGC | |
| | | CGCTATATCCTGGTCTGGAGGTTCAACCTATTACCCCAATTC | |
| | | CATGAAGGGTAGGTTTACGATATCCAGGGACAATGCAAAGA | |
| | | ATACTGTCTACCTTCAAATGAACTCCTTGAAACCGGAAGATA | |
| | | CTGCTGTCTATTATTGTGCGGCCGACGAATCCTTTCCCGCTTA | |
| | | CTACAGTGACTATGCTCTGTACCGCTACTGGGGACAAGGCAC | |
| | | CCTCGTAACCGTAAGTAGCAGTGCTGCTGCCTTTGTCCCGGT | |
| | | ATTTCTCCCAGCCAAACCGACCACGACTCCCGCCCCGCGCCC | |
| | | TCCGACACCCGCTCCCACCATCGCCTCTCAACCTCTTAGTCTT | |
| | | CGCCCCGAGGCATGCCGACCCGCCGCCGGGGGTGCTGTTCAT | |
| | | ACGAGGGGCTTGGACTTCGCTTGTGATATTTACATTTGGGCT | |
| | | CCGTTGGCGGGTACGTGCGGCGTCCTTTTGTTGTCACTCGTTA | |
| | | TTACTTTGTATTGTAATCACAGGAATCGCTCAAAGCGGAGTA | |
| | | GGTTGTTGCATTCCGATTACATGAATATGACTCCTCGCCGGC | |
| | | CTGGGCCGACAAGAAAACATTACCAACCCTATGCCCCCCCAC | |
| | | GAGACTTCGCTGCGTACAGGTCCCGAGTGAAGTTTTCCCGAA | |
| | | GCGCAGACGCTCCGGCATATCAGCAAGGACAGAATCAGCTG | |
| | | TATAACGAACTGAATTTGGGACGCCGCGAGGAGTATGACGT | |
| | | GCTTGATAAACGCCGGGGGAGAGACCCGGAAATGGGGGGTA | |
| | | AACCCCGAAGAAAGAATCCCCAAGAAGGACTCTACAATGAA | |
| | | CTCCAGAAGGATAAGATGGCGGAGGCCTACTCAGAAATAGG | |
| | | TATGAAGGGCGAACGACGACGGGGAAAAGGTCACGATGGCC | |
| | | TCTACCAAGGGTTGAGTACGGCAACCAAAGATACGTACGAT | |
| | | GCACTGCATATGCAGGCCCTGCCTCCCAGA | |
| CTX-2872 | | MALPVTALLLPLALLLHAARPEVQLVESGGGLVRAGGSLRLSCAA | 162 |
| (P31 VHH; 4-1BB | | SGRTFTYDARAWFRQAPGKEREFVAAISWSGGSTYYPNSMKGR | |
| CoS with signal | | FTISRDNAKNTVYLQMNSLKPEDTAVYYCAADESFPAYYSDYA | |
| peptide) | | LYRYWGQGTLVTVSSSAAAFVPVFLPAKPTTTPAPRPPTPAPTIA | |
| | | SQPLSLRPEACRPAAGGAVHTRGLDFACDIYIWAPLAGTCGVLL | |
| | | LSLVITLYCNHRNRKRGRKKLLYIFKQPFMRPVQTTQEEDGCSC | |
| | | RFPEEEEGGCELRVKFSRSADAPAYQQGQNQLYNELNLGRREE | |
| | | YDVLDKRRGRDPEMGGKPRRKNPQEGLYNELQKDKMAEAYS | |
| | | EIGMKGERRRGKGHDGLYQGLSTATKDTYDALHMQALPPR | |
| CTX-2872 | | EVQLVESGGGLVRAGGSLRLSCAASGRTFTYDARAWFRQAPG | 163 |
| (no signal peptide) | | KEREFVAAISWSGGSTYYPNSMKGRFTISRDNAKNTVYLQMNS | |
| | | LKPEDTAVYYCAADESFPAYYSDYALYRYWGQGTLVTVSSSA | |
| | | AAFVPVFLPAKPTTTPAPRPPTPAPTIASQPLSLRPEACRPAAGG | |
| | | AVHTRGLDFACDIYIWAPLAGTCGVLLLSLVITLYCNHRNRKR | |
| | | GRKKLLYIFKQPFMRPVQTTQEEDGCSCRFPEEEEGGCELRVKF | |
| | | SRSADAPAYQQGQNQLYNELNLGRREEYDVLDKRRGRDPEMG | |
| | | GKPRRKNPQEGLYNELQKDKMAEAYSEIGMKGERRRGKGHDG | |
| | | LYQGLSTATKDTYDALHMQALPPR | |
| CTX-2872 coding | | ATGGCGCTTCCGGTGACAGCACTGCTCCTCCCCTTGGCGCTG | 164 |
| | | TTGCTCCACGCAGCAAGGCCGGAGGTCCAGTTGGTCGAATCA | |
| | | GGTGGGGGACTTGTTCGAGCTGGCGGTTCTCTGCGGCTTTCC | |
| | | TGCGCGGCCAGTGGACGGACCTTTACCTACGATGCTAGGGCA | |
| | | TGGTTTAGACAAGCGCCCGGCAAAGAGCGCGAGTTCGTAGC | |
| | | CGCTATATCCTGGTCTGGAGGTTCAACCTATTACCCCAATTC | |
| | | CATGAAGGGTAGGTTTACGATATCCAGGGACAATGCAAAGA | |
| | | ATACTGTCTACCTTCAAATGAACTCCTTGAAACCGGAAGATA | |
| | | CTGCTGTCTATTATTGTGCGGCCGACGAATCCTTTCCCGCTTA | |
| | | CTACAGTGACTATGCTCTGTACCGCTACTGGGGACAAGGCAC | |
| | | CCTCGTAACCGTAAGTAGCAGTGCTGCTGCCTTTGTCCCGGT | |
| | | ATTTCTCCCAGCCAAACCGACCACGACTCCCGCCCCGCGCCC | |
| | | TCCGACACCCGCTCCCACCATCGCCTCTCAACCTCTTAGTCTT | |
| | | CGCCCCGAGGCATGCCGACCCGCCGCCGGGGGTGCTGTTCAT | |
| | | ACGAGGGGCTTGGACTTCGCTTGTGATATTTACATTTGGGCT | |
| | | CCGTTGGCGGGTACGTGCGGCGTCCTTTTGTTGTCACTCGTTA | |
| | | TTACTTTGTATTGTAATCACAGGAATCGCAAACGGGGCAGAA | |
| | | AGAAACTCCTGTATATATTCAAACAACCATTTATGAGACCAG | |
| | | TACAAACTACTCAAGAGGAAGATGGCTGTAGCTGCCGATTTC | |
| | | CAGAAGAAGAAGAAGGAGGATGTGAACTGCGAGTGAAGTTT | |
| | | TCCCGAAGCGCAGACGCTCCGGCATATCAGCAAGGACAGAA | |
| | | TCAGCTGTATAACGAACTGAATTTGGGACGCCGCGAGGAGT | |
| | | ATGACGTGCTTGATAAACGCCGGGGGAGAGACCCGGAAATG | |
| | | GGGGGTAAACCCCGAAGAAAGAATCCCCAAGAAGGACTCTA | |
| | | CAATGAACTCCAGAAGGATAAGATGGCGGAGGCCTACTCAG | |
| | | AAATAGGTATGAAGGGCGAACGACGACGGGGAAAAGGTCA | |
| | | CGATGGCCTCTACCAAGGGTTGAGTACGGCAACCAAAGATA | |
| | | CGTACGATGCACTGCATATGCAGGCCCTGCCTCCCAGA | |
| Anti- | HCDR1 | GRTLSNYF | 18 |
| CD117 | HCDR2 | IHWSLGST | 45 |
| VHH | HCDR3 | GQHLSGLGGSAWSIEG | 72 |
| P32 | VHH | QVQLVESGGGLVQAGDSLRLSCAVSGRTLSNYFMAWLRQAPG | 165 |
| | | KEREFVAAIHWSLGSTYYQDPVKGRFTISRDKAKNTVYLQMN | |
| | | TLKPEDTATYICAAGQHLSGLGGSAWSIEGDYWGQGTQVTVS | |
| | | S | |
| CTX-2873 | | MALPVTALLLPLALLLHAARPQVQLVESGGGLVQAGDSLRLSCA | 166 |
| (P32 VHH; CD28 | | VSGRTLSNYFMAWLRQAPGKEREFVAAIHWSLGSTYYQDPVK | |
| CoS with signal | | GRFTISRDKAKNTVYLQMNTLKPEDTATYICAAGQHLSGLGGS | |
| peptide) | | AWSIEGDYWGQGTQVTVSSSAAAFVPVFLPAKPTTTPAPRPPTP | |
| | | APTIASQPLSLRPEACRPAAGGAVHTRGLDFACDIYIWAPLAGT | |
| | | CGVLLLSLVITLYCNHRNRSKRSRLLHSDYMNMTPRRPGPTRK | |
| | | HYQPYAPPRDFAAYRSRVKFSRSADAPAYQQGQNQLYNELNL | |
| | | GRREEYDVLDKRRGRDPEMGGKPRRKNPQEGLYNELQKDKM | |
| | | AEAYSEIGMKGERRRGKGHDGLYQGLSTATKDTYDALHMQAL | |
| | | PPR | |
| CTX-2873 | | QVQLVESGGGLVQAGDSLRLSCAVSGRTLSNYFMAWLRQAPG | 167 |
| (no signal peptide) | | KEREFVAAIHWSLGSTYYQDPVKGRFTISRDKAKNTVYLQMNT | |
| | | LKPEDTATYICAAGQHLSGLGGSAWSIEGDYWGQGTQVTVSSS | |
| | | AAAFVPVFLPAKPTTTPAPRPPTPAPTIASQPLSLRPEACRPAAG | |
| | | GAVHTRGLDFACDIYIWAPLAGTCGVLLLSLVITLYCNHRNRSK | |
| | | RSRLLHSDYMNMTPRRPGPTRKHYQPYAPPRDFAAYRSRVKFS | |
| | | RSADAPAYQQGQNQLYNELNLGRREEYDVLDKRRGRDPEMGG | |
| | | KPRRKNPQEGLYNELQKDKMAEAYSEIGMKGERRRGKGHDGL | |
| | | YQGLSTATKDTYDALHMQALPPR | |
| CTX-2873 coding | | ATGGCGCTTCCGGTGACAGCACTGCTCCTCCCCTTGGCGCTG | 168 |
| | | TTGCTCCACGCAGCAAGGCCGCAAGTGCAGCTCGTTGAAAGT | |
| | | GGGGGCGGACTTGTTCAGGCAGGTGACAGCCTTCGATTGAG | |
| | | CTGTGCAGTGAGCGGAAGAACTCTGAGCAACTACTTCATGGC | |
| | | GTGGCTGAGACAAGCACCTGGCAAAGAACGGGAGTTCGTCG | |
| | | CTGCGATACACTGGAGCCTCGGCTCAACATACTACCAAGACC | |
| | | CTGTAAAGGGGAGGTTTACGATAAGTAGAGATAAGGCTAAG | |
| | | AATACGGTTTATCTGCAAATGAACACATTGAAGCCTGAGGAT | |
| | | ACAGCAACGTATATATGCGCAGCGGGGCAACACCTGTCTGG | |
| | | GCTGGGTGGGTCTGCATGGTCCATAGAAGGAGACTACTGGG | |
| | | GTCAAGGGACCCAAGTAACAGTCAGCAGCAGTGCTGCTGCC | |
| | | TTTGTCCCGGTATTTCTCCCAGCCAAACCGACCACGACTCCC | |
| | | GCCCCGCGCCCTCCGACACCCGCTCCCACCATCGCCTCTCAA | |
| | | CCTCTTAGTCTTCGCCCCGAGGCATGCCGACCCGCCGCCGGG | |
| | | GGTGCTGTTCATACGAGGGGCTTGGACTTCGCTTGTGATATT | |
| | | TACATTTGGGCTCCGTTGGCGGGTACGTGCGGCGTCCTTTTG | |
| | | TTGTCACTCGTTATTACTTTGTATTGTAATCACAGGAATCGCT | |
| | | CAAAGCGGAGTAGGTTGTTGCATTCCGATTACATGAATATGA | |
| | | CTCCTCGCCGGCCTGGGCCGACAAGAAAACATTACCAACCCT | |
| | | ATGCCCCCCCACGAGACTTCGCTGCGTACAGGTCCCGAGTGA | |
| | | AGTTTTCCCGAAGCGCAGACGCTCCGGCATATCAGCAAGGA | |
| | | CAGAATCAGCTGTATAACGAACTGAATTTGGGACGCCGCGA | |
| | | GGAGTATGACGTGCTTGATAAACGCCGGGGGAGAGACCCGG | |
| | | AAATGGGGGGTAAACCCCGAAGAAAGAATCCCCAAGAAGG | |
| | | ACTCTACAATGAACTCCAGAAGGATAAGATGGCGGAGGCCT | |
| | | ACTCAGAAATAGGTATGAAGGGCGAACGACGACGGGGAAA | |
| | | AGGTCACGATGGCCTCTACCAAGGGTTGAGTACGGCAACCA | |
| | | AAGATACGTACGATGCACTGCATATGCAGGCCCTGCCTCCCA | |
| | | GA | |
| CTX-2874 | | MALPVTALLLPLALLLHAARPQVQLVESGGGLVQAGDSLRLSCA | 169 |
| (P32 VHH; 4-1BB | | VSGRTLSNYFMAWLRQAPGKEREFVAAIHWSLGSTYYQDPVK | |
| CoS with signal | | GRFTISRDKAKNTVYLQMNTLKPEDTATYICAAGQHLSGLGGS | |
| peptide) | | AWSIEGDYWGQGTQVTVSSSAAAFVPVFLPAKPTTTPAPRPPTP | |
| | | APTIASQPLSLRPEACRPAAGGAVHTRGLDFACDIYIWAPLAGT | |
| | | CGVLLLSLVITLYCNHRNRKRGRKKLLYIFKQPFMRPVQTTQEE | |
| | | DGCSCRFPEEEEGGCELRVKFSRSADAPAYQQGQNQLYNELNL | |
| | | GRREEYDVLDKRRGRDPEMGGKPRRKNPQEGLYNELQKDKM | |
| | | AEAYSEIGMKGERRRGKGHDGLYQGLSTATKDTYDALHMQAL | |
| | | PPR | |
| CTX-2874 | | QVQLVESGGGLVQAGDSLRLSCAVSGRTLSNYFMAWLRQAPG | 170 |
| (no signal peptide) | | KEREFVAAIHWSLGSTYYQDPVKGRFTISRDKAKNTVYLQMNT | |
| | | LKPEDTATYICAAGQHLSGLGGSAWSIEGDYWGQGTQVTVSSS | |
| | | AAAFVPVFLPAKPTTTPAPRPPTPAPTIASQPLSLRPEACRPAAG | |
| | | GAVHTRGLDFACDIYIWAPLAGTCGVLLLSLVITLYCNHRNRK | |
| | | RGRKKLLYIFKQPFMRPVQTTQEEDGCSCRFPEEEEGGCELRVK | |
| | | FSRSADAPAYQQGQNQLYNELNLGRREEYDVLDKRRGRDPEM | |
| | | GGKPRRKNPQEGLYNELQKDKMAEAYSEIGMKGERRRGKGHD | |
| | | GLYQGLSTATKDTYDALHMQALPPR | |
| CTX-2874 coding | | ATGGCGCTTCCGGTGACAGCACTGCTCCTCCCCTTGGCGCTG | 171 |
| | | TTGCTCCACGCAGCAAGGCCGCAAGTGCAGCTCGTTGAAAGT | |
| | | GGGGGCGGACTTGTTCAGGCAGGTGACAGCCTTCGATTGAG | |
| | | CTGTGCAGTGAGCGGAAGAACTCTGAGCAACTACTTCATGGC | |
| | | GTGGCTGAGACAAGCACCTGGCAAAGAACGGGAGTTCGTCG | |
| | | CTGCGATACACTGGAGCCTCGGCTCAACATACTACCAAGACC | |
| | | CTGTAAAGGGGAGGTTTACGATAAGTAGAGATAAGGCTAAG | |
| | | AATACGGTTTATCTGCAAATGAACACATTGAAGCCTGAGGAT | |
| | | ACAGCAACGTATATATGCGCAGCGGGGCAACACCTGTCTGG | |
| | | GCTGGGTGGGTCTGCATGGTCCATAGAAGGAGACTACTGGG | |
| | | GTCAAGGGACCCAAGTAACAGTCAGCAGCAGTGCTGCTGCC | |
| | | TTTGTCCCGGTATTTCTCCCAGCCAAACCGACCACGACTCCC | |
| | | GCCCCGCGCCCTCCGACACCCGCTCCCACCATCGCCTCTCAA | |
| | | CCTCTTAGTCTTCGCCCCGAGGCATGCCGACCCGCCGCCGGG | |
| | | GGTGCTGTTCATACGAGGGGCTTGGACTTCGCTTGTGATATT | |
| | | TACATTTGGGCTCCGTTGGCGGGTACGTGCGGCGTCCTTTTG | |
| | | TTGTCACTCGTTATTACTTTGTATTGTAATCACAGGAATCGCA | |
| | | AACGGGGCAGAAAGAAACTCCTGTATATATTCAAACAACCA | |
| | | TTTATGAGACCAGTACAAACTACTCAAGAGGAAGATGGCTG | |
| | | TAGCTGCCGATTTCCAGAAGAAGAAGAAGGAGGATGTGAAC | |
| | | TGCGAGTGAAGTTTTCCCGAAGCGCAGACGCTCCGGCATATC | |
| | | AGCAAGGACAGAATCAGCTGTATAACGAACTGAATTTGGGA | |
| | | CGCCGCGAGGAGTATGACGTGCTTGATAAACGCCGGGGGAG | |
| | | AGACCCGGAAATGGGGGGTAAACCCCGAAGAAAGAATCCCC | |
| | | AAGAAGGACTCTACAATGAACTCCAGAAGGATAAGATGGCG | |
| | | GAGGCCTACTCAGAAATAGGTATGAAGGGCGAACGACGACG | |
| | | GGGAAAAGGTCACGATGGCCTCTACCAAGGGTTGAGTACGG | |
| | | CAACCAAAGATACGTACGATGCACTGCATATGCAGGCCCTG | |
| | | CCTCCCAGA | |
| Anti- | HCDR1 | GGTFSIYP | 17 |
| CD117 | HCDR2 | IGWSASGT | 44 |
| VHH | HCDR3 | ASGSNWRLGAIDEY | 71 |
| P35 | VHH | QVQLVESGGGLVQAGGSLRLSCAASGGTFSIYPVGWFRQAPG | 172 |
| | | KQREFVAAIGWSASGTYYGDSVEGRFTVSRDNARSTVYLRMS | |
| | | SLKPDDTAVYYCAAASGSNWRLGAIDEYDSWGQGTQVTVSS | |
| CTX-2875 | | MALPVTALLLPLALLLHAARPQVQLVESGGGLVQAGGSLRLSCA | 173 |
| (P35 VHH; CD28 | | ASGGTFSIYPVGWFRQAPGKQREFVAAIGWSASGTYYGDSVEG | |
| CoS with signal | | RFTVSRDNARSTVYLRMSSLKPDDTAVYYCAAASGSNWRLGAI | |
| peptide) | | DEYDSWGQGTQVTVSSSAAAFVPVFLPAKPTTTPAPRPPTPAPT | |
| | | IASQPLSLRPEACRPAAGGAVHTRGLDFACDIYIWAPLAGTCGV | |
| | | LLLSLVITLYCNHRNRSKRSRLLHSDYMNMTPRRPGPTRKHYQ | |
| | | PYAPPRDFAAYRSRVKFSRSADAPAYQQGQNQLYNELNLGRRE | |
| | | EYDVLDKRRGRDPEMGGKPRRKNPQEGLYNELQKDKMAEAY | |
| | | SEIGMKGERRRGKGHDGLYQGLSTATKDTYDALHMQALPPR | |
| CTX-2875 | | QVQLVESGGGLVQAGGSLRLSCAASGGTFSIYPVGWFRQAPGK | 174 |
| (no signal peptide) | | QREFVAAIGWSASGTYYGDSVEGRFTVSRDNARSTVYLRMSSL | |
| | | KPDDTAVYYCAAASGSNWRLGAIDEYDSWGQGTQVTVSSSAA | |
| | | AFVPVFLPAKPTTTPAPRPPTPAPTIASQPLSLRPEACRPAAGGA | |
| | | VHTRGLDFACDIYIWAPLAGTCGVLLLSLVITLYCNHRNRSKRS | |
| | | RLLHSDYMNMTPRRPGPTRKHYQPYAPPRDFAAYRSRVKFSRS | |
| | | ADAPAYQQGQNQLYNELNLGRREEYDVLDKRRGRDPEMGGK | |
| | | PRRKNPQEGLYNELQKDKMAEAYSEIGMKGERRRGKGHDGLY | |
| | | QGLSTATKDTYDALHMQALPPR | |
| CTX-2875 coding | | ATGGCGCTTCCGGTGACAGCACTGCTCCTCCCCTTGGCGCTG | 175 |
| | | TTGCTCCACGCAGCAAGGCCGCAAGTGCAACTCGTTGAATCT | |
| | | GGAGGAGGTCTGGTACAAGCGGGGGGTTCACTGAGACTCTC | |
| | | TTGTGCAGCGTCAGGTGGCACTTTTAGTATATATCCGGTTGG | |
| | | ATGGTTCAGACAAGCACCAGGAAAACAAAGGGAATTTGTTG | |
| | | CTGCAATCGGTTGGTCCGCCTCCGGGACGTATTATGGTGATA | |
| | | GTGTCGAGGGCAGATTCACAGTGTCACGCGACAATGCGAGA | |
| | | TCTACGGTCTATCTCAGGATGAGTTCACTTAAACCTGACGAC | |
| | | ACCGCCGTCTATTATTGTGCAGCAGCAAGCGGTTCTAACTGG | |
| | | CGATTGGGAGCAATTGATGAATATGATAGCTGGGGACAAGG | |
| | | TACTCAGGTTACTGTTTCCAGTAGTGCTGCTGCCTTTGTCCCG | |
| | | GTATTTCTCCCAGCCAAACCGACCACGACTCCCGCCCCGCGC | |
| | | CCTCCGACACCCGCTCCCACCATCGCCTCTCAACCTCTTAGTC | |
| | | TTCGCCCCGAGGCATGCCGACCCGCCGCCGGGGGTGCTGTTC | |
| | | ATACGAGGGGCTTGGACTTCGCTTGTGATATTTACATTTGGG | |
| | | CTCCGTTGGCGGGTACGTGCGGCGTCCTTTTGTTGTCACTCGT | |
| | | TATTACTTTGTATTGTAATCACAGGAATCGCTCAAAGCGGAG | |
| | | TAGGTTGTTGCATTCCGATTACATGAATATGACTCCTCGCCG | |
| | | GCCTGGGCCGACAAGAAAACATTACCAACCCTATGCCCCCCC | |
| | | ACGAGACTTCGCTGCGTACAGGTCCCGAGTGAAGTTTTCCCG | |
| | | AAGCGCAGACGCTCCGGCATATCAGCAAGGACAGAATCAGC | |
| | | TGTATAACGAACTGAATTTGGGACGCCGCGAGGAGTATGAC | |
| | | GTGCTTGATAAACGCCGGGGGAGAGACCCGGAAATGGGGGG | |
| | | TAAACCCCGAAGAAAGAATCCCCAAGAAGGACTCTACAATG | |
| | | AACTCCAGAAGGATAAGATGGCGGAGGCCTACTCAGAAATA | |
| | | GGTATGAAGGGCGAACGACGACGGGGAAAAGGTCACGATG | |
| | | GCCTCTACCAAGGGTTGAGTACGGCAACCAAAGATACGTAC | |
| | | GATGCACTGCATATGCAGGCCCTGCCTCCCAGA | |
| CTX-2876 | | MALPVTALLLPLALLLHAARPQVQLVESGGGLVQAGGSLRLSCA | 176 |
| (P35 VHH; 4-1BB | | ASGGTFSIYPVGWFRQAPGKQREFVAAIGWSASGTYYGDSVEG | |
| CoS with signal | | RFTVSRDNARSTVYLRMSSLKPDDTAVYYCAAASGSNWRLGAI | |
| peptide) | | DEYDSWGQGTQVTVSSSAAAFVPVFLPAKPTTTPAPRPPTPAPTI | |
| Anti-CD117 CAR | | ASQPLSLRPEACRPAAGGAVHTRGLDFACDIYIWAPLAGTCGV | |
| | | LLLSLVITLYCNHRNRKRGRKKLLYIFKQPFMRPVQTTQEEDGC | |
| | | SCRFPEEEEGGCELRVKFSRSADAPAYQQGQNQLYNELNLGRR | |
| | | EEYDVLDKRRGRDPEMGGKPRRKNPQEGLYNELQKDKMAEA | |
| | | YSEIGMKGERRRGKGHDGLYQGLSTATKDTYDALHMQALPPR | |
| CTX-2876 | | QVQLVESGGGLVQAGGSLRLSCAASGGTFSIYPVGWFRQAPGK | 177 |
| (no signal peptide) | | QREFVAAIGWSASGTYYGDSVEGRFTVSRDNARSTVYLRMSSL | |
| | | KPDDTAVYYCAAASGSNWRLGAIDEYDSWGQGTQVTVSSSAA | |
| | | AFVPVFLPAKPTTTPAPRPPTPAPTIASQPLSLRPEACRPAAGGA | |
| | | VHTRGLDFACDIYIWAPLAGTCGVLLLSLVITLYCNHRNRKRGR | |
| | | KKLLYIFKQPFMRPVQTTQEEDGCSCRFPEEEEGGCELRVKFSR | |
| | | SADAPAYQQGQNQLYNELNLGRREEYDVLDKRRGRDPEMGG | |
| | | KPRRKNPQEGLYNELQKDKMAEAYSEIGMKGERRRGKGHDGL | |
| | | YQGLSTATKDTYDALHMQALPPR | |
| CTX-2876 coding | | ATGGCGCTTCCGGTGACAGCACTGCTCCTCCCCTTGGCGCTG | 178 |
| | | TTGCTCCACGCAGCAAGGCCGCAAGTGCAACTCGTTGAATCT | |
| | | GGAGGAGGTCTGGTACAAGCGGGGGGTTCACTGAGACTCTC | |
| | | TTGTGCAGCGTCAGGTGGCACTTTTAGTATATATCCGGTTGG | |
| | | ATGGTTCAGACAAGCACCAGGAAAACAAAGGGAATTTGTTG | |
| | | CTGCAATCGGTTGGTCCGCCTCCGGGACGTATTATGGTGATA | |
| | | GTGTCGAGGGCAGATTCACAGTGTCACGCGACAATGCGAGA | |
| | | TCTACGGTCTATCTCAGGATGAGTTCACTTAAACCTGACGAC | |
| | | ACCGCCGTCTATTATTGTGCAGCAGCAAGCGGTTCTAACTGG | |
| | | CGATTGGGAGCAATTGATGAATATGATAGCTGGGGACAAGG | |
| | | TACTCAGGTTACTGTTTCCAGTAGTGCTGCTGCCTTTGTCCCG | |
| | | GTATTTCTCCCAGCCAAACCGACCACGACTCCCGCCCCGCGC | |
| | | CCTCCGACACCCGCTCCCACCATCGCCTCTCAACCTCTTAGTC | |
| | | TTCGCCCCGAGGCATGCCGACCCGCCGCCGGGGGTGCTGTTC | |
| | | ATACGAGGGGCTTGGACTTCGCTTGTGATATTTACATTTGGG | |
| | | CTCCGTTGGCGGGTACGTGCGGCGTCCTTTTGTTGTCACTCGT | |
| | | TATTACTTTGTATTGTAATCACAGGAATCGCAAACGGGGCAG | |
| | | AAAGAAACTCCTGTATATATTCAAACAACCATTTATGAGACC | |
| | | AGTACAAACTACTCAAGAGGAAGATGGCTGTAGCTGCCGAT | |
| | | TTCCAGAAGAAGAAGAAGGAGGATGTGAACTGCGAGTGAAG | |
| | | TTTTCCCGAAGCGCAGACGCTCCGGCATATCAGCAAGGACA | |
| | | GAATCAGCTGTATAACGAACTGAATTTGGGACGCCGCGAGG | |
| | | AGTATGACGTGCTTGATAAACGCCGGGGGAGAGACCCGGAA | |
| | | ATGGGGGGTAAACCCCGAAGAAAGAATCCCCAAGAAGGACT | |
| | | CTACAATGAACTCCAGAAGGATAAGATGGCGGAGGCCTACT | |
| | | CAGAAATAGGTATGAAGGGCGAACGACGACGGGGAAAAGG | |
| | | TCACGATGGCCTCTACCAAGGGTTGAGTACGGCAACCAAAG | |
| | | ATACGTACGATGCACTGCATATGCAGGCCCTGCCTCCCAGA | |
| Anti- | HCDR1 | GRTFSSYA | 19 |
| CD117 | HCDR2 | ITSSGLVA | 46 |
| VHH | HCDR3 | RQYVGSGSYYLKKEGGY | 73 |
| P37 | VHH | QVQLVESGGGLVQAGDSLRLSCAASGRTFSSYAMGWFRQAPG | 179 |
| | | KEREFVASITSSGLVAYYGDSVEARFTISRDNAKNTVYLQMDS | |
| | | LKPEDTAVYYCAARQYVGSGSYYLKKEGGYEYWGPGTQVTV | |
| | | SS | |
| CTX-2877 | | MALPVTALLLPLALLLHAARPQVQLVESGGGLVQAGDSLRLSCA | 180 |
| (P37 VHH; CD28 | | ASGRTFSSYAMGWFRQAPGKEREFVASITSSGLVAYYGDSVEA | |
| CoS with signal | | RFTISRDNAKNTVYLQMDSLKPEDTAVYYCAARQYVGSGSYY | |
| peptide) | | LKKEGGYEYWGPGTQVTVSSSAAAFVPVFLPAKPTTTPAPRPPT | |
| | | PAPTIASQPLSLRPEACRPAAGGAVHTRGLDFACDIYIWAPLAG | |
| | | TCGVLLLSLVITLYCNHRNRSKRSRLLHSDYMNMTPRRPGPTR | |
| | | KHYQPYAPPRDFAAYRSRVKFSRSADAPAYQQGQNQLYNELN | |
| | | LGRREEYDVLDKRRGRDPEMGGKPRRKNPQEGLYNELQKDKM | |
| | | AEAYSEIGMKGERRRGKGHDGLYQGLSTATKDTYDALHMQAL | |
| | | PPR | |
| CTX-2877 | | QVQLVESGGGLVQAGDSLRLSCAASGRTFSSYAMGWFRQAPG | 181 |
| (no signal peptide) | | KEREFVASITSSGLVAYYGDSVEARFTISRDNAKNTVYLQMDSL | |
| | | KPEDTAVYYCAARQYVGSGSYYLKKEGGYEYWGPGTQVTVSS | |
| | | SAAAFVPVFLPAKPTTTPAPRPPTPAPTIASQPLSLRPEACRPAAG | |
| | | GAVHTRGLDFACDIYIWAPLAGTCGVLLLSLVITLYCNHRNRSK | |
| | | RSRLLHSDYMNMTPRRPGPTRKHYQPYAPPRDFAAYRSRVKFS | |
| | | RSADAPAYQQGQNQLYNELNLGRREEYDVLDKRRGRDPEMGG | |
| | | KPRRKNPQEGLYNELQKDKMAEAYSEIGMKGERRRGKGHDGL | |
| | | YQGLSTATKDTYDALHMQALPPR | |
| CTX-2877 coding | | ATGGCGCTTCCGGTGACAGCACTGCTCCTCCCCTTGGCGCTG | 182 |
| | | TTGCTCCACGCAGCAAGGCCGCAGGTTCAGCTCGTAGAGAGT | |
| | | GGTGGGGGACTGGTTCAGGCTGGCGATTCTCTTCGGCTTTCC | |
| | | TGCGCGGCTAGTGGACGAACCTTTAGCAGTTATGCTATGGGC | |
| | | TGGTTCCGCCAAGCCCCCGGAAAAGAACGCGAGTTTGTAGC | |
| | | CAGCATCACGAGTTCTGGACTTGTTGCGTACTACGGCGATAG | |
| | | CGTTGAGGCAAGGTTCACCATCAGTCGAGACAACGCGAAAA | |
| | | ATACCGTTTACTTGCAGATGGATAGTCTGAAACCGGAAGACA | |
| | | CCGCCGTCTACTACTGCGCTGCGCGGCAGTACGTCGGGAGCG | |
| | | GAAGCTATTATTTGAAAAAGGAAGGCGGTTATGAATACTGG | |
| | | GGGCCTGGCACACAAGTAACGGTTTCAAGCAGTGCTGCTGCC | |
| | | TTTGTCCCGGTATTTCTCCCAGCCAAACCGACCACGACTCCC | |
| | | GCCCCGCGCCCTCCGACACCCGCTCCCACCATCGCCTCTCAA | |
| | | CCTCTTAGTCTTCGCCCCGAGGCATGCCGACCCGCCGCCGGG | |
| | | GGTGCTGTTCATACGAGGGGCTTGGACTTCGCTTGTGATATT | |
| | | TACATTTGGGCTCCGTTGGCGGGTACGTGCGGCGTCCTTTTG | |
| | | TTGTCACTCGTTATTACTTTGTATTGTAATCACAGGAATCGCT | |
| | | CAAAGCGGAGTAGGTTGTTGCATTCCGATTACATGAATATGA | |
| | | CTCCTCGCCGGCCTGGGCCGACAAGAAAACATTACCAACCCT | |
| | | ATGCCCCCCCACGAGACTTCGCTGCGTACAGGTCCCGAGTGA | |
| | | AGTTTTCCCGAAGCGCAGACGCTCCGGCATATCAGCAAGGA | |
| | | CAGAATCAGCTGTATAACGAACTGAATTTGGGACGCCGCGA | |
| | | GGAGTATGACGTGCTTGATAAACGCCGGGGGAGAGACCCGG | |
| | | AAATGGGGGGTAAACCCCGAAGAAAGAATCCCCAAGAAGG | |
| | | ACTCTACAATGAACTCCAGAAGGATAAGATGGCGGAGGCCT | |
| | | ACTCAGAAATAGGTATGAAGGGCGAACGACGACGGGGAAA | |
| | | AGGTCACGATGGCCTCTACCAAGGGTTGAGTACGGCAACCA | |
| | | AAGATACGTACGATGCACTGCATATGCAGGCCCTGCCTCCCA | |
| | | GA | |
| CTX-2878 | | MALPVTALLLPLALLLHAARPQVQLVESGGGLVQAGDSLRLSCA | 183 |
| (P37 VHH; 4-1BB | | ASGRTFSSYAMGWFRQAPGKEREFVASITSSGLVAYYGDSVEA | |
| CoS with signal | | RFTISRDNAKNTVYLQMDSLKPEDTAVYYCAARQYVGSGSYY | |
| peptide) | | LKKEGGYEYWGPGTQVTVSSSAAAFVPVFLPAKPTTTPAPRPPT | |
| | | PAPTIASQPLSLRPEACRPAAGGAVHTRGLDFACDIYIWAPLAG | |
| | | TCGVLLLSLVITLYCNHRNRKRGRKKLLYIFKQPFMRPVQTTQE | |
| | | EDGCSCRFPEEEEGGCELRVKFSRSADAPAYQQGQNQLYNELN | |
| | | LGRREEYDVLDKRRGRDPEMGGKPRRKNPQEGLYNELQKDKM | |
| | | AEAYSEIGMKGERRRGKGHDGLYQGLSTATKDTYDALHMQAL | |
| | | PPR | |
| CTX-2878 | | QVQLVESGGGLVQAGDSLRLSCAASGRTFSSYAMGWFRQAPG | 228 |
| (no signal peptide) | | KEREFVASITSSGLVAYYGDSVEARFTISRDNAKNTVYLQMDSL | |
| | | KPEDTAVYYCAARQYVGSGSYYLKKEGGYEYWGPGTQVTVSS | |
| | | SAAAFVPVFLPAKPTTTPAPRPPTPAPTIASQPLSLRPEACRPAAG | |
| | | GAVHTRGLDFACDIYIWAPLAGTCGVLLLSLVITLYCNHRNRK | |
| | | RGRKKLLYIFKQPFMRPVQTTQEEDGCSCRFPEEEEGGCELRVK | |
| | | FSRSADAPAYQQGQNQLYNELNLGRREEYDVLDKRRGRDPEM | |
| | | GGKPRRKNPQEGLYNELQKDKMAEAYSEIGMKGERRRGKGHD | |
| | | GLYQGLSTATKDTYDALHMQALPPR | |
| CTX-2878 coding | | ATGGCGCTTCCGGTGACAGCACTGCTCCTCCCCTTGGCGCTG | 184 |
| | | TTGCTCCACGCAGCAAGGCCGCAGGTTCAGCTCGTAGAGAGT | |
| | | GGTGGGGGACTGGTTCAGGCTGGCGATTCTCTTCGGCTTTCC | |
| | | TGCGCGGCTAGTGGACGAACCTTTAGCAGTTATGCTATGGGC | |
| | | TGGTTCCGCCAAGCCCCCGGAAAAGAACGCGAGTTTGTAGC | |
| | | CAGCATCACGAGTTCTGGACTTGTTGCGTACTACGGCGATAG | |
| | | CGTTGAGGCAAGGTTCACCATCAGTCGAGACAACGCGAAAA | |
| | | ATACCGTTTACTTGCAGATGGATAGTCTGAAACCGGAAGACA | |
| | | CCGCCGTCTACTACTGCGCTGCGCGGCAGTACGTCGGGAGCG | |
| | | GAAGCTATTATTTGAAAAAGGAAGGCGGTTATGAATACTGG | |
| | | GGGCCTGGCACACAAGTAACGGTTTCAAGCAGTGCTGCTGCC | |
| | | TTTGTCCCGGTATTTCTCCCAGCCAAACCGACCACGACTCCC | |
| | | GCCCCGCGCCCTCCGACACCCGCTCCCACCATCGCCTCTCAA | |
| | | CCTCTTAGTCTTCGCCCCGAGGCATGCCGACCCGCCGCCGGG | |
| | | GGTGCTGTTCATACGAGGGGCTTGGACTTCGCTTGTGATATT | |
| | | TACATTTGGGCTCCGTTGGCGGGTACGTGCGGCGTCCTTTTG | |
| | | TTGTCACTCGTTATTACTTTGTATTGTAATCACAGGAATCGCA | |
| | | AACGGGGCAGAAAGAAACTCCTGTATATATTCAAACAACCA | |
| | | TTTATGAGACCAGTACAAACTACTCAAGAGGAAGATGGCTG | |
| | | TAGCTGCCGATTTCCAGAAGAAGAAGAAGGAGGATGTGAAC | |
| | | TGCGAGTGAAGTTTTCCCGAAGCGCAGACGCTCCGGCATATC | |
| | | AGCAAGGACAGAATCAGCTGTATAACGAACTGAATTTGGGA | |
| | | CGCCGCGAGGAGTATGACGTGCTTGATAAACGCCGGGGGAG | |
| | | AGACCCGGAAATGGGGGGTAAACCCCGAAGAAAGAATCCCC | |
| | | AAGAAGGACTCTACAATGAACTCCAGAAGGATAAGATGGCG | |
| | | GAGGCCTACTCAGAAATAGGTATGAAGGGCGAACGACGACG | |
| | | GGGAAAAGGTCACGATGGCCTCTACCAAGGGTTGAGTACGG | |
| | | CAACCAAAGATACGTACGATGCACTGCATATGCAGGCCCTG | |
| | | CCTCCCAGA | |
| Anti- | HCDR1 | GRTFSSYA | 19 |
| CD117 | HCDR2 | ILSNGLTT | 48 |
| VHH | HCDR3 | AFTPEFRDGGIWDDASV | 75 |
| P10 | VHH | QVQLVESGGGLVQAGGSLRLSCVASGRTFSSYAMGWFRQGPG | 185 |
| | | KEREFAAAILSNGLTTYYADSVKGRFTISRDNAKDTVYLQMN | |
| | | SLKPEDTANYYCAAAFTPEFRDGGIWDDASVDYWGQGTQVTV | |
| | | SS | |
| CTX-2879 | | MALPVTALLLPLALLLHAARPQVQLVESGGGLVQAGGSLRLSCV | 186 |
| (P37 VHH; CD28 | | ASGRTFSSYAMGWFRQGPGKEREFAAAILSNGLTTYYADSVKG | |
| CoS with signal | | RFTISRDNAKDTVYLQMNSLKPEDTANYYCAAAFTPEFRDGGI | |
| peptide) | | WDDASVDYWGQGTQVTVSSSAAAFVPVFLPAKPTTTPAPRPPT | |
| | | PAPTIASQPLSLRPEACRPAAGGAVHTRGLDFACDIYIWAPLAG | |
| | | TCGVLLLSLVITLYCNHRNRSKRSRLLHSDYMNMTPRRPGPTR | |
| | | KHYQPYAPPRDFAAYRSRVKFSRSADAPAYQQGQNQLYNELN | |
| | | LGRREEYDVLDKRRGRDPEMGGKPRRKNPQEGLYNELQKDKM | |
| | | AEAYSEIGMKGERRRGKGHDGLYQGLSTATKDTYDALHMQAL | |
| | | PPR | |
| CTX-2879 | | QVQLVESGGGLVQAGGSLRLSCVASGRTFSSYAMGWFRQGPG | 187 |
| (no signal peptide) | | KEREFAAAILSNGLTTYYADSVKGRFTISRDNAKDTVYLQMNS | |
| | | LKPEDTANYYCAAAFTPEFRDGGIWDDASVDYWGQGTQVTVS | |
| | | SSAAAFVPVFLPAKPTTTPAPRPPTPAPTIASQPLSLRPEACRPAA | |
| | | GGAVHTRGLDFACDIYIWAPLAGTCGVLLLSLVITLYCNHRNRS | |
| | | KRSRLLHSDYMNMTPRRPGPTRKHYQPYAPPRDFAAYRSRVKF | |
| | | SRSADAPAYQQGQNQLYNELNLGRREEYDVLDKRRGRDPEMG | |
| | | GKPRRKNPQEGLYNELQKDKMAEAYSEIGMKGERRRGKGHDG | |
| | | LYQGLSTATKDTYDALHMQALPPR | |
| CTX-2879 coding | | ATGGCGCTTCCGGTGACAGCACTGCTCCTCCCCTTGGCGCTG | 188 |
| | | TTGCTCCACGCAGCAAGGCCGCAAGTCCAACTGGTAGAGTCT | |
| | | GGGGGCGGCCTCGTGCAAGCCGGAGGTTCCCTCAGGCTCTCT | |
| | | TGTGTGGCATCAGGTAGGACCTTTAGTTCCTATGCCATGGGT | |
| | | TGGTTCCGGCAAGGGCCTGGAAAAGAGAGGGAATTTGCGGC | |
| | | AGCCATACTTTCAAACGGACTTACCACCTACTATGCCGACAG | |
| | | TGTGAAAGGGCGATTTACCATATCTCGCGATAATGCGAAGG | |
| | | ACACGGTCTATCTCCAGATGAATAGCCTCAAGCCGGAAGAC | |
| | | ACCGCTAACTACTATTGTGCTGCTGCCTTTACGCCCGAATTTC | |
| | | GGGACGGAGGCATCTGGGATGACGCATCAGTTGATTATTGG | |
| | | GGCCAGGGAACCCAAGTAACGGTGTCATCCAGTGCTGCTGC | |
| | | CTTTGTCCCGGTATTTCTCCCAGCCAAACCGACCACGACTCC | |
| | | CGCCCCGCGCCCTCCGACACCCGCTCCCACCATCGCCTCTCA | |
| | | ACCTCTTAGTCTTCGCCCCGAGGCATGCCGACCCGCCGCCGG | |
| | | GGGTGCTGTTCATACGAGGGGCTTGGACTTCGCTTGTGATAT | |
| | | TTACATTTGGGCTCCGTTGGCGGGTACGTGCGGCGTCCTTTT | |
| | | GTTGTCACTCGTTATTACTTTGTATTGTAATCACAGGAATCGC | |
| | | TCAAAGCGGAGTAGGTTGTTGCATTCCGATTACATGAATATG | |
| | | ACTCCTCGCCGGCCTGGGCCGACAAGAAAACATTACCAACC | |
| | | CTATGCCCCCCCACGAGACTTCGCTGCGTACAGGTCCCGAGT | |
| | | GAAGTTTTCCCGAAGCGCAGACGCTCCGGCATATCAGCAAG | |
| | | GACAGAATCAGCTGTATAACGAACTGAATTTGGGACGCCGC | |
| | | GAGGAGTATGACGTGCTTGATAAACGCCGGGGGAGAGACCC | |
| | | GGAAATGGGGGGTAAACCCCGAAGAAAGAATCCCCAAGAA | |
| | | GGACTCTACAATGAACTCCAGAAGGATAAGATGGCGGAGGC | |
| | | CTACTCAGAAATAGGTATGAAGGGCGAACGACGACGGGGAA | |
| | | AAGGTCACGATGGCCTCTACCAAGGGTTGAGTACGGCAACC | |
| | | AAAGATACGTACGATGCACTGCATATGCAGGCCCTGCCTCCC | |
| | | AGA | |
| CTX-2880 | | MALPVTALLLPLALLLHAARPQVQLVESGGGLVQAGGSLRLSCV | 189 |
| (P10 VHH; 4-1BB | | ASGRTFSSYAMGWFRQGPGKEREFAAAILSNGLTTYYADSVKG | |
| CoS with signal | | RFTISRDNAKDTVYLQMNSLKPEDTANYYCAAAFTPEFRDGGI | |
| peptide) | | WDDASVDYWGQGTQVTVSSSAAAFVPVFLPAKPTTTPAPRPPT | |
| | | PAPTIASQPLSLRPEACRPAAGGAVHTRGLDFACDIYIWAPLAG | |
| | | TCGVLLLSLVITLYCNHRNRKRGRKKLLYIFKQPFMRPVQTTQE | |
| | | EDGCSCRFPEEEEGGCELRVKFSRSADAPAYQQGQNQLYNELN | |
| | | LGRREEYDVLDKRRGRDPEMGGKPRRKNPQEGLYNELQKDKM | |
| | | AEAYSEIGMKGERRRGKGHDGLYQGLSTATKDTYDALHMQAL | |
| | | PPR | |
| CTX-2880 | | QVQLVESGGGLVQAGGSLRLSCVASGRTFSSYAMGWFRQGPG | 190 |
| (no signal peptide) | | KEREFAAAILSNGLTTYYADSVKGRFTISRDNAKDTVYLQMNS | |
| | | LKPEDTANYYCAAAFTPEFRDGGIWDDASVDYWGQGTQVTVS | |
| | | SSAAAFVPVFLPAKPTTTPAPRPPTPAPTIASQPLSLRPEACRPAA | |
| | | GGAVHTRGLDFACDIYIWAPLAGTCGVLLLSLVITLYCNHRNR | |
| | | KRGRKKLLYIFKQPFMRPVQTTQEEDGCSCRFPEEEEGGCELRV | |
| | | KFSRSADAPAYQQGQNQLYNELNLGRREEYDVLDKRRGRDPE | |
| | | MGGKPRRKNPQEGLYNELQKDKMAEAYSEIGMKGERRRGKG | |
| | | HDGLYQGLSTATKDTYDALHMQALPPR | |
| CTX-2880 coding | | ATGGCGCTTCCGGTGACAGCACTGCTCCTCCCCTTGGCGCTG | 191 |
| | | TTGCTCCACGCAGCAAGGCCGCAAGTCCAACTGGTAGAGTCT | |
| | | GGGGGCGGCCTCGTGCAAGCCGGAGGTTCCCTCAGGCTCTCT | |
| | | TGTGTGGCATCAGGTAGGACCTTTAGTTCCTATGCCATGGGT | |
| | | TGGTTCCGGCAAGGGCCTGGAAAAGAGAGGGAATTTGCGGC | |
| | | AGCCATACTTTCAAACGGACTTACCACCTACTATGCCGACAG | |
| | | TGTGAAAGGGCGATTTACCATATCTCGCGATAATGCGAAGG | |
| | | ACACGGTCTATCTCCAGATGAATAGCCTCAAGCCGGAAGAC | |
| | | ACCGCTAACTACTATTGTGCTGCTGCCTTTACGCCCGAATTTC | |
| | | GGGACGGAGGCATCTGGGATGACGCATCAGTTGATTATTGG | |
| | | GGCCAGGGAACCCAAGTAACGGTGTCATCCAGTGCTGCTGC | |
| | | CTTTGTCCCGGTATTTCTCCCAGCCAAACCGACCACGACTCC | |
| | | CGCCCCGCGCCCTCCGACACCCGCTCCCACCATCGCCTCTCA | |
| | | ACCTCTTAGTCTTCGCCCCGAGGCATGCCGACCCGCCGCCGG | |
| | | GGGTGCTGTTCATACGAGGGGCTTGGACTTCGCTTGTGATAT | |
| | | TTACATTTGGGCTCCGTTGGCGGGTACGTGCGGCGTCCTTTT | |
| | | GTTGTCACTCGTTATTACTTTGTATTGTAATCACAGGAATCGC | |
| | | AAACGGGGCAGAAAGAAACTCCTGTATATATTCAAACAACC | |
| | | ATTTATGAGACCAGTACAAACTACTCAAGAGGAAGATGGCT | |
| | | GTAGCTGCCGATTTCCAGAAGAAGAAGAAGGAGGATGTGAA | |
| | | CTGCGAGTGAAGTTTTCCCGAAGCGCAGACGCTCCGGCATAT | |
| | | CAGCAAGGACAGAATCAGCTGTATAACGAACTGAATTTGGG | |
| | | ACGCCGCGAGGAGTATGACGTGCTTGATAAACGCCGGGGGA | |
| | | GAGACCCGGAAATGGGGGGTAAACCCCGAAGAAAGAATCCC | |
| | | CAAGAAGGACTCTACAATGAACTCCAGAAGGATAAGATGGC | |
| | | GGAGGCCTACTCAGAAATAGGTATGAAGGGCGAACGACGAC | |
| | | GGGGAAAAGGTCACGATGGCCTCTACCAAGGGTTGAGTACG | |
| | | GCAACCAAAGATACGTACGATGCACTGCATATGCAGGCCCT | |
| | | GCCTCCCAGA | |
| Anti- | HCDR1 | GDTFSSYS | 24 |
| CD117 | HCDR2 | IRWSGGTT | 51 |
| VHH | HCDR3 | VRRRWLIWQEEEY | 78 |
| P12 | VHH | QVQLVESGGGLVQAGGSLRLSCTASGDTFSSYSMGWFRQGPG | 192 |
| | | KEREFVGGIRWSGGTTYYADSVKGRFTISRDNAKNTVYLQMN | |
| | | SLKREDTAVYYCAAVRRRWLIWQEEEYDYWGQGTLVTVSS | |

| | | |
|---|---|---|
| CTX-2881 | MALPVTALLLPLALLLHAARPQVQLVESGGGLVQAGGSLRLSC | 193 |
| (P12 VHH; CD28 | TASGDTFSSYSMGWFRQGPGKEREFVGGIRWSGGTTYYADSVK | |
| CoS with signal | GRFTISRDNAKNTVYLQMNSLKREDTAVYYCAAVRRRWLIWQE | |
| peptide) | EEYDYWGQGTLVTVSSSAAAFVPVFLPAKPTTTPAPRPPTPAP | |
| | TIASQPLSLRPEACRPAAGGAVHTRGLDFACDIYIWAPLAGTC | |
| | GVLLLSLVITLYCNHRNRSKRSRLLHSDYMNMTPRRPGPTRKH | |
| | YQPYAPPRDFAAYRSRVKFSRSADAPAYQQGQNQLYNELNLGR | |
| | REEYDVLDKRRGRDPEMGGKPRRKNPQEGLYNELQKDKMAEAY | |
| | SEIGMKGERRRGKGHDGLYQGLSTATKDTYDALHMQALPPR | |
| CTX-2881 | QVQLVESGGGLVQAGGSLRLSCTASGDTFSSYSMGWFRQGPG | 194 |
| (no signal peptide) | KEREFVGGIRWSGGTTYYADSVKGRFTISRDNAKNTVYLQMNS | |
| | LKREDTAVYYCAAVRRRWLIWQEEEYDYWGQGTLVTVSSSAAA | |
| | FVPVFLPAKPTTTPAPRPPTPAPTIASQPLSLRPEACRPAAGGA | |
| | VHTRGLDFACDIYIWAPLAGTCGVLLLSLVITLYCNHRNRSKRS | |
| | RLLHSDYMNMTPRRPGPTRKHYQPYAPPRDFAAYRSRVKFSRS | |
| | ADAPAYQQGQNQLYNELNLGRREEYDVLDKRRGRDPEMGGK | |
| | PRRKNPQEGLYNELQKDKMAEAYSEIGMKGERRRGKGHDGLY | |
| | QGLSTATKDTYDALHMQALPPR | |
| CTX-2881 coding | ATGGCGCTTCCGGTGACAGCACTGCTCCTCCCCTTGGCGCTG | 195 |
| | TTGCTCCACGCAGCAAGGCCGCAAGTACAATTGGTGGAGTCT | |
| | GGGGGTGGTTTGGTTCAAGCTGGTGGGAGTCTCAGACTGTCA | |
| | TGTACGGCCAGCGGGGACACTTTCTCATCTTATAGCATGGGT | |
| | TGGTTTCGACAGGGTCCCGGAAAGGAACGAGAGTTCGTAGG | |
| | CGGCATTCGATGGAGTGGGGGCACGACTTACTACGCCGATTC | |
| | TGTGAAGGGTCGGTTCACAATAAGTCGAGACAACGCCAAGA | |
| | ATACAGTGTACCTCCAGATGAATTCCTTGAAAAGGGAGGAT | |
| | ACGGCCGTCTATTATTGCGCGGCCGTGAGACGGCGATGGCTC | |
| | ATCTGGCAAGAAGAAGAGTACGATTATTGGGGCCAAGGGAC | |
| | TCTTGTAACCGTGAGTTCTAGTGCTGCTGCCTTTGTCCCGGTA | |
| | TTTCTCCCAGCCAAACCGACCACGACTCCCGCCCCGCGCCCT | |
| | CCGACACCCGCTCCCACCATCGCCTCTCAACCTCTTAGTCTTC | |
| | GCCCCGAGGCATGCCGACCCGCCGCCGGGGGTGCTGTTCATA | |
| | CGAGGGGCTTGGACTTCGCTTGTGATATTTACATTTGGGCTC | |
| | CGTTGGCGGGTACGTGCGGCGTCCTTTTGTTGTCACTCGTTAT | |
| | TACTTTGTATTGTAATCACAGGAATCGCTCAAAGCGGAGTAG | |
| | GTTGTTGCATTCCGATTACATGAATATGACTCCTCGCCGGCC | |
| | TGGGCCGACAAGAAAACATTACCAACCCTATGCCCCCCCAC | |
| | GAGACTTCGCTGCGTACAGGTCCCGAGTGAAGTTTTCCCGAA | |
| | GCGCAGACGCTCCGGCATATCAGCAAGGACAGAATCAGCTG | |
| | TATAACGAACTGAATTTGGGACGCCGCGAGGAGTATGACGT | |
| | GCTTGATAAACGCCGGGGGAGAGACCCGGAAATGGGGGGTA | |
| | AACCCCGAAGAAAGAATCCCCAAGAAGGACTCTACAATGAA | |
| | CTCCAGAAGGATAAGATGGCGGAGGCCTACTCAGAAATAGG | |
| | TATGAAGGGCGAACGACGACGGGGAAAAGGTCACGATGGCC | |
| | TCTACCAAGGGTTGAGTACGGCAACCAAAGATACGTACGAT | |
| | GCACTGCATATGCAGGCCCTGCCTCCCAGA | |
| CTX-2882 | MALPVTALLLPLALLLHAARPQVQLVESGGGLVQAGGSLRLSC | 196 |
| (P12 VHH; 4-1BB | TASGDTFSSYSMGWFRQGPGKEREFVGGIRWSGGTTYYADSVK | |
| CoS with signal | GRFTISRDNAKNTVYLQMNSLKREDTAVYYCAAVRRRWLIWQE | |
| peptide) | EEYDYWGQGTLVTVSSSAAAFVPVFLPAKPTTTPAPRPPTPAP | |
| | TIASQPLSLRPEACRPAAGGAVHTRGLDFACDIYIWAPLAGTC | |
| | GVLLLSLVITLYCNHRNRKRGRKKLLYIFKQPFMRPVQTTQEE | |
| | DGCSCRFPEEEEGGCELRVKFSRSADAPAYQQGQNQLYNELNL | |
| | GRREEYDVLDKRRGRDPEMGGKPRRKNPQEGLYNELQKDKMAE | |
| | AYSEIGMKGERRRGKGHDGLYQGLSTATKDTYDALHMQALPPR | |
| CTX-2882 | QVQLVESGGGLVQAGGSLRLSCTASGDTFSSYSMGWFRQGPG | 197 |
| (no signal peptide) | KEREFVGGIRWSGGTTYYADSVKGRFTISRDNAKNTVYLQMNS | |
| | LKREDTAVYYCAAVRRRWLIWQEEEYDYWGQGTLVTVSSSAA | |
| | AFVPVFLPAKPTTTPAPRPPTPAPTIASQPLSLRPEACRPAAGGA | |
| | VHTRGLDFACDIYIWAPLAGTCGVLLLSLVITLYCNHRNRKRGR | |
| | KKLLYIFKQPFMRPVQTTQEEDGCSCRFPEEEEGGCELRVKFSR | |
| | SADAPAYQQGQNQLYNELNLGRREEYDVLDKRRGRDPEMGG | |
| | KPRRKNPQEGLYNELQKDKMAEAYSEIGMKGERRRGKGHDGL | |
| | YQGLSTATKDTYDALHMQALPPR | |
| CTX-2882 coding | ATGGCGCTTCCGGTGACAGCACTGCTCCTCCCCTTGGCGCTG | 198 |
| | TTGCTCCACGCAGCAAGGCCGCAAGTACAATTGGTGGAGTCT | |
| | GGGGGTGGTTTGGTTCAAGCTGGTGGGAGTCTCAGACTGTCA | |
| | TGTACGGCCAGCGGGGACACTTTCTCATCTTATAGCATGGGT | |
| | TGGTTTCGACAGGGTCCCGGAAAGGAACGAGAGTTCGTAGG | |
| | CGGCATTCGATGGAGTGGGGGCACGACTTACTACGCCGATTC | |
| | TGTGAAGGGTCGGTTCACAATAAGTCGAGACAACGCCAAGA | |
| | ATACAGTGTACCTCCAGATGAATTCCTTGAAAAGGGAGGAT | |
| | ACGGCCGTCTATTATTGCGCGGCCGTGAGACGGCGATGGCTC | |
| | ATCTGGCAAGAAGAAGAGTACGATTATTGGGGCCAAGGGAC | |
| | TCTTGTAACCGTGAGTTCTAGTGCTGCTGCCTTTGTCCCGGTA | |
| | TTTCTCCCAGCCAAACCGACCACGACTCCCGCCCCGCGCCCT | |
| | CCGACACCCGCTCCCACCATCGCCTCTCAACCTCTTAGTCTTC | |
| | GCCCCGAGGCATGCCGACCCGCCGCCGGGGGTGCTGTTCATA | |
| | CGAGGGGCTTGGACTTCGCTTGTGATATTTACATTTGGGCTC | |
| | CGTTGGCGGGTACGTGCGGCGTCCTTTTGTTGTCACTCGTTAT | |
| | TACTTTGTATTGTAATCACAGGAATCGCAAACGGGGCAGAA | |
| | AGAAACTCCTGTATATATTCAAACAACCATTTATGAGACCAG | |
| | TACAAACTACTCAAGAGGAAGATGGCTGTAGCTGCCGATTTC | |
| | CAGAAGAAGAAGAAGGAGGATGTGAACTGCGAGTGAAGTTT | |
| | TCCCGAAGCGCAGACGCTCCGGCATATCAGCAAGGACAGAA | |
| | TCAGCTGTATAACGAACTGAATTTGGGACGCCGCGAGGAGT | |
| | ATGACGTGCTTGATAAACGCCGGGGGAGAGACCCGGAAATG | |
| | GGGGGTAAACCCCGAAGAAAGAATCCCCAAGAAGGACTCTA | |
| | CAATGAACTCCAGAAGGATAAGATGGCGGAGGCCTACTCAG | |
| | AAATAGGTATGAAGGGCGAACGACGACGGGGAAAAGGTCA | |
| | CGATGGCCTCTACCAAGGGTTGAGTACGGCAACCAAAGATA | |
| | CGTACGATGCACTGCATATGCAGGCCCTGCCTCCCAGA | |

Also within the scope of the present disclosure are nucleic acids coding for any of the anti-CD117 CAR constructs disclosed herein. The nucleic acids may be located in a suitable vector, for example, a viral vector such as an AAV vector. Alternatively, the nucleic acids may be RNA molecules such as messenger RNA (mRNA) molecules. Host cells comprising such a nucleic acid or a vector are also within the scope of the present disclosure.

### II. Genetically Engineered T Cells Expressing Anti-CD117 CAR

Another aspect of the present disclosure provides a genetically engineered T cell or a population of genetically engineered T cells expressing an anti-CD117 CAR such as those disclosed herein. In some embodiments, the T cells are human T cells. An expression cassette for producing the anti-CD117 CAR may be inserted in a genomic site of interest. In addition to the nucleotide sequence encoding the anti-CD117 CAR, the expression cassette may further comprise a promoter in operable linkage to the CAR coding sequence and optionally one or more regulatory elements for modulating expression of the CAR. Examples include enhancers, silencers, transcriptional factor binding site, polyadenylation signal sequence, or any combination thereof.

Any of the genetically engineered T cells expressing an anti-CD117 CAR may comprise one or more additional genetic modifications. In some embodiments, the genetically engineered T cells expressing an anti-CD117 CAR may further have a disrupted TRAC gene, a disrupted B2M gene, or a combination thereof. The disruption of the TRAC locus results in loss of expression of the T cell receptor (TCR) and is intended to reduce the probability of Graft versus Host Disease (GvHD), while the disruption of the β2M locus results in lack of expression of the major histocompatibility complex type I (MHC I) proteins and is intended to improve persistence by reducing the probability of host rejection.

As used herein, the term “a disrupted gene” refers to a gene containing one or more mutations (e.g., insertion, deletion, or nucleotide substitution, etc.) relative to the wild-type counterpart so as to substantially reduce or completely eliminate the activity of the encoded gene product. The one or more mutations may be located in a non-coding region, for example, a promoter region, a regulatory region that regulates transcription or translation; or an intron region. Alternatively, the one or more mutations may be located in a coding region (e.g., in an exon). In some instances, the disrupted gene does not express or expresses a substantially reduced level of the encoded protein. In other instances, the disrupted gene expresses the encoded protein in a mutated form, which is either not functional or has substantially reduced activity. In some embodiments, a disrupted gene is a gene that does not encode functional protein. In some embodiments, a cell that comprises a disrupted gene does not express (e.g., at the cell surface) a detectable level (e.g., by antibody, e.g., by flow cytometry) of the protein encoded by the gene. A cell that does not express a detectable level of the protein may be referred to as a knockout cell. For example, a cell having a β2M gene edit may be considered a 82M knockout cell if β2M protein cannot be detected at the cell surface using an antibody that specifically binds 82M protein.

In some embodiments, a disrupted gene may be described as comprising a mutated fragment relative to the wild-type counterpart. The mutated fragment may comprise a deletion, a nucleotide substitution, an addition, or a combination thereof. In other embodiments, a disrupted gene may be described as having a deletion of a fragment that is present in the wild-type counterpart. In some instances, the 5′ end of the deleted fragment may be located within the gene region targeted by a designed guide RNA such as those disclosed herein (known as on-target sequence) and the 3′ end of the deleted fragment may go beyond the targeted region. Alternatively, the 3′ end of the deleted fragment may be located within the targeted region and the 5′ end of the deleted fragment may go beyond the targeted region.

In some instances, the disrupted TRAC gene in the genetically engineered T cells disclosed herein may comprise a deletion, for example, a deletion of a fragment in Exon 1 of the TRAC gene locus. In some examples, the disrupted TRAC gene comprises a deletion of a fragment comprising the nucleotide sequence of SEQ ID NO: 40, which is the target site of TRAC guide RNA TA-1. See Table 3 below. In some examples, the fragment of SEQ ID NO: 12 may be replaced by a nucleic acid encoding the anti-CD117 CAR.

The disrupted B2M gene in the genetically engineered T cells disclosed herein may be generated using the CRISPR/Cas technology. In some examples, a B2M gRNA provided in Table 3 may be used. The disrupted β2M gene may comprise a nucleotide sequence of any one of SEQ ID NOs: 206-211. See Table 3 below.

In some embodiments, provided herein is a population of genetically engineered immune cells (e.g., T cells such as human T cells), which collectively (i.e., in the whole cell population) express any of the anti-CD117 CAR disclosed herein (e.g., the anti-CD117 CAR provided in Table 2 above), a disrupted TRAC gene, and a disrupted B2M gene as also disclosed herein. The nucleic acid encoding the anti-CD117 CAR can be inserted in a genomic site of interest, for example, in the disrupted TRAC gene, thereby disrupting expression of the TRAC gene. In some examples, the CAR-coding sequence can be inserted at the site of SEQ ID NO: 12, e.g., replacing a fragment in the TRAC gene that comprise SEQ ID NO: 12.

The population of genetically engineered T cells disclosed herein may be a heterogeneous cell population comprising T cells having one or more of the genetic modifications disclosed herein, for example, expressing the anti-CD117 CAR, having a disrupted TRAC gene, having a disrupted β2M gene, or a combination thereof.

In some examples, at least 30% of a population of the genetically engineered T cells express a detectable level of the anti-CD117 CAR. For example, at least 40%, at least 50%, at least 60%, at least 70%, at least 75%, at least 80%, at least 85%, at least 90%, or at least 95% of the genetically engineered T cells express a detectable level of the anti-CD117 CAR.

In some embodiments, at least 30% of the T cells in the population of genetically engineered T cells may not express a detectable level of β2M surface protein. For example, at least 40%, at least 50%, at least 60%, at least 70% or more of the T cells in the population may not express a detectable level of β2M surface protein.

Alternatively or in addition, at least 50% of the T cells in the population of genetically engineered T cells may not express a detectable level of TCR surface protein. For example, at least 60%, at least 70%, at least 80%, at least 90%, at least 95% or more of the T cells in the population may not express a detectable level of TCR surface protein.

In some embodiments, a substantial percentage of the cells in the population of genetically engineered T cells may comprise more than one gene edit, which results in a certain percentage of cells not expressing more than one gene and/or protein. For example, at least 50% of the cells in the population of genetically engineered T cells may not express a detectable level of two surface proteins, e.g., does not express a detectable level of 02M and TRAC proteins. In some examples, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the cells in the population do not express a detectable level of TRAC and B2M surface proteins.

In some embodiments, a substantial percentage of the cells in the population of genetically engineered T cells may express any of the anti-CD117 CAR, have a disrupted TRAC gene, and a disrupted B2M gene. The expression cassette coding for the anti-CD117 CAR may be inserted in the disrupted TRAC gene, thereby disrupting its expression. In some examples, the disrupted TRAC gene comprises a deletion of a fragment comprising the nucleotide sequence of SEQ ID NO: 12. The CAR expression cassette may be inserted at the deletion site, for example, replacing the fragment comprising SEQ ID NO: 12.

Alternatively, the population of genetically engineered T cells disclosed herein may contain a wild-type TRAC gene, a wild-type B2M gene, or a combination thereof.

In some instances, the population of genetically engineered T cells may transiently express any of the anti-CD117 CAR polypeptides as disclosed herein. Transient expression of the anti-CD117 CAR can be achieved by methods known in the art, for example, via mRNA transfection.

### III. Preparation of Genetically Engineered Immune Cells

Any suitable gene editing methods known in the art can be used for making the genetically engineered immune cells (e.g., T cells such as human T cells expressing a anti-CD117 CAR) disclosed herein, for example, nuclease-dependent targeted editing using zinc-finger nucleases (ZFNs), transcription activator-like effector nucleases (TALENs), or RNA-guided CRISPR-Cas9 nucleases (CRISPR/Cas9; Clustered Regular Interspaced Short Palindromic Repeats Associated 9). In specific examples, the genetically engineered immune cells such as T cells are produced by the CRISPR technology in combination with homologous recombination using an adeno-associated viral vector (AAV) as a donor template.

### (i) CRISPR-Cas9-Mediated Gene Editing System

The CRISPR-Cas9 system is a naturally-occurring defense mechanism in prokaryotes that has been repurposed as an RNA-guided DNA-targeting platform used for gene editing. It relies on the DNA nuclease Cas9, and two noncoding RNAs, crisprRNA (crRNA) and trans-activating RNA (tracrRNA), to target the cleavage of DNA. CRISPR is an abbreviation for Clustered Regularly Interspaced Short Palindromic Repeats, a family of DNA sequences found in the genomes of bacteria and archaea that contain fragments of DNA (spacer DNA) with similarity to foreign DNA previously exposed to the cell, for example, by viruses that have infected or attacked the prokaryote. These fragments of DNA are used by the prokaryote to detect and destroy similar foreign DNA upon re-introduction, for example, from similar viruses during subsequent attacks. Transcription of the CRISPR locus results in the formation of an RNA molecule comprising the spacer sequence, which associates with and targets Cas (CRISPR-associated) proteins able to recognize and cut the foreign, exogenous DNA. Numerous types and classes of CRISPR/Cas systems have been described (see, e.g., Koonin et al., (2017) Curr Opin Microbiol 37:67-78).

crRNA drives sequence recognition and specificity of the CRISPR-Cas9 complex through Watson-Crick base pairing typically with a 20 nucleotide (nt) sequence in the target DNA. Changing the sequence of the 5′ 20 nt in the crRNA allows targeting of the CRISPR-Cas9 complex to specific loci. The CRISPR-Cas9 complex only binds DNA sequences that contain a sequence match to the first 20 nt of the crRNA, if the target sequence is followed by a specific short DNA motif (with the sequence NGG) referred to as a protospacer adjacent motif (PAM).

TracrRNA hybridizes with the 3′ end of crRNA to form an RNA-duplex structure that is bound by the Cas9 endonuclease to form the catalytically active CRISPR-Cas9 complex, which can then cleave the target DNA.

Once the CRISPR-Cas9 complex is bound to DNA at a target site, two independent nuclease domains within the Cas9 enzyme each cleave one of the DNA strands upstream of the PAM site, leaving a double-strand break (DSB) where both strands of the DNA terminate in a base pair (a blunt end).

After binding of CRISPR-Cas9 complex to DNA at a specific target site and formation of the site-specific DSB, the next key step is repair of the DSB. Cells use two main DNA repair pathways to repair the DSB: non-homologous end joining (NHEJ) and homology-directed repair (HDR).

NHEJ is a robust repair mechanism that appears highly active in the majority of cell types, including non-dividing cells. NHEJ is error-prone and can often result in the removal or addition of between one and several hundred nucleotides at the site of the DSB, though such modifications are typically <20 nt. The resulting insertions and deletions (indels) can disrupt coding or noncoding regions of genes. Alternatively, HDR uses a long stretch of homologous donor DNA, provided endogenously or exogenously, to repair the DSB with high fidelity. HDR is active only in dividing cells, and occurs at a relatively low frequency in most cell types. In many embodiments of the present disclosure, NHEJ is utilized as the repair operant.

### (a) Cas9

In some embodiments, the Cas9 (CRISPR associated protein 9) endonuclease is used in a CRISPR method for making the genetically engineered T cells as disclosed herein. The Cas9 enzyme may be one from Streptococcus pyogenes, although other Cas9 homologs may also be used. It should be understood, that wild-type Cas9 may be used or modified versions of Cas9 may be used (e.g., evolved versions of Cas9, or Cas9 orthologues or variants), as provided herein. In some embodiments, Cas9 comprises a Streptococcus pyogenes-derived Cas9 nuclease protein that has been engineered to include C- and N-terminal SV40 large T antigen nuclear localization sequences (NLS). The resulting Cas9 nuclease (sNLS-spCas9-sNLS) is a 162 kDa protein that is produced by recombinant E. coli fermentation and purified by chromatography. The spCas9 amino acid sequence can be found as UniProt Accession No. Q99ZW2, which is provided herein as SEQ ID NO: 222 provided in Table 3 below.

(b) Guide RNAs (gRNAs)

CRISPR-Cas9-mediated gene editing as described herein includes the use of a guide RNA or a gRNA. As used herein, a “gRNA” refers to a genome-targeting nucleic acid that can direct the Cas9 to a specific target sequence within a TRAC gene or a β2M gene for gene editing at the specific target sequence. A guide RNA comprises at least a spacer sequence that hybridizes to a target nucleic acid sequence within a target gene for editing, and a CRISPR repeat sequence.

An exemplary gRNA targeting a TRAC gene is provided in Table 3 below. See also WO 2019/097305A2, the relevant disclosures of which are incorporated by reference herein for the subject matter and purpose referenced herein. Other gRNA sequences may be designed using the TRAC gene sequence located on chromosome 14 (GRCh38: chromosome 14: 22,547,506-22,552,154; Ensembl; ENSG00000277734). In some embodiments, gRNAs targeting the TRAC genomic region and Cas9 create breaks in the TRAC genomic region resulting Indels in the TRAC gene disrupting expression of the mRNA or protein.

An exemplary gRNA targeting a β2M gene is provided in Table 3 below. See also WO 2019/097305A2, the relevant disclosures of which are incorporated by reference herein for the purpose and subject matter referenced herein. Other gRNA sequences may be designed using the β2M gene sequence located on Chromosome 15 (GRCh38 coordinates: Chromosome 15: 44,711,477-44,718,877; Ensembl: ENSG00000166710). In some embodiments, gRNAs targeting the β2M genomic region and RNA-guided nuclease create breaks in the β2M genomic region resulting in Indels in the β2M gene disrupting expression of the mRNA or protein.

In Type II systems, the gRNA also comprises a second RNA called the tracrRNA sequence. In the Type II gRNA, the CRISPR repeat sequence and tracrRNA sequence hybridize to each other to form a duplex. In the Type V gRNA, the crRNA forms a duplex. In both systems, the duplex binds a site-directed polypeptide, such that the guide RNA and site-direct polypeptide form a complex. In some embodiments, the genome-targeting nucleic acid provides target specificity to the complex by virtue of its association with the site-directed polypeptide. The genome-targeting nucleic acid thus directs the activity of the site-directed polypeptide.

As is understood by the person of ordinary skill in the art, each guide RNA is designed to include a spacer sequence complementary to its genomic target sequence. See Jinek et al., Science, 337, 816-821 (2012) and Deltcheva et al., Nature, 471, 602-607 (2011).

In some embodiments, the genome-targeting nucleic acid (e.g., gRNA) is a double-molecule guide RNA. In some embodiments, the genome-targeting nucleic acid (e.g., gRNA) is a single-molecule guide RNA.

A double-molecule guide RNA comprises two strands of RNA molecules. The first strand comprises in the 5′ to 3′ direction, an optional spacer extension sequence, a spacer sequence and a minimum CRISPR repeat sequence. The second strand comprises a minimum tracrRNA sequence (complementary to the minimum CRISPR repeat sequence), a 3′ tracrRNA sequence and an optional tracrRNA extension sequence.

A single-molecule guide RNA (referred to as a “sgRNA”) in a Type II system comprises, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension may comprise elements that contribute additional functionality (e.g., stability) to the guide RNA. The single-molecule guide linker links the minimum CRISPR repeat and the minimum tracrRNA sequence to form a hairpin structure. The optional tracrRNA extension comprises one or more hairpins. A single-molecule guide RNA in a Type V system comprises, in the 5′ to 3′ direction, a minimum CRISPR repeat sequence and a spacer sequence.

The “target sequence” is in a target gene that is adjacent to a PAM sequence and is the sequence to be modified by Cas9. The “target sequence” is on the so-called PAM-strand in a “target nucleic acid,” which is a double-stranded molecule containing the PAM-strand and a complementary non-PAM strand. One of skill in the art recognizes that the gRNA spacer sequence hybridizes to the complementary sequence located in the non-PAM strand of the target nucleic acid of interest. Thus, the gRNA spacer sequence is the RNA equivalent of the target sequence.

For example, if the TRAC target sequence is 5′-AGAGCAACAGTGCTGTGGCC-3′ (SEQ ID NO: 12), then the gRNA spacer sequence is 5′-AGAGCAACAGUGCUGUGGCC-3′ (SEQ ID NO: 23). In another example, if the 02M target sequence is 5′-GCTACTCTCTCTTTCTGGCC-3′ (SEQ ID NO: 200), then the gRNA spacer sequence is 5′-GCUACUCUCUCUUUCUGGCC-3′ (SEQ ID NO: 203). The spacer of a gRNA interacts with a target nucleic acid of interest in a sequence-specific manner via hybridization (i.e., base pairing). The nucleotide sequence of the spacer thus varies depending on the target sequence of the target nucleic acid of interest.

In a CRISPR/Cas system herein, the spacer sequence is designed to hybridize to a region of the target nucleic acid that is located 5′ of a PAM recognizable by a Cas9 enzyme used in the system. The spacer may perfectly match the target sequence or may have mismatches. Each Cas9 enzyme has a particular PAM sequence that it recognizes in a target DNA. For example, S. pyogenes recognizes in a target nucleic acid a PAM that comprises the sequence 5′-NRG-3′, where R comprises either A or G, where N is any nucleotide and N is immediately 3′ of the target nucleic acid sequence targeted by the spacer sequence.

In some embodiments, the target nucleic acid sequence has 20 nucleotides in length. In some embodiments, the target nucleic acid has less than 20 nucleotides in length. In some embodiments, the target nucleic acid has more than 20 nucleotides in length. In some embodiments, the target nucleic acid has at least: 5, 10, 15, 16, 17, 18, 19, 20, 21, 22, 23, 24, 25, 30 or more nucleotides in length. In some embodiments, the target nucleic acid has at most: 5, 10, 15, 16, 17, 18, 19, 20, 21, 22, 23, 24, 25, 30 or more nucleotides in length. In some embodiments, the target nucleic acid sequence has 20 bases immediately 5′ of the first nucleotide of the PAM. For example, in a sequence comprising 5′-NNNNNNNNNNNNNNNNNNNNNRG-3′, the target nucleic acid can be the sequence that corresponds to the Ns, wherein N can be any nucleotide, and the underlined NRG sequence is the S. pyogenes PAM. Examples are provided as SEQ ID NOs: 41 and 55.

The guide RNA disclosed herein may target any sequence of interest via the spacer sequence in the crRNA. In some embodiments, the degree of complementarity between the spacer sequence of the guide RNA and the target sequence in the target gene can be about 60%, 65%, 70%, 75%, 80%, 85%, 90%, 95%, 97%, 98%, 99%, or 100%. In some embodiments, the spacer sequence of the guide RNA and the target sequence in the target gene is 100% complementary. In other embodiments, the spacer sequence of the guide RNA and the target sequence in the target gene may contain up to 10 mismatches, e.g., up to 9, up to 8, up to 7, up to 6, up to 5, up to 4, up to 3, up to 2, or up to 1 mismatch.

Non-limiting examples of gRNAs that may be used as provided herein are provided in WO 2019/097305A2, and WO2019/215500, the relevant disclosures of each of which are herein incorporated by reference for the purposes and subject matter referenced herein. For any of the gRNA sequences provided herein, those that do not explicitly indicate modifications are meant to encompass both unmodified sequences and sequences having any suitable modifications.

The length of the spacer sequence in any of the gRNAs disclosed herein may depend on the CRISPR/Cas9 system and components used for editing any of the target genes also disclosed herein. For example, different Cas9 proteins from different bacterial species have varying optimal spacer sequence lengths. Accordingly, the spacer sequence may have 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 nucleotides in length. In some embodiments, the spacer sequence may have 18-24 nucleotides in length. In some embodiments, the targeting sequence may have 19-21 nucleotides in length. In some embodiments, the spacer sequence may comprise 20 nucleotides in length.

In some embodiments, the gRNA can be a sgRNA, which may comprise a 20-nucleotide spacer sequence at the 5′ end of the sgRNA sequence. In some embodiments, the sgRNA may comprise a less than 20 nucleotide spacer sequence at the 5′ end of the sgRNA sequence. In some embodiments, the sgRNA may comprise a more than 20 nucleotide spacer sequence at the 5′ end of the sgRNA sequence. In some embodiments, the sgRNA comprises a variable length spacer sequence with 17-30 nucleotides at the 5′ end of the sgRNA sequence.

In some embodiments, the sgRNA comprises no uracil at the 3′ end of the sgRNA sequence. In other embodiments, the sgRNA may comprise one or more uracil at the 3′ end of the sgRNA sequence. For example, the sgRNA can comprise 1-8 uracil residues, at the 3′ end of the sgRNA sequence, e.g., 1, 2, 3, 4, 5, 6, 7, or 8 uracil residues at the 3′ end of the sgRNA sequence.

Any of the gRNAs disclosed herein, including any of the sgRNAs, may be unmodified. Alternatively, it may contain one or more modified nucleotides and/or modified backbones. For example, a modified gRNA such as a sgRNA can comprise one or more 2′-O-methyl phosphorothioate nucleotides, which may be located at either the 5′ end, the 3′ end, or both.

In certain embodiments, more than one guide RNAs can be used with a CRISPR/Cas nuclease system. Each guide RNA may contain a different targeting sequence, such that the CRISPR/Cas system cleaves more than one target nucleic acid. In some embodiments, one or more guide RNAs may have the same or differing properties such as activity or stability within the Cas9 RNP complex. Where more than one guide RNA is used, each guide RNA can be encoded on the same or on different vectors. The promoters used to drive expression of the more than one guide RNA is the same or different.

It should be understood that more than one suitable Cas9 and more than one suitable gRNA can be used in methods described herein, for example, those known in the art or disclosed herein. In some embodiments, methods comprise a Cas9 enzyme and/or a gRNA known in the art. Examples can be found in, e.g., WO 2019/097305A2, and WO2019/215500, the relevant disclosures of each of which are herein incorporated by reference for the purposes and subject matter referenced herein.

Table 3 below provides exemplary components for gene editing of TRAC and B2M genes.

TABLE 3
Exemplary Components for Genetic Modification of TRAC and B2M
Genes

| | | SEQ ID |
|---|---|---|
| Description | Sequence | NO |
| TRAC target | AGAGCAACAGTGCTGTGGCC | 12 |
| sequence | | |
| TRAC target | 5′-AGAGCAACAGTGCTGTGGCC (TGG)-3′ | 14 |
| sequence with | | |
| (PAM) | | |
| TRAC sgRNA (TA-1) | AGAGCAACAGUGCUGUGGCCguuuuagagcuagaaauagcaaguu | 21 |
| unmodified | aaaauaaggcuaguccguuaucaacuugaaaaaguggcaccgagu | |
| | cggugcUUUU | |
| TRAC sgRNA | AGAGCAACAGUGCUGUGGCC | 23 |
| spacer unmodified | | |
| TRAC sgRNA | A*G*A*GCAACAGUGCUGUGGCC | 39 |
| spacer modified | | |
| TRAC sgRNA (TA-1) | A*G*A*GCAACAGUGCUGUGGCCguuuuagagcuagaaauagcaa | 40 |
| modified | guuaaaauaaggcuaguccguuaucaacuugaaaaaguggcaccg | |
| | agucggugcU*U*U*U | |
| TRAC gene-edit | AAGAGCAACAAATCTGACT | 54 |
| TRAC gene-edit | AAGAGCAACAGTGCTGTGCCTGGAGCAACAAATCTGACT | 67 |
| | AAGAGCAACAAATCTGACT | |
| TRAC gene-edit | AAGAGCAACAGTGCTGGAGCAACAAATCTGACT | 89 |
| | AAGAGCAACAAATCTGACT | |
| TRAC gene-edit | AAGAGCAACAGTGCCTGGAGCAACAAATCTGACT | 90 |
| | AAGAGCAACAAATCTGACT | |
| TRAC gene-edit | AAGAGCAACAGTGCTGACTAAGAGCAACAAATCTGACT | 92 |
| TRAC gene-edit | AAGAGCAACAGTGCTGTGGGCCTGGAGCAACAAATCTGACT | 93 |
| | AAGAGCAACAAATCTGACT | |
| TRAC gene-edit | AAGAGCAACAGTGCTGGCCTGGAGCAACAAATCTGACT | 94 |
| | AAGAGCAACAAATCTGACT | |
| TRAC gene-edit | AAGAGCAACAGTGCTGTGTGCCTGGAGCAACAAATCTGACT | 199 |
| | AAGAGCAACAAATCTGACT | |
| B2M target sequence | GCTACTCTCTCTTTCTGGCC | 200 |
| B2M target | GCTACTCTCTCTTTCTGGCC (TGG) | 201 |
| sequence with | | |
| (PAM) | | |
| 56B2M sgRNA | GCUACUCUCUCUUUCUGGCC | 202 |
| spacer unmodified | | |
| B2M sgRNA spacer | G*C*U*ACUCUCUCUUUCUGGCC | 203 |
| modified | | |
| B2M sgRNA | GCUACUCUCUCUUUCUGGCCguuuuagagcuagaaauagcaaguu | 204 |
| unmodified | aaaauaaggcuaguccguuaucaacuugaaaaaguggcaccgagu | |
| | cggugcUUUU | |
| B2M sgRNA | G*C*U*ACUCUCUCUUUCUGGCCguuuuagagcuagaaauagcaa | 205 |
| modified | guuaaaauaaggcuaguccguuaucaacuugaaaaaguggcaccg | |
| | agucggugcU*U*U*U | |
| B2M gene-edit | CGTGGCCTTAGCTGTGCTCGCGCTACTCTCTCTTTCTGCCTGGAG | 206 |
| | GCTATCCAGCGTGAGTCTCTCCTACCCTCCCGCT | |
| B2M gene-edit | CGTGGCCTTAGCTGTGCTCGCGCTACTCTCTCTTTCGCCTGGAGG | 207 |
| | CTATCCAGCGTGAGTCTCTCCTACCCTCCCGCT | |
| B2M gene-edit | CGTGGCCTTAGCTGTGCTCGCGCTACTCTCTCTTTCTGGAGGCTA | 208 |
| | TCCAGCGTGAGTCTCTCCTACCCTCCCGCT | |
| B2M gene-edit | CGTGGCCTTAGCTGTGCTCGCGCTACTCTCTCTTTCTGGATAGCC | 209 |
| | TGGAGGCTATCCAGCGTGAGTCTCTCCTACCCTCCCGCT | |
| B2M gene-edit | CGTGGCCTTAGCTGTGCTCGCGCTATCCAGCGTGAGTCTCTCCTA | 210 |
| | CCCTCCCGCT | |
| B2M gene-edit | CGTGGCCTTAGCTGTGCTCGCGCTACTCTCTCTTTCTGTGGCCTG | 211 |
| | GAGGCTATCCAGCGTGAGTCTCTCCTACCCTCCCGCT | |
| sgRNA | nnnnnnnnnnnnnnnnnnnnguuuuagagcuagaaauagcaaguu | 212 |
| | aaaauaaggcuaguccguuaucaacuugaaaaaguggcaccgagu | |
| | cggugcuuuu | |
| sgRNA | nnnnnnnnnnnnnnnnnnnnguuuuagagcuagaaauagcaaguu | 213 |
| | aaaauaaggcuaguccguuaucaacuugaaaaaguggcaccgagu | |
| | cggugc | |
| sgRNA | guuuuagagcuagaaauagcaaguuaaaauaaggcuaguc | 214 |
| | cguuaucaacuugaaaaaguggcaccgagucggugcu | |
| sgRNA | guuuuagagcuagaaauagcaaguuaaaauaaggcuaguccguua | 215 |
| | ucaacuugaaaaaguggcaccgagucggugcu | |
| | guuuuagagcuagaaauagcaaguuaaaauaaggcuaguccguua | |
| | ucaacuugaaaaaguggcaccgagucggugcu | |
| sgRNA | guuuuagagcuagaaauagcaaguuaaaauaaggcuaguccguua | 216 |
| | ucaacuugaaaaaguggcaccgagucggugcu | |
| | guuuuagagcuagaaauagcaaguuaaaauaaggcuaguccguua | |
| | ucaacuugaaaaaguggcaccgagucggugcu | |
| | guuuuagagcuagaaauagcaaguuaaaauaaggcuaguccguua | |
| | ucaacuugaaaaaguggcaccgagucggugcu | |
| sgRNA | guuuuagagcuagaaauagcaaguuaaaauaaggcuaguccguua | 217 |
| | ucaacuugaaaaaguggcaccgagucggugcu | |
| | guuuuagagcuagaaauagcaaguuaaaauaaggcuaguccguua | |
| | ucaacuugaaaaaguggcaccgagucggugcu | |
| | guuuuagagcuagaaauagcaaguuaaaauaaggcuaguccguua | |
| | ucaacuugaaaaaguggcaccgagucggugcu | |
| | guuuuagagcuagaaauagcaaguuaaaauaaggcuaguccguua | |
| | ucaacuugaaaaaguggcaccgagucggugcu | |
| sgRNA | guuuuagagcuagaaauagcaaguuaaaauaaggcuaguccguua | 218 |
| | ucaacuugaaaaaguggcaccgagucggugcu | |
| | guuuuagagcuagaaauagcaaguuaaaauaaggcuaguccguua | |
| | ucaacuugaaaaaguggcaccgagucggugcu | |
| | guuuuagagcuagaaauagcaaguuaaaauaaggcuaguccguua | |
| | ucaacuugaaaaaguggcaccgagucggugcu | |
| | guuuuagagcuagaaauagcaaguuaaaauaaggcuaguccguua | |
| | ucaacuugaaaaaguggcaccgagucggugcu | |
| | guuuuagagcuagaaauagcaaguuaaaauaaggcuaguccguua | |
| | ucaacuugaaaaaguggcaccgagucggugcu | |
| sgRNA | guuuuagagcuagaaauagcaaguuaaaauaaggcuaguccguua | 219 |
| | ucaacuugaaaaaguggcaccgagucggugcu | |
| | guuuuagagcuagaaauagcaaguuaaaauaaggcuaguccguua | |
| | ucaacuugaaaaaguggcaccgagucggugcu | |
| | guuuuagagcuagaaauagcaaguuaaaauaaggcuaguccguua | |
| | ucaacuugaaaaaguggcaccgagucggugcu | |
| | guuuuagagcuagaaauagcaaguuaaaauaaggcuaguccguua | |
| | ucaacuugaaaaaguggcaccgagucggugcu | |
| | guuuuagagcuagaaauagcaaguuaaaauaaggcuaguccguua | |
| | ucaacuugaaaaaguggcaccgagucggugcu | |
| | guuuuagagcuagaaauagcaaguuaaaauaaggcuaguccguua | |
| | ucaacuugaaaaaguggcaccgagucggugcu | |
| sgRNA | guuuuagagcuagaaauagcaaguuaaaauaaggcuaguccguua | 220 |
| | ucaacuugaaaaaguggcaccgagucggugcu | |
| | guuuuagagcuagaaauagcaaguuaaaauaaggcuaguccguua | |
| | ucaacuugaaaaaguggcaccgagucggugcu | |
| | guuuuagagcuagaaauagcaaguuaaaauaaggcuaguccguua | |
| | ucaacuugaaaaaguggcaccgagucggugcu | |
| | guuuuagagcuagaaauagcaaguuaaaauaaggcuaguccguua | |
| | ucaacuugaaaaaguggcaccgagucggugcu | |
| | guuuuagagcuagaaauagcaaguuaaaauaaggcuaguccguua | |
| | ucaacuugaaaaaguggcaccgagucggugcu | |
| | guuuuagagcuagaaauagcaaguuaaaauaaggcuaguccguua | |
| | ucaacuugaaaaaguggcaccgagucggugcu | |
| | guuuuagagcuagaaauagcaaguuaaaauaaggcuaguccguua | |
| | ucaacuugaaaaaguggcaccgagucggugcu | |
| sgRNA | guuuuagagcuagaaauagcaaguuaaaauaaggcuaguccguua | 221 |
| | ucaacuugaaaaaguggcaccgagucggugcu | |
| | guuuuagagcuagaaauagcaaguuaaaauaaggcuaguccguua | |
| | ucaacuugaaaaaguggcaccgagucggugcu | |
| | guuuuagagcuagaaauagcaaguuaaaauaaggcuaguccguua | |
| | ucaacuugaaaaaguggcaccgagucggugcu | |
| | guuuuagagcuagaaauagcaaguuaaaauaaggcuaguccguua | |
| | ucaacuugaaaaaguggcaccgagucggugcu | |
| | guuuuagagcuagaaauagcaaguuaaaauaaggcuaguccguua | |
| | ucaacuugaaaaaguggcaccgagucggugcu | |
| | guuuuagagcuagaaauagcaaguuaaaauaaggcuaguccguua | |
| | ucaacuugaaaaaguggcaccgagucggugcu | |
| | guuuuagagcuagaaauagcaaguuaaaauaaggcuaguccguua | |
| | ucaacuugaaaaaguggcaccgagucggugcu | |
| | guuuuagagcuagaaauagcaaguuaaaauaaggcuaguccguua | |
| | ucaacuugaaaaaguggcaccgagucggugcu | |
| sgRNA | n(17-30)-SEQ ID NOS: 214-221 | |
| spCas9 | MDKKYSIGLDIGTNSVGWAVITDEYKVPSKKFKVLGNTDRHSIKK | 222 |
| | NLIGALLFDSGETAEATRLKRTARRRYTRRKNRICYLQEIFSNEM | |
| | AKVDDSFFHRLEESFLVEEDKKHERHPIFGNIVDEVAYHEKYPTI | |
| | YHLRKKLVDSTDKADLRLIYLALAHMIKFRGHFLIEGDLNPDNSD | |
| | VDKLFIQLVQTYNQLFEENPINASGVDAKAILSARLSKSRRLENL | |
| | IAQLPGEKKNGLFGNLIALSLGLTPNFKSNFDLAEDAKLQLSKDT | |
| | YDDDLDNLLAQIGDQYADLFLAAKNLSDAILLSDILRVNTEITKA | |
| | PLSASMIKRYDEHHQDLTLLKALVRQQLPEKYKEIFFDQSKNGYA | |
| | GYIDGGASQEEFYKFIKPILEKMDGTEELLVKLNREDLLRKQRTF | |
| | DNGSIPHQIHLGELHAILRRQEDFYPFLKDNREKIEKILTFRIPY | |
| | YVGPLARGNSRFAWMTRKSEETITPWNFEEVVDKGASAQSFIERM | |
| | TNFDKNLPNEKVLPKHSLLYEYFTVYNELTKVKYVTEGMRKPAFL | |
| | SGEQKKAIVDLLFKTNRKVTVKQLKEDYFKKIECFDSVEISGVED | |
| | RFNASLGTYHDLLKIIKDKDFLDNEENEDILEDIVLTLTLFEDRE | |
| | MIEERLKTYAHLFDDKVMKQLKRRRYTGWGRLSRKLINGIRDKQS | |
| | GKTILDFLKSDGFANRNFMQLIHDDSLTFKEDIQKAQVSGQGDSL | |
| | HEHIANLAGSPAIKKGILQTVKVVDELVKVMGRHKPENIVIEMAR | |
| | ENQTTQKGQKNSRERMKRIEEGIKELGSQILKEHPVENTQLQNEK | |
| | LYLYYLQNGRDMYVDQELDINRLSDYDVDHIVPQSFLKDDSIDNK | |
| | VLTRSDKNRGKSDNVPSEEVVKKMKNYWRQLLNAKLITQRKFDNL | |
| | TKAERGGLSELDKAGFIKRQLVETRQITKHVAQILDSRMNTKYDE | |
| | NDKLIREVKVITLKSKLVSDFRKDFQFYKVREINNYHHAHDAYLN | |
| | AVVGTALIKKYPKLESEFVYGDYKVYDVRKMIAKSEQEIGKATAK | |
| | YFFYSNIMNFFKTEITLANGEIRKRPLIETNGETGEIVWDKGRDF | |
| | ATVRKVLSMPQVNIVKKTEVQTGGFSKESILPKRNSDKLIARKKD | |
| | WDPKKYGGFDSPTVAYSVLVVAKVEKGKSKKLKSVKELLGITIME | |
| | RSSFEKNPIDFLEAKGYKEVKKDLIIKLPKYSLFELENGRKRMLA | |
| | SAGELQKGNELALPSKYVNFLYLASHYEKLKGSPEDNEQKQLFVE | |
| | QHKHYLDEIIEQISEFSKRVILADANLDKVLSAYNKHRDKPIREQ | |
| | AENIIHLFTLINLGAPAAFKYFDTTIDRKRYTSTKEVLDATLIHQ | |
| | SITGLYETRIDLSQLGGD | |

*indicates a nucleotide with a 2′-O-methyl phosphorothioate modification.
“n” refers to the spacer sequence at the 5′ end.

### (ii) AAV Vectors for Delivery of CAR Constructs to T Cells

A nucleic acid encoding any of the anti-CD117 CAR constructs as disclosed herein can be delivered to a cell using an adeno-associated virus (AAV). AAVs are small viruses which integrate site-specifically into the host genome and can therefore deliver a transgene, such as CAR. Inverted terminal repeats (ITRs) are present flanking the AAV genome and/or the transgene of interest and serve as origins of replication. Also present in the AAV genome are rep and cap proteins which, when transcribed, form capsids which encapsulate the AAV genome for delivery into target cells. Surface receptors on these capsids which confer AAV serotype, which determines which target organs the capsids will primarily bind and thus what cells the AAV will most efficiently infect. There are twelve currently known human AAV serotypes. In some embodiments, the AAV for use in delivering the CAR-coding nucleic acid is AAV serotype 6 (AAV6).

Adeno-associated viruses are among the most frequently used viruses for gene therapy for several reasons. First, AAVs do not provoke an immune response upon administration to mammals, including humans. Second, AAVs are effectively delivered to target cells, particularly when consideration is given to selecting the appropriate AAV serotype. Finally, AAVs have the ability to infect both dividing and non-dividing cells because the genome can persist in the host cell without integration. This trait makes them an ideal candidate for gene therapy.

A nucleic acid encoding the anti-CD117 CAR can be designed to insert into a genomic site of interest in the host T cells. In some embodiments, the target genomic site can be in a safe harbor locus.

In some embodiments, a nucleic acid encoding the anti-CD117 CAR (e.g., via a donor template, which can be carried by a viral vector such as an adeno-associated viral (AAV) vector) can be designed such that it can insert into a location within a TRAC gene to disrupt the TRAC gene in the genetically engineered T cells and express the CAR polypeptide. Disruption of TRAC leads to loss of function of the endogenous TCR. For example, a disruption in the TRAC gene can be created with an endonuclease such as those described herein and one or more gRNAs targeting one or more TRAC genomic regions. Any of the gRNAs specific to a TRAC gene and the target regions can be used for this purpose, e.g., those disclosed herein.

In some examples, a genomic deletion in the TRAC gene and replacement by a CAR coding segment can be created by homology directed repair or HDR (e.g., using a donor template, which may be part of a viral vector such as an adeno-associated viral (AAV) vector). In some embodiments, a disruption in the TRAC gene can be created with an endonuclease as those disclosed herein and one or more gRNAs targeting one or more TRAC genomic regions, and inserting a CAR coding segment into the TRAC gene.

A donor template as disclosed herein can contain a coding sequence for a CAR. In some examples, the CAR-coding sequence may be flanked by two regions of homology to allow for efficient HDR at a genomic location of interest, for example, at a TRAC gene using CRISPR-Cas9 gene editing technology. In this case, both strands of the DNA at the target locus can be cut by a CRISPR Cas9 enzyme guided by gRNAs specific to the target locus. HDR then occurs to repair the double-strand break (DSB) and insert the donor DNA coding for the CAR. For this to occur correctly, the donor sequence is designed with flanking residues which are complementary to the sequence surrounding the DSB site in the target gene (hereinafter “homology arms”), such as the TRAC gene. These homology arms serve as the template for DSB repair and allow HDR to be an essentially error-free mechanism. The rate of homology directed repair (HDR) is a function of the distance between the mutation and the cut site so choosing overlapping or nearby target sites is important. Templates can include extra sequences flanked by the homologous regions or can contain a sequence that differs from the genomic sequence, thus allowing sequence editing.

Alternatively, a donor template may have no regions of homology to the targeted location in the DNA and may be integrated by NHEJ-dependent end joining following cleavage at the target site.

A donor template can be DNA or RNA, single-stranded and/or double-stranded, and can be introduced into a cell in linear or circular form. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al., (1987) Proc. Natl. Acad. Sci. USA 84:4959-4963; Nehls et al., (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A donor template can be introduced into a cell as part of a vector molecule having additional sequences such as, for example, replication origins, promoters and genes encoding antibiotic resistance. Moreover, a donor template can be introduced into a cell as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

A donor template, in some embodiments, can be inserted at a site nearby an endogenous promoter (e.g., downstream or upstream) so that its expression can be driven by the endogenous promoter. In other embodiments, the donor template may comprise an exogenous promoter and/or enhancer, for example, a constitutive promoter, an inducible promoter, or tissue-specific promoter to control the expression of the CAR gene. In some embodiments, the exogenous promoter is an EF1α promoter. Other promoters may be used.

Furthermore, exogenous sequences may also include transcriptional or translational regulatory sequences, for example, promoters, enhancers, insulators, internal ribosome entry sites, sequences encoding 2A peptides and/or polyadenylation signals.

Table 4 below provides exemplary donor template components for inserting a nucleic acid encoding a anti-CD117 CAR in the TRAC gene locus. An exemplary donor structure may comprise, from 5′ end to 3′ end: TRAC[LHA]-EF1a[promoter]-CAR-polyA-TRAC[RHA].

TABLE 4
Sequences of Donor Template Components

| | | SEQ |
|---|---|---|
| Name | Sequence | ID NO |
| TRAC-LHA | GAGATGTAAGGAGCTGCTGTGACTTGCTCAAGGCCTTATATCGAGTAAACGGTAG | 223 |
| | TGCTGGGGCTTAGACGCAGGTGTTCTGATTTATAGTTCAAAACCTCTATCAATGA | |
| | GAGAGCAATCTCCTGGTAATGTGATAGATTTCCCAACTTAATGCCAACATACCAT | |
| | AAACCTCCCATTCTGCTAATGCCCAGCCTAAGTTGGGGAGACCACTCCAGATTCC | |
| | AAGATGTACAGTTTGCTTTGCTGGGCCTTTTTCCCATGCCTGCCTTTACTCTGCC | |
| | AGAGTTATATTGCTGGGGTTTTGAAGAAGATCCTATTAAATAAAAGAATAAGCAG | |
| | TATTATTAAGTAGCCCTGCATTTCAGGTTTCCTTGAGTGGCAGGCCAGGCCTGGC | |
| | CGTGAACGTTCACTGAAATCATGGCCTCTTGGCCAAGATTGATAGCTTGTGCCTG | |
| | TCCCTGAGTCCCAGTCCATCACGAGCAGCTGGTTTCTAAGATGCTATTTCCCGTA | |
| | TAAAGCATGAGACCGTGACTTGCCAGCCCCACAGAGCCCCGCCCTTGTCCATCAC | |
| | TGGCATCTGGACTCCAGCCTGGGTTGGGGCAAAGAGGGAAATGAGATCATGTCCT | |
| | AACCCTGATCCTCTTGTCCCACAGATATCCAGAACCCTGACCCTGCCGTGTACCA | |
| | GCTGAGAGACTCTAAATCCAGTGACAAGTCTGTCTGCCTATTCACCGATTTTGAT | |
| | TCTCAAACAAATGTGTCACAAAGTAAGGATTCTGATGTGTATATCACAGACAAAA | |
| | CTGTGCTAGACATGAGGTCTATGGACTTCA | |
| EF1a | GGCTCCGGTGCCCGTCAGTGGGCAGAGCGCACATCGCCCACAGTCCCCGAGAAGT | 224 |
| promoter | TGGGGGGAGGGGTCGGCAATTGAACCGGTGCCTAGAGAAGGTGGCGCGGGGTAAA | |
| | CTGGGAAAGTGATGTCGTGTACTGGCTCCGCCTTTTTCCCGAGGGTGGGGGAGAA | |
| | CCGTATATAAGTGCAGTAGTCGCCGTGAACGTTCTTTTTCGCAACGGGTTTGCCG | |
| | CCAGAACACAGGTAAGTGCCGTGTGTGGTTCCCGCGGGCCTGGCCTCTTTACGGG | |
| | TTATGGCCCTTGCGTGCCTTGAATTACTTCCACTGGCTGCAGTACGTGATTCTTG | |
| | ATCCCGAGCTTCGGGTTGGAAGTGGGTGGGAGAGTTCGAGGCCTTGCGCTTAAGG | |
| | AGCCCCTTCGCCTCGTGCTTGAGTTGAGGCCTGGCCTGGGCGCTGGGGCCGCCGC | |
| | GTGCGAATCTGGTGGCACCTTCGCGCCTGTCTCGCTGCTTTCGATAAGTCTCTAG | |
| | CCATTTAAAATTTTTGATGACCTGCTGCGACGCTTTTTTTCTGGCAAGATAGTCT | |
| | TGTAAATGCGGGCCAAGATCTGCACACTGGTATTTCGGTTTTTGGGGCCGCGGGC | |
| | GGCGACGGGGCCCGTGCGTCCCAGCGCACATGTTCGGCGAGGCGGGGCCTGCGAG | |
| | CGCGGCCACCGAGAATCGGACGGGGGTAGTCTCAAGCTGGCCGGCCTGCTCTGGT | |
| | GCCTGGCCTCGCGCCGCCGTGTATCGCCCCGCCCTGGGCGGCAAGGCTGGCCCGG | |
| | TCGGCACCAGTTGCGTGAGCGGAAAGATGGCCGCTTCCCGGCCCTGCTGCAGGGA | |
| | GCTCAAAATGGAGGACGCGGCGCTCGGGAGAGCGGGCGGGTGAGTCACCCACACA | |
| | AAGGAAAAGGGCCTTTCCGTCCTCAGCCGTCGCTTCATGTGACTCCACGGAGTAC | |
| | CGGGCGCCGTCCAGGCACCTCGATTAGTTCTCGAGCTTTTGGAGTACGTCGTCTT | |
| | TAGGTTGGGGGGAGGGGTTTTATGCGATGGAGTTTCCCCACACTGAGTGGGTGGA | |
| | GACTGAAGTTAGGCCAGCTTGGCACTTGATGTAATTCTCCTTGGAATTTGCCCTT | |
| | TTTGAGTTTGGATCTTGGTTCATTCTCAAGCCTCAGACAGTGGTTCAAAGTTTTT | |
| | TTCTTCCATTTCAGGTGTCGTGA | |
| Synthetic | AATAAAATCGCTATCCATCGAAGATGGATGTGTGTTGGTTTTTTGTGTG | 225 |
| poly(A) | | |
| signal | | |
| TRAC- | TGGAGCAACAAATCTGACTTTGCATGTGCAAACGCCTTCAACAACAGCATTATTC | 226 |
| RHA | CAGAAGACACCTTCTTCCCCAGCCCAGGTAAGGGCAGCTTTGGTGCCTTCGCAGG | |
| | CTGTTTCCTTGCTTCAGGAATGGCCAGGTTCTGCCCAGAGCTCTGGTCAATGATG | |
| | TCTAAAACTCCTCTGATTGGTGGTCTCGGCCTTATCCATTGCCACCAAAACCCTC | |
| | TTTTTACTAAGAAACAGTGAGCCTTGTTCTGGCAGTCCAGAGAATGACACGGGAA | |
| | AAAAGCAGATGAAGAGAAGGTGGCAGGAGAGGGCACGTGGCCCAGCCTCAGTCTC | |
| | TCCAACTGAGTTCCTGCCTGCCTGCCTTTGCTCAGACTGTTTGCCCCTTACTGCT | |
| | CTTCTAGGCCTCATTCTAAGCCCCTTCTCCAAGTTGCCTCTCCTTATTTCTCCCT | |
| | GTCTGCCAAAAAATCTTTCCCAGCTCACTAAGTCAGTCTCACGCAGTCACTCATT | |
| | AACCCACCAATCACTGATTGTGCCGGCACATGAATGCACCAGGTGTTGAAGTGGA | |
| | GGAATTAAAAAGTCAGATGAGGGGTGTGCCCAGAGGAAGCACCATTCTAGTTGGG | |
| | GGAGCCCATCTGTCAGCTGGGAAAAGTCCAAATAACTTCAGATTGGAATGTGTTT | |
| | TAACTCAGGGTTGAGAAAACAGCTACCTTCAGGACAAAAGTCAGGGAAGGGCTCT | |
| | CTGAAGAAATGCTACTTGAAGATACCAGCCCTACCAAGGGCAGGGAGAGGACCCT | |
| | ATAGAGGCCTGGGACAGGAGCTCAATGAGAAAGG | |

To prepare the genetically engineered immune cells (e.g., T cells disclosed herein), immune cells such as T cells from a suitable source may be obtained, e.g., blood cells from a human donor, who may be a healthy donor or a patient need CAR-T cell therapy. The genetically engineered cells can be made using blood cells from one or more healthy human donors. Manufacturing from healthy donor cells minimizes the risk of unintentionally transducing malignant lymphoma/leukemia cells and potentially may improve the functionality of the CAR T cells. The components of the CRISPR system (e.g., Cas9 protein and the gRNAs), optionally the AAV donor template, may be delivered into the host immune cells via conventional approaches. In some examples, the Cas9 and the gRNAs can form a ribonucleoprotein complex (RNP), which can be delivered to the host immune cells by electroporation. Optionally, the AAV donor template may be delivered to the immune cells concurrently with the RNP complex. Alternatively, delivery of the RNPs and the AAV donor template can be performed sequentially. In some examples, the T cells may be activated prior to delivery of the gene editing components.

After delivery of the gene editing components and optionally the donor template, the cells may be recovered and expanded in vitro. Gene editing efficiency can be evaluated using routine methods for confirm knock-in of the anti-CD117 CAR and knock-out of the target genes (e.g., TRAC, B2M, or both). In some examples, TCRαβ⁺ T cells may be removed.

In some embodiments, the nucleic acid encoding any of the anti-CD117 CARs disclosed herein may be an RNA molecule such as a messenger RNA molecule. When genetic editing is desired, e.g., disrupting TRAC and/or B2M genes, procedures for disrupting the target genes may be performed on suitable parent immune cells such as T cells. The mRNA molecules encoding the anti-CD117 CAR may then be delivered (e.g., via electroporation) to the immune cells having the desired genetic edits to provide CAR-T cells that transiently express the anti-CD117 CAR.

### IV. Treatment Methods and Compositions

In another aspect, provided herein are therapeutic applications of any of the genetically engineered immune cells such as T cells disclosed herein that express an anti-CD117 CAR. Such therapeutic applications include eliminating disease cells expressing CD117, for example, CD117′ cancer cells (e.g., hematopoietic cells such as hematopoietic stem cells. Alternatively, the anti-CD117 CAR-T cells may also be used for myeloablation in subjects in need of hematopoietic stem cell transplantation.

Any of the genetically engineered immune cells such as T cells as disclosed herein (e.g., those expressing an anti-CD117 CAR as also disclosed herein and having one or more additional genetic edits such as a disrupted TRAC gene and/or a disrupted β2M gene) may be formulated in a pharmaceutical composition, which may further comprise one or more pharmaceutically acceptable excipients. Such pharmaceutical compositions are also within the scope of the present disclosure. The pharmaceutical compositions can be used in therapeutic applications, for example, cancer treatment in human patients, which is also disclosed herein.

As used herein, the term “pharmaceutically acceptable” refers to those compounds, materials, compositions, and/or dosage forms which are, within the scope of sound medical judgment, suitable for use in contact with the tissues, organs, and/or bodily fluids of the subject without excessive toxicity, irritation, allergic response, or other problems or complications commensurate with a reasonable benefit/risk ratio. As used herein, the term “pharmaceutically acceptable carrier” refers to solvents, dispersion media, coatings, antibacterial agents, antifungal agents, isotonic and absorption delaying agents, or the like that are physiologically compatible. The compositions can include a pharmaceutically acceptable salt, e.g., an acid addition salt or a base addition salt. See, e.g., Berge et al., (1977) J Pharm Sci 66:1-19.

In some embodiments, the pharmaceutical composition further comprises a pharmaceutically acceptable salt. Non-limiting examples of pharmaceutically acceptable salts include acid addition salts (formed from a free amino group of a polypeptide with an inorganic acid, or an organic acid. In some embodiments, the salt formed with the free carboxyl groups is derived from an inorganic base, or an organic base. In some embodiments, the pharmaceutical composition disclosed herein comprises a population of the genetically engineered CAR-T cells expressing an anti-CD117 CAR as disclosed herein suspended in a cryopreservation solution (e.g., CryoStor® C55).

In some embodiments, any of the genetically engineered T cells expressing an anti-CD117 CAR as disclosed herein can be used for reducing or eliminating disease cells expressing CD117 and thus treating diseases involving such disease cells. For example, the treatment method disclosed herein may be applied to patients (e.g., human patients) having a cancer such as those disclosed herein. Alternatively, the treatment method disclosed herein may be applied to patients (e.g., human patients) who need hematopoietic stem cell transplantation. Such patients may have been received hematopoietic stem cell transplantation. Alternatively, the treatment method disclosed herein may further comprise administering a population of hematopoietic stem cells to the patients in need thereof.

In some examples, the target cancer can be a solid tumor. Examples include, but are not limited to, gastrointestinal stromal tumors, and testicular seminoma. In other examples, the target cancer can be a hematological cancer. Examples include, but are not limited to, leukemia such as acute myeloid leukemia, mast cell disease, and melanoma.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease or reducing disease severity or prolonging survival. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

To perform the method disclosed herein, an effective amount of the genetically engineered T cells expressing an anti-CD117 CAR and optionally one or more additional genetic modifications (e.g., disrupted TRAC gene and/or disrupted β2M gene) can be administered to a subject in need of the treatment (e.g., a human patient having a target cancer as disclosed herein). A subject may be any subject for whom diagnosis, treatment, or therapy is desired. In some embodiments, the subject is a mammal. In some embodiments, the subject is a human.

As used herein, “an effective amount” refers to the amount of each active agent required to confer therapeutic effect on the subject, either alone or in combination with one or more other active agents. Determination of whether an amount of the antibody achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

In some embodiments, an effective amount refers to the amount of a population of genetically engineered T cells as disclosed herein needed to prevent or alleviate at least one or more signs or symptoms of a medical condition (e.g., cancer), and relates to a sufficient amount of a composition to provide the desired effect, e.g., to treat a subject having a medical condition. An effective amount also includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

For use in the various aspects described herein, an effective amount of cells (e.g., engineered T cells) may comprise at least 5×10⁵ cells, at least 1×10⁶ cells, at least 5×10⁶ cells, at least 1×10⁷ cells, or at least 5×10⁷ cells.

In some examples, the genetically engineered T cells are derived from the patient to be treated, i.e., the cells are autologous cells; that is, the engineered T cells are obtained or isolated from a subject and administered to the same subject.

In other examples, the genetically engineered T cells are derived from one or more donors (e.g., healthy human donors) for allogeneic adoptive cell therapy. Allogeneic refers to a cell, cell population, or biological samples comprising cells, obtained from one or more different donors of the same species, where the genes at one or more loci are not identical to the recipient. For example, an engineered T cell population being administered to a subject can be derived from one or more unrelated donors, or from one or more non-identical siblings. A donor is an individual who is not the subject being treated. In some embodiments, a donor is an individual who does not have or is not suspected of having the cancer being treated.

In some embodiments, multiple donors, e.g., two or more donors, are used. In some examples described herein, the cells are expanded in culture prior to administration to a subject in need thereof.

The step of administering may include the placement (e.g., transplantation) of cells, e.g., engineered T cells, into a subject, by a method or route that results in at least partial localization of the introduced cells at a desired site, such as tumor, such that a desired effect(s) is produced. Engineered T cells can be administered by any appropriate route that results in delivery to a desired location in the subject where at least a portion of the implanted cells or components of the cells remain viable. The period of viability of the cells after administration to a subject can be as short as a few hours, e.g., twenty-four hours, to a few days, to as long as several years, or even the life time of the subject, i.e., long-term engraftment. For example, in some aspects described herein, an effective amount of engineered T cells is administered via a systemic route of administration, such as an intraperitoneal or intravenous route.

Modes of administration include injection, infusion, instillation, or ingestion. Injection includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some embodiments, the route is intravenous.

In some embodiments, engineered T cells are administered systemically, which refers to the administration of a population of cells other than directly into a target site, tissue, or organ, such that it enters, instead, the subject's circulatory system and, thus, is subject to metabolism and other like processes.

Any subjects (e.g., human patients) suitable for the treatment methods disclosed herein may receive a lymphodepleting therapy to reduce or deplete the endogenous lymphocyte of the subject. Lymphodepletion refers to the destruction of endogenous lymphocytes and/or T cells, which is commonly used prior to immunotransplantation and immunotherapy. Lymphodepletion can be achieved by irradiation and/or chemotherapy. A “lymphodepleting agent” can be any molecule capable of reducing, depleting, or eliminating endogenous lymphocytes and/or T cells when administered to a subject. In some embodiments, the lymphodepleting agents are administered in an amount effective in reducing the number of lymphocytes by at least 10%, 20%, 30%, 40%, 50%, 60%, 70%, 80%, 90%, 95%, 96%, 96%, 97%, 98%, or at least 99% as compared to the number of lymphocytes prior to administration of the agents. In some embodiments, the lymphodepleting agents are administered in an amount effective in reducing the number of lymphocytes such that the number of lymphocytes in the subject is below the limits of detection. In some embodiments, the subject is administered at least one (e.g., 2, 3, 4, 5 or more) lymphodepleting agents.

In some embodiments, the lymphodepleting agents are cytotoxic agents that specifically kill lymphocytes. Examples of lymphodepleting agents include, without limitation, fludarabine, cyclophosphamide, bendamustin, 5-fluorouracil, gemcitabine, methotrexate, dacarbazine, melphalan, doxorubicin, vinblastine, cisplatin, oxaliplatin, paclitaxel, docetaxel, irinotecan, etopside phosphate, mitoxantrone, cladribine, denileukin diftitox, or DAB-IL2. In some instances, the lymphodepleting agent may be accompanied with low-dose irradiation. The lymphodepletion effect of the conditioning regimen can be monitored via routine practice.

The efficacy of a treatment as disclosed herein can be determined by the skilled clinician. A treatment can be considered “effective treatment,” if any one or all of the signs or symptoms of, as but one example, levels of functional target are altered in a beneficial manner (e.g., increased by at least 10%), or other clinically accepted symptoms or markers of disease (e.g., cancer) are improved or ameliorated. Efficacy can also be measured by failure of a subject to worsen as assessed by hospitalization or need for medical interventions (e.g., progression of the disease is halted or at least slowed). Methods of measuring these indicators are known to those of skill in the art and/or described herein. Treatment efficacy includes, but are not limited to, (1) inhibiting the disease, e.g., arresting, or slowing the progression of symptoms; or (2) relieving the disease, e.g., causing regression of symptoms; and (3) preventing or reducing the likelihood of the development of symptoms.

### V. Kit for CAR-T Cell Therapy

The present disclosure also provides kits for use of a population of genetically engineered immune cells such as T cells that express an anti-CD117 CAR and optionally have one or more additional genetic modifications such as disrupted TRAC and/or disrupted B2M as described herein in methods for treating a target disease, e.g., a cancer such as those disclosed herein or for myeloablation in patients who have received or will receive hematopoietic stem cell transplantation. Such kits may include one or more containers comprising a first pharmaceutical composition that comprises one or more lymphodepleting agents, and a second pharmaceutical composition that comprises any nucleic acid or population of genetically engineered T cells (e.g., those described herein), and a pharmaceutically acceptable carrier.

In some embodiments, the kit can comprise instructions for use in any of the methods described herein. The included instructions can comprise a description of administration of the first and/or second pharmaceutical compositions to a subject to achieve the intended activity in a human patient. The kit may further comprise a description of selecting a human patient suitable for treatment based on identifying whether the human patient is in need of the treatment. In some embodiments, the instructions comprise a description of administering the first and second pharmaceutical compositions to a human patient who is in need of the treatment.

The instructions relating to the use of a population of genetically engineered T cells described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the population of genetically engineered T cells is used for treating, delaying the onset, and/or alleviating a cancer in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port. At least one active agent in the pharmaceutical composition is a population of the genetically engineered T cells as disclosed herein.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### GENERAL TECHNIQUES

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1: Screening for and Characterization of Anti-CD117 VHH Antibodies

Nucleotide sequences encoding a total of 28 anti-CD117 VHH binders were each cloned into a vector for expression and secretion of the encoded VHH antibody in Expi293 cells according to manufacturer's protocol (ThermoFischer Scientific). The host cells were transfected with the expression vector and the transfected cells were maintained in a shaking incubator for about one week to allow expression and secretion of the VHH antibody into the supernatant. The cell supernatants were harvested by centrifugation and filtered to remove cell debris. Standard protein analytics were performed to measure supernatant protein expression and yield.

Nine VHH binders, P10, P12, P27, P29, P31, P32, P35, P37, or P38, identified herein as having high binding activity to human CD117 and optionally cyno CD117 were expressed as fusion polypeptides with an Fc fragment. The amino acid sequences of these VHH binders are provided in Tables 1 and 2 above.

Human and Cynomolgous monkey (Cyno) CD117 Ectodomain was expressed by the same approach as disclosed herein. The CD117 ectodomain was expressed as a fusion polypeptide to an AviTag added to the C terminus to allow capture on Octet Biosensors.

Various assays were performed for characterization of the anti-CD117 VHH binders, including biolayer interferometry Octet® assay, ELISA, and flow cytometry assays, following manufacturers' protocols.

Upon analyzing binding activities of the VHH binders tested in this example, P10, P12, P27, P29, P31, P32, P35, P37, and P38 were identified as the top binders. Amino acid sequences of these VHH binders are provided in the sequence Tables 1 and 2. Binding affinities (Kd) of the VHH binders to human CD117 range from 0.28 to 1.91 nM in monovalent form and range from 0.16 to 0.54 nM in divalent form. High binding activities to cell surface CD117 by these VHH binders were also detected using SKMEL cells and CD34 cells (both CD117+ cells) as determined by Flow Cytometry.

Results from flow cytometry and Octet® analysis show that P10, P29, P31, P35, P37, and P38 in monovalent form cross-react between human and cyno CD117 and all of the top nine binders in bivalent form cross-react between human and cyno CD117.

Further, the VHHs binders were assessed to see whether they compete with Stem Cell Factor (SCF) for a binding location with CD117, using the biolayer interferometry method disclosed herein. Full length AviTagged CD117 ectodomain was bound to an SA biosensor (Sartorius). The biosensor was either (1) incubated first with SCF (R&D systems), followed by one of the 9 VHH binders of interest or, (2) incubated first with one of the VHH binders, followed by SCF. Competition was most easily visualized when SCF bound to the CD117 ectodomain first, as it had a stable interaction with a slow off-rate. Two VHHs, P35 and P37 showed completion with SCF when SCF bound first. Some interference between VHHs and SCF was also observed when the VHHs bound to the CD117 ectodomain first. 5 VHHs P10, P12, P31, P35 and P37, showed some potential to block SCF binding when they were allowed to bind to the CD117 Ectodomain first.

The 9 top VHH binders (in bivalent form) were investigated for their impact on the growth of CD34+ HSCs and ligand-induced C-kit phosphorylation. Most of the tested VHH binders (e.g., P10, P31, and P38) showed no impact on CD34+ cell growth. Similarly, these VHH binders (e.g., P10, P31, and P38) also showed no impact on inhibition of ligand-induced C-kit pTyr phosphorylation.

### Example 2: Generation and Characterization of Antibody (scFv)-Derived Anti-CD117 CAR T Cells

This example describes the production and characterization of human T cells expressing exemplary anti-CD117 CARs that contain an anti-CD117 scFv and have disrupted TRAC and β2M genes.

Activated primary human T cells were electroporated with Cas9/sgRNA RNP complexes (200 pmol Cas9, 1000 pmol gRNA) to generate cells edited for TRAC⁻/β2M⁻. Sequences encoding 8 anti-CD117 CARs were each inserted into the TRAC locus using recombinant AAV6 carrying the DNA sequences for the CARs. The following sgRNAs were used: TRAC (SEQ ID NO: 40) and β2M (SEQ ID NO: 205). See Table 3 above. The resultant CAR T cells were designated as CTX-2840 to CTX-2847. See sequence Table 2 for structure information of these exemplary anti-CD117 CARs.

### (i) Assessment of CAR Expression and TRAC, β2M Editing

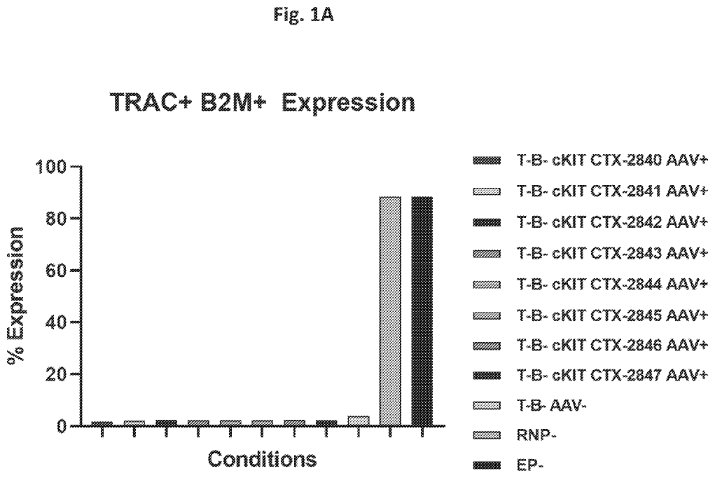

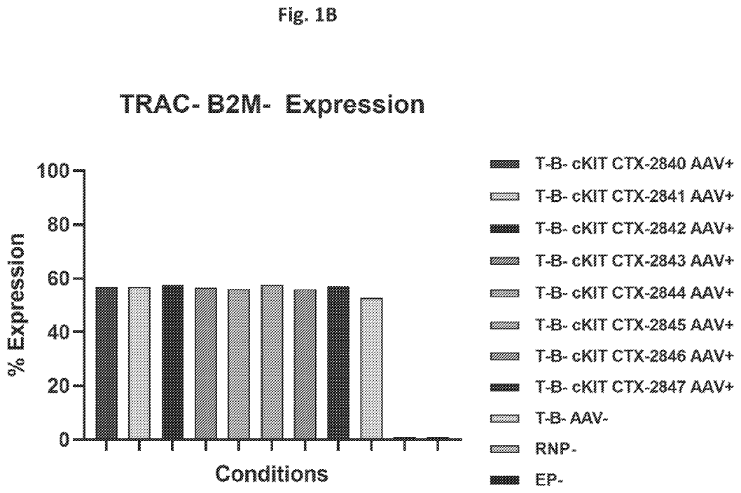

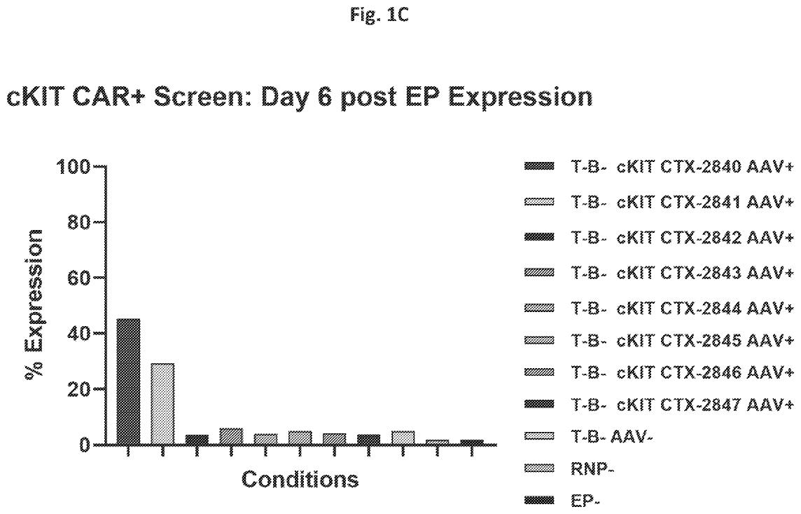

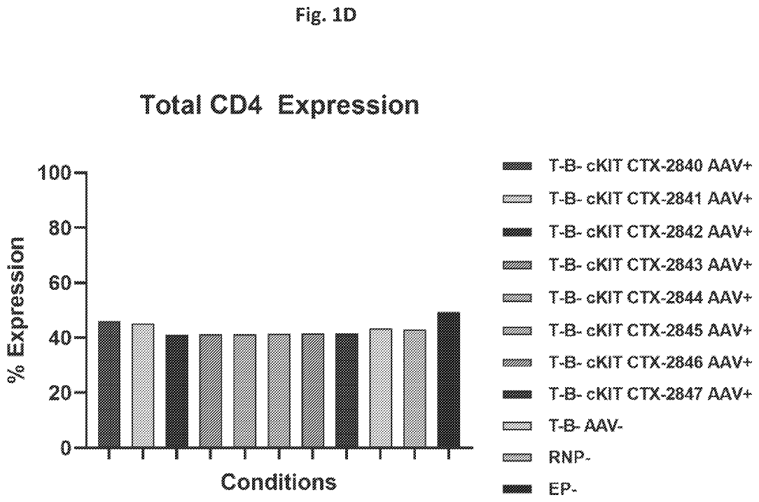

Flow cytometry was used to verify editing of the TRAC and β2M locus and, insertion and expression of anti-CD117 VHH CAR. Briefly, about one-week post-electroporation, cells were stained with anti-human TCR, anti-human β2M, and biotinylated recombinant CD117 protein/SA-APC conjugate to assess the levels of editing for TRAC and 82M, and insertion of the nucleotide sequence encoding anti-CD117 CAR. Within the T cell population, >90% of viable cells lacked expression of TCR and >55% lacked expression of β2M (FIGS. 1A-1B). The cells also had a high percentage of viable cells expressing the anti-CD117 CAR (FIG. 1C). CTX-2840 and CTX-2841 showed significant rates of CAR insertion (30-45%). Unedited RNP Tells had no detectable staining for anti-CD117 CAR.

Cells were also stained with anti-human CD4 and anti-human CD8 to determine the ratios of CD4: CD8 T cells in the samples. Flow cytometry analysis showed that the ratios of CD4: CD8 T cells were uniform across the various constructs tested (FIGS. 1D-1E).

### (ii) Cytotoxicity

Cytotoxicity assays were used to assess the ability of the TRAC⁻/β2M⁻ anti-CD117 CAR T cells to cause cell lysis in target cell lines.

### (1) Assay 1

A CD117-positive melanoma cell line, SKMEL-3, and a CD117-negative tongue carcinoma cell line, CAL-27, were selected as target cells for the assay. Unedited RNP cells without CAR were used as a negative control to determine the CD117 specific lysis of target cells by CAR+ T cells.

Briefly, SKMEL-3 and CAL-27 cells were plated in 96-well plates at 30,000 cells per well. After 24 hours, CAR T cells or RNP T cells were added to the wells at T cell: target cell ratios of 0.5:1, 1:1, 2:1, or 4:1 and incubated further for approximately 20 hours. One set of wells that did not receive any T cells served as a control. After the incubation period, the plates were centrifuged at 300×g for 10 minutes and 100 μL of supernatant was removed for cytokine quantification. The cell monolayers were washed twice with PBS to remove T cells. ATP in metabolically active live SKMEL-3 or CAL-27 cells was then measured by adding 100 μL of Cell Titer-Glo luminescent reagent (Promega, Cat: G8461) to each well, incubating the plates for 10 mins in the dark at 37° C., and quantifying live cell viability from the luminescent signal (BioTek Synergy H1 luminescence plate reader).

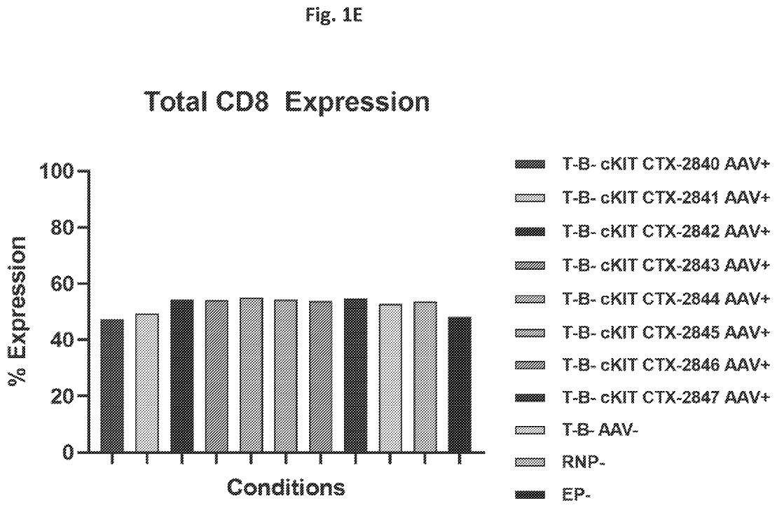

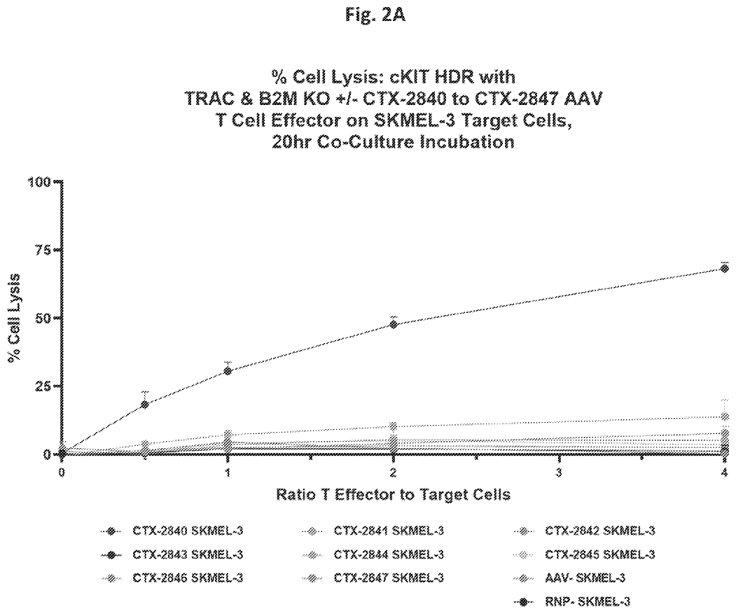

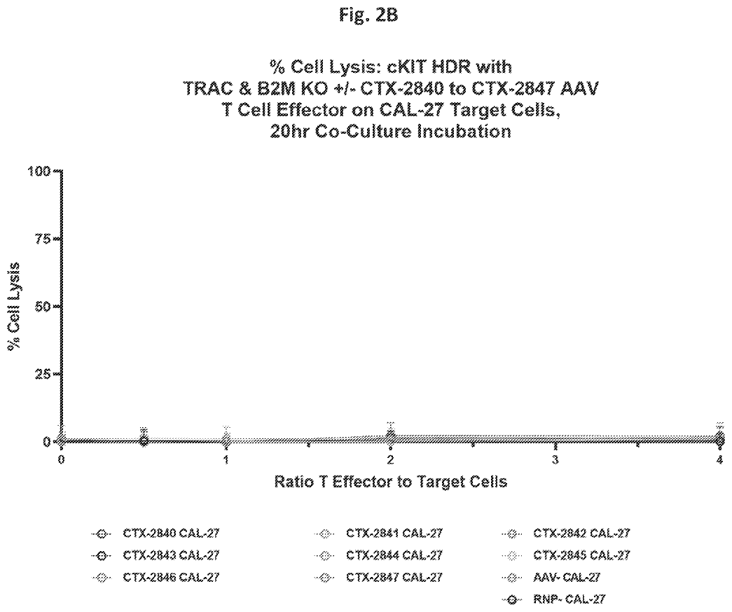

CTX-2840 and CTX-2841 cells exhibited greater cytotoxicity towards CD117-positive SKMEL-3 cells (FIG. 2A) compared to CD117-negative CAL-27 cells (FIG. 2B).

### (2) Assay 2

A CD117-positive acute myeloid leukemia cell line, Kasumi-1, and CD117-negative tongue carcinoma cell line, CAL-27, were selected as target cells for the cytotoxicity assayCTX-2840CTX-2841. Unedited RNP cells without CAR were used as a negative control to determine the CD117-specific lysis of target cells by CAR+ T cells.

Briefly, target cells were stained with eBioscience™ Cell Proliferation Dye eFluor™ 670 (ThermoFisher Scientific; Cat #65-0840-85) per the manufacturer's instructions. The cells were then seeded into 96-well plates at 50,000 cells per well. Next, CAR-T cells or RNP-T cells were added to the wells at T cell: target cell ratios of 0.5:1, 1:1, 2:1, or 4:1, and incubated for approximately 20 hours. One set of wells that did not receive any T cells served as a control. After the incubation period, the plates were centrifuged at 300×g for 10 minutes and 100 μL of supernatant was removed for cytokine quantification. Cells were then washed once with PBS and stained with DAPI (5 μg/mL, Invitrogen; Cat #D3571) in 150 μL of PBS supplemented with 0.5% BSA and incubated for 15 min in the dark. Post-incubation, the cells were washed to remove excess DAPI, resuspended in 150 μL PBS supplemented with 0.5% BSA, and analyzed using a flow cytometer. Target cells were identified via eFluor™ 670-based fluorescence and then divided into live and dead cells based on their DAPI fluorescence.

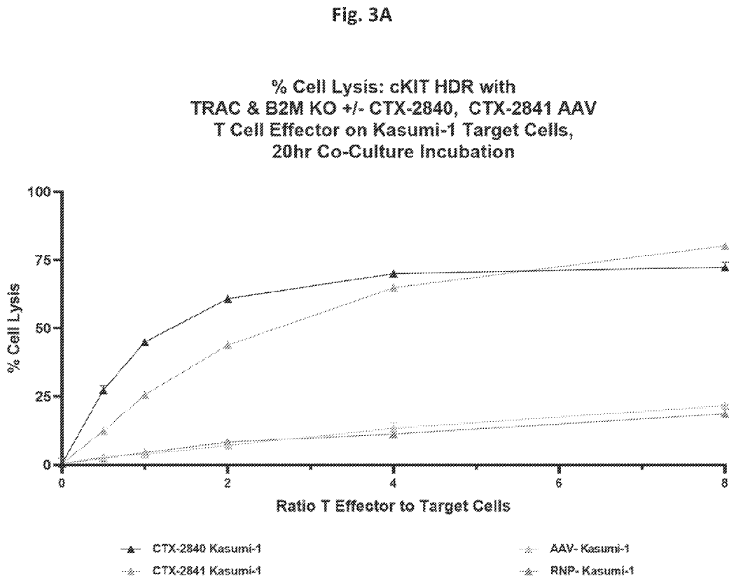

It was observed that CTX-2840 and CTX-2841 T cells exhibited greater cytotoxicity towards cKIT-positive Kasumi-1 cells (FIG. 3A) compared to cKIT-negative CAL-27 cells (FIG. 3B).

### (3) Assay 3

CD117-positive CD34+ hematopoietic stem and progenitor cells (HSPCs), CD34+ HSPCs, the CD117+ acute myelod leukemia (AML) cell line, Kasumi-1, and CD117 negative tongue carcinoma cell line, CAL-27, were used as target cells for the cytotoxicity assay. Unedited RNP cells without CAR were used as a negative control to determine CD117-specific lysis by CAR+ T cells.

Briefly, target cells were stained with eBioscience™ Cell Proliferation Dye eFluor™ 670 (Thermofisher Scientific; Cat #65-0840-85) per the manufacturer's instructions. The cells were then seeded into 96-well plates at 50,000 cells per well. Next, CAR-T cells or RNP-T cells were added to the wells at T cell: target cell ratios of 0.5:1, 1:1, 2:1, or 4:1 and incubated further for approximately 18 hours. One set of wells did not receive any T cells and served as a control. After the incubation period, the plates were centrifuged at 300×g for 10 minutes and 100 μL of supernatant was removed for cytokine quantification. Cells were then washed once with PBS and stained with DAPI as described above for Assay 2, and analyzed by flow cytometry. Target cells were identified via eFluor-based fluorescence and then divided into live and dead cells based on DAPI fluorescence.

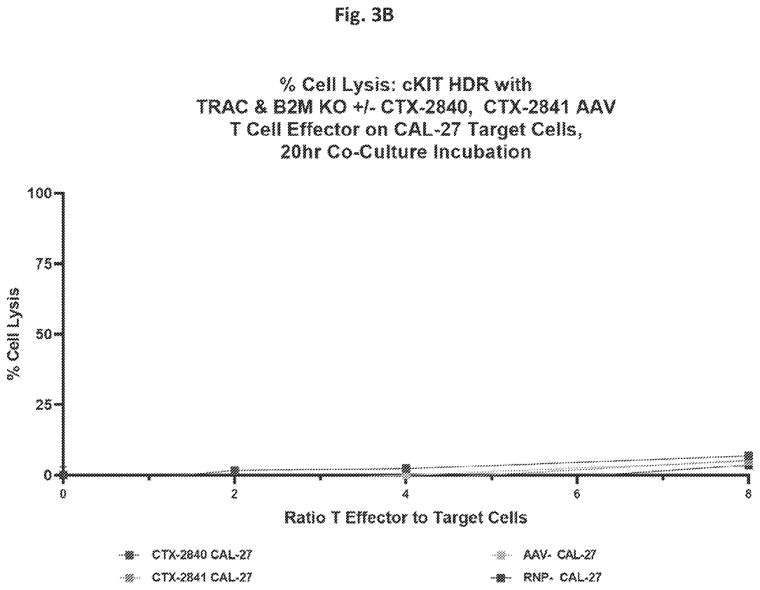

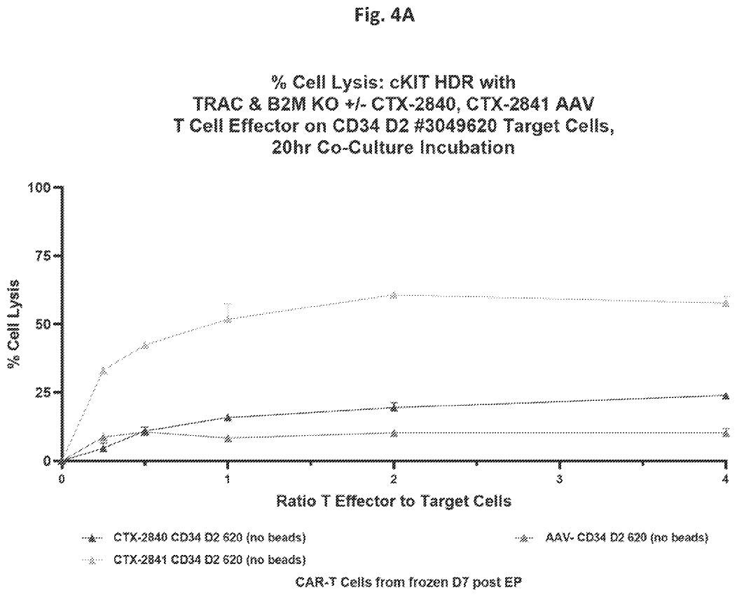

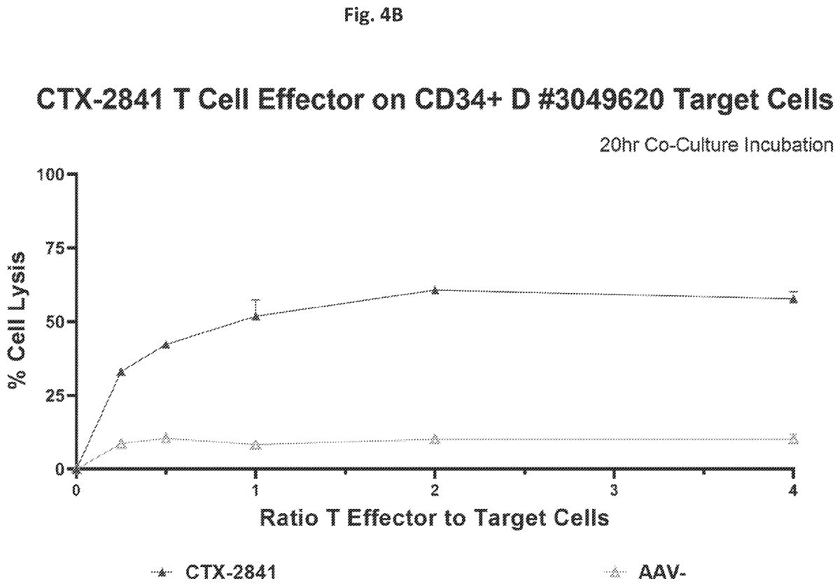

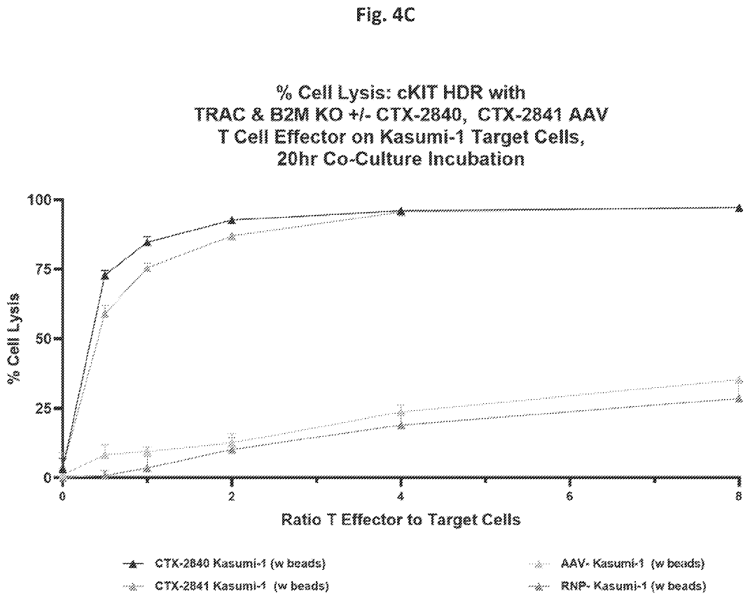

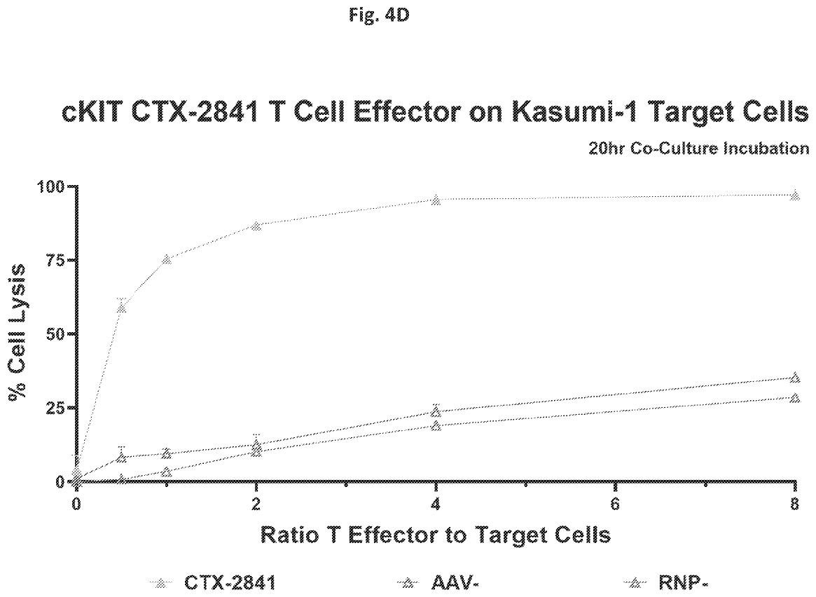

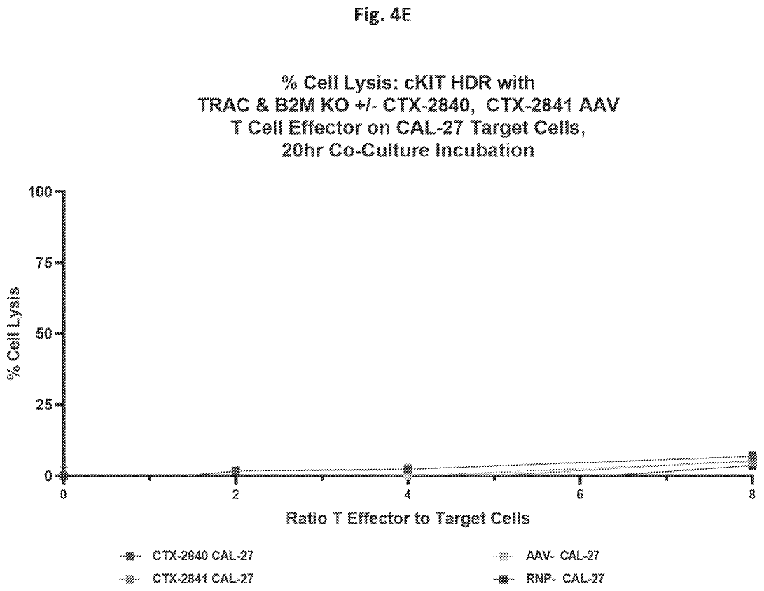

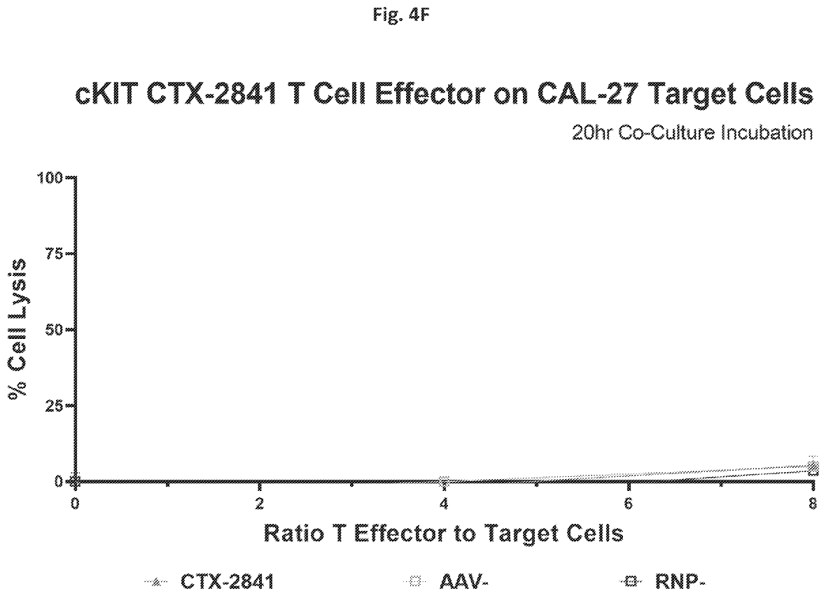

It was observed that CTX-2840 and CTX-2841 T cells exhibited greater cytotoxicity towards CD117-positive CD34+ HSPCs (FIGS. 4A-4B) and Kasumi-1 cells (FIGS. 4C-4D) compared to CD117-negative CAL-27 cells (FIGS. 4E-4F).

The results reported in this Example demonstrate successful construction of TRAC−/β2M−/anti-CD117-CAR-T cells, which showed cytotoxicity against CD117+ target cells.

### Example 3: Generation and Characterization of VHH-Derived Anti-CD117 CAR T Cells

This example describes the production and characterization of exemplary human T cells expressing ant-CD117 CARs that comprise an VHH anti-CD117 moiety and having disrupted TRAC and β2M genes.

Activated primary human T cells were electroporated with Cas9/sgRNA RNP complexes (200 pmol Cas9, 1000 pmol gRNA) to generate cells edited for TRAC⁻/β2M⁻. Sequences encoding 16 anti-CD117 VHH CARs were each inserted into the TRAC locus using recombinant AAV6 carrying the DNA sequences for anti-CD117 VHH CARs. The following sgRNAs were used: TRAC (SEQ ID NO: 40) and β2M (SEQ ID NO: 205). See Table 3 above. The resultant CAR T cells were designated as CTX-2867 to CTX-2882.

### (i) Assessment of CAR Expression and TRAC, β2M Editing

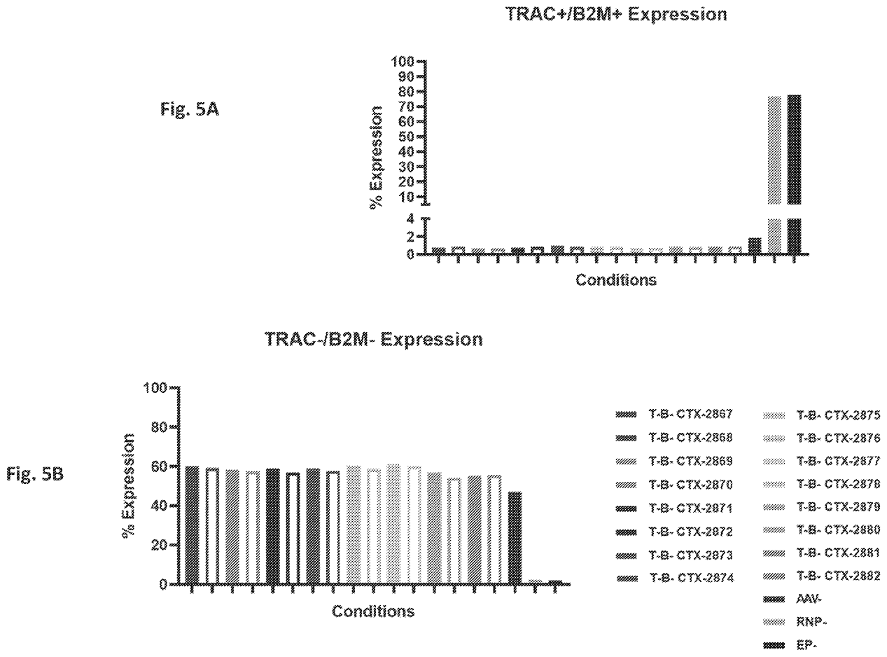

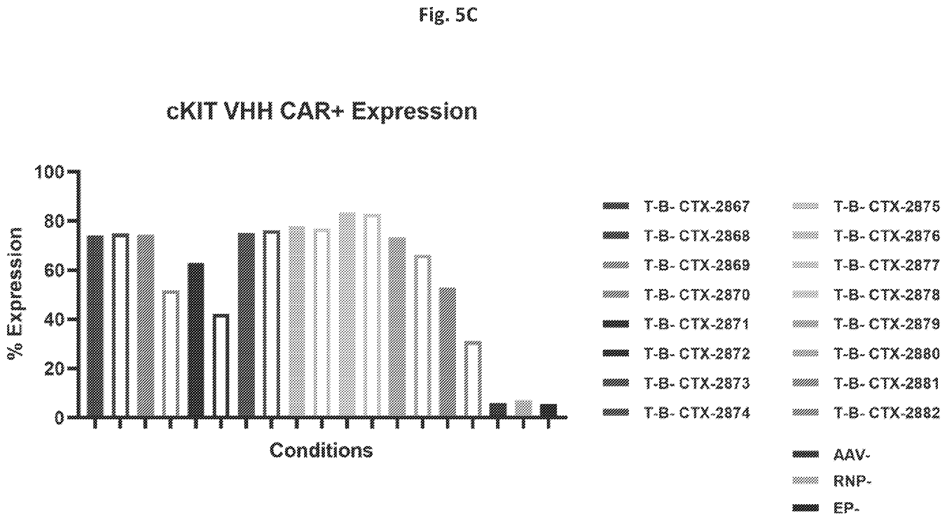

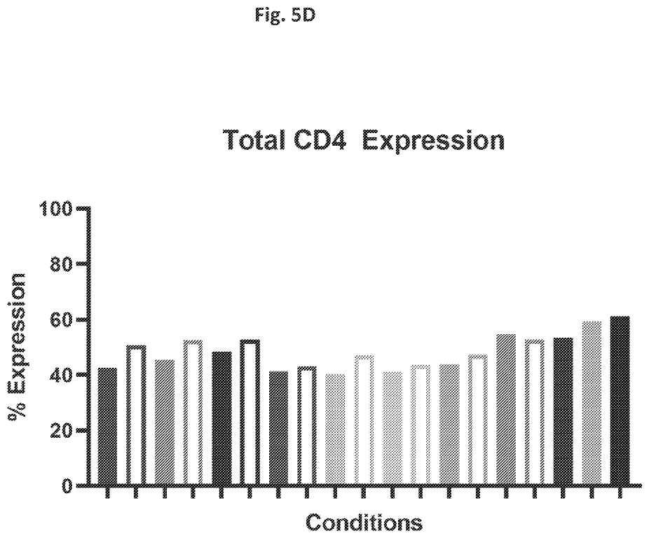

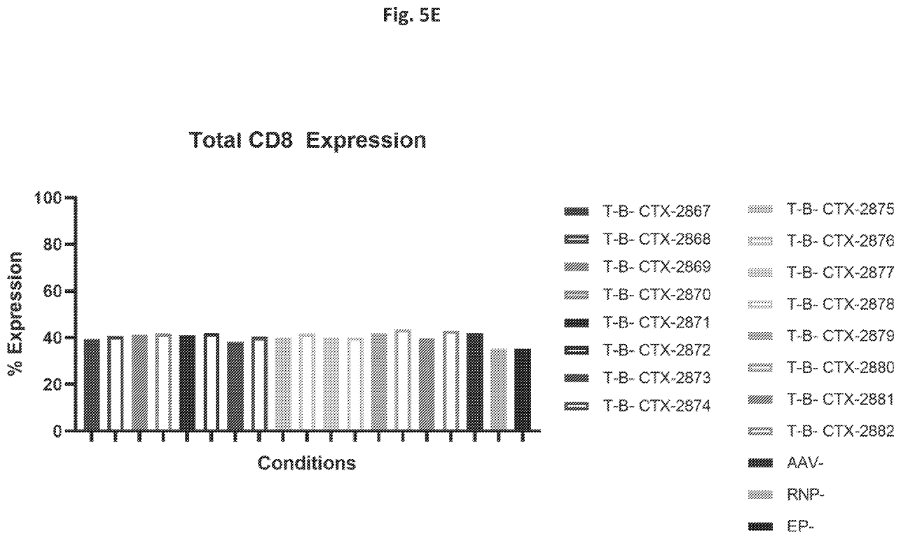

Flow cytometry was used to verify editing of the TRAC and β2M locus and the insertion and expression of anti-CD117 VHH CAR. Briefly, about one-week post-electroporation, cells were stained with anti-human TCR, anti-human β2M, and anti-Camelid-APC conjugate to assess the levels of editing for TRAC and β2M, and insertion of the nucleotide sequence encoding anti-CD117 VHH CAR. Within the T cell population, >90% of viable cells lacked expression of TCR and ˜60% lacked expression of β2M (FIGS. 5A-5B). The cells also had a high percentage (>31-84%) of viable cells expressing the anti-cKIT CAR (FIG. 5C).

Cells were also stained with anti-human CD4 and anti-human CD8 to determine the ratios of CD4: CD8 T cells in the samples. Flow cytometry analysis showed that the ratios of CD4: CD8 T cells were uniform across the constructs tested (FIGS. 5D-5E).

### (ii) Cytotoxicity

Cytotoxicity assays were used to assess the ability of the TRAC⁻/β2M⁻ anti-CD117 VHH CAR T cells to cause cell lysis in target cell lines.

CD117-positive CD34+ HSPCs, the CD117-positive acute myeloid leukemia cell line, Kasumi-1, and the CD117-negative tongue carcinoma cell line, CAL-27, were selected as target cells for a cytotoxicity assay. Unedited RNP cells without CAR were used as negative control to determine CD117-specific lysis by CAR+ T cells.

Briefly, target cells were stained with eBioscience™ Cell Proliferation Dye eFluor™ 670 (ThermoFisher Scientific; Cat #65-0840-85) per the manufacturer's instructions. The cells were then seeded into 96-well plates at 50,000 cells per well. Next, CAR-T cells or RNP-T cells were added to the wells at T cell: target cell ratios of 0.5:1, 1:1, or 2:1 and incubated further for approximately 18 hours. One set of wells did not receive any T cells and served as a control. Cells were then processed as described above.

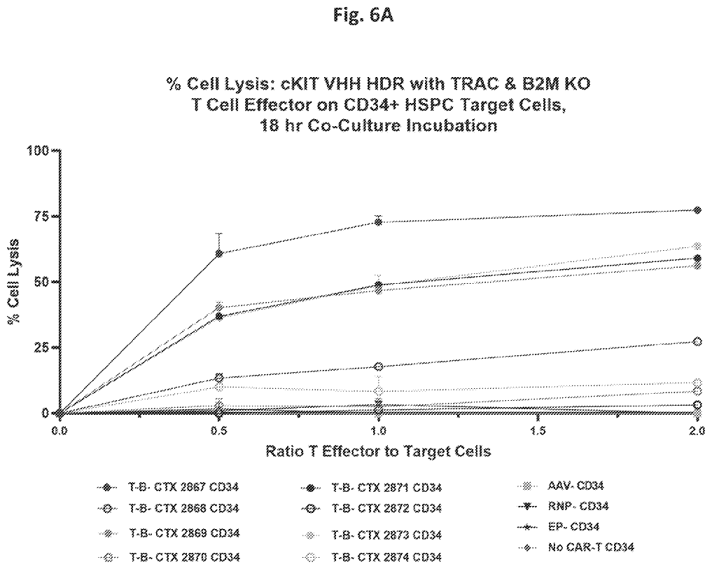

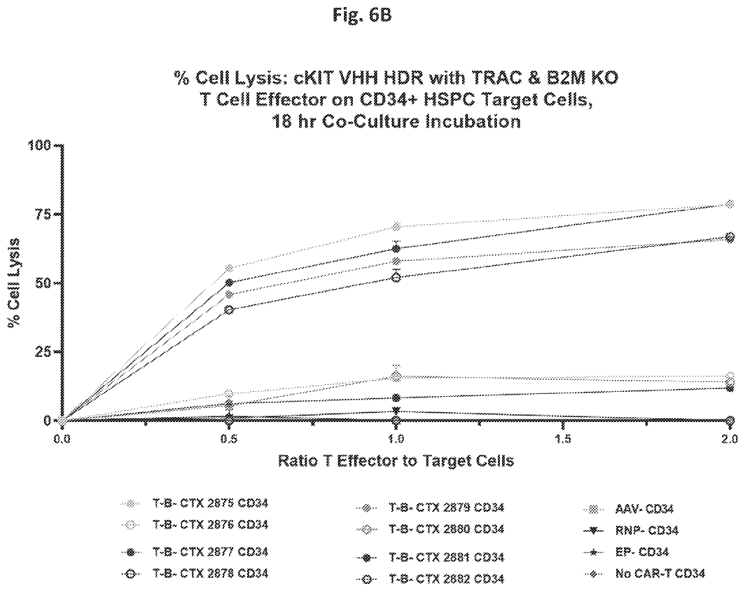

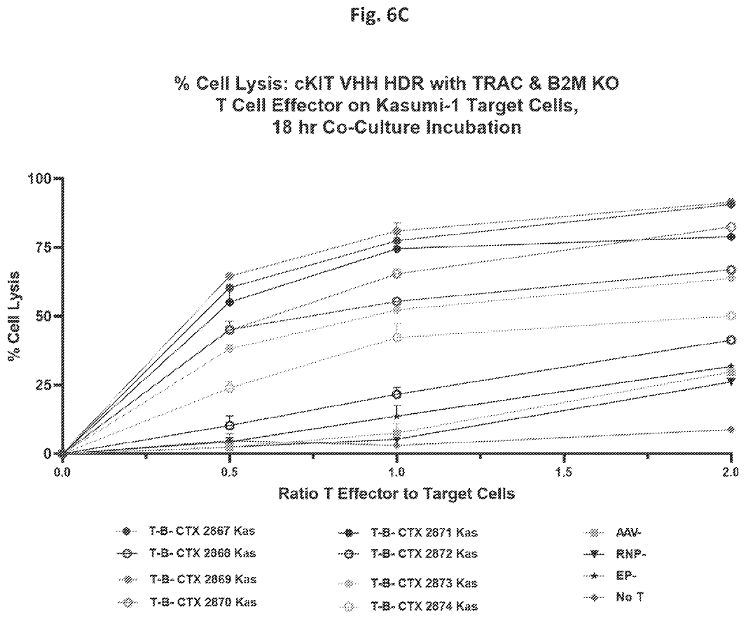

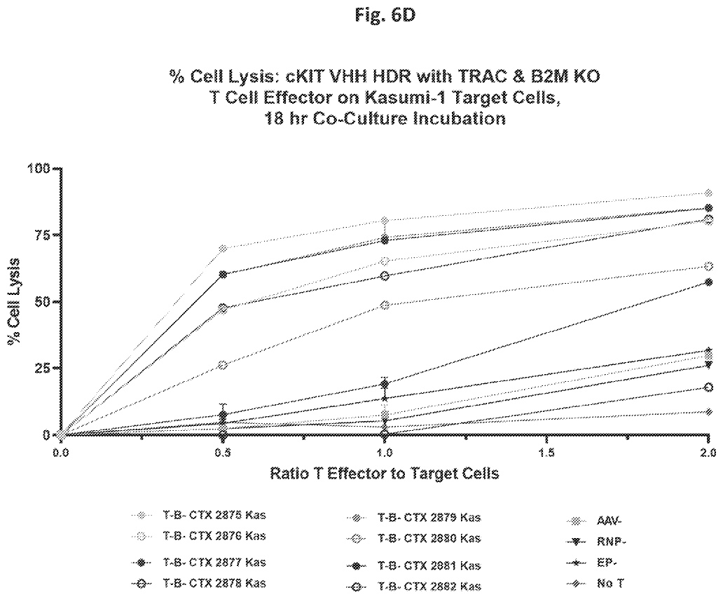

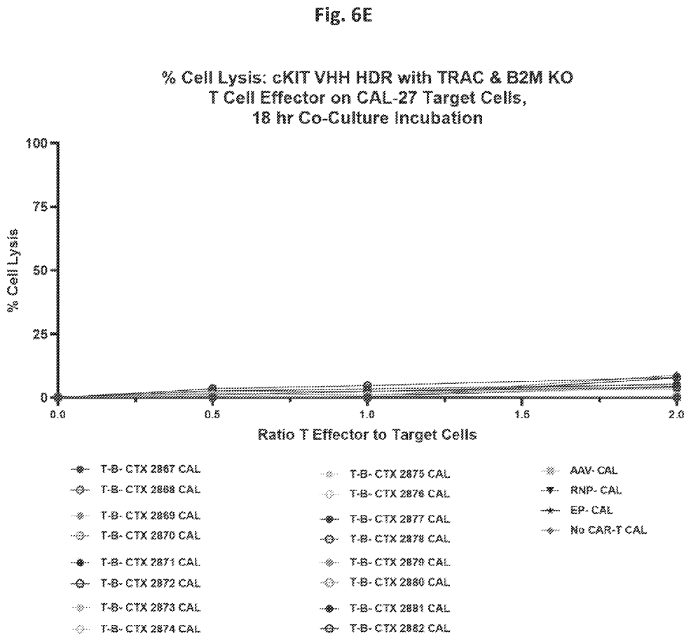

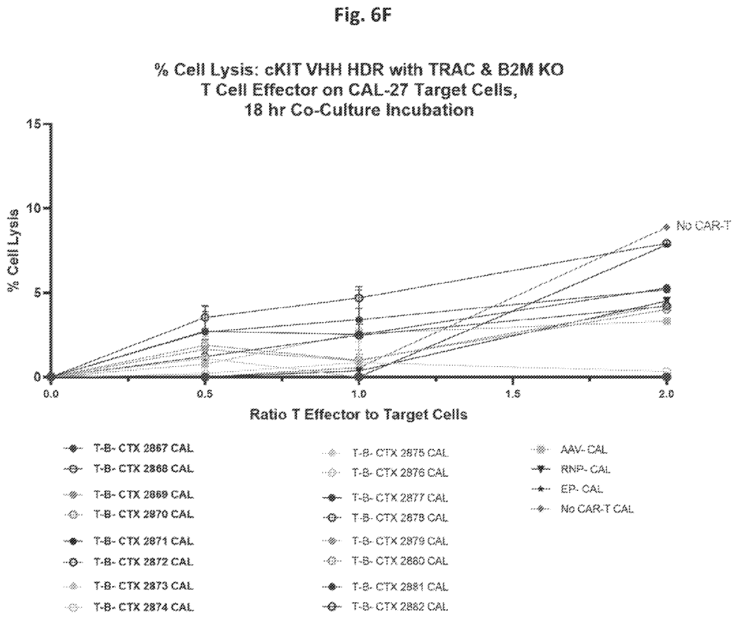

It was observed that the TRAC⁻/β2M⁻ anti-CD117 VHH CAR T cells exhibited greater cytotoxicity towards CD117-positive CD34+ HSPCs (FIGS. 6A-6B) and Kasumi-1 cells (FIGS. 6C-6D) compared to CD117-negative CAL-27 cells (FIGS. 6E-6F). Specifically, T cells expressing anti-CD117 CARs with the CD28 costimulatory domain were generally more active than those with the 4-1BB costimulatory molecule. CTX-2877 and CTX-2878 showed cytotoxicity irrespective of whether the costimulatory molecule was CD28 or 4-1BB.

### Example 4: In Vivo Cytotoxicity of Anti-CD117 CAR T Cells

This example provides demonstration of in vivo cytotoxicity of three allogeneic CD117− targeted CAR T cells.

CTX-2841 with scFv anti-CD117 CAR and CD28 costimulatory molecule (“CAR41”), CTX-2867 with VHH anti-CD117 and CD28 costimulatory molecule (“CAR67”), and CTX-2878 VHH anti-CD117 with 4-1BB costimulatory molecule (“CAR78”) were used as examples for this study.

Humanized NSG mice (huCD34+ engrafted cells) were randomly divided into 3 groups of 6 mice each. Each group received one of the three CAR T cells described above (1×10⁷ cells) by intravenous injection. A control group of 9 mice were received the vehicle. On days 7 and 14, 3 mice from each group were euthanized and CD34+/CD117+ cells in the bone marrow quantified by flow cytometry. Drug product distribution in blood, bone marrow, and spleen were also assessed.

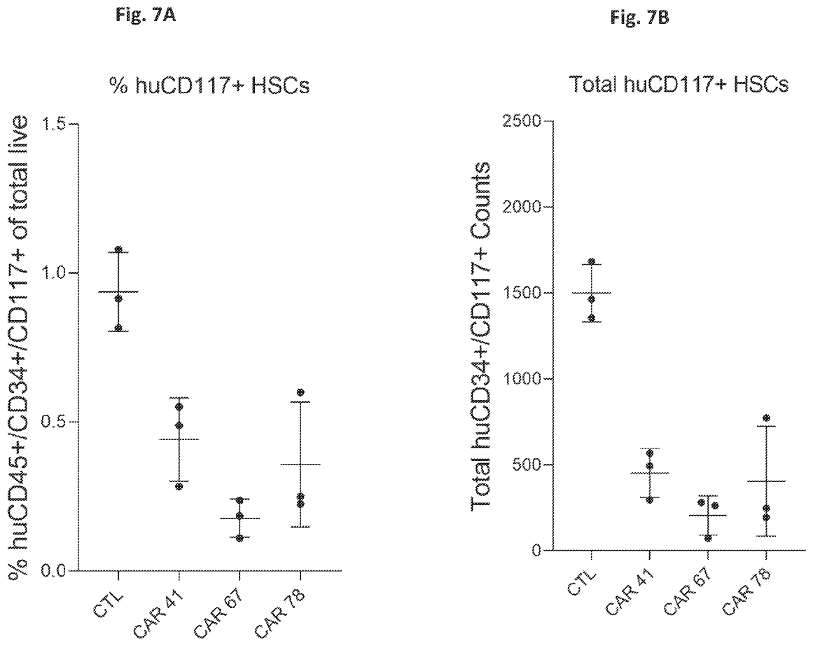

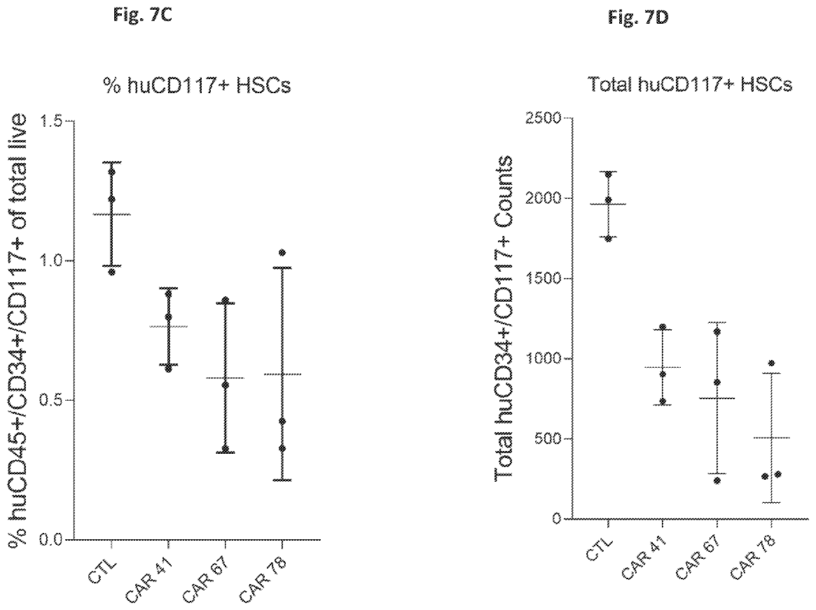

On both day 7 and 14, it was observed that CAR T cell-treated animals showed reduced CD117⁺ HSCs, demonstrating the efficacy of the treatment. FIGS. 7A-7D.

### Example 5: Generation and Characterization of CAR T Cells Transiently Expressing an Anti-CD117 CAR

This example describes generation of CAR-T cells that express an anti-CD117 CAR transiently and characterization of such anti-CD117 CAR-T cells in vitro.

### A. Generation of Anti-CD117 CAR T Cells Transiently Expressing Anti-CD117 CAR

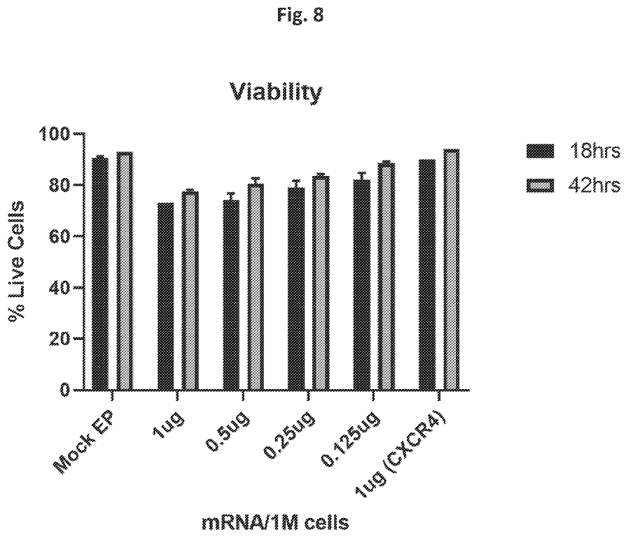

Activated primary human T cells (e.g., using anti-CD3 and anti-CD28 agonists conjugated to beads such as TransAct™) were electroporated (EP) with mRNA molecules encoding the anti-CD117 CAR of SEQ ID NO: 111 (CTX-2840) at concentrations from 0.125 μg to 1 μg per 10⁶ cells. At 18 hours and 42 hours pose EP, the cells were tested for viability using APOI dye and CAR expression by flow cytometry. As shown in FIG. 8, electroporation of anti-CD117 CAR-encoding mRNA to activated T cells showed no significant impact on cell viability at all tested concentrations. Expression of the anti-CD117 CAR was detected in the T cells at least 42 hours post EP.

Based on the viability and expression data, the concentration of 0.25 μg/10⁶ T cells of the anti-CD117 CAR mRNA was found to be optimal since it yielded high CAR expression without compromising cell viability post electroporation (EP).

B. Generation of Anti-CD117 CAR T Cells with TRAC and β2M Disruptions Activated primary human T cells (20×10⁶ cells) were first electroporated with Cas9/sgRNA RNP complexes (60 μM Cas9, 200 μM of each of TRAC and β2M gRNAs) to generate cells TRAC⁻/β2M⁻ cells. The following sgRNAs were used: TRAC (SEQ ID NO: 40) and β2M (SEQ ID NO: 205). See Table 3 above. Disruptions of the TRAC and #82M genes were confirmed using the method described in Example 2. The edited cells were then electroporated with mRNA molecules encoding each of the anti-CD117 CAR constructs listed in Table 5 below at a concentration of 0.25 μg/10⁶ T cells. Amino acid sequences of these anti-CD117 CAR constructs are provided in Table 2 above.

TABLE 5
Exemplary Anti-CD117 CAR Constructs

| No. | Name | Features |
|---|---|---|
| 1 | CTX-2840 | Ab-1; VL/VH; CD28 CoS |
| 2 | CTX-2841 | Ab-1; VL/VH; 4-1BB CoS |
| 3 | CTX-2842 | Ab-1; VH/VL; CD28 CoS |
| 4 | CTX-2843 | Ab-1; VH/VL; 4-1BB CoS |

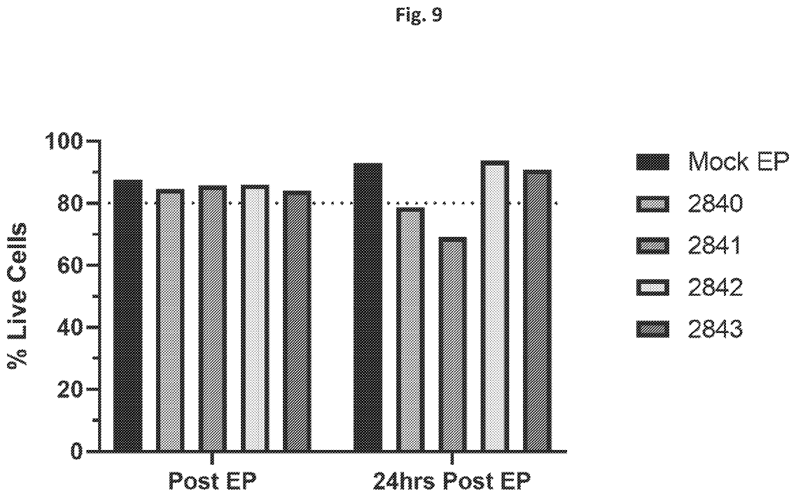

CAR T cell viability and CAR expression analysis were examined following the methods disclosed above. As shown in FIG. 9, no significant reduction of cell viability was observed in any of the transfected T cells at least 24 hours post EP. Expression of the anti-CD117 CAR was also observed in the transfected T cells.

Among the four anti-CD117 CAR tested, constructs having the V_{L}-V_{H} configuration (CTX-2840 and CTX-2841) show higher levels of CAR expression as compared with constructs having the V_{H}-V_{L} configuration (CTX-2842 and CTX-2843).

### C. Kinetics of Anti-CD117 CAR Expression

TRAC⁻/β2M⁻ T cells (˜91.2% TRAC KO and ˜76% β2M KO) generated as described above were electroporated with the mRNA encoding CTX-2840 anti-CD117 CAR at 0.25 μg/10⁶ T cells. CAR expression was assessed by flow cytometry at different time intervals from 5 hours to 96 hours post electroporation. The data shows that the expression of the anti-CD117 CAR was transient and declined at 48 hours post-EP, although the anti-CD117 expression was not completely abolished even at 96 hours.

D. Effect of Anti-CD117 CAR mRNA Transfection on Expression of T cell Activation and Exhaustion Markers

The effects of anti-CD117 CAR mRNA transfection on expression of markers for T cell activation, differentiation, and exhaustion were investigated at 48 hours post electroporation.

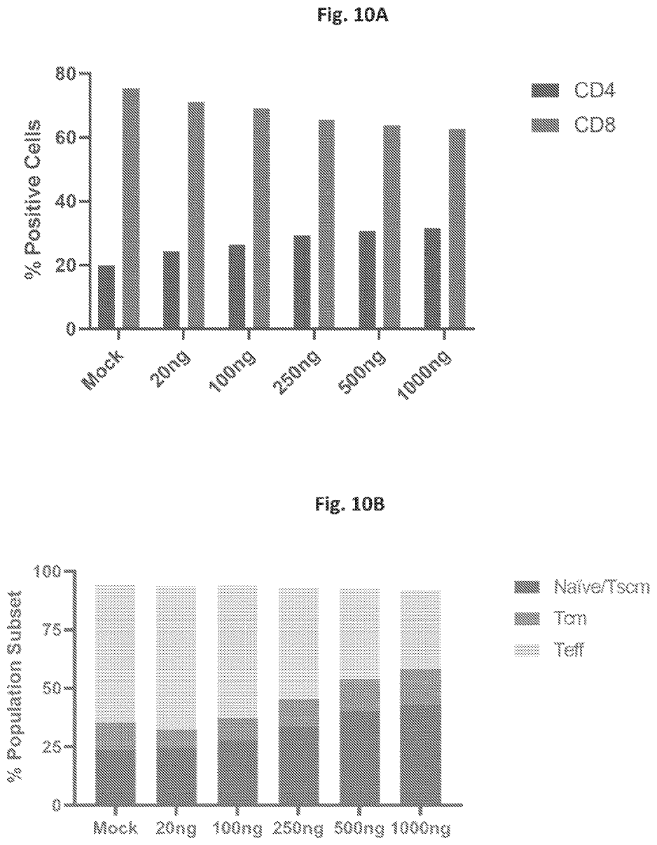

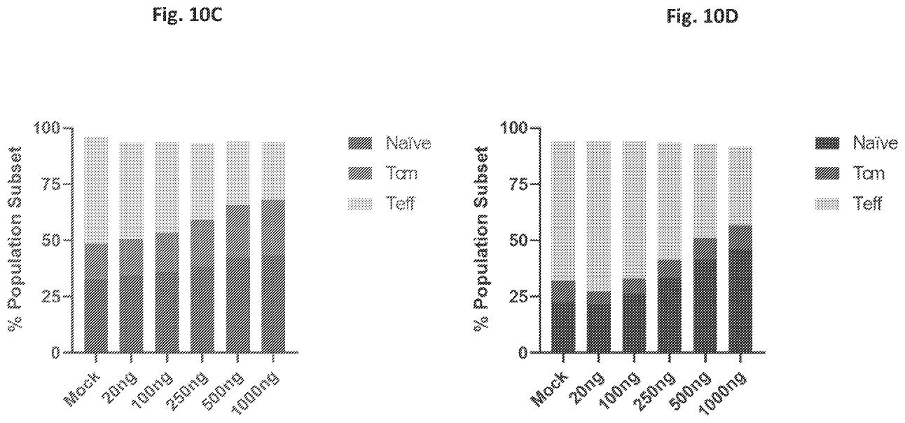

Anti-CD117 CAR T cells (TRAC⁻/β2M⁻/CTX-2840) generated as described above were stained with anti-human CD4 and anti-human CD8 antibodies to determine the ratios of CD4⁺/CD8⁺ T cells in the samples. Flow cytometry analysis showed an RNA dose responsive effect with a higher CD8⁺ cell population at higher mRNA concentrations. FIG. 10A.

Analysis of T cell differentiation status using CD27 and CD45RO staining revealed a higher proportion of stem memory T cells (Tscm) as compared to effector T (Teff). FIG. 10B. Among the differentiated T cells, CD4⁺ T cells were higher in the Tcm population at lower mRNA concentrations. FIG. 10C. Differentiated CD8⁺ T cells showed a progressively higher proportion of Tcm cells and lower proportions of Teff cells as the mRNA concentration increases. FIG. 10D.

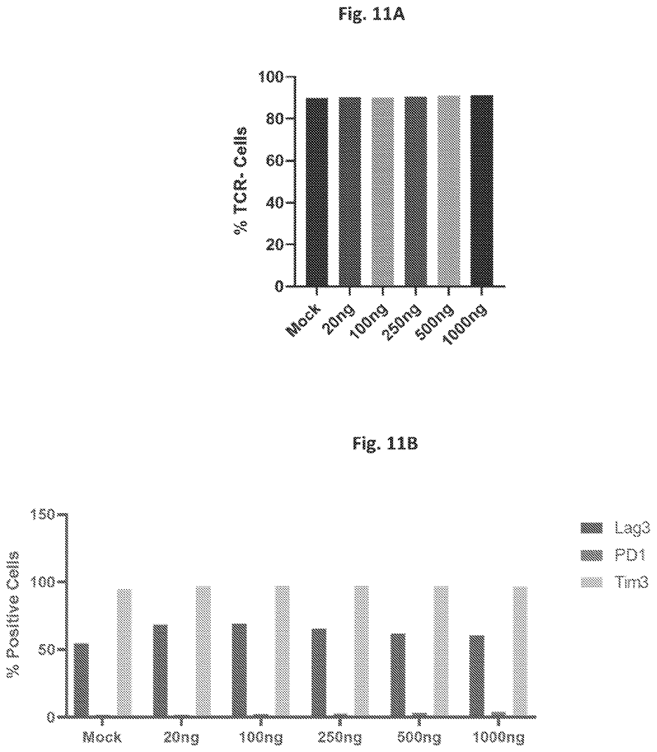

The Anti-CD117 CAR T cells were also tested for expression of TCR and exhaustion markers. The proportion of TCR⁻ cells or expression of exhaustion markers Lymphocyte Activating 3 (Lag3), Programmed Death-1 (PD-1), and T cell immunoglobulin and mucin domain-containing protein 3 (TIM3) did not significantly vary with mRNA concentrations, indicating that transient expression of the anti-CD117 CAR via mRNA transfection would not induce T cell exhaustion. FIGS. 11A-11B.

### Example 6: In Vivo Myeloablation Studies of Anti-CD117 CAR T Cells

This example investigates myeloablation efficacy of TRAC⁺/β2M⁺ anti-CD117 CAR T cells (autologous CAR T) or and TRAC/β2M⁻ anti-CD117 CAR T cells (allogenic CAR T) using a humanized mouse model.

(A) In vivo Myeloablation Efficacy

The autologous or allogenic anti-CD117 CAR T cells transiently expressing the CTX-2840 anti-CD117 CAR generated as described in Example 5 were injected (50×10⁶ cells (total)/mouse) into humanized NSG mice (huCD34+ cell engrafted mice). After 72 hours, the animals were sacrificed and blood cells in the bone marrow analyzed by flow cytometry for evaluating chimerism (hCD45 vs total CD45 cells) and presence of CD34⁺/CD117⁺ cells. As shown in FIG. 12A, slight reduction of chimerism was observed in mice receiving the autologous anti-CD117 CAR-T cells or the allogeneic anti-CD117 CAR-T cells. The autologous and allogenic anti-CD117 CAR T cells showed similar efficiency in reducing the CD34⁺/CD117⁺ cell population. FIG. 12B.

These data demonstrate that CAR-T cells transiently expressing the anti-CD117 CAR, having wild-type TRAC and B2M genes (autologous cells) or disrupted TRAC and B2M genes (allogeneic cells), are effective in myeloablation.

### B. Dose Optimization Studies

TRAC⁻/β2M⁻ CAR T cells transiently expressing the CTX-2840 anti-CD117 CAR, or Busulfan were injected into humanized NSG mice (huCD34+ cell engrafted mice). The study design is provided in Table 6. After 72 hours, the animals were sacrificed and blood cells in the bone marrow analyzed by flow cytometry for evaluating chimerism (hCD45 vs total CD45 cells); presence of hCD45 positive cells; and CD34⁺/CD117⁺ cells.

TABLE 6
Study Design for Myeloablation Investigation

| | | | | | Sacrifice | |
|---|---|---|---|---|---|---|
| | | | | | Time Post | |
| Group | Mice | Engraft | Treatment | Dose | Dosing | Readout |
| KO CAR T | 3 | Yes | Anti-CD117 | 10 × 10⁶, 3 × | 72 hours | (a) Bone marrow |
| | | | CAR T cells | (total of 30 × | | blood |
| | | | | 10⁶ cells) | | CD34+/CD117+ |
| KO CAR T | 3 | Yes | Anti-CD117 | 10 × 10⁶ cells | 72 hours | cells |
| | | | CAR T cells | | | (b) Weights |
| KO CAR T | 3 | Yes | Anti-CD117 | 20 × 10⁶ cells | 72 hours | (c) Clinical |
| | | | CAR T cells | | | Observations |
| KO T cells | 3 | Yes | Anti-CD117 | 30 × 10⁶ cells | 72 hours | |
| | | | CAR T cells | | | |
| Busulfan | 3 | Yes | Busulfan | 2 doses of | 72 Hours | |
| | | | | 25 mg/kg | | |
| Ctl | 3 | Yes | X | X | 72 Hours | |

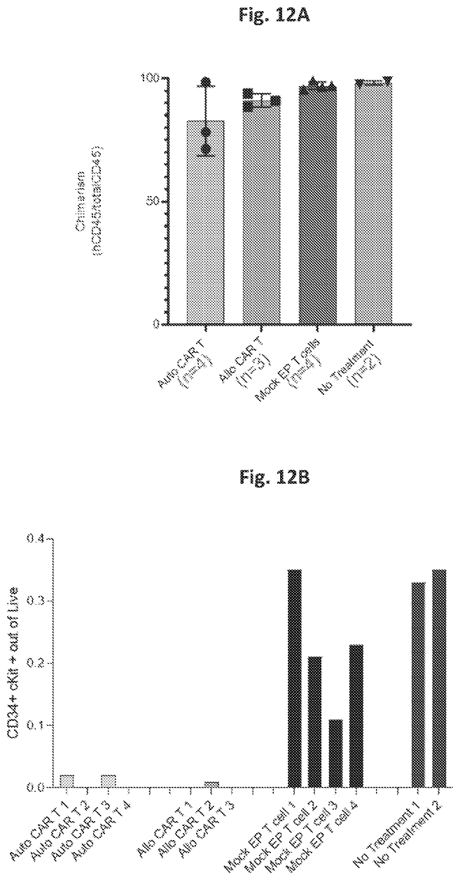

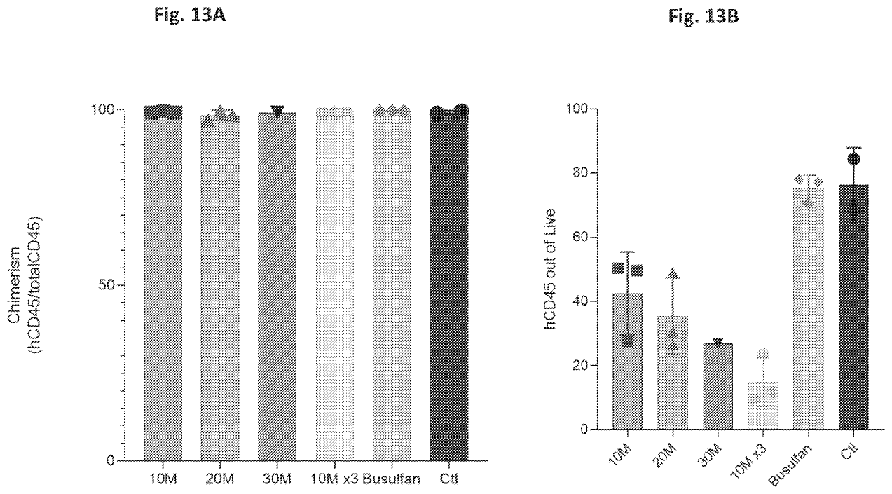

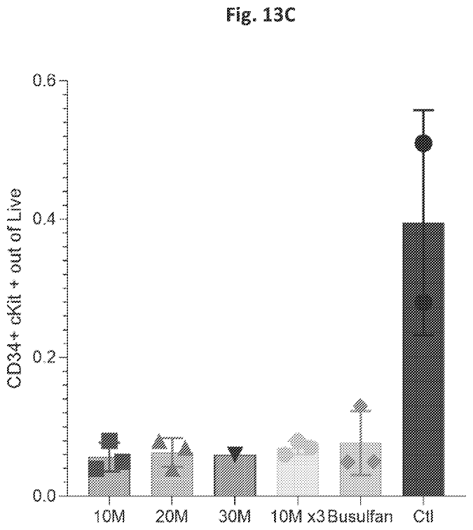

Treatment of the anti-CD117 CAR-T cells showed no significant impact on chimerism as shown in FIG. 13A but significantly reduced the level of hCD45+ cells in the treated mice in a dose dependent manner as compared with those treated with busulfan or with no treatment. FIG. 13B. A significant reduction in CD34⁺/CD117⁺ cells was also observed in the anti-CD17 CAR T treated animals. FIG. 13C.

Based on these observations, an anti-CD117 CAR T cell dose of 20×10⁶ cells (total) was considered optimal for myeloablation.

### C. Re-Engraftment Studies

The ability of transient anti-CD117 CAR expressing T cells to sufficiently deplete hematopoietic stem and progenitor cells (HSPCs) in the bone marrow to allow for edited (CCR5) HSPCs to engraft was examined. Table 7 summarizes the study design.

TABLE 7
Study Design

| | | | | | CCR5 | | | |
|---|---|---|---|---|---|---|---|---|
| | | | | | edited | | | |
| | | | | | CD34+ | | | |
| | | | | | cells per | Sacrifice | Editing | |
| Group | Mice | Engraft | Treatment | Dose | mouse | Time | Prediction | Readout |
| KO CAR T | 16 | Yes | Anti- | 20 × | 1 × 10⁶ | 4 weeks | High | (a) TIDE |
| | | | CD117 | 10⁶ | | (6) | | (b) BM |
| | | | CAR T | | | 6 weeks | | CD34⁺/CD1 |
| | | | cells | | | | | 17⁺ cells |
| −Ctrl | 8 | Yes | Mock EP | 20 × | 1 × 10⁶ | 4 weeks | Low | (c) Weights |
| | (2 | | CAR T- | 10⁶ | | (4) | | (d) Clinical |
| | without | | cells | | | 6 weeks | | Observations |
| | Mock T | | | | | | | |
| | Cells) | | | | | | | |
| +Ctrl | 10 | No | Irradiation | 100 | 1 × 10⁶ | 4 weeks | 100% | |
| (Nongrafted | | | | cgy | | (3) | | |
| mice) | | | | | | 6 weeks | | |
| Ctrl | 6 | Yes | | | | 4 weeks | 0% | |
| (Engrafted) | | | | | | (2) | | |
| | | | | | | 6 weeks | | |
| Depletion | 2 | Yes | CD117 | 20 × | | 72 hours | | |
| positive | | | CAR T | 10⁶ | | | | |
| control | | | cells | | | | | |
| Depletion | 2 | Yes | | | | 72 hours | | |
| negative | | | | | | | | |
| control | | | | | | | | |

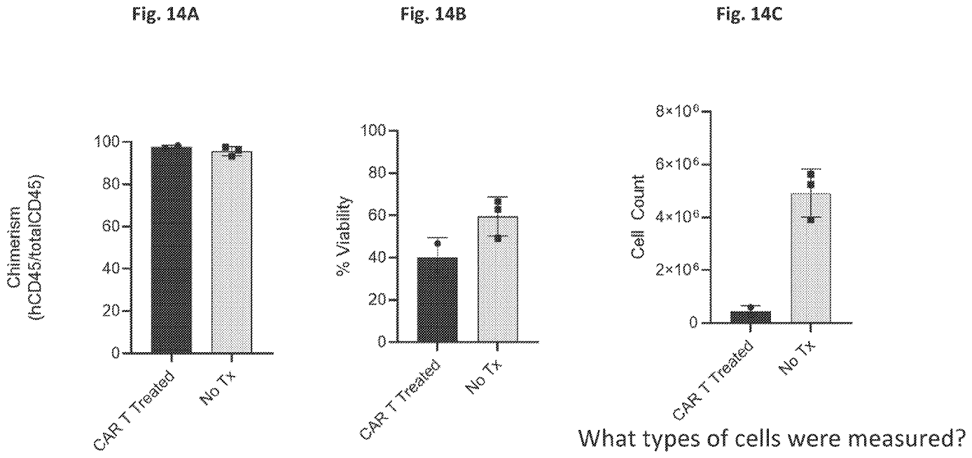

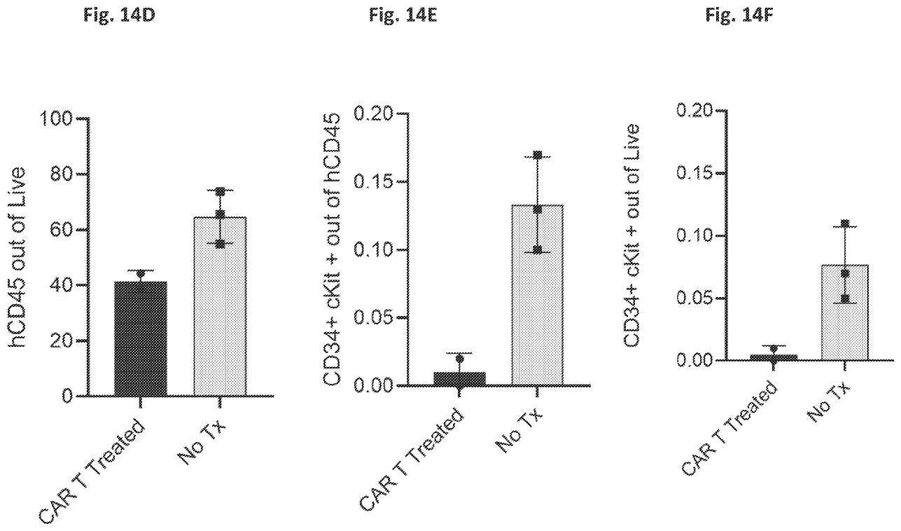

TRAC⁻/β2M⁻ CAR T cells transiently expressing the CTX-2840 anti-CD117 CAR were injected into humanized NSG mice (huCD34+ cell engrafted mice) and depletion of the CD34⁺/CD117⁺ cell population were verified on day 3 in a subset of treated mice. Treatment of the anti-CD117 CAR-T cells showed no impact on chimerism and a mild level of impact on cell viability. FIGS. 14A and 14B. On the other hand, the anti-CD117 CAR-T treated mice showed reduced cell count in the bone marrow and successful depletion of the CD34⁺/CD117⁺ cell population and reduced the level of hCD45⁺ cells in the treated mice as shown in FIGS. 14C-4E.

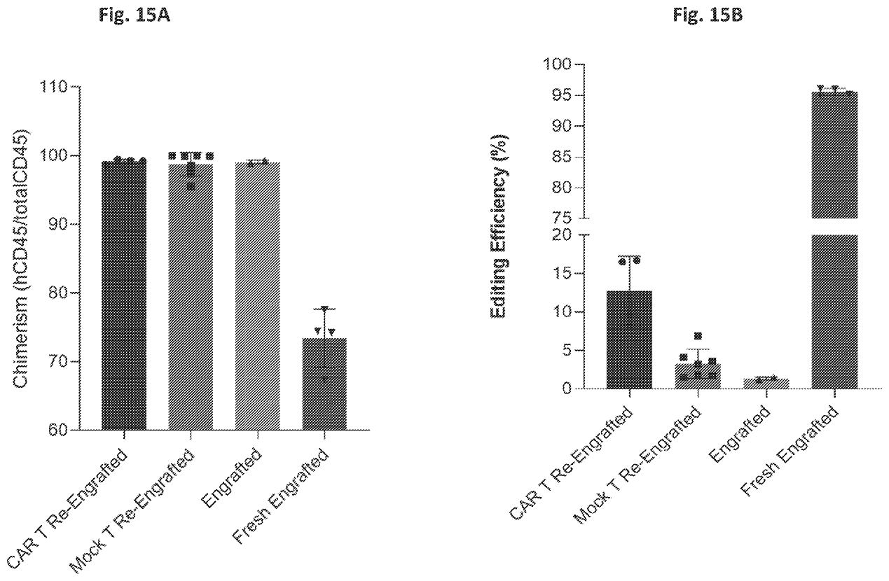

On day 5, the animals were injected with CCR5 edited CD34⁺ cells. At 3 weeks after the initial anti-CD117 CAR T cell injection, animals were sacrificed and assessed for CCR5 editing efficiency and chimerism and lineage distribution. Assessment of the bone marrow in live animals at weeks 4 and 6 revealed that chimerism was very high in the engrafted/re-engrafted mice, suggesting that new CD34⁺ cells were able to engraft. FIG. 15A.

Lineage analysis revealed that lineage distribution was similar across re-engrafted groups, with the appearance of CD3⁺ cells in the anti-CD117 CAR T cell treatment groups. A 4-fold higher CCR5 editing efficiency was observed in the bone marrow of the anti-CD117 CAR T cell treated group, suggesting that transient anti-CD117 expression ablated the original bone marrow while allowing donor HSPCs to engraft. FIG. 15B.

In conclusion, these data demonstrate that CAR T cells transiently expressing an anti-CD117 CAR can successfully ablate human bone marrow cells to allow for engraftment and expansion of new donor CD34⁺ cells.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A chimeric antigen receptor (CAR) that binds CD117 (anti-CD117 CAR), wherein the anti-CD117 CAR comprises:
(a) an ectodomain that binds CD117;
(b) a transmembrane domain; and
(c) an endodomain comprising a costimulatory domain and a CD3ζ signaling domain;
wherein the ectodomain comprises comprising a single chain variable fragment (scFv) that binds CD117 (anti-CD117 scFv), which comprises a heavy chain variable region (V_{H}) and a light chain variable region (V_{L}), the V_{H} comprising the same heavy chain complementarity determining regions (CDRs) and the same light chain CDRs as those in Reference anti-CD117 antibody 1 or Reference anti-CD117 antibody 2; or
wherein the ectodomain comprises a single domain antibody fragment comprising:
(i) a CDR1 set forth as GX₁X₂TX₃X₄X₅X₆X₇, in which X₁ is T, R, G, H, or D; X₂ is T or absent; X₃ is F, L, S, or V; X₄ is G, S, or T; X₅ is I, N, S, T or Y; X₆ is D, V, or Y; and X₇ is A, F, P, S, V or W;
(ii) CDR2 set forth as X₈X₉X₁₀X₁₁X₁₂X₁₃X₁₄X₁₅, in which X₈ is I or V; X₉ is A, G, H, L, R, S, T, or V, X₁₀ is R, S, or W; X₁₁ is G, N, S, or Y; X₁₂ is A, G, L, or absent; X₁₃ is A, D, G, L, or S; X₁₄ is G, M, S, T, or V; and X₁₅ is A, L, or T; and
(iii) CDR3 set forth as:
(a)
(SEQ ID NO: 70)
GRFHPIRVDTA;
(b)
(SEQ ID NO: 71)
ASGSNWRLGAIDEY;
(c)
(SEQ ID NO: 72)
GQHLSGLGGSAWSIEG;
(d)
(SEQ ID NO: 73)
RQYVGSGSYYLKKEGGY;
(e)
(SEQ ID NO: 74)
DSTGVYGTGYVSSRKGRY;
(f)
(SEQ ID NO: 75)
AFTPEFRDGGIWDDASV;
(g)
(SEQ ID NO: 78)
VRRRWLIWQEEEY;
(h)
(SEQ ID NO: 80)
DQRGVPAYYSDYALY;
(i)
(SEQ ID NO: 81)
DESFPAYYSDYALY;
(j)
(SEQ ID NO: 69)
VLRTGM;
(k)
(SEQ ID NO: 76)
SDSYFYASPHLY;
(l)
(SEQ ID NO: 77)
SDTYFYASPHLY;
or
(m)
(SEQ ID NO: 79)
RRGTILVVQEYEY.

2. The anti-CD117 CAR of claim 1, wherein the ectodomain comprises the anti-CD117 scFv.

3. The anti-CD117 CAR of claim 2, wherein the scFv comprises:
(a) the V_{H} comprising SEQ ID NO: 108, and the V_{L} comprising SEQ ID NO: 107; or
(b) the V_{H} comprising SEQ ID NO: 130, and the V_{L} comprising SEQ ID NO: 129.

4. The anti-CD117 CAR of claim 2, wherein the scFv comprises the amino acid sequence of any one of SEQ ID NOs: 109, 110, 131, and 132, optionally wherein the scFv comprises SEQ ID NO: 109.

5. The anti-CD117 CAR of claim 2, wherein the transmembrane domain is a CD8 transmembrane domain.

6. The anti-CD117 CAR of claim 2, wherein the co-stimulatory domain is a CD28 co-stimulatory domain or a 4-1BB co-stimulatory domain.

7. The anti-CD117 CAR of claim 2, which comprises the amino acid sequence of any one of the anti-CD117 CARs provided in Table 2 that contains an anti-CD117 scFv fragment, optionally wherein the anti-CD117 CAR comprises the amino acid sequence of SEQ ID NO: 112 or SEQ ID NO: 115.

8. The anti-CD117 CAR of claim 1, wherein the ectodomain comprises the single domain antibody fragment.

9. The anti-CD117 CAR of claim 8, wherein the CDR3 of the single domain antibody fragment is set forth as any one of (a)-(i).

10. The anti-CD117 CAR of claim 8, wherein the CDR1 of the single domain antibody fragment is set forth as:
(a)
(SEQ ID NO: 16)
GRTTFSTYW;
(b)
(SEQ ID NO: 17)
GGTFSIYP;
(c)
(SEQ ID NO: 18)
GRTLSNYF;
(d)
(SEQ ID NO: 19)
GRTFSSYA;
(e)
(SEQ ID NO: 20)
GHTFSNYA;
(f)
(SEQ ID NO: 19)
GRTFSSYA;
(g)
(SEQ ID NO: 24)
GDTFSSYS;
(h)
(SEQ ID NO: 26)
GRTSGSYV;
or
(i)
(SEQ ID NO: 27)
GRTFTYDA.

11. The anti-CD117 CAR of claim 8, wherein the CDR2 of the single domain antibody fragment is set forth as:
(a)
(SEQ ID NO: 43)
ISWSAGMA;
(b)
(SEQ ID NO: 44)
IGWSASGT;
(c)
(SEQ ID NO: 45)
IHWSLGST;
(d)
(SEQ ID NO: 46)
ITSSGLVA;
(e)
(SEQ ID NO: 47)
ISWSGGST;
(f)
(SEQ ID NO: 48)
ILSNGLTT;
(g)
(SEQ ID NO: 51)
IRWSGGTT;
(h)
(SEQ ID NO: 53)
ISWSAGMT;
or
(i)
(SEQ ID NO: 47)
ISWSGGST.

12. The anti-CD117 CAR of claim 8, wherein the single domain antibody fragment comprises:
(a) a CDR1 of GRTTFSTYW (SEQ ID NO:16), a CDR2 of ISWSAGMA (SEQ ID NO:43), and a CDR3 of GRFHPIRVDTA (SEQ ID NO:70);
(b) a CDR1 of GGTFSIYP (SEQ ID NO:17); a CDR2 of IGWSASGT (SEQ ID NO:44); and a CDR3 of ASGSNWRLGAIDEY (SEQ ID NO:71);
(c) a CDR1 of GRTLSNYF (SEQ ID NO:18), a CDR2 of IHWSLGST (SEQ ID NO:45), and a CDR3 of GQHLSGLGGSAWSIEG (SEQ ID NO:72);
(d) a CDR1 of GRTFSSYA (SEQ ID NO:19), a CDR2 of ITSSGLVA (SEQ ID NO:46), and a CDR3 of RQYVGSGSYYLKKEGGY (SEQ ID NO:73);
(e) a CDR1 of GHTFSNYA (SEQ ID NO:20), a CDR2 of ISWSGGST (SEQ ID NO:47), and a CDR3 of DSTGVYGTGYVSSRKGRY (SEQ ID NO:74);
(f) a CDR1 of GRTFSSYA (SEQ ID NO:19), a CDR2 of ILSNGLTT (SEQ ID NO:48), and a CDR3 of AFTPEFRDGGIWDDASV (SEQ ID NO:75);
(g) a CDR1 of GDTFSSYS (SEQ ID NO:24), a CDR2 of IRWSGGTT (SEQ ID NO:51), and a CDR3 of VRRRWLIWQEEEY (SEQ ID NO:78);
(h) a CDR1 of GRTSGSYV (SEQ ID NO:26), a CDR2 of ISWSAGMT (SEQ ID NO:53), and a CDR3 of DQRGVPAYYSDYALY (SEQ ID NO:80); or
(i) a CDR1 of GRTFTYDA (SEQ ID NO:27), a CDR2 of ISWSGGST (SEQ ID NO:47), and a CDR3 of DESFPAYYSDYALY (SEQ ID NO:81).

13. The anti-CD117 CAR of claim 8, wherein the single domain antibody fragment is a heavy chain variable domain antibody (VHH).

14. (canceled)

15. (canceled)

16. (canceled)

17. (canceled)

18. A nucleic acid comprising a nucleotide sequence encoding an anti-CD117 CAR set forth in claim 1.

19. (canceled)

20. (canceled)

21. A population of genetically engineered immune cells, wherein the genetically engineered immune cells comprise a nucleic acid encoding a chimeric antigen receptor (CAR) that binds CD117 (anti-CD117 CAR) and express the anti-CD117 CAR; wherein the anti-CD117 CAR is set forth in claim 1.

22. (canceled)

23. (canceled)

24. The population of genetically engineered immune cells of claim 21, wherein the genetically engineered immune cells comprises a wild-type T cell receptor alpha chain constant region (TRAC) gene, a wild-type beta-2-microglobulin (β2M) gene, or a combination thereof.

25. The population of genetically engineered immune cells of claim 21, wherein the genetically engineered immune cells further comprise a disrupted T cell receptor alpha chain constant region (TRAC) gene, a disrupted beta-2-microglobulin (β2M) gene, or a combination thereof.

26. (canceled)

27. The population of genetically engineered immune cells of claim 21, wherein the nucleic acid encoding the anti-CD117 CAR is inserted into the disrupted TRAC gene.

28. (canceled)

29. A method for inhibiting CD117+ cells in a subject, the method comprising administering to a subject in need thereof an effective amount of a population of genetically engineered immune cells set forth in claim 21.

30.-36. (canceled)

37. A method for preparing a population of genetically engineered immune cells, the method comprising:
(a) delivering to a plurality of immune cells a nucleic acid encoding an anti-CD117 CAR set forth in claim 1; and
(b) producing a population of genetically engineered immune cells expressing the anti-CD117 CAR.

38.-52. (canceled)


SEQUENCE LISTING (229 sequences)

SEQ ID NO: 1 - protein, 25 aa, Synthetic construct
  SITE 1: note = E or Q
  SITE 13: note = Q or R
  SITE 16: note = G or D
  SITE 23: note = A, T, or V
  SITE 24: note = A, G, or V
ZVQLVESGGGLVZAGZSLRLSCZZS

SEQ ID NO: 2 - protein, 25 aa, Synthetic construct
EVQLVESGGGLVQAGGSLRLSCVAS

SEQ ID NO: 3 - protein, 25 aa, Synthetic construct
EVQLVESGGGLVRAGGSLRLSCAAS

SEQ ID NO: 4 - protein, 25 aa, Synthetic construct
QVQLVESGGGLVQAGGSLRLSCAAS

SEQ ID NO: 5 - protein, 25 aa, Synthetic construct
QVQLVESGGGLVQAGDSLRLSCAVS

SEQ ID NO: 6 - protein, 25 aa, Synthetic construct
QVQLVESGGGLVQAGDSLRLSCAAS

SEQ ID NO: 7 - protein, 25 aa, Synthetic construct
QVHLVESGGGLVQAGGSLGLSCAAS

SEQ ID NO: 8 - protein, 25 aa, Synthetic construct
QVQLVESGGGLVQAGGSLRLSCVAS

SEQ ID NO: 9 - protein, 25 aa, Synthetic construct
QVQLVESGGGLVQAGGSLRLSCAGS

SEQ ID NO: 10 - protein, 25 aa, Synthetic construct
QVQLVESGGGLVQAGGSLRLSCVGS

SEQ ID NO: 11 - protein, 25 aa, Synthetic construct
QVQLVESGGGLVQAGGSLRLSCTAS

SEQ ID NO: 12 - DNA, 20 bp, Synthetic construct
  source 1..20: mol_type = other DNA
agagcaacagtgctgtggcc

SEQ ID NO: 13 - protein, 25 aa, Synthetic construct
EVQLVESGGGLVQAGGSLRLSCAAS

SEQ ID NO: 14 - DNA, 20 bp, Synthetic construct
  source 1..20: mol_type = other DNA
agagcaacagtgctgtggcc

SEQ ID NO: 15 - protein, 8 aa, Synthetic construct
GTTSGIVA

SEQ ID NO: 16-19 - skipped (4 entries)

SEQ ID NO: 20 - protein, 8 aa, Synthetic construct
GHTFSNYA

SEQ ID NO: 21 - RNA, 100 nt, Synthetic construct
  source 1..100: mol_type = other RNA
agagcaacagtgctgtggccgttttagagctagaaatagcaagttaaaataaggctagtccgttatcaacttgaaaaagtggcaccgagtcggtgctttt

SEQ ID NO: 22 - protein, 8 aa, Synthetic construct
GRTVSTYA

SEQ ID NO: 23 - RNA, 20 nt, Synthetic construct
  source 1..20: mol_type = other RNA
agagcaacagtgctgtggcc

SEQ ID NO: 24 - protein, 8 aa, Synthetic construct
GDTFSSYS

SEQ ID NO: 25 - protein, 8 aa, Synthetic construct
GRTFSSYV

SEQ ID NO: 26 - protein, 8 aa, Synthetic construct
GRTSGSYV

SEQ ID NO: 27 - protein, 8 aa, Synthetic construct
GRTFTYDA

SEQ ID NO: 28 - protein, 17 aa, Synthetic construct
  SITE 1: note = L, M, R, or V
  SITE 2: note = A, G, or H
  SITE 4: note = F, L, or Y
  SITE 7: note = A or G
  SITE 11: note = E, N, Q, or R
  SITE 14: note = F or L
  SITE 16: note = A, G, or S
  SITE 17: note = A, G or S
ZZWZRQZPGKZREZVZZ

SEQ ID NO: 29 - protein, 17 aa, Synthetic construct
MHWYRQAPGKRRELVAS

SEQ ID NO: 30 - protein, 17 aa, Synthetic construct
LGWFRQAPGKNREFVAA

SEQ ID NO: 31 - protein, 17 aa, Synthetic construct
VGWFRQAPGKQREFVAA

SEQ ID NO: 32 - protein, 17 aa, Synthetic construct
MAWLRQAPGKEREFVAA

SEQ ID NO: 33 - protein, 17 aa, Synthetic construct
MGWFRQAPGKEREFVAS

SEQ ID NO: 34 - protein, 17 aa, Synthetic construct
MGWFRQAPGKEREFVAA

SEQ ID NO: 35 - protein, 17 aa, Synthetic construct
MGWFRQGPGKEREFAAA

SEQ ID NO: 36 - protein, 17 aa, Synthetic construct
MGWFRQAPGKEREFVSA

SEQ ID NO: 37 - protein, 17 aa, Synthetic construct
MGWFRQAPGKERELVAA

SEQ ID NO: 38 - protein, 17 aa, Synthetic construct
MGWFRQGPGKEREFVGG

SEQ ID NO: 39 - RNA, 20 nt, Synthetic construct
  source 1..20: mol_type = other RNA
agagcaacagtgctgtggcc

SEQ ID NO: 40 - RNA, 100 nt, Synthetic construct; identical to SEQ ID NO: 21
  source 1..100: mol_type = other RNA

SEQ ID NO: 41 - protein, 17 aa, Synthetic construct
RAWFRQAPGKEREFVAA

SEQ ID NO: 42 - protein, 7 aa, Synthetic construct
VVRGASL

SEQ ID NO: 43 - protein, 8 aa, Synthetic construct
ISWSAGMA

SEQ ID NO: 44 - protein, 8 aa, Synthetic construct
IGWSASGT

SEQ ID NO: 45 - protein, 8 aa, Synthetic construct
IHWSLGST

SEQ ID NO: 46 - protein, 8 aa, Synthetic construct
ITSSGLVA

SEQ ID NO: 47 - protein, 8 aa, Synthetic construct
ISWSGGST

SEQ ID NO: 48 - protein, 8 aa, Synthetic construct
ILSNGLTT

SEQ ID NO: 49 - protein, 8 aa, Synthetic construct
IARSGGGT

SEQ ID NO: 50 - protein, 8 aa, Synthetic construct
IGRSGGST

SEQ ID NO: 51 - protein, 8 aa, Synthetic construct
IRWSGGTT

SEQ ID NO: 52 - protein, 8 aa, Synthetic construct
ISWYGDST

SEQ ID NO: 53 - protein, 8 aa, Synthetic construct
ISWSAGMT

SEQ ID NO: 54 - DNA, 19 bp, Synthetic construct
  source 1..19: mol_type = other DNA
aagagcaacaaatctgact

SEQ ID NO: 55 - protein, 40 aa, Synthetic construct
  SITE 1: note = L, N, or Y
  SITE 3: note = A, G, L, P, or Q
  SITE 6: note = M, V, or absent
  SITE 7: note = E or K
  SITE 16: note = G, K, or N
  SITE 17: note = A, G, T, or V
  SITE 18: note = E, K, or R
  SITE 19: note = D, N, or S
  SITE 24: note = H, Q or R
  SITE 26: note = D, N, or S
  SITE 35: note = N, T, or V
ZYZDSZZGRFTISRDZZZZTVYLZMZSLKPEDTAZYYCAA

SEQ ID NO: 56 - protein, 40 aa, Synthetic construct
NYLDSVKGRFIISEDGARNTVYLQMNSLKPEDTAVYYCNA

SEQ ID NO: 57 - protein, 39 aa, Synthetic construct
YYQDSKGRFTISRDNTKNTAYLQMNSLQPEDTAVYYCAA

SEQ ID NO: 58 - protein, 40 aa, Synthetic construct
YYGDSVEGRFTVSRDNARSTVYLRMSSLKPDDTAVYYCAA

SEQ ID NO: 59 - protein, 40 aa, Synthetic construct
YYQDPVKGRFTISRDKAKNTVYLQMNTLKPEDTATYICAA

SEQ ID NO: 60 - protein, 40 aa, Synthetic construct
YYGDSVEARFTISRDNAKNTVYLQMDSLKPEDTAVYYCAA

SEQ ID NO: 61 - protein, 40 aa, Synthetic construct
LYADSVKGRFTISRDNGENTVYLQMNSLKPEDTAVYYCAL

SEQ ID NO: 62 - protein, 40 aa, Synthetic construct
YYADSVKGRFTISRDNAKDTVYLQMNSLKPEDTANYYCAA

SEQ ID NO: 63 - protein, 40 aa, Synthetic construct
YYADSVKGRFTISRDNAKNTVYLQMNSLKPEDTAVYYCAA

SEQ ID NO: 64 - protein, 40 aa, Synthetic construct
YYADSVKGRFTISRDNVRNTVYLQMNSLKPEDAAVYYCAA

SEQ ID NO: 65 - protein, 40 aa, Synthetic construct
YYADSVKGRFTISRDNAKNTVYLQMNSLKREDTAVYYCAA

SEQ ID NO: 66 - protein, 40 aa, Synthetic construct
YYADSVKGRFTISRDNAKNTVYLHMNSLKPEDTAVYYCAA

SEQ ID NO: 67 - DNA, 58 bp, Synthetic construct
  source 1..58: mol_type = other DNA
aagagcaacagtgctgtgcctggagcaacaaatctgactaagagcaacaaatctgact

SEQ ID NO: 68 - protein, 40 aa, Synthetic construct
YYPNSMKGRFTISRDNAKNTVYLQMNSLKPEDTAVYYCAA

SEQ ID NO: 69 - protein, 6 aa, Synthetic construct
VLRTGM

SEQ ID NO: 70 - protein, 11 aa, Synthetic construct
GRFHPIRVDTA

SEQ ID NO: 71 - protein, 14 aa, Synthetic construct
ASGSNWRLGAIDEY

SEQ ID NO: 72 - protein, 16 aa, Synthetic construct
GQHLSGLGGSAWSIEG

SEQ ID NO: 73 - protein, 17 aa, Synthetic construct
RQYVGSGSYYLKKEGGY

SEQ ID NO: 74 - protein, 18 aa, Synthetic construct
DSTGVYGTGYVSSRKGRY

SEQ ID NO: 75 - protein, 17 aa, Synthetic construct
AFTPEFRDGGIWDDASV

SEQ ID NO: 76 - protein, 12 aa, Synthetic construct
SDSYFYASPHLY

SEQ ID NO: 77 - protein, 12 aa, Synthetic construct
SDTYFYASPHLY

SEQ ID NO: 78 - protein, 13 aa, Synthetic construct
VRRRWLIWQEEEY

SEQ ID NO: 79 - protein, 13 aa, Synthetic construct
RRGTILVVQEYEY

SEQ ID NO: 80 - protein, 15 aa, Synthetic construct
DQRGVPAYYSDYALY

SEQ ID NO: 81 - protein, 14 aa, Synthetic construct
DESFPAYYSDYALY

SEQ ID NO: 82 - protein, 13 aa, Synthetic construct
  SITE 1: note = D, E, L, R, or T
  SITE 2: note = D, S, or Y
  SITE 4: note = G or A
  SITE 8: note = L or Q
ZZWZQGTZVTVSS

SEQ ID NO: 83 - protein, 13 aa, Synthetic construct
DYWGQGTLVTVSS

SEQ ID NO: 84 - protein, 13 aa, Synthetic construct
LYWAQGTQVTVSS

SEQ ID NO: 85 - protein, 13 aa, Synthetic construct
DSWGQGTQVTVSS

SEQ ID NO: 86 - protein, 13 aa, Synthetic construct
DYWGQGTQVTVSS

SEQ ID NO: 87 - protein, 13 aa, Synthetic construct
EYWGPGTQVTVSS

SEQ ID NO: 88 - protein, 13 aa, Synthetic construct
EYWGQGTLVTVSS

SEQ ID NO: 89 - DNA, 52 bp, Synthetic construct
  source 1..52: mol_type = other DNA
aagagcaacagtgctggagcaacaaatctgactaagagcaacaaatctgact

SEQ ID NO: 90 - DNA, 53 bp, Synthetic construct
  source 1..53: mol_type = other DNA
aagagcaacagtgcctggagcaacaaatctgactaagagcaacaaatctgact

SEQ ID NO: 91 - protein, 13 aa, Synthetic construct
TDWGQGTQVTVSS

SEQ ID NO: 92 - DNA, 38 bp, Synthetic construct
  source 1..38: mol_type = other DNA
aagagcaacagtgctgactaagagcaacaaatctgact

SEQ ID NO: 93 - DNA, 60 bp, Synthetic construct
  source 1..60: mol_type = other DNA
aagagcaacagtgctgtgggcctggagcaacaaatctgactaagagcaacaaatctgact

SEQ ID NO: 94 - DNA, 57 bp, Synthetic construct
  source 1..57: mol_type = other DNA
aagagcaacagtgctggcctggagcaacaaatctgactaagagcaacaaatctgact

SEQ ID NO: 95 - protein, 13 aa, Synthetic construct
RYWGQGTLVTVSS

SEQ ID NO: 96 - protein, 21 aa, Synthetic construct
MALPVTALLLPLALLLHAARP

SEQ ID NO: 97 - protein, 84 aa, Synthetic construct
FVPVFLPAKPTTTPAPRPPTPAPTIASQPLSLRPEACRPAAGGAVHTRGLDFACDIYIWAPLAGTCGVLLLSLVITLYCNHRNR

SEQ ID NO: 98 - protein, 40 aa, Synthetic construct
SKRSRLLHSDYMNMTPRRPGPTRKHYQPYAPPRDFAAYRS

SEQ ID NO: 99 - protein, 42 aa, Synthetic construct
KRGRKKLLYIFKQPFMRPVQTTQEEDGCSCRFPEEEEGGCEL

SEQ ID NO: 100 - protein, 112 aa, Synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 101 - protein, 15 aa, Synthetic construct
RASESVDIYGNSFMH

SEQ ID NO: 102 - protein, 7 aa, Synthetic construct
LASNLES

SEQ ID NO: 103 - protein, 9 aa, Synthetic construct
QQNNEDPYT

SEQ ID NO: 104 - protein, 5 aa, Synthetic construct
SYNMH

SEQ ID NO: 105 - protein, 17 aa, Synthetic construct
VIYSGNGDTSYNQKFKG

SEQ ID NO: 106 - protein, 8 aa, Synthetic construct
ERDTRFGN

SEQ ID NO: 107 - protein, 111 aa, Synthetic construct
DIVMTQSPDSLAVSLGERATINCRASESVDIYGNSFMHWYQQKPGQPPKLLIYLASNLESGVPDRFSGSGSGTDFTLTISSLQAEDVAVYYCQQNNEDPYTFGGGTKVEIK

SEQ ID NO: 108 - protein, 117 aa, Synthetic construct
QVQLVQSGAEVKKPGASVKVSCKASGYTFTSYNMHWVRQAPGQGLEWMGVIYSGNGDTSYNQKFKGRVTITADKSTSTAYMELSSLRSEDTAVYYCARERDTRFGNWGQGTLVTVSS

SEQ ID NO: 109 - protein, 243 aa, Synthetic construct
DIVMTQSPDSLAVSLGERATINCRASESVDIYGNSFMHWYQQKPGQPPKLLIYLASNLESGVPDRFSGSGSGTDFTLTISSLQAEDVAVYYCQQNNEDPYTFGGGTKVEIKGGGGSGGGGSGGGGSQVQLVQSGAEVKKPGASVKVSCKASGYTFTSYNMHWVRQAPGQGLEWMGVIYSGNGDTSYNQKFKGRVTITADKSTSTAYMELSSLRSEDTAVYYCARERDTRFGNWGQGTLVTVSS

SEQ ID NO: 110 - protein, 243 aa, Synthetic construct
QVQLVQSGAEVKKPGASVKVSCKASGYTFTSYNMHWVRQAPGQGLEWMGVIYSGNGDTSYNQKFKGRVTITADKSTSTAYMELSSLRSEDTAVYYCARERDTRFGNWGQGTLVTVSSGGGGSGGGGSGGGGSDIVMTQSPDSLAVSLGERATINCRASESVDIYGNSFMHWYQQKPGQPPKLLIYLASNLESGVPDRFSGSGSGTDFTLTISSLQAEDVAVYYCQQNNEDPYTFGGGTKVEIK

SEQ ID NO: 111 - protein, 504 aa, Synthetic construct
MALPVTALLLPLALLLHAARPDIVMTQSPDSLAVSLGERATINCRASESVDIYGNSFMHWYQQKPGQPPKLLIYLASNLESGVPDRFSGSGSGTDFTLTISSLQAEDVAVYYCQQNNEDPYTFGGGTKVEIKGGGGSGGGGSGGGGSQVQLVQSGAEVKKPGASVKVSCKASGYTFTSYNMHWVRQAPGQGLEWMGVIYSGNGDTSYNQKFKGRVTITADKSTSTAYMELSSLRSEDTAVYYCARERDTRFGNWGQGTLVTVSSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 112 - protein, 483 aa, Synthetic construct
DIVMTQSPDSLAVSLGERATINCRASESVDIYGNSFMHWYQQKPGQPPKLLIYLASNLESGVPDRFSGSGSGTDFTLTISSLQAEDVAVYYCQQNNEDPYTFGGGTKVEIKGGGGSGGGGSGGGGSQVQLVQSGAEVKKPGASVKVSCKASGYTFTSYNMHWVRQAPGQGLEWMGVIYSGNGDTSYNQKFKGRVTITADKSTSTAYMELSSLRSEDTAVYYCARERDTRFGNWGQGTLVTVSSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 113 - DNA, 1512 bp, Synthetic construct
  source 1..1512: mol_type = other DNA
atggcgcttccggtgacagcactgctcctccccttggcgctgttgctccacgcagcaaggccggacatagttatgacccaatcaccagactcactcgcggtcagcttgggggagcgcgccacaattaactgtcgcgcgtctgagtctgtcgacatctacggtaattcttttatgcattggtatcaacaaaagccaggacagccgccgaaattgttgatatatctcgctagcaaccttgagtcaggtgtgcctgatcggttcagtggttctgggtcagggaccgactttacgctgacaattagttctctgcaagcagaagatgtggcagtgtattattgccagcaaaataatgaggacccctacaccttcgggggtggcacgaaagtcgaaattaaagggggaggcggcagtggcggtggcggtagcggaggtggtgggtctcaggtccaactggttcaatccggagcggaagttaagaagccaggtgcaagtgtcaaagtctcatgcaaagcgtccgggtacaccttcacgtcctataacatgcattgggttcggcaggcgcctggccaggggctcgagtggatgggcgtcatctactccggaaatggcgataccagttacaatcagaagtttaagggccgagtaaccattaccgctgacaaaagtacttcaaccgcttacatggaactgtcatcacttaggtcagaagacacggcagtttattactgcgcgcgcgagagggacacaaggtttggcaattgggggcaaggcacactcgttactgtaagtagtagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 114 - protein, 506 aa, Synthetic construct
MALPVTALLLPLALLLHAARPDIVMTQSPDSLAVSLGERATINCRASESVDIYGNSFMHWYQQKPGQPPKLLIYLASNLESGVPDRFSGSGSGTDFTLTISSLQAEDVAVYYCQQNNEDPYTFGGGTKVEIKGGGGSGGGGSGGGGSQVQLVQSGAEVKKPGASVKVSCKASGYTFTSYNMHWVRQAPGQGLEWMGVIYSGNGDTSYNQKFKGRVTITADKSTSTAYMELSSLRSEDTAVYYCARERDTRFGNWGQGTLVTVSSSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 115 - protein, 485 aa, Synthetic construct
DIVMTQSPDSLAVSLGERATINCRASESVDIYGNSFMHWYQQKPGQPPKLLIYLASNLESGVPDRFSGSGSGTDFTLTISSLQAEDVAVYYCQQNNEDPYTFGGGTKVEIKGGGGSGGGGSGGGGSQVQLVQSGAEVKKPGASVKVSCKASGYTFTSYNMHWVRQAPGQGLEWMGVIYSGNGDTSYNQKFKGRVTITADKSTSTAYMELSSLRSEDTAVYYCARERDTRFGNWGQGTLVTVSSSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 116 - DNA, 1518 bp, Synthetic construct
  source 1..1518: mol_type = other DNA
atggcgcttccggtgacagcactgctcctccccttggcgctgttgctccacgcagcaaggccggacatagttatgacccaatcaccagactcactcgcggtcagcttgggggagcgcgccacaattaactgtcgcgcgtctgagtctgtcgacatctacggtaattcttttatgcattggtatcaacaaaagccaggacagccgccgaaattgttgatatatctcgctagcaaccttgagtcaggtgtgcctgatcggttcagtggttctgggtcagggaccgactttacgctgacaattagttctctgcaagcagaagatgtggcagtgtattattgccagcaaaataatgaggacccctacaccttcgggggtggcacgaaagtcgaaattaaagggggaggcggcagtggcggtggcggtagcggaggtggtgggtctcaggtccaactggttcaatccggagcggaagttaagaagccaggtgcaagtgtcaaagtctcatgcaaagcgtccgggtacaccttcacgtcctataacatgcattgggttcggcaggcgcctggccaggggctcgagtggatgggcgtcatctactccggaaatggcgataccagttacaatcagaagtttaagggccgagtaaccattaccgctgacaaaagtacttcaaccgcttacatggaactgtcatcacttaggtcagaagacacggcagtttattactgcgcgcgcgagagggacacaaggtttggcaattgggggcaaggcacactcgttactgtaagtagtagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 117 - protein, 504 aa, Synthetic construct
MALPVTALLLPLALLLHAARPQVQLVQSGAEVKKPGASVKVSCKASGYTFTSYNMHWVRQAPGQGLEWMGVIYSGNGDTSYNQKFKGRVTITADKSTSTAYMELSSLRSEDTAVYYCARERDTRFGNWGQGTLVTVSSGGGGSGGGGSGGGGSDIVMTQSPDSLAVSLGERATINCRASESVDIYGNSFMHWYQQKPGQPPKLLIYLASNLESGVPDRFSGSGSGTDFTLTISSLQAEDVAVYYCQQNNEDPYTFGGGTKVEIKSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 118 - protein, 483 aa, Synthetic construct
QVQLVQSGAEVKKPGASVKVSCKASGYTFTSYNMHWVRQAPGQGLEWMGVIYSGNGDTSYNQKFKGRVTITADKSTSTAYMELSSLRSEDTAVYYCARERDTRFGNWGQGTLVTVSSGGGGSGGGGSGGGGSDIVMTQSPDSLAVSLGERATINCRASESVDIYGNSFMHWYQQKPGQPPKLLIYLASNLESGVPDRFSGSGSGTDFTLTISSLQAEDVAVYYCQQNNEDPYTFGGGTKVEIKSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 119 - DNA, 1512 bp, Synthetic construct
  source 1..1512: mol_type = other DNA
atggcgcttccggtgacagcactgctcctccccttggcgctgttgctccacgcagcaaggccgcaggttcagcttgtccaatcaggagcagaggtgaaaaagccaggagcgagcgtcaaagtaagttgcaaagccagcgggtacacatttaccagctacaacatgcactgggtaagacaagcgccaggacaaggcctcgagtggatgggcgtgatatatagcggtaacggtgacaccagttataaccagaaattcaagggtcgggtcacaataaccgccgataaaagcacttctacagcatatatggagctctcatccctgagatcagaagacactgcggtgtattactgcgcgcgcgagagggatacaaggttcggcaattggggccaaggtactttggtcaccgtgagtagcggtggtggtggtagtggtggcggtggcagtggtgggggtggttcagacatcgtaatgacccaatcacctgatagcctggcagtaagcctcggcgaacgggccacgataaactgtagagcatccgagtcagtggacatttatggcaattctttcatgcattggtaccagcaaaagccaggtcagcccccgaaattgcttatctatcttgcgagtaatcttgaatcaggggtacccgaccgcttctctggctctggcagtggcactgattttaccctgacgatctccagcctccaagctgaagacgtggccgtatattactgccagcagaataacgaggatccctatacgttcggaggtggtacgaaggtagagataaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 120 - protein, 506 aa, Synthetic construct
MALPVTALLLPLALLLHAARPQVQLVQSGAEVKKPGASVKVSCKASGYTFTSYNMHWVRQAPGQGLEWMGVIYSGNGDTSYNQKFKGRVTITADKSTSTAYMELSSLRSEDTAVYYCARERDTRFGNWGQGTLVTVSSGGGGSGGGGSGGGGSDIVMTQSPDSLAVSLGERATINCRASESVDIYGNSFMHWYQQKPGQPPKLLIYLASNLESGVPDRFSGSGSGTDFTLTISSLQAEDVAVYYCQQNNEDPYTFGGGTKVEIKSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 121 - protein, 485 aa, Synthetic construct
QVQLVQSGAEVKKPGASVKVSCKASGYTFTSYNMHWVRQAPGQGLEWMGVIYSGNGDTSYNQKFKGRVTITADKSTSTAYMELSSLRSEDTAVYYCARERDTRFGNWGQGTLVTVSSGGGGSGGGGSGGGGSDIVMTQSPDSLAVSLGERATINCRASESVDIYGNSFMHWYQQKPGQPPKLLIYLASNLESGVPDRFSGSGSGTDFTLTISSLQAEDVAVYYCQQNNEDPYTFGGGTKVEIKSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 122 - DNA, 1518 bp, Synthetic construct
  source 1..1518: mol_type = other DNA
atggcgcttccggtgacagcactgctcctccccttggcgctgttgctccacgcagcaaggccgcaggttcagcttgtccaatcaggagcagaggtgaaaaagccaggagcgagcgtcaaagtaagttgcaaagccagcgggtacacatttaccagctacaacatgcactgggtaagacaagcgccaggacaaggcctcgagtggatgggcgtgatatatagcggtaacggtgacaccagttataaccagaaattcaagggtcgggtcacaataaccgccgataaaagcacttctacagcatatatggagctctcatccctgagatcagaagacactgcggtgtattactgcgcgcgcgagagggatacaaggttcggcaattggggccaaggtactttggtcaccgtgagtagcggtggtggtggtagtggtggcggtggcagtggtgggggtggttcagacatcgtaatgacccaatcacctgatagcctggcagtaagcctcggcgaacgggccacgataaactgtagagcatccgagtcagtggacatttatggcaattctttcatgcattggtaccagcaaaagccaggtcagcccccgaaattgcttatctatcttgcgagtaatcttgaatcaggggtacccgaccgcttctctggctctggcagtggcactgattttaccctgacgatctccagcctccaagctgaagacgtggccgtatattactgccagcagaataacgaggatccctatacgttcggaggtggtacgaaggtagagataaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 123 - protein, 11 aa, Synthetic construct
RASQGISSALA

SEQ ID NO: 124 - protein, 7 aa, Synthetic construct
DASSLES

SEQ ID NO: 125 - protein, 9 aa, Synthetic construct
QQFNSYPLT

SEQ ID NO: 126 - protein, 5 aa, Synthetic construct
SYWIG

SEQ ID NO: 127 - protein, 17 aa, Synthetic construct
IIYPGDSDTRYSPSFQG

SEQ ID NO: 128 - protein, 14 aa, Synthetic construct
HGRGYNGYEGAFDI

SEQ ID NO: 129 - protein, 107 aa, Synthetic construct [sequence omitted]

SEQ ID NO: 130 - protein, 123 aa, Synthetic construct
QVQLVQSGAAVKKPGESLKISCKGSGYRFTSYWIGWVRQMPGKGLEWMGIIYPGDSDTRYSPSFQGQVTISAGKSISTAYLQWSSLKASDTAMYYCARHGRGYNGYEGAFDIWGQGTMVTVSS

SEQ ID NO: 131 - protein, 245 aa, Synthetic construct
AIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIKGGGGSGGGGSGGGGSQVQLVQSGAAVKKPGESLKISCKGSGYRFTSYWIGWVRQMPGKGLEWMGIIYPGDSDTRYSPSFQGQVTISAGKSISTAYLQWSSLKASDTAMYYCARHGRGYNGYEGAFDIWGQGTMVTVSS

SEQ ID NO: 132 - protein, 245 aa, Synthetic construct
QVQLVQSGAAVKKPGESLKISCKGSGYRFTSYWIGWVRQMPGKGLEWMGIIYPGDSDTRYSPSFQGQVTISAGKSISTAYLQWSSLKASDTAMYYCARHGRGYNGYEGAFDIWGQGTMVTVSSGGGGSGGGGSGGGGSAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIK

SEQ ID NO: 133 - protein, 506 aa, Synthetic construct
MALPVTALLLPLALLLHAARPAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIKGGGGSGGGGSGGGGSQVQLVQSGAAVKKPGESLKISCKGSGYRFTSYWIGWVRQMPGKGLEWMGIIYPGDSDTRYSPSFQGQVTISAGKSISTAYLQWSSLKASDTAMYYCARHGRGYNGYEGAFDIWGQGTMVTVSSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 134 - protein, 485 aa, Synthetic construct
AIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIKGGGGSGGGGSGGGGSQVQLVQSGAAVKKPGESLKISCKGSGYRFTSYWIGWVRQMPGKGLEWMGIIYPGDSDTRYSPSFQGQVTISAGKSISTAYLQWSSLKASDTAMYYCARHGRGYNGYEGAFDIWGQGTMVTVSSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 135 - DNA, 1518 bp, Synthetic construct
  source 1..1518: mol_type = other DNA
atggcgcttccggtgacagcactgctcctccccttggcgctgttgctccacgcagcaaggccggccatacaactgactcaaagcccctcatctctgtcagcatcagtgggcgaccgagttactattacgtgccgcgccagtcaaggtatttccagcgctcttgcttggtatcagcaaaagcccggtaaggcccccaaactcctgatatacgacgccagttcattggagtccggagtaccgtcacggttctctggatctgggtcaggcaccgactttacgcttacaattagctcactccaaccggaagattttgctacctactattgccagcagttcaattcatacccacttacgtttgggggaggaacgaaagtagaaattaaaggaggtggcgggagtggtggcggtggttctggcggcggcggttctcaggttcagcttgtgcaatcaggggctgcggtaaaaaaacccggggaatcacttaagatttcctgtaaaggctcagggtatcgattcacttcatattggatagggtgggttcgccagatgccaggaaaagggctggagtggatgggcataatttacccaggagacagcgataccagatatagtccgtcctttcagggccaggttacaattagcgccgggaagagtatcagcacagcgtacctgcaatggtcatccctcaaagcctcagatacagcaatgtattactgtgcgcgacacggcaggggttacaatgggtacgaaggggctttcgatatttgggggcagggcaccatggttacggtcagctcaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 136 - protein, 508 aa, Synthetic construct
MALPVTALLLPLALLLHAARPAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIKGGGGSGGGGSGGGGSQVQLVQSGAAVKKPGESLKISCKGSGYRFTSYWIGWVRQMPGKGLEWMGIIYPGDSDTRYSPSFQGQVTISAGKSISTAYLQWSSLKASDTAMYYCARHGRGYNGYEGAFDIWGQGTMVTVSSSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 137 - protein, 487 aa, Synthetic construct
AIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIKGGGGSGGGGSGGGGSQVQLVQSGAAVKKPGESLKISCKGSGYRFTSYWIGWVRQMPGKGLEWMGIIYPGDSDTRYSPSFQGQVTISAGKSISTAYLQWSSLKASDTAMYYCARHGRGYNGYEGAFDIWGQGTMVTVSSSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 138 - DNA, 1524 bp, Synthetic construct
  source 1..1524: mol_type = other DNA
atggcgcttccggtgacagcactgctcctccccttggcgctgttgctccacgcagcaaggccggccatacaactgactcaaagcccctcatctctgtcagcatcagtgggcgaccgagttactattacgtgccgcgccagtcaaggtatttccagcgctcttgcttggtatcagcaaaagcccggtaaggcccccaaactcctgatatacgacgccagttcattggagtccggagtaccgtcacggttctctggatctgggtcaggcaccgactttacgcttacaattagctcactccaaccggaagattttgctacctactattgccagcagttcaattcatacccacttacgtttgggggaggaacgaaagtagaaattaaaggaggtggcgggagtggtggcggtggttctggcggcggcggttctcaggttcagcttgtgcaatcaggggctgcggtaaaaaaacccggggaatcacttaagatttcctgtaaaggctcagggtatcgattcacttcatattggatagggtgggttcgccagatgccaggaaaagggctggagtggatgggcataatttacccaggagacagcgataccagatatagtccgtcctttcagggccaggttacaattagcgccgggaagagtatcagcacagcgtacctgcaatggtcatccctcaaagcctcagatacagcaatgtattactgtgcgcgacacggcaggggttacaatgggtacgaaggggctttcgatatttgggggcagggcaccatggttacggtcagctcaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 139 - protein, 506 aa, Synthetic construct
MALPVTALLLPLALLLHAARPQVQLVQSGAAVKKPGESLKISCKGSGYRFTSYWIGWVRQMPGKGLEWMGIIYPGDSDTRYSPSFQGQVTISAGKSISTAYLQWSSLKASDTAMYYCARHGRGYNGYEGAFDIWGQGTMVTVSSGGGGSGGGGSGGGGSAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIKSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 140 - protein, 485 aa, Synthetic construct
QVQLVQSGAAVKKPGESLKISCKGSGYRFTSYWIGWVRQMPGKGLEWMGIIYPGDSDTRYSPSFQGQVTISAGKSISTAYLQWSSLKASDTAMYYCARHGRGYNGYEGAFDIWGQGTMVTVSSGGGGSGGGGSGGGGSAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIKSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 141 - DNA, 1518 bp, Synthetic construct
  source 1..1518: mol_type = other DNA
atggcgcttccggtgacagcactgctcctccccttggcgctgttgctccacgcagcaaggccgcaagttcaactcgtacaatctggtgcagcagtgaaaaaacccggtgaatctttgaaaatcagctgtaaaggctctggctacaggtttacaagctattggatcggctgggtccgacaaatgcccggcaagggtctggagtggatgggcatcatatatccgggagactccgatacgcgatatagtcccagcttccaaggacaggttacgatctccgcgggtaaaagtatatctacggcgtaccttcagtggtcttctctcaaagcctcagatacggctatgtattactgtgcccgccacggtcgaggatacaatggttacgaaggcgctttcgatatatggggacagggtaccatggtaaccgttagcagtggtggaggtgggagtggtggtggtgggtcaggcgggggaggtagcgcaatccagttgactcagagcccctcatctctttccgcgtctgtaggcgaccgcgttaccataacctgcagagcctctcagggcatctcaagtgcacttgcctggtatcaacagaagccaggtaaggcccccaagctcctgatttatgatgccagtagtcttgagtctggtgtccctagccgctttagcggttctggctctggtactgacttcacgctgacgatctcaagcctccagccggaggacttcgcaacatactattgtcaacagtttaactcatacccgctgacgttcgggggtggtacaaaagttgagatcaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 142 - protein, 508 aa, Synthetic construct
MALPVTALLLPLALLLHAARPQVQLVQSGAAVKKPGESLKISCKGSGYRFTSYWIGWVRQMPGKGLEWMGIIYPGDSDTRYSPSFQGQVTISAGKSISTAYLQWSSLKASDTAMYYCARHGRGYNGYEGAFDIWGQGTMVTVSSGGGGSGGGGSGGGGSAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIKSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 143 - protein, 487 aa, Synthetic construct
QVQLVQSGAAVKKPGESLKISCKGSGYRFTSYWIGWVRQMPGKGLEWMGIIYPGDSDTRYSPSFQGQVTISAGKSISTAYLQWSSLKASDTAMYYCARHGRGYNGYEGAFDIWGQGTMVTVSSGGGGSGGGGSGGGGSAIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIKSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 144 - DNA, 1524 bp, Synthetic construct
  source 1..1524: mol_type = other DNA
atggcgcttccggtgacagcactgctcctccccttggcgctgttgctccacgcagcaaggccgcaagttcaactcgtacaatctggtgcagcagtgaaaaaacccggtgaatctttgaaaatcagctgtaaaggctctggctacaggtttacaagctattggatcggctgggtccgacaaatgcccggcaagggtctggagtggatgggcatcatatatccgggagactccgatacgcgatatagtcccagcttccaaggacaggttacgatctccgcgggtaaaagtatatctacggcgtaccttcagtggtcttctctcaaagcctcagatacggctatgtattactgtgcccgccacggtcgaggatacaatggttacgaaggcgctttcgatatatggggacagggtaccatggtaaccgttagcagtggtggaggtgggagtggtggtggtgggtcaggcgggggaggtagcgcaatccagttgactcagagcccctcatctctttccgcgtctgtaggcgaccgcgttaccataacctgcagagcctctcagggcatctcaagtgcacttgcctggtatcaacagaagccaggtaaggcccccaagctcctgatttatgatgccagtagtcttgagtctggtgtccctagccgctttagcggttctggctctggtactgacttcacgctgacgatctcaagcctccagccggaggacttcgcaacatactattgtcaacagtttaactcatacccgctgacgttcgggggtggtacaaaagttgagatcaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 145 - protein, 122 aa, Synthetic construct
EVQLVESGGGLVRAGGSLRLSCAASGRTTFSTYWLGWFRQAPGKNREFVAAISWSAGMAYYQDSKGRFTISRDNTKNTAYLQMNSLQPEDTAVYYCAAGRFHPIRVDTALYWAQGTQVTVSS

SEQ ID NO: 146 - protein, 383 aa, Synthetic construct
MALPVTALLLPLALLLHAARPEVQLVESGGGLVRAGGSLRLSCAASGRTTFSTYWLGWFRQAPGKNREFVAAISWSAGMAYYQDSKGRFTISRDNTKNTAYLQMNSLQPEDTAVYYCAAGRFHPIRVDTALYWAQGTQVTVSSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 147 - protein, 362 aa, Synthetic construct
EVQLVESGGGLVRAGGSLRLSCAASGRTTFSTYWLGWFRQAPGKNREFVAAISWSAGMAYYQDSKGRFTISRDNTKNTAYLQMNSLQPEDTAVYYCAAGRFHPIRVDTALYWAQGTQVTVSSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 148 - DNA, 1149 bp, Synthetic construct
  source 1..1149: mol_type = other DNA
atggcgcttccggtgacagcactgctcctccccttggcgctgttgctccacgcagcaaggccggaggttcagcttgtggaatcaggcgggggcctcgtgcgggccggtggtagtttgcgacttagctgtgcggcgtcagggaggacaacattcagcacatattggcttgggtggttccggcaggctccgggaaagaatagagagttcgtcgccgcaatatcatggtcagccgggatggcttattatcaggatagcaagggtcggttcaccatctcccgcgacaatacaaagaatacggcttatctccagatgaacagtctccaaccggaagataccgctgtatattactgcgctgccggtcgctttcaccccatccgcgtagatacggcattgtactgggcgcaaggcactcaagtgacagtaagcagtagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 149 - protein, 385 aa, Synthetic construct
MALPVTALLLPLALLLHAARPEVQLVESGGGLVRAGGSLRLSCAASGRTTFSTYWLGWFRQAPGKNREFVAAISWSAGMAYYQDSKGRFTISRDNTKNTAYLQMNSLQPEDTAVYYCAAGRFHPIRVDTALYWAQGTQVTVSSSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 150 - DNA, 1155 bp, Synthetic construct
  source 1..1155: mol_type = other DNA
atggcgcttccggtgacagcactgctcctccccttggcgctgttgctccacgcagcaaggccggaggttcagcttgtggaatcaggcgggggcctcgtgcgggccggtggtagtttgcgacttagctgtgcggcgtcagggaggacaacattcagcacatattggcttgggtggttccggcaggctccgggaaagaatagagagttcgtcgccgcaatatcatggtcagccgggatggcttattatcaggatagcaagggtcggttcaccatctcccgcgacaatacaaagaatacggcttatctccagatgaacagtctccaaccggaagataccgctgtatattactgcgctgccggtcgctttcaccccatccgcgtagatacggcattgtactgggcgcaaggcactcaagtgacagtaagcagtagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 151 - protein, 129 aa, Synthetic construct
QVHLVESGGGLVQAGGSLGLSCAASGHTFSNYAMGWFRQAPGKEREFVAAISWSGGSTLYADSVKGRFTISRDNGENTVYLQMNSLKPEDTAVYYCALDSTGVYGTGYVSSRKGRYEYWGQGTLVTVSS

SEQ ID NO: 152 - protein, 390 aa, Synthetic construct
MALPVTALLLPLALLLHAARPQVHLVESGGGLVQAGGSLGLSCAASGHTFSNYAMGWFRQAPGKEREFVAAISWSGGSTLYADSVKGRFTISRDNGENTVYLQMNSLKPEDTAVYYCALDSTGVYGTGYVSSRKGRYEYWGQGTLVTVSSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 153 - protein, 369 aa, Synthetic construct
QVHLVESGGGLVQAGGSLGLSCAASGHTFSNYAMGWFRQAPGKEREFVAAISWSGGSTLYADSVKGRFTISRDNGENTVYLQMNSLKPEDTAVYYCALDSTGVYGTGYVSSRKGRYEYWGQGTLVTVSSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 154 - DNA, 1170 bp, Synthetic construct
  source 1..1170: mol_type = other DNA
atggcgcttccggtgacagcactgctcctccccttggcgctgttgctccacgcagcaaggccgcaggttcatttggttgagtcaggcggagggctcgtgcaagctggcgggtccctggggctgtcttgtgctgcgagcggtcacaccttctccaactatgcgatgggatggtttcgccaagcaccgggtaaggagcgcgagttcgtggctgccattagttggtccggtggctccacgctctatgctgattcagtgaagggtagattcacgatatcacgggataatggcgaaaatacggtctatttgcaaatgaacagtctgaaaccagaggacacggcggtgtattactgcgcccttgatagtaccggtgtctatggaacgggatacgtgagctctaggaaagggagatacgaatactggggacagggaacattggtaactgtctcatctagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 155 - protein, 392 aa, Synthetic construct
MALPVTALLLPLALLLHAARPQVHLVESGGGLVQAGGSLGLSCAASGHTFSNYAMGWFRQAPGKEREFVAAISWSGGSTLYADSVKGRFTISRDNGENTVYLQMNSLKPEDTAVYYCALDSTGVYGTGYVSSRKGRYEYWGQGTLVTVSSSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 156 - protein, 371 aa, Synthetic construct
QVHLVESGGGLVQAGGSLGLSCAASGHTFSNYAMGWFRQAPGKEREFVAAISWSGGSTLYADSVKGRFTISRDNGENTVYLQMNSLKPEDTAVYYCALDSTGVYGTGYVSSRKGRYEYWGQGTLVTVSSSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 157 - DNA, 1176 bp, Synthetic construct
  source 1..1176: mol_type = other DNA
atggcgcttccggtgacagcactgctcctccccttggcgctgttgctccacgcagcaaggccgcaggttcatttggttgagtcaggcggagggctcgtgcaagctggcgggtccctggggctgtcttgtgctgcgagcggtcacaccttctccaactatgcgatgggatggtttcgccaagcaccgggtaaggagcgcgagttcgtggctgccattagttggtccggtggctccacgctctatgctgattcagtgaagggtagattcacgatatcacgggataatggcgaaaatacggtctatttgcaaatgaacagtctgaaaccagaggacacggcggtgtattactgcgcccttgatagtaccggtgtctatggaacgggatacgtgagctctaggaaagggagatacgaatactggggacagggaacattggtaactgtctcatctagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 158 - protein, 125 aa, Synthetic construct
EVQLVESGGGLVRAGGSLRLSCAASGRTFTYDARAWFRQAPGKEREFVAAISWSGGSTYYPNSMKGRFTISRDNAKNTVYLQMNSLKPEDTAVYYCAADESFPAYYSDYALYRYWGQGTLVTVSS

SEQ ID NO: 159 - protein, 386 aa, Synthetic construct
MALPVTALLLPLALLLHAARPEVQLVESGGGLVRAGGSLRLSCAASGRTFTYDARAWFRQAPGKEREFVAAISWSGGSTYYPNSMKGRFTISRDNAKNTVYLQMNSLKPEDTAVYYCAADESFPAYYSDYALYRYWGQGTLVTVSSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 160 - protein, 365 aa, Synthetic construct
EVQLVESGGGLVRAGGSLRLSCAASGRTFTYDARAWFRQAPGKEREFVAAISWSGGSTYYPNSMKGRFTISRDNAKNTVYLQMNSLKPEDTAVYYCAADESFPAYYSDYALYRYWGQGTLVTVSSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 161 - DNA, 1158 bp, Synthetic construct
  source 1..1158: mol_type = other DNA
atggcgcttccggtgacagcactgctcctccccttggcgctgttgctccacgcagcaaggccggaggtccagttggtcgaatcaggtgggggacttgttcgagctggcggttctctgcggctttcctgcgcggccagtggacggacctttacctacgatgctagggcatggtttagacaagcgcccggcaaagagcgcgagttcgtagccgctatatcctggtctggaggttcaacctattaccccaattccatgaagggtaggtttacgatatccagggacaatgcaaagaatactgtctaccttcaaatgaactccttgaaaccggaagatactgctgtctattattgtgcggccgacgaatcctttcccgcttactacagtgactatgctctgtaccgctactggggacaaggcaccctcgtaaccgtaagtagcagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 162 - protein, 388 aa, Synthetic construct
MALPVTALLLPLALLLHAARPEVQLVESGGGLVRAGGSLRLSCAASGRTFTYDARAWFRQAPGKEREFVAAISWSGGSTYYPNSMKGRFTISRDNAKNTVYLQMNSLKPEDTAVYYCAADESFPAYYSDYALYRYWGQGTLVTVSSSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 163 - protein, 367 aa, Synthetic construct
EVQLVESGGGLVRAGGSLRLSCAASGRTFTYDARAWFRQAPGKEREFVAAISWSGGSTYYPNSMKGRFTISRDNAKNTVYLQMNSLKPEDTAVYYCAADESFPAYYSDYALYRYWGQGTLVTVSSSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 164 - DNA, 1164 bp, Synthetic construct
  source 1..1164: mol_type = other DNA
atggcgcttccggtgacagcactgctcctccccttggcgctgttgctccacgcagcaaggccggaggtccagttggtcgaatcaggtgggggacttgttcgagctggcggttctctgcggctttcctgcgcggccagtggacggacctttacctacgatgctagggcatggtttagacaagcgcccggcaaagagcgcgagttcgtagccgctatatcctggtctggaggttcaacctattaccccaattccatgaagggtaggtttacgatatccagggacaatgcaaagaatactgtctaccttcaaatgaactccttgaaaccggaagatactgctgtctattattgtgcggccgacgaatcctttcccgcttactacagtgactatgctctgtaccgctactggggacaaggcaccctcgtaaccgtaagtagcagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 165 - protein, 127 aa, Synthetic construct
QVQLVESGGGLVQAGDSLRLSCAVSGRTLSNYFMAWLRQAPGKEREFVAAIHWSLGSTYYQDPVKGRFTISRDKAKNTVYLQMNTLKPEDTATYICAAGQHLSGLGGSAWSIEGDYWGQGTQVTVSS

SEQ ID NO: 166 - protein, 388 aa, Synthetic construct
MALPVTALLLPLALLLHAARPQVQLVESGGGLVQAGDSLRLSCAVSGRTLSNYFMAWLRQAPGKEREFVAAIHWSLGSTYYQDPVKGRFTISRDKAKNTVYLQMNTLKPEDTATYICAAGQHLSGLGGSAWSIEGDYWGQGTQVTVSSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 167 - protein, 367 aa, Synthetic construct
QVQLVESGGGLVQAGDSLRLSCAVSGRTLSNYFMAWLRQAPGKEREFVAAIHWSLGSTYYQDPVKGRFTISRDKAKNTVYLQMNTLKPEDTATYICAAGQHLSGLGGSAWSIEGDYWGQGTQVTVSSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 168 - DNA, 1164 bp, Synthetic construct
  source 1..1164: mol_type = other DNA
atggcgcttccggtgacagcactgctcctccccttggcgctgttgctccacgcagcaaggccgcaagtgcagctcgttgaaagtgggggcggacttgttcaggcaggtgacagccttcgattgagctgtgcagtgagcggaagaactctgagcaactacttcatggcgtggctgagacaagcacctggcaaagaacgggagttcgtcgctgcgatacactggagcctcggctcaacatactaccaagaccctgtaaaggggaggtttacgataagtagagataaggctaagaatacggtttatctgcaaatgaacacattgaagcctgaggatacagcaacgtatatatgcgcagcggggcaacacctgtctgggctgggtgggtctgcatggtccatagaaggagactactggggtcaagggacccaagtaacagtcagcagcagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 169 - protein, 390 aa, Synthetic construct
MALPVTALLLPLALLLHAARPQVQLVESGGGLVQAGDSLRLSCAVSGRTLSNYFMAWLRQAPGKEREFVAAIHWSLGSTYYQDPVKGRFTISRDKAKNTVYLQMNTLKPEDTATYICAAGQHLSGLGGSAWSIEGDYWGQGTQVTVSSSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 170 - protein, 369 aa, Synthetic construct
QVQLVESGGGLVQAGDSLRLSCAVSGRTLSNYFMAWLRQAPGKEREFVAAIHWSLGSTYYQDPVKGRFTISRDKAKNTVYLQMNTLKPEDTATYICAAGQHLSGLGGSAWSIEGDYWGQGTQVTVSSSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 171 - DNA, 1170 bp, Synthetic construct
  source 1..1170: mol_type = other DNA
atggcgcttccggtgacagcactgctcctccccttggcgctgttgctccacgcagcaaggccgcaagtgcagctcgttgaaagtgggggcggacttgttcaggcaggtgacagccttcgattgagctgtgcagtgagcggaagaactctgagcaactacttcatggcgtggctgagacaagcacctggcaaagaacgggagttcgtcgctgcgatacactggagcctcggctcaacatactaccaagaccctgtaaaggggaggtttacgataagtagagataaggctaagaatacggtttatctgcaaatgaacacattgaagcctgaggatacagcaacgtatatatgcgcagcggggcaacacctgtctgggctgggtgggtctgcatggtccatagaaggagactactggggtcaagggacccaagtaacagtcagcagcagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 172 - protein, 125 aa, Synthetic construct
QVQLVESGGGLVQAGGSLRLSCAASGGTFSIYPVGWFRQAPGKQREFVAAIGWSASGTYYGDSVEGRFTVSRDNARSTVYLRMSSLKPDDTAVYYCAAASGSNWRLGAIDEYDSWGQGTQVTVSS

SEQ ID NO: 173 - protein, 386 aa, Synthetic construct
MALPVTALLLPLALLLHAARPQVQLVESGGGLVQAGGSLRLSCAASGGTFSIYPVGWFRQAPGKQREFVAAIGWSASGTYYGDSVEGRFTVSRDNARSTVYLRMSSLKPDDTAVYYCAAASGSNWRLGAIDEYDSWGQGTQVTVSSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 174 - protein, 365 aa, Synthetic construct
QVQLVESGGGLVQAGGSLRLSCAASGGTFSIYPVGWFRQAPGKQREFVAAIGWSASGTYYGDSVEGRFTVSRDNARSTVYLRMSSLKPDDTAVYYCAAASGSNWRLGAIDEYDSWGQGTQVTVSSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 175 - DNA, 1158 bp, Synthetic construct
  source 1..1158: mol_type = other DNA
atggcgcttccggtgacagcactgctcctccccttggcgctgttgctccacgcagcaaggccgcaagtgcaactcgttgaatctggaggaggtctggtacaagcggggggttcactgagactctcttgtgcagcgtcaggtggcacttttagtatatatccggttggatggttcagacaagcaccaggaaaacaaagggaatttgttgctgcaatcggttggtccgcctccgggacgtattatggtgatagtgtcgagggcagattcacagtgtcacgcgacaatgcgagatctacggtctatctcaggatgagttcacttaaacctgacgacaccgccgtctattattgtgcagcagcaagcggttctaactggcgattgggagcaattgatgaatatgatagctggggacaaggtactcaggttactgtttccagtagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 176 - protein, 388 aa, Synthetic construct
MALPVTALLLPLALLLHAARPQVQLVESGGGLVQAGGSLRLSCAASGGTFSIYPVGWFRQAPGKQREFVAAIGWSASGTYYGDSVEGRFTVSRDNARSTVYLRMSSLKPDDTAVYYCAAASGSNWRLGAIDEYDSWGQGTQVTVSSSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 177 - protein, 367 aa, Synthetic construct
QVQLVESGGGLVQAGGSLRLSCAASGGTFSIYPVGWFRQAPGKQREFVAAIGWSASGTYYGDSVEGRFTVSRDNARSTVYLRMSSLKPDDTAVYYCAAASGSNWRLGAIDEYDSWGQGTQVTVSSSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 178 - DNA, 1164 bp, Synthetic construct
  source 1..1164: mol_type = other DNA
atggcgcttccggtgacagcactgctcctccccttggcgctgttgctccacgcagcaaggccgcaagtgcaactcgttgaatctggaggaggtctggtacaagcggggggttcactgagactctcttgtgcagcgtcaggtggcacttttagtatatatccggttggatggttcagacaagcaccaggaaaacaaagggaatttgttgctgcaatcggttggtccgcctccgggacgtattatggtgatagtgtcgagggcagattcacagtgtcacgcgacaatgcgagatctacggtctatctcaggatgagttcacttaaacctgacgacaccgccgtctattattgtgcagcagcaagcggttctaactggcgattgggagcaattgatgaatatgatagctggggacaaggtactcaggttactgtttccagtagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 179 - protein, 128 aa, Synthetic construct
QVQLVESGGGLVQAGDSLRLSCAASGRTFSSYAMGWFRQAPGKEREFVASITSSGLVAYYGDSVEARFTISRDNAKNTVYLQMDSLKPEDTAVYYCAARQYVGSGSYYLKKEGGYEYWGPGTQVTVSS

SEQ ID NO: 180 - protein, 389 aa, Synthetic construct
MALPVTALLLPLALLLHAARPQVQLVESGGGLVQAGDSLRLSCAASGRTFSSYAMGWFRQAPGKEREFVASITSSGLVAYYGDSVEARFTISRDNAKNTVYLQMDSLKPEDTAVYYCAARQYVGSGSYYLKKEGGYEYWGPGTQVTVSSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 181 - protein, 368 aa, Synthetic construct
QVQLVESGGGLVQAGDSLRLSCAASGRTFSSYAMGWFRQAPGKEREFVASITSSGLVAYYGDSVEARFTISRDNAKNTVYLQMDSLKPEDTAVYYCAARQYVGSGSYYLKKEGGYEYWGPGTQVTVSSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 182 - DNA, 1167 bp, Synthetic construct
  source 1..1167: mol_type = other DNA
atggcgcttccggtgacagcactgctcctccccttggcgctgttgctccacgcagcaaggccgcaggttcagctcgtagagagtggtgggggactggttcaggctggcgattctcttcggctttcctgcgcggctagtggacgaacctttagcagttatgctatgggctggttccgccaagcccccggaaaagaacgcgagtttgtagccagcatcacgagttctggacttgttgcgtactacggcgatagcgttgaggcaaggttcaccatcagtcgagacaacgcgaaaaataccgtttacttgcagatggatagtctgaaaccggaagacaccgccgtctactactgcgctgcgcggcagtacgtcgggagcggaagctattatttgaaaaaggaaggcggttatgaatactgggggcctggcacacaagtaacggtttcaagcagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 183 - protein, 391 aa, Synthetic construct
MALPVTALLLPLALLLHAARPQVQLVESGGGLVQAGDSLRLSCAASGRTFSSYAMGWFRQAPGKEREFVASITSSGLVAYYGDSVEARFTISRDNAKNTVYLQMDSLKPEDTAVYYCAARQYVGSGSYYLKKEGGYEYWGPGTQVTVSSSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 184 - DNA, 1173 bp, Synthetic construct
  source 1..1173: mol_type = other DNA
atggcgcttccggtgacagcactgctcctccccttggcgctgttgctccacgcagcaaggccgcaggttcagctcgtagagagtggtgggggactggttcaggctggcgattctcttcggctttcctgcgcggctagtggacgaacctttagcagttatgctatgggctggttccgccaagcccccggaaaagaacgcgagtttgtagccagcatcacgagttctggacttgttgcgtactacggcgatagcgttgaggcaaggttcaccatcagtcgagacaacgcgaaaaataccgtttacttgcagatggatagtctgaaaccggaagacaccgccgtctactactgcgctgcgcggcagtacgtcgggagcggaagctattatttgaaaaaggaaggcggttatgaatactgggggcctggcacacaagtaacggtttcaagcagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 185 - protein, 128 aa, Synthetic construct
QVQLVESGGGLVQAGGSLRLSCVASGRTFSSYAMGWFRQGPGKEREFAAAILSNGLTTYYADSVKGRFTISRDNAKDTVYLQMNSLKPEDTANYYCAAAFTPEFRDGGIWDDASVDYWGQGTQVTVSS

SEQ ID NO: 186 - protein, 389 aa, Synthetic construct
MALPVTALLLPLALLLHAARPQVQLVESGGGLVQAGGSLRLSCVASGRTFSSYAMGWFRQGPGKEREFAAAILSNGLTTYYADSVKGRFTISRDNAKDTVYLQMNSLKPEDTANYYCAAAFTPEFRDGGIWDDASVDYWGQGTQVTVSSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 187 - protein, 368 aa, Synthetic construct
QVQLVESGGGLVQAGGSLRLSCVASGRTFSSYAMGWFRQGPGKEREFAAAILSNGLTTYYADSVKGRFTISRDNAKDTVYLQMNSLKPEDTANYYCAAAFTPEFRDGGIWDDASVDYWGQGTQVTVSSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 188 - DNA, 1167 bp, Synthetic construct
  source 1..1167: mol_type = other DNA
atggcgcttccggtgacagcactgctcctccccttggcgctgttgctccacgcagcaaggccgcaagtccaactggtagagtctgggggcggcctcgtgcaagccggaggttccctcaggctctcttgtgtggcatcaggtaggacctttagttcctatgccatgggttggttccggcaagggcctggaaaagagagggaatttgcggcagccatactttcaaacggacttaccacctactatgccgacagtgtgaaagggcgatttaccatatctcgcgataatgcgaaggacacggtctatctccagatgaatagcctcaagccggaagacaccgctaactactattgtgctgctgcctttacgcccgaatttcgggacggaggcatctgggatgacgcatcagttgattattggggccagggaacccaagtaacggtgtcatccagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 189 - protein, 391 aa, Synthetic construct
MALPVTALLLPLALLLHAARPQVQLVESGGGLVQAGGSLRLSCVASGRTFSSYAMGWFRQGPGKEREFAAAILSNGLTTYYADSVKGRFTISRDNAKDTVYLQMNSLKPEDTANYYCAAAFTPEFRDGGIWDDASVDYWGQGTQVTVSSSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 190 - protein, 370 aa, Synthetic construct
QVQLVESGGGLVQAGGSLRLSCVASGRTFSSYAMGWFRQGPGKEREFAAAILSNGLTTYYADSVKGRFTISRDNAKDTVYLQMNSLKPEDTANYYCAAAFTPEFRDGGIWDDASVDYWGQGTQVTVSSSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 191 - DNA, 1173 bp, Synthetic construct
  source 1..1173: mol_type = other DNA
atggcgcttccggtgacagcactgctcctccccttggcgctgttgctccacgcagcaaggccgcaagtccaactggtagagtctgggggcggcctcgtgcaagccggaggttccctcaggctctcttgtgtggcatcaggtaggacctttagttcctatgccatgggttggttccggcaagggcctggaaaagagagggaatttgcggcagccatactttcaaacggacttaccacctactatgccgacagtgtgaaagggcgatttaccatatctcgcgataatgcgaaggacacggtctatctccagatgaatagcctcaagccggaagacaccgctaactactattgtgctgctgcctttacgcccgaatttcgggacggaggcatctgggatgacgcatcagttgattattggggccagggaacccaagtaacggtgtcatccagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 192 - protein, 124 aa, Synthetic construct
QVQLVESGGGLVQAGGSLRLSCTASGDTFSSYSMGWFRQGPGKEREFVGGIRWSGGTTYYADSVKGRFTISRDNAKNTVYLQMNSLKREDTAVYYCAAVRRRWLIWQEEEYDYWGQGTLVTVSS

SEQ ID NO: 193 - protein, 385 aa, Synthetic construct
MALPVTALLLPLALLLHAARPQVQLVESGGGLVQAGGSLRLSCTASGDTFSSYSMGWFRQGPGKEREFVGGIRWSGGTTYYADSVKGRFTISRDNAKNTVYLQMNSLKREDTAVYYCAAVRRRWLIWQEEEYDYWGQGTLVTVSSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 194 - protein, 364 aa, Synthetic construct
QVQLVESGGGLVQAGGSLRLSCTASGDTFSSYSMGWFRQGPGKEREFVGGIRWSGGTTYYADSVKGRFTISRDNAKNTVYLQMNSLKREDTAVYYCAAVRRRWLIWQEEEYDYWGQGTLVTVSSSAAAFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 195 - DNA, 1155 bp, Synthetic construct
  source 1..1155: mol_type = other DNA
atggcgcttccggtgacagcactgctcctccccttggcgctgttgctccacgcagcaaggccgcaagtacaattggtggagtctgggggtggtttggttcaagctggtgggagtctcagactgtcatgtacggccagcggggacactttctcatcttatagcatgggttggtttcgacagggtcccggaaaggaacgagagttcgtaggcggcattcgatggagtgggggcacgacttactacgccgattctgtgaagggtcggttcacaataagtcgagacaacgccaagaatacagtgtacctccagatgaattccttgaaaagggaggatacggccgtctattattgcgcggccgtgagacggcgatggctcatctggcaagaagaagagtacgattattggggccaagggactcttgtaaccgtgagttctagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgctcaaagcggagtaggttgttgcattccgattacatgaatatgactcctcgccggcctgggccgacaagaaaacattaccaaccctatgcccccccacgagacttcgctgcgtacaggtcccgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 196 - protein, 387 aa, Synthetic construct
MALPVTALLLPLALLLHAARPQVQLVESGGGLVQAGGSLRLSCTASGDTFSSYSMGWFRQGPGKEREFVGGIRWSGGTTYYADSVKGRFTISRDNAKNTVYLQMNSLKREDTAVYYCAAVRRRWLIWQEEEYDYWGQGTLVTVSSSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 197 - protein, 366 aa, Synthetic construct
QVQLVESGGGLVQAGGSLRLSCTASGDTFSSYSMGWFRQGPGKEREFVGGIRWSGGTTYYADSVKGRFTISRDNAKNTVYLQMNSLKREDTAVYYCAAVRRRWLIWQEEEYDYWGQGTLVTVSSSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 198 - DNA, 1161 bp, Synthetic construct
  source 1..1161: mol_type = other DNA
atggcgcttccggtgacagcactgctcctccccttggcgctgttgctccacgcagcaaggccgcaagtacaattggtggagtctgggggtggtttggttcaagctggtgggagtctcagactgtcatgtacggccagcggggacactttctcatcttatagcatgggttggtttcgacagggtcccggaaaggaacgagagttcgtaggcggcattcgatggagtgggggcacgacttactacgccgattctgtgaagggtcggttcacaataagtcgagacaacgccaagaatacagtgtacctccagatgaattccttgaaaagggaggatacggccgtctattattgcgcggccgtgagacggcgatggctcatctggcaagaagaagagtacgattattggggccaagggactcttgtaaccgtgagttctagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 199 - DNA, 60 bp, Synthetic construct
  source 1..60: mol_type = other DNA
aagagcaacagtgctgtgtgcctggagcaacaaatctgactaagagcaacaaatctgact

SEQ ID NO: 200 - DNA, 20 bp, Synthetic construct
  source 1..20: mol_type = other DNA
gctactctctctttctggcc

SEQ ID NO: 201 - DNA, 23 bp, Synthetic construct
  source 1..23: mol_type = other DNA
gctactctctctttctggcctgg

SEQ ID NO: 202 - RNA, 20 nt, Synthetic construct
  source 1..20: mol_type = other RNA
gctactctctctttctggcc

SEQ ID NO: 203 - RNA, 20 nt, Synthetic construct
  source 1..20: mol_type = other RNA
gctactctctctttctggcc

SEQ ID NO: 204 - RNA, 100 nt, Synthetic construct
  source 1..100: mol_type = other RNA
gctactctctctttctggccgttttagagctagaaatagcaagttaaaataaggctagtccgttatcaacttgaaaaagtggcaccgagtcggtgctttt

SEQ ID NO: 205 - RNA, 100 nt, Synthetic construct; identical to SEQ ID NO: 204
  source 1..100: mol_type = other RNA

SEQ ID NO: 206 - DNA, 79 bp, Synthetic construct
  source 1..79: mol_type = other DNA
cgtggccttagctgtgctcgcgctactctctctttctgcctggaggctatccagcgtgagtctctcctaccctcccgct

SEQ ID NO: 207 - DNA, 78 bp, Synthetic construct
  source 1..78: mol_type = other DNA
cgtggccttagctgtgctcgcgctactctctctttcgcctggaggctatccagcgtgagtctctcctaccctcccgct

SEQ ID NO: 208 - DNA, 75 bp, Synthetic construct
  source 1..75: mol_type = other DNA
cgtggccttagctgtgctcgcgctactctctctttctggaggctatccagcgtgagtctctcctaccctcccgct

SEQ ID NO: 209 - DNA, 84 bp, Synthetic construct
  source 1..84: mol_type = other DNA
cgtggccttagctgtgctcgcgctactctctctttctggatagcctggaggctatccagcgtgagtctctcctaccctcccgct

SEQ ID NO: 210 - DNA, 55 bp, Synthetic construct
  source 1..55: mol_type = other DNA
cgtggccttagctgtgctcgcgctatccagcgtgagtctctcctaccctcccgct

SEQ ID NO: 211 - DNA, 82 bp, Synthetic construct
  source 1..82: mol_type = other DNA
cgtggccttagctgtgctcgcgctactctctctttctgtggcctggaggctatccagcgtgagtctctcctaccctcccgct

SEQ ID NO: 212 - RNA, 100 nt, Synthetic construct
  source 1..100: mol_type = other RNA
nnnnnnnnnnnnnnnnnnnngttttagagctagaaatagcaagttaaaataaggctagtccgttatcaacttgaaaaagtggcaccgagtcggtgctttt

SEQ ID NO: 213 - RNA, 96 nt, Synthetic construct
  source 1..96: mol_type = other RNA
nnnnnnnnnnnnnnnnnnnngttttagagctagaaatagcaagttaaaataaggctagtccgttatcaacttgaaaaagtggcaccgagtcggtgc

SEQ ID NO: 214 - RNA, 77 nt, Synthetic construct
  source 1..77: mol_type = other RNA
gttttagagctagaaatagcaagttaaaataaggctagtccgttatcaacttgaaaaagtggcaccgagtcggtgct

SEQ ID NO: 215 - RNA, 154 nt, Synthetic construct
  source 1..154: mol_type = other RNA
gttttagagctagaaatagcaagttaaaataaggctagtccgttatcaacttgaaaaagtggcaccgagtcggtgctgttttagagctagaaatagcaagttaaaataaggctagtccgttatcaacttgaaaaagtggcaccgagtcggtgct

SEQ ID NO: 216 - RNA, 231 nt, Synthetic construct
  source 1..231: mol_type = other RNA
gttttagagctagaaatagcaagttaaaataaggctagtccgttatcaacttgaaaaagtggcaccgagtcggtgctgttttagagctagaaatagcaagttaaaataaggctagtccgttatcaacttgaaaaagtggcaccgagtcggtgctgttttagagctagaaatagcaagttaaaataaggctagtccgttatcaacttgaaaaagtggcaccgagtcggtgct

SEQ ID NO: 217 - RNA, 308 nt, Synthetic construct
  source 1..308: mol_type = other RNA
gttttagagctagaaatagcaagttaaaataaggctagtccgttatcaacttgaaaaagtggcaccgagtcggtgctgttttagagctagaaatagcaagttaaaataaggctagtccgttatcaacttgaaaaagtggcaccgagtcggtgctgttttagagctagaaatagcaagttaaaataaggctagtccgttatcaacttgaaaaagtggcaccgagtcggtgctgttttagagctagaaatagcaagttaaaataaggctagtccgttatcaacttgaaaaagtggcaccgagtcggtgct

SEQ ID NO: 218 - RNA, 385 nt, Synthetic construct
  source 1..385: mol_type = other RNA
gttttagagctagaaatagcaagttaaaataaggctagtccgttatcaacttgaaaaagtggcaccgagtcggtgctgttttagagctagaaatagcaagttaaaataaggctagtccgttatcaacttgaaaaagtggcaccgagtcggtgctgttttagagctagaaatagcaagttaaaataaggctagtccgttatcaacttgaaaaagtggcaccgagtcggtgctgttttagagctagaaatagcaagttaaaataaggctagtccgttatcaacttgaaaaagtggcaccgagtcggtgctgttttagagctagaaatagcaagttaaaataaggctagtccgttatcaacttgaaaaagtggcaccgagtcggtgct

SEQ ID NO: 219 - RNA, 462 nt, Synthetic construct
  source 1..462: mol_type = other RNA
gttttagagctagaaatagcaagttaaaataaggctagtccgttatcaacttgaaaaagtggcaccgagtcggtgctgttttagagctagaaatagcaagttaaaataaggctagtccgttatcaacttgaaaaagtggcaccgagtcggtgctgttttagagctagaaatagcaagttaaaataaggctagtccgttatcaacttgaaaaagtggcaccgagtcggtgctgttttagagctagaaatagcaagttaaaataaggctagtccgttatcaacttgaaaaagtggcaccgagtcggtgctgttttagagctagaaatagcaagttaaaataaggctagtccgttatcaacttgaaaaagtggcaccgagtcggtgctgttttagagctagaaatagcaagttaaaataaggctagtccgttatcaacttgaaaaagtggcaccgagtcggtgct

SEQ ID NO: 220 - RNA, 539 nt, Synthetic construct
  source 1..539: mol_type = other RNA
gttttagagctagaaatagcaagttaaaataaggctagtccgttatcaacttgaaaaagtggcaccgagtcggtgctgttttagagctagaaatagcaagttaaaataaggctagtccgttatcaacttgaaaaagtggcaccgagtcggtgctgttttagagctagaaatagcaagttaaaataaggctagtccgttatcaacttgaaaaagtggcaccgagtcggtgctgttttagagctagaaatagcaagttaaaataaggctagtccgttatcaacttgaaaaagtggcaccgagtcggtgctgttttagagctagaaatagcaagttaaaataaggctagtccgttatcaacttgaaaaagtggcaccgagtcggtgctgttttagagctagaaatagcaagttaaaataaggctagtccgttatcaacttgaaaaagtggcaccgagtcggtgctgttttagagctagaaatagcaagttaaaataaggctagtccgttatcaacttgaaaaagtggcaccgagtcggtgct

SEQ ID NO: 221 - RNA, 616 nt, Synthetic construct
  source 1..616: mol_type = other RNA
gttttagagctagaaatagcaagttaaaataaggctagtccgttatcaacttgaaaaagtggcaccgagtcggtgctgttttagagctagaaatagcaagttaaaataaggctagtccgttatcaacttgaaaaagtggcaccgagtcggtgctgttttagagctagaaatagcaagttaaaataaggctagtccgttatcaacttgaaaaagtggcaccgagtcggtgctgttttagagctagaaatagcaagttaaaataaggctagtccgttatcaacttgaaaaagtggcaccgagtcggtgctgttttagagctagaaatagcaagttaaaataaggctagtccgttatcaacttgaaaaagtggcaccgagtcggtgctgttttagagctagaaatagcaagttaaaataaggctagtccgttatcaacttgaaaaagtggcaccgagtcggtgctgttttagagctagaaatagcaagttaaaataaggctagtccgttatcaacttgaaaaagtggcaccgagtcggtgctgttttagagctagaaatagcaagttaaaataaggctagtccgttatcaacttgaaaaagtggcaccgagtcggtgct

SEQ ID NO: 222 - protein, 1368 aa, Synthetic construct [sequence omitted]

SEQ ID NO: 223 - DNA, 800 bp, Synthetic construct
  source 1..800: mol_type = other DNA
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttca

SEQ ID NO: 224 - DNA, 1178 bp, Synthetic construct
  source 1..1178: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 225 - DNA, 49 bp, Synthetic construct
  source 1..49: mol_type = other DNA
aataaaatcgctatccatcgaagatggatgtgtgttggttttttgtgtg

SEQ ID NO: 226 - DNA, 804 bp, Synthetic construct
  source 1..804: mol_type = other DNA
tggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 227 - protein, 364 aa, Synthetic construct
EVQLVESGGGLVRAGGSLRLSCAASGRTTFSTYWLGWFRQAPGKNREFVAAISWSAGMAYYQDSKGRFTISRDNTKNTAYLQMNSLQPEDTAVYYCAAGRFHPIRVDTALYWAQGTQVTVSSSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 228 - protein, 370 aa, Synthetic construct
QVQLVESGGGLVQAGDSLRLSCAASGRTFSSYAMGWFRQAPGKEREFVASITSSGLVAYYGDSVEARFTISRDNAKNTVYLQMDSLKPEDTAVYYCAARQYVGSGSYYLKKEGGYEYWGPGTQVTVSSSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 229 - protein, 22 aa, Synthetic construct
MLLLVTSLLLCELPHPAFLLIP